SEQUENCE LISTING (1046 sequences)

SEQ ID NO: 1 - protein, 17 aa, Unknown
  Possibly HERV
LQNRRGLGLSILLNEEC

SEQ ID NO: 2 - protein, 17 aa, Unknown
  Possibly HERV
  misc_feature (1)..(1): Xaa can be any naturally occurring amino acid
XQNRRGLGLSILLNEEC

SEQ ID NO: 3 - protein, 17 aa, Unknown
  Possibly HERV
  misc_feature (2)..(2): Xaa can be any naturally occurring amino acid
LXNRRGLGLSILLNEEC

SEQ ID NO: 4 - protein, 17 aa, Unknown
  Possibly HERV
  misc_feature (3)..(3): Xaa can be any naturally occurring amino acid
LQXRRGLGLSILLNEEC

SEQ ID NO: 5 - protein, 17 aa, Unknown
  Possibly HERV
  misc_feature (4)..(4): Xaa can be any naturally occurring amino acid
LQNXRGLGLSILLNEEC

SEQ ID NO: 6 - protein, 17 aa, Unknown
  Possibly HERV
  misc_feature (5)..(5): Xaa can be any naturally occurring amino acid
LQNRXGLGLSILLNEEC

SEQ ID NO: 7 - protein, 17 aa, Unknown
  Possibly HERV
  misc_feature (6)..(6): Xaa can be any naturally occurring amino acid
LQNRRXLGLSILLNEEC

SEQ ID NO: 8 - protein, 17 aa, Unknown
  Possibly HERV
  misc_feature (7)..(7): Xaa can be any naturally occurring amino acid
LQNRRGXGLSILLNEEC

SEQ ID NO: 9 - protein, 17 aa, Unknown
  Possibly HERV
  misc_feature (8)..(8): Xaa can be any naturally occurring amino acid
LQNRRGLXLSILLNEEC

SEQ ID NO: 10 - protein, 17 aa, Unknown
  Possibly HERV
  misc_feature (9)..(9): Xaa can be any naturally occurring amino acid
LQNRRGLGXSILLNEEC

SEQ ID NO: 11 - protein, 17 aa, Unknown
  Possibly HERV
  misc_feature (10)..(10): Xaa can be any naturally occurring amino acid
LQNRRGLGLXILLNEEC

SEQ ID NO: 12 - protein, 17 aa, Unknown
  Possibly HERV
  misc_feature (11)..(11): Xaa can be any naturally occurring amino acid
LQNRRGLGLSXLLNEEC

SEQ ID NO: 13 - protein, 17 aa, Unknown
  Possibly HERV
  misc_feature (12)..(12): Xaa can be any naturally occurring amino acid
LQNRRGLGLSIXLNEEC

SEQ ID NO: 14 - protein, 17 aa, Unknown
  Possibly HERV
  misc_feature (13)..(13): Xaa can be any naturally occurring amino acid
LQNRRGLGLSILXNEEC

SEQ ID NO: 15 - protein, 17 aa, Unknown
  Possibly HERV
  misc_feature (14)..(14): Xaa can be any naturally occurring amino acid
LQNRRGLGLSILLXEEC

SEQ ID NO: 16 - protein, 17 aa, Unknown
  Possibly HERV
  misc_feature (15)..(15): Xaa can be any naturally occurring amino acid
LQNRRGLGLSILLNXEC

SEQ ID NO: 17 - protein, 17 aa, Unknown
  Possibly HERV
  misc_feature (16)..(16): Xaa can be any naturally occurring amino acid
LQNRRGLGLSILLNEXC

SEQ ID NO: 18 - protein, 17 aa, Unknown
  Possibly HERV
  misc_feature (17)..(17): Xaa can be any naturally occurring amino acid
LQNRRGLGLSILLNEEX

SEQ ID NO: 19 - protein, 23 aa, Unknown
  Possibly HERV
LQNRRGLGLSILLNEECGPGPGP

SEQ ID NO: 20 - protein, 25 aa, Unknown
  Possibly HERV
LQNRRGLGLSILLNEECGGGPGPGP

SEQ ID NO: 21 - protein, 23 aa, Unknown
  Possibly HERV
LQNRRGLGLSILLNEECHHHHHH

SEQ ID NO: 22 - protein, 25 aa, Unknown
  Possibly HERV
LQNRRGLGLSILLNEECGGHHHHHH

SEQ ID NO: 23 - protein, 25 aa, Unknown
  Possibly HERV
LQNRRGLGLSILLNEECGGEKEKEK

SEQ ID NO: 24 - protein, 17 aa, Unknown
  Possibly HERV
LQNRRGLGLSIFLNEEC

SEQ ID NO: 25 - protein, 10 aa, Unknown
  Possibly HERV
GLSILLNEEC

SEQ ID NO: 26 - protein, 8 aa, Unknown
  Possibly HERV
LSILLNEE

SEQ ID NO: 27 - protein, 25 aa, Unknown
  Possibly HERV
LQNRRGLGLSILLNEECEEGPGPGP

SEQ ID NO: 28 - protein, 23 aa, Unknown
  Possibly HERV
LQNRRGLDLSILLNEECGPGPGP

SEQ ID NO: 29 - protein, 16 aa, Unknown
  Possibly HERV
GLSILLNEECGPGPGP

SEQ ID NO: 30 - protein, 23 aa, Unknown
  Possibly HERV
LQNRRGLLQNRRGLGLSILLNEE

SEQ ID NO: 31 - protein, 25 aa, Unknown
  Possibly HERV
LQNRRGLGLSILLNEECKKGPGPGP

SEQ ID NO: 32 - protein, 15 aa, Unknown
  Possibly HERV
LQNRRGLGLSILLNC

SEQ ID NO: 33 - protein, 22 aa, Unknown
  Possibly HERV
LNRKAIGLSILLNEECGPGPGP

SEQ ID NO: 34 - protein, 23 aa, Unknown
  Possibly HERV
LQARILAGLSILLNEECGPGPGP

SEQ ID NO: 35 - protein, 23 aa, Unknown
  Possibly HERV
LQNKRGLGLSILLNEECGPGPGP

SEQ ID NO: 36 - protein, 23 aa, Unknown
  Possibly HERV
LQNKKGLGLSILLNEECGPGPGP

SEQ ID NO: 37 - protein, 21 aa, Unknown
  Possibly HERV
LQNRRGLGLSILLNEECGPKK

SEQ ID NO: 38 - protein, 24 aa, Unknown
  Possibly HERV
LQNRRGLGLSILLNEELQNRRGLC

SEQ ID NO: 39 - protein, 16 aa, Unknown
  Possibly HERV
LQNRRGLGLSILLNEE

SEQ ID NO: 40 - protein, 23 aa, Unknown
  Possibly HERV
LQNRRGLGLSILLNEELQNRRGL

SEQ ID NO: 41 - protein, 24 aa, Unknown
  Possibly HERV
LQNRRGLLQNRRGLGLSILLNEEC

SEQ ID NO: 42 - protein, 17 aa, Unknown
  Possibly HERV
AQNRRALDLLTAEKGGT

SEQ ID NO: 43 - protein, 17 aa, Unknown
  Possibly HERV
AQNRRALDLLTAEKGGT

SEQ ID NO: 44 - protein, 17 aa, Unknown
  Possibly HERV
AQNRRALDLLTAEKGGT

SEQ ID NO: 45 - protein, 17 aa, Unknown
  Possibly HERV
AQNRQALDLLMAEKGRT

SEQ ID NO: 46 - protein, 17 aa, Unknown
  Possibly HERV
AKNRWALDLLMAEKGGT

SEQ ID NO: 47 - protein, 17 aa, Unknown
  Possibly HERV
AKNRWALDLLMAEKGGT

SEQ ID NO: 48 - protein, 17 aa, Unknown
  Possibly HERV
MQNRRALDLLTADKGGT

SEQ ID NO: 49 - protein, 17 aa, Unknown
  Possibly HERV
YQNRLALGYLLVSEGGV

SEQ ID NO: 50 - protein, 17 aa, Unknown
  Possibly HERV
YQNRLALDYLLASGGGV

SEQ ID NO: 51 - protein, 17 aa, Unknown
  Possibly HERV
YHNRLALDYLLASEGGV

SEQ ID NO: 52 - protein, 17 aa, Unknown
  Possibly HERV
YQNRLALDYLLALEGGV

SEQ ID NO: 53 - protein, 17 aa, Unknown
  Possibly HERV
YQNRLGLDYLLASEGGV

SEQ ID NO: 54 - protein, 17 aa, Unknown
  Possibly HERV
YQNRLALDYLLASEGGV

SEQ ID NO: 55 - protein, 17 aa, Unknown
  Possibly HERV
YQNRLALDYLLASKRRV

SEQ ID NO: 56 - protein, 17 aa, Unknown
  Possibly HERV
YQNRLALDFLLASEGRV

SEQ ID NO: 57 - protein, 17 aa, Unknown
  Possibly HERV
YQNRLALDYLLASEGGV

SEQ ID NO: 58 - protein, 17 aa, Unknown
  Possibly HERV
YQNRLALNYLLSSEGGI

SEQ ID NO: 59 - protein, 17 aa, Unknown
  Possibly HERV
HQNRLALDYLLASEGGV

SEQ ID NO: 60 - protein, 17 aa, Unknown
  Possibly HERV
YQNRLALDYLLASEGGV

SEQ ID NO: 61 - protein, 17 aa, Unknown
  Possibly HERV
YQNRLALDYLLASEGGV

SEQ ID NO: 62 - protein, 17 aa, Unknown
  Possibly HERV
LQNRRALDLLTAKRGGT

SEQ ID NO: 63 - protein, 17 aa, Unknown
  Possibly HERV
LQNQTALDLLTAKRDGT

SEQ ID NO: 64 - protein, 17 aa, Unknown
  Possibly HERV
LQNRRALNLLTAERGGT

SEQ ID NO: 65 - protein, 17 aa, Unknown
  Possibly HERV
LQNRTALDLLTAERGGT

SEQ ID NO: 66 - protein, 17 aa, Unknown
  Possibly HERV
LQNQRALDLLTTESGGI

SEQ ID NO: 67 - protein, 17 aa, Unknown
  Possibly HERV
LQNRRDLDLLTTKRGGT

SEQ ID NO: 68 - protein, 17 aa, Unknown
  Possibly HERV
LQNRRALDLLTAERSGT

SEQ ID NO: 69 - protein, 17 aa, Unknown
  Possibly HERV
LQNRRALDLLTAERGGT

SEQ ID NO: 70 - protein, 17 aa, Unknown
  Possibly HERV
LQNQRVLDLLTTERGGI

SEQ ID NO: 71 - protein, 17 aa, Unknown
  Possibly HERV
VQNRRALDLLAAERGGT

SEQ ID NO: 72 - protein, 17 aa, Unknown
  Possibly HERV
LQNRRALDLLTAESGGT

SEQ ID NO: 73 - protein, 17 aa, Unknown
  Possibly HERV
LQNQRALDLLTTERGGI

SEQ ID NO: 74 - protein, 17 aa, Unknown
  Possibly HERV
LQNQTALDLLTAERGGI

SEQ ID NO: 75 - protein, 17 aa, Unknown
  Possibly HERV
LQNWKALNLLTAERSGT

SEQ ID NO: 76 - protein, 17 aa, Unknown
  Possibly HERV
LQNQRALDLLTAKREGT

SEQ ID NO: 77 - protein, 17 aa, Unknown
  Possibly HERV
LQNRRALDLLTAKRGGT

SEQ ID NO: 78 - protein, 17 aa, Unknown
  Possibly HERV
LQNRRALELLITERGGT

SEQ ID NO: 79 - protein, 17 aa, Unknown
  Possibly HERV
LQNRRALDLLTAKRGGT

SEQ ID NO: 80 - protein, 17 aa, Unknown
  Possibly HERV
LQNRRALDLLTTERGGT

SEQ ID NO: 81 - protein, 17 aa, Unknown
  Possibly HERV
LQNQSALDLLTAERGGT

SEQ ID NO: 82 - protein, 17 aa, Unknown
  Possibly HERV
LQNRRALDLLTAERGGT

SEQ ID NO: 83 - protein, 17 aa, Unknown
  Possibly HERV
LQNQRALDLLTAKRGGN

SEQ ID NO: 84 - protein, 17 aa, Unknown
  Possibly HERV
LQNRRALDLLTAERGGI

SEQ ID NO: 85 - protein, 17 aa, Unknown
  Possibly HERV
LQNQRVLDLLTAERGET

SEQ ID NO: 86 - protein, 17 aa, Unknown
  Possibly HERV
LQNRCGLNLLTAAQGLI

SEQ ID NO: 87 - protein, 17 aa, Unknown
  Possibly HERV
PQNRRALDLLTAERGGT

SEQ ID NO: 88 - protein, 17 aa, Unknown
  Possibly HERV
LQNRRALDLLTAETGGT

SEQ ID NO: 89 - protein, 17 aa, Unknown
  Possibly HERV
LQNQRALDLLTAERGGT

SEQ ID NO: 90 - protein, 17 aa, Unknown
  Possibly HERV
LQNRRALDLLTAERGGT

SEQ ID NO: 91 - protein, 17 aa, Unknown
  Possibly HERV
LQNRRALDLLTAKRCGT

SEQ ID NO: 92 - protein, 17 aa, Unknown
  Possibly HERV
LQNRRAVDLSATEKGRT

SEQ ID NO: 93 - protein, 17 aa, Unknown
  Possibly HERV
LQNRRALDLLTAERGGS

SEQ ID NO: 94 - protein, 17 aa, Unknown
  Possibly HERV
LQNQRALDLLTAKRGGS

SEQ ID NO: 95 - protein, 17 aa, Unknown
  Possibly HERV
LQNRRALDLLTSERGGT

SEQ ID NO: 96 - protein, 17 aa, Unknown
  Possibly HERV
LQNRRALDLLTAKRGGT

SEQ ID NO: 97 - protein, 17 aa, Unknown
  Possibly HERV
LQNWRALDLLIAKRGGT

SEQ ID NO: 98 - protein, 17 aa, Unknown
  Possibly HERV
LQNRRGLDILTIEKDGL

SEQ ID NO: 99 - protein, 17 aa, Unknown
  Possibly HERV
YQNRLALDHLLASEGGV

SEQ ID NO: 100 - protein, 17 aa, Unknown
  Possibly HERV
YQNRLALDYLLASEGGV

SEQ ID NO: 101 - protein, 17 aa, Unknown
  Possibly HERV
YQNRLALDYLLASGGVC

SEQ ID NO: 102 - protein, 17 aa, Unknown
  Possibly HERV
CQNRLALDYLLALGGVC

SEQ ID NO: 103 - protein, 17 aa, Unknown
  Possibly HERV
YQNRLALDYLLASEGRV

SEQ ID NO: 104 - protein, 17 aa, Unknown
  Possibly HERV
YQNRLVLNYVLASEGGL

SEQ ID NO: 105 - protein, 17 aa, Unknown
  Possibly HERV
YQNRLALDYLLASEGGV

SEQ ID NO: 106 - protein, 17 aa, Unknown
  Possibly HERV
YQNRLALDYLLASKGGV

SEQ ID NO: 107 - protein, 17 aa, Unknown
  Possibly HERV
YQNRLALDYLLDSKGVC

SEQ ID NO: 108 - protein, 17 aa, Unknown
  Possibly HERV
YQNSLALDYLLVSKGGV

SEQ ID NO: 109 - protein, 17 aa, Unknown
  Possibly HERV
YQNRLAINYLLAQEGEV

SEQ ID NO: 110 - protein, 17 aa, Unknown
  Possibly HERV
YQNRLALDYLLAQEGGV

SEQ ID NO: 111 - protein, 17 aa, Unknown
  Possibly HERV
YQNRLALDYLLAQEGGV

SEQ ID NO: 112 - protein, 17 aa, Unknown
  Possibly HERV
YQNRLALDYLLALEGVV

SEQ ID NO: 113 - protein, 17 aa, Unknown
  Possibly HERV
YQNRLALDHLLAQEGGV

SEQ ID NO: 114 - protein, 17 aa, Unknown
  Possibly HERV
YQNRLGLDYLLAQEGGI

SEQ ID NO: 115 - protein, 17 aa, Unknown
  Possibly HERV
YQNRLVLDCLLAQEGEY

SEQ ID NO: 116 - protein, 17 aa, Unknown
  Possibly HERV
YQNRLALDYLLAQEGGV

SEQ ID NO: 117 - protein, 17 aa, Unknown
  Possibly HERV
YQNSLALDYLLAQEEGI

SEQ ID NO: 118 - protein, 17 aa, Unknown
  Possibly HERV
YQNRRALDYFLASEEGV

SEQ ID NO: 119 - protein, 17 aa, Unknown
  Possibly HERV
YQNRLALDYLLAQEGGV

SEQ ID NO: 120 - protein, 17 aa, Unknown
  Possibly HERV
YQNRLAFDYLLAQEGGV

SEQ ID NO: 121 - protein, 17 aa, Unknown
  Possibly HERV
YQNRLALDYLLAQEGGV

SEQ ID NO: 122 - protein, 17 aa, Unknown
  Possibly HERV
YQNRLALDYLLAQEAGV

SEQ ID NO: 123 - protein, 17 aa, Unknown
  Possibly HERV
YQNRLALDYLLAREGGA

SEQ ID NO: 124 - protein, 17 aa, Unknown
  Possibly HERV
YENSLALNYLLASEGRV

SEQ ID NO: 125 - protein, 17 aa, Unknown
  Possibly HERV
YQNRLALDYLLAQEEGV

SEQ ID NO: 126 - protein, 17 aa, Unknown
  Possibly HERV
YQNRLALDYLLAQKGGV

SEQ ID NO: 127 - protein, 17 aa, Unknown
  Possibly HERV
YQNRLALDCLLAQEGGG

SEQ ID NO: 128 - protein, 17 aa, Unknown
  Possibly HERV
YQNRLALDYLLASEGCV

SEQ ID NO: 129 - protein, 17 aa, Unknown
  Possibly HERV
HQNRLALNYLLVQEGGA

SEQ ID NO: 130 - protein, 17 aa, Unknown
  Possibly HERV
HQNRLALNYLLVQEGGA

SEQ ID NO: 131 - protein, 17 aa, Unknown
  Possibly HERV
LQNRRALNLLIAERGGT

SEQ ID NO: 132 - protein, 17 aa, Unknown
  Possibly HERV
LQNQRALDLLTAERGET

SEQ ID NO: 133 - protein, 17 aa, Unknown
  Possibly HERV
LQNQRPLELLTAERGGT

SEQ ID NO: 134 - protein, 17 aa, Unknown
  Possibly HERV
LQNRRALDLLTAKKGGT

SEQ ID NO: 135 - protein, 17 aa, Unknown
  Possibly HERV
LQNQRALDLLTTERGGT

SEQ ID NO: 136 - protein, 17 aa, Unknown
  Possibly HERV
LQNRRALDLLTVERGGT

SEQ ID NO: 137 - protein, 17 aa, Unknown
  Possibly HERV
LQNWRALDLLTAKRGGT

SEQ ID NO: 138 - protein, 17 aa, Unknown
  Possibly HERV
LQNWRALDLLTAERGGT

SEQ ID NO: 139 - protein, 17 aa, Unknown
  Possibly HERV
LQNQRALDLLTTERGGT

SEQ ID NO: 140 - protein, 17 aa, Unknown
  Possibly HERV
LQKRRALDLLTAKRRRT

SEQ ID NO: 141 - protein, 17 aa, Unknown
  Possibly HERV
LQNRRALVLLTAEKGGT

SEQ ID NO: 142 - protein, 17 aa, Unknown
  Possibly HERV
LQNQRSLDLLTAKRRGT

SEQ ID NO: 143 - protein, 17 aa, Unknown
  Possibly HERV
LQNQRSLDLLTAKRRGT

SEQ ID NO: 144 - protein, 17 aa, Unknown
  Possibly HERV
LQNWRALDLLTAERGGA

SEQ ID NO: 145 - protein, 17 aa, Unknown
  Possibly HERV
LDQRLALDYLLAEQGGE

SEQ ID NO: 146 - protein, 17 aa, Unknown
  Possibly HERV
LKHGRGLDMLAAAQGGT

SEQ ID NO: 147 - protein, 17 aa, Unknown
  Possibly HERV
LQNNRRLDFLAAEKGGI

SEQ ID NO: 148 - protein, 17 aa, Unknown
  Possibly HERV
LQNRSEPDLLAAEKGCL

SEQ ID NO: 149 - protein, 17 aa, Unknown
  Possibly HERV
VQNRRRLDLLTAQKDGL

SEQ ID NO: 150 - protein, 17 aa, Unknown
  Possibly HERV
LQNRRGLGLLEANRRGL

SEQ ID NO: 151 - protein, 17 aa, Unknown
  Possibly HERV
LQNRRGLDLITTEKGGL

SEQ ID NO: 152 - protein, 17 aa, Unknown
  Possibly HERV
LTNRRGLDLLTAEKVGL

SEQ ID NO: 153 - protein, 17 aa, Unknown
  Possibly HERV
LQNRRGLDTLTAEKGGL

SEQ ID NO: 154 - protein, 17 aa, Unknown
  Possibly HERV
LQNRMGLDLLTAEKGGL

SEQ ID NO: 155 - protein, 17 aa, Unknown
  Possibly HERV
LQNRRGLDLLTAGKGGL

SEQ ID NO: 156 - protein, 17 aa, Unknown
  Possibly HERV
LQNRRELDLLTAKKKKV

SEQ ID NO: 157 - protein, 17 aa, Unknown
  Possibly HERV
LQNRRGLDLLTAEKGGL

SEQ ID NO: 158 - protein, 17 aa, Unknown
  Possibly HERV
LQNCRGLDLLTAEKGGL

SEQ ID NO: 159 - protein, 17 aa, Unknown
  Possibly HERV
LQNRRGLDLLTAEKGGL

SEQ ID NO: 160 - protein, 17 aa, Unknown
  Possibly HERV
FQNHQGLDLLSGEKERL

SEQ ID NO: 161 - protein, 17 aa, Unknown
  Possibly HERV
LQNRQGLDLHTAEKGGL

SEQ ID NO: 162 - protein, 17 aa, Unknown
  Possibly HERV
LQNHRGLDLLTAEKGGL

SEQ ID NO: 163 - protein, 17 aa, Unknown
  Possibly HERV
LQNCRGLDLLTAEKGGL

SEQ ID NO: 164 - protein, 17 aa, Unknown
  Possibly HERV
LQNHRGIDLLSAVKGGL

SEQ ID NO: 165 - protein, 17 aa, Unknown
  Possibly HERV
LQNRQGLDLLTAEKGGL

SEQ ID NO: 166 - protein, 17 aa, Unknown
  Possibly HERV
LQNRRSLDLLTAEKGGL

SEQ ID NO: 167 - protein, 17 aa, Unknown
  Possibly HERV
LQNRRGLDLLTAEKGGL

SEQ ID NO: 168 - protein, 17 aa, Unknown
  Possibly HERV
LQNHRGLDLLTAEKGGL

SEQ ID NO: 169 - protein, 17 aa, Unknown
  Possibly HERV
LQNHRGLDLLTAEKGGL

SEQ ID NO: 170 - protein, 17 aa, Unknown
  Possibly HERV
VQNCQGLNLPTAEKGGL

SEQ ID NO: 171 - protein, 17 aa, Unknown
  Possibly HERV
LQNHRGLDFLTVEKGGL

SEQ ID NO: 172 - protein, 17 aa, Unknown
  Possibly HERV
LQNYRGLDLLTAEKGGL

SEQ ID NO: 173 - protein, 17 aa, Unknown
  Possibly HERV
LQNGRGLDLLTAEKGGL

SEQ ID NO: 174 - protein, 17 aa, Unknown
  Possibly HERV
LQNCQGLDLLTAEKGGL

SEQ ID NO: 175 - protein, 17 aa, Unknown
  Possibly HERV
LQNRQGLDFLTAEKGGL

SEQ ID NO: 176 - protein, 17 aa, Unknown
  Possibly HERV
LQNYRGLDLLTAEKGGL

SEQ ID NO: 177 - protein, 17 aa, Unknown
  Possibly HERV
LQNCQSLDLLTAEKGGL

SEQ ID NO: 178 - protein, 17 aa, Unknown
  Possibly HERV
LQNRRGLDLLTAEKGGL

SEQ ID NO: 179 - protein, 17 aa, Unknown
  Possibly HERV
LQNRQGLDLLNAEKGGL

SEQ ID NO: 180 - protein, 17 aa, Unknown
  Possibly HERV
LQNCQGLDCLTAEKGGL

SEQ ID NO: 181 - protein, 17 aa, Unknown
  Possibly HERV
LQNRSGLDLLTAEKGGL

SEQ ID NO: 182 - protein, 17 aa, Unknown
  Possibly HERV
VQNRQGLDLLTAEKGGL

SEQ ID NO: 183 - protein, 17 aa, Unknown
  Possibly HERV
LQNHRGLDLLTAEKGGL

SEQ ID NO: 184 - protein, 17 aa, Unknown
  Possibly HERV
PQNRRGLDLLTAEKGGL

SEQ ID NO: 185 - protein, 17 aa, Unknown
  Possibly HERV
LQNRRGLDLLTAKKGGL

SEQ ID NO: 186 - protein, 17 aa, Unknown
  Possibly HERV
LQNHRGLDLLTAEKGGL

SEQ ID NO: 187 - protein, 17 aa, Unknown
  Possibly HERV
LQNRRGLDLLTAEKGGL

SEQ ID NO: 188 - protein, 17 aa, Unknown
  Possibly HERV
LQNRRGVDLLTAEKGGL

SEQ ID NO: 189 - protein, 17 aa, Unknown
  Possibly HERV
LQNRRGLDLLTVEKGGF

SEQ ID NO: 190 - protein, 17 aa, Unknown
  Possibly HERV
LQNHQGLDLLTAEKGEL

SEQ ID NO: 191 - protein, 17 aa, Unknown
  Possibly HERV
LQNRQGLVLLTAEKGGL

SEQ ID NO: 192 - protein, 17 aa, Unknown
  Possibly HERV
LQNHRGLDLLTAEKGGL

SEQ ID NO: 193 - protein, 17 aa, Unknown
  Possibly HERV
LQNHQGLDLLAAEKRGL

SEQ ID NO: 194 - protein, 17 aa, Unknown
  Possibly HERV
LQNRQGLDLLTAEKGGL

SEQ ID NO: 195 - protein, 17 aa, Unknown
  Possibly HERV
LQNHRGLDLLAAEKGGL

SEQ ID NO: 196 - protein, 17 aa, Unknown
  Possibly HERV
LQNHRGLVLLTAEKGGL

SEQ ID NO: 197 - protein, 17 aa, Unknown
  Possibly HERV
LQNHRGLDLLTAEKGGL

SEQ ID NO: 198 - protein, 17 aa, Unknown
  Possibly HERV
LQNRQGLDLLTAEKGGF

SEQ ID NO: 199 - protein, 17 aa, Unknown
  Possibly HERV
LQNPRGLDLLTAEKGGL

SEQ ID NO: 200 - protein, 17 aa, Unknown
  Possibly HERV
LQNCRGLDRLTAEKGGL

SEQ ID NO: 201 - protein, 17 aa, Unknown
  Possibly HERV
LQNCRGLDFLTAEKGGL

SEQ ID NO: 202 - protein, 17 aa, Unknown
  Possibly HERV
LQNCRGLDFLTAEKGGL

SEQ ID NO: 203 - protein, 17 aa, Unknown
  Possibly HERV
LQNCRGLDLLTAEKGGL

SEQ ID NO: 204 - protein, 17 aa, Unknown
  Possibly HERV
LQNCQGLDLLTAEKGGL

SEQ ID NO: 205 - protein, 17 aa, Unknown
  Possibly HERV
HQNRGGLDLLTAEKGGL

SEQ ID NO: 206 - protein, 17 aa, Unknown
  Possibly HERV
LQNCQGLDLLTAEKGGL

SEQ ID NO: 207 - protein, 17 aa, Unknown
  Possibly HERV
LQNCQGLDLLTSEKGGL

SEQ ID NO: 208 - protein, 17 aa, Unknown
  Possibly HERV
TLQNQGLDILTAEKGGL

SEQ ID NO: 209 - protein, 17 aa, Unknown
  Possibly HERV
LQNCQGLDLLTAEKGGL

SEQ ID NO: 210 - protein, 17 aa, Unknown
  Possibly HERV
LQNCQGLDLLTAEKGGL

SEQ ID NO: 211 - protein, 17 aa, Unknown
  Possibly HERV
LQNRQGLDLLTAEKGGL

SEQ ID NO: 212 - protein, 17 aa, Unknown
  Possibly HERV
LQNHRGLDLLTAAKGGL

SEQ ID NO: 213 - protein, 17 aa, Unknown
  Possibly HERV
LQNHQGLDRLTAEKGGL

SEQ ID NO: 214 - protein, 17 aa, Unknown
  Possibly HERV
LQNRRGLDLLTAEKGGL

SEQ ID NO: 215 - protein, 17 aa, Unknown
  Possibly HERV
LQNCRGLDLLTAEKGGH

SEQ ID NO: 216 - protein, 17 aa, Unknown
  Possibly HERV
LQNCQGLDLLTAEKGGL

SEQ ID NO: 217 - protein, 17 aa, Unknown
  Possibly HERV
LQNHQGLDLFTAEKGGL

SEQ ID NO: 218 - protein, 17 aa, Unknown
  Possibly HERV
LQNRWGLDLFTAEKGGL

SEQ ID NO: 219 - protein, 17 aa, Unknown
  Possibly HERV
LQNRRGLDLLTAEKGGL

SEQ ID NO: 220 - protein, 17 aa, Unknown
  Possibly HERV
LQNHRGLDILTAEKGGL

SEQ ID NO: 221 - protein, 17 aa, Unknown
  Possibly HERV
LQNCRGLDLLTAEKGGL

SEQ ID NO: 222 - protein, 17 aa, Unknown
  Possibly HERV
LQNHRGLDLLTAEKGGL

SEQ ID NO: 223 - protein, 17 aa, Unknown
  Possibly HERV
LQNRQGLDLLTVEKGGL

SEQ ID NO: 224 - protein, 17 aa, Unknown
  Possibly HERV
LQNPRGLDFLTAEKGGL

SEQ ID NO: 225 - protein, 17 aa, Unknown
  Possibly HERV
LQNCQGLDLLTAEKGGL

SEQ ID NO: 226 - protein, 17 aa, Unknown
  Possibly HERV
LQNCQGLDLLTAEKGGL

SEQ ID NO: 227 - protein, 17 aa, Unknown
  Possibly HERV
LQNHRGLDLLTAEKGRL

SEQ ID NO: 228 - protein, 17 aa, Unknown
  Possibly HERV
LQNHQGLDLLTAEKGGL

SEQ ID NO: 229 - protein, 17 aa, Unknown
  Possibly HERV
LQNCQGLDLLTAEKGRL

SEQ ID NO: 230 - protein, 17 aa, Unknown
  Possibly HERV
LQNCQGLDLLTAEKGGL

SEQ ID NO: 231 - protein, 17 aa, Unknown
  Possibly HERV
LQNCRSLDLLTAEKGGL

SEQ ID NO: 232 - protein, 17 aa, Unknown
  Possibly HERV
LQNRQGLNLLTAENGGL

SEQ ID NO: 233 - protein, 17 aa, Unknown
  Possibly HERV
LQNHQGLDLLTAEKGGL

SEQ ID NO: 234 - protein, 17 aa, Unknown
  Possibly HERV
LQNCRGLDFLTAEKGGL

SEQ ID NO: 235 - protein, 17 aa, Unknown
  Possibly HERV
LQNRRGLDLLTAEKGGL

SEQ ID NO: 236 - protein, 17 aa, Unknown
  Possibly HERV
LQNHRGLDLLTAEKGGL

SEQ ID NO: 237 - protein, 17 aa, Unknown
  Possibly HERV
LQNRRGLDLLTVEKGGL

SEQ ID NO: 238 - protein, 17 aa, Unknown
  Possibly HERV
LQNCRCLDLLTAEKGGL

SEQ ID NO: 239 - protein, 17 aa, Unknown
  Possibly HERV
LQNRLGLDLLTAEKGGL

SEQ ID NO: 240 - protein, 17 aa, Unknown
  Possibly HERV
LQNHRGLDILTAKKGGL

SEQ ID NO: 241 - protein, 17 aa, Unknown
  Possibly HERV
SPNCQGLDLLTAEKEGL

SEQ ID NO: 242 - protein, 17 aa, Unknown
  Possibly HERV
LQNHQGLDLLTAEKGGL

SEQ ID NO: 243 - protein, 17 aa, Unknown
  Possibly HERV
LQNRQDLDLLTAEKGGL

SEQ ID NO: 244 - protein, 17 aa, Unknown
  Possibly HERV
LQNCQSLDFLIAEKGGL

SEQ ID NO: 245 - protein, 17 aa, Unknown
  Possibly HERV
LQNCRGLGLLTVEKGGL

SEQ ID NO: 246 - protein, 17 aa, Unknown
  Possibly HERV
LQNHRGLDLLTAEKGGL

SEQ ID NO: 247 - protein, 17 aa, Unknown
  Possibly HERV
LQNHQGLDLLTVEKGGL

SEQ ID NO: 248 - protein, 17 aa, Unknown
  Possibly HERV
LQNCGGLGLLIAEKGGF

SEQ ID NO: 249 - protein, 17 aa, Unknown
  Possibly HERV
LQNRQGLDLFAAEKGLC

SEQ ID NO: 250 - protein, 17 aa, Unknown
  Possibly HERV
LQNHQGLDPLTAEKGGL

SEQ ID NO: 251 - protein, 17 aa, Unknown
  Possibly HERV
LQNCRGLDFLIVEKGGL

SEQ ID NO: 252 - protein, 17 aa, Unknown
  Possibly HERV
LQKRQGLDLLTAEKGGL

SEQ ID NO: 253 - protein, 17 aa, Unknown
  Possibly HERV
LQNRRGLDVLTAEKGGL

SEQ ID NO: 254 - protein, 17 aa, Unknown
  Possibly HERV
LQNCQGLDLLTAEKGGL

SEQ ID NO: 255 - protein, 17 aa, Unknown
  Possibly HERV
LQNCQGLDLLTAEKGGL

SEQ ID NO: 256 - protein, 17 aa, Unknown
  Possibly HERV
LQNCQGLDLLTAEKGRL

SEQ ID NO: 257 - protein, 17 aa, Unknown
  Possibly HERV
IQNRLGLDFLTAEKGGL

SEQ ID NO: 258 - protein, 17 aa, Unknown
  Possibly HERV
LQNRRGLDLLTAEKGGL

SEQ ID NO: 259 - protein, 17 aa, Unknown
  Possibly HERV
LQNRQGLDLLTAEKGGL

SEQ ID NO: 260 - protein, 17 aa, Unknown
  Possibly HERV
LQNRQGLELLTAEKGGL

SEQ ID NO: 261 - protein, 17 aa, Unknown
  Possibly HERV
LQNRQGLDLLTAEKGGL

SEQ ID NO: 262 - protein, 17 aa, Unknown
  Possibly HERV
LQNRRGVDLLTAEKGDL

SEQ ID NO: 263 - protein, 17 aa, Unknown
  Possibly HERV
LQNHQVLDLLAAEKGGL

SEQ ID NO: 264 - protein, 17 aa, Unknown
  Possibly HERV
LQNHRGLDLLTAEKGRL

SEQ ID NO: 265 - protein, 17 aa, Unknown
  Possibly HERV
TQNLRGLDLLTAEKGQL

SEQ ID NO: 266 - protein, 17 aa, Unknown
  Possibly HERV
LQNHRGLDLLTVEKGGL

SEQ ID NO: 267 - protein, 17 aa, Unknown
  Possibly HERV
LQNHRGLVLLTAEKRGL

SEQ ID NO: 268 - protein, 17 aa, Unknown
  Possibly HERV
LQNCQGLDLLTAEKGGL

SEQ ID NO: 269 - protein, 17 aa, Unknown
  Possibly HERV
LQNRRGLNMLTAEKRGL

SEQ ID NO: 270 - protein, 17 aa, Unknown
  Possibly HERV
IQNRQGLDLLTAEKGRL

SEQ ID NO: 271 - protein, 17 aa, Unknown
  Possibly HERV
LQNRQGLDLLTAEKGGL

SEQ ID NO: 272 - protein, 17 aa, Unknown
  Possibly HERV
LQYCRGLDLLTAEKGGL

SEQ ID NO: 273 - protein, 17 aa, Unknown
  Possibly HERV
LQNCRGLDLLTAEKEGL

SEQ ID NO: 274 - protein, 17 aa, Unknown
  Possibly HERV
LQNCQGLDLLKAEKGGL

SEQ ID NO: 275 - protein, 17 aa, Unknown
  Possibly HERV
LQNLQGLDLLTAEKGGL

SEQ ID NO: 276 - protein, 17 aa, Unknown
  Possibly HERV
LQNRQGLDLLTAEKGRL

SEQ ID NO: 277 - protein, 17 aa, Unknown
  Possibly HERV
LQNRQALDLLTAERGRT

SEQ ID NO: 278 - protein, 17 aa, Unknown
  Possibly HERV
LQNHQGLDLLTAEKGGL

SEQ ID NO: 279 - protein, 17 aa, Unknown
  Possibly HERV
FQNCRGLDLLTAEKGGL

SEQ ID NO: 280 - protein, 17 aa, Unknown
  Possibly HERV
LQNCQGLDLFTAEKGGL

SEQ ID NO: 281 - protein, 17 aa, Unknown
  Possibly HERV
FQNHRGLDLLAVEKGGL

SEQ ID NO: 282 - protein, 17 aa, Unknown
  Possibly HERV
LQNHRGLDLFTAEKGEL

SEQ ID NO: 283 - protein, 17 aa, Unknown
  Possibly HERV
LQNRRGLDLLTAEKEGL

SEQ ID NO: 284 - protein, 17 aa, Unknown
  Possibly HERV
LQNRRGLDLLTAEKEGL

SEQ ID NO: 285 - protein, 17 aa, Unknown
  Possibly HERV
LQNHRGLDLFTAEKGEL

SEQ ID NO: 286 - protein, 17 aa, Unknown
  Possibly HERV
LQNRRGLDLLTAEKEGL

SEQ ID NO: 287 - protein, 17 aa, Unknown
  Possibly HERV
LQNHQGLDLLTAEKGGL

SEQ ID NO: 288 - protein, 17 aa, Unknown
  Possibly HERV
LQNRQGLDLLTAEKGGL

SEQ ID NO: 289 - protein, 17 aa, Unknown
  Possibly HERV
LQNCRGLDLLTAKKGGL

SEQ ID NO: 290 - protein, 17 aa, Unknown
  Possibly HERV
LQNCRGLDPLTAEKGGL

SEQ ID NO: 291 - protein, 17 aa, Unknown
  Possibly HERV
LQNCRGLDLLNAEKGEL

SEQ ID NO: 292 - protein, 17 aa, Unknown
  Possibly HERV
LQNRRGPHLLTAEKGGL

SEQ ID NO: 293 - protein, 17 aa, Unknown
  Possibly HERV
LQNHRGLDLLTAEKGGL

SEQ ID NO: 294 - protein, 17 aa, Unknown
  Possibly HERV
LQNCRGLDLLTAGKGGL

SEQ ID NO: 295 - protein, 17 aa, Unknown
  Possibly HERV
LQNHWVLDLLTAEKGGP

SEQ ID NO: 296 - protein, 17 aa, Unknown
  Possibly HERV
SPNHQDLDLLTAEKGGL

SEQ ID NO: 297 - protein, 17 aa, Unknown
  Possibly HERV
LQNHGGLDLLTAEKGGF

SEQ ID NO: 298 - protein, 17 aa, Unknown
  Possibly HERV
FQNRRGLDLLTAEKGGL

SEQ ID NO: 299 - protein, 17 aa, Unknown
  Possibly HERV
LQKCRGLDLLTTEKRGL

SEQ ID NO: 300 - protein, 17 aa, Unknown
  Possibly HERV
LQDCRGLDSLTAEKGGL

SEQ ID NO: 301 - protein, 17 aa, Unknown
  Possibly HERV
LQNHQGLYLLAAEKGGL

SEQ ID NO: 302 - protein, 17 aa, Unknown
  Possibly HERV
LQDCWGLDLLTAERGVL

SEQ ID NO: 303 - protein, 17 aa, Unknown
  Possibly HERV
LQNCQGLDPLTAEKGGL

SEQ ID NO: 304 - protein, 17 aa, Unknown
  Possibly HERV
LQNCRGLDLLTAEKGGL

SEQ ID NO: 305 - protein, 17 aa, Unknown
  Possibly HERV
LQNRRGLDLLTAEKGGL

SEQ ID NO: 306 - protein, 17 aa, Unknown
  Possibly HERV
SKNRRGLDLLTAKKGLC

SEQ ID NO: 307 - protein, 17 aa, Unknown
  Possibly HERV
LQKRRNLDLLTAEKGSV

SEQ ID NO: 308 - protein, 17 aa, Unknown
  Possibly HERV
LQNRRGLDLLTAEKGGL

SEQ ID NO: 309 - protein, 17 aa, Unknown
  Possibly HERV
LQNCRGLDLLTAEKGGL

SEQ ID NO: 310 - protein, 17 aa, Unknown
  Possibly HERV
LQNHRGLDLLTAEKGGL

SEQ ID NO: 311 - protein, 17 aa, Unknown
  Possibly HERV
VQNRQGLDLLTTEKGEL

SEQ ID NO: 312 - protein, 17 aa, Unknown
  Possibly HERV
LQNCRGLDLLTAEKGGL

SEQ ID NO: 313 - protein, 17 aa, Unknown
  Possibly HERV
LQNHQGLDLLIAEKGGL

SEQ ID NO: 314 - protein, 17 aa, Unknown
  Possibly HERV
LQNRQGLDLLTAEKGGL

SEQ ID NO: 315 - protein, 17 aa, Unknown
  Possibly HERV
LQNRQGLDLLTAEKGGL

SEQ ID NO: 316 - protein, 17 aa, Unknown
  Possibly HERV
LQNHQGLDLLTAEKGGL

SEQ ID NO: 317 - protein, 17 aa, Unknown
  Possibly HERV
LQNHQGLDLLTAEKGGL

SEQ ID NO: 318 - protein, 17 aa, Unknown
  Possibly HERV
LQNRRGLDLLTAEKGGL

SEQ ID NO: 319 - protein, 17 aa, Unknown
  Possibly HERV
LQNCQGLDFLTAEKGKL

SEQ ID NO: 320 - protein, 17 aa, Unknown
  Possibly HERV
LQNRQSLDLLTAEKGGL

SEQ ID NO: 321 - protein, 17 aa, Unknown
  Possibly HERV
LQNCRGLDFLTAEKGKL

SEQ ID NO: 322 - protein, 17 aa, Unknown
  Possibly HERV
LQNHQDLDFLTAEKGGL

SEQ ID NO: 323 - protein, 17 aa, Unknown
  Possibly HERV
LQNCQSLDILTAEKGGL

SEQ ID NO: 324 - protein, 17 aa, Unknown
  Possibly HERV
LQNRRGLDCLTAEKGGL

SEQ ID NO: 325 - protein, 17 aa, Unknown
  Possibly HERV
LQKHQGLDLLTAEKGVL

SEQ ID NO: 326 - protein, 17 aa, Unknown
  Possibly HERV
LQNRQGPDLLIAEQGGL

SEQ ID NO: 327 - protein, 17 aa, Unknown
  Possibly HERV
AGTLQGLDLLTAEKGGL

SEQ ID NO: 328 - protein, 17 aa, Unknown
  Possibly HERV
AGTLQGLDLLTAEKGGL

SEQ ID NO: 329 - protein, 17 aa, Unknown
  Possibly HERV
LQNHQGLDLLTAEKGGL

SEQ ID NO: 330 - protein, 17 aa, Unknown
  Possibly HERV
LQNRQGLDLFTAEKGEL

SEQ ID NO: 331 - protein, 17 aa, Unknown
  Possibly HERV
LQNHQGLDLLTAEKGGL

SEQ ID NO: 332 - protein, 17 aa, Unknown
  Possibly HERV
LQNCRGLDHLTAEKGGL

SEQ ID NO: 333 - protein, 17 aa, Unknown
  Possibly HERV
LQNCRGLDHLTAEKGGL

SEQ ID NO: 334 - protein, 17 aa, Unknown
  Possibly HERV
LQNCRGLDHLTAEKGGL

SEQ ID NO: 335 - protein, 17 aa, Unknown
  Possibly HERV
YQNRLALDYLLVAEGGV

SEQ ID NO: 336 - protein, 17 aa, Unknown
  Possibly HERV
YQNRLALDYLLVAEGGV

SEQ ID NO: 337 - protein, 17 aa, Unknown
  Possibly HERV
YQNRLVLDYLLVAEGGV

SEQ ID NO: 338 - protein, 17 aa, Unknown
  Possibly HERV
YQNRLALDYLLVAEGGV

SEQ ID NO: 339 - protein, 17 aa, Unknown
  Possibly HERV
CQNRLALDCLVVAEGGV

SEQ ID NO: 340 - protein, 17 aa, Unknown
  Possibly HERV
YQNRLALDCLLAAEGGV

SEQ ID NO: 341 - protein, 17 aa, Unknown
  Possibly HERV
SQNRLALDYLLAAEGGV

SEQ ID NO: 342 - protein, 17 aa, Unknown
  Possibly HERV
YQNRLALDYLLAAKGGV

SEQ ID NO: 343 - protein, 17 aa, Unknown
  Possibly HERV
YQNRLALDYLLAAEGGV

SEQ ID NO: 344 - protein, 17 aa, Unknown
  Possibly HERV
YQNRLALDYLLATEGGV

SEQ ID NO: 345 - protein, 17 aa, Unknown
  Possibly HERV
YQNRLALDYLLAAEGGV

SEQ ID NO: 346 - protein, 17 aa, Unknown
  Possibly HERV
YQNRLALDYLLVAEGGV

SEQ ID NO: 347 - protein, 17 aa, Unknown
  Possibly HERV
YQNRLALDYLLVAEGGV

SEQ ID NO: 348 - protein, 17 aa, Unknown
  Possibly HERV
YQNRLALDYLLAAEGGV

SEQ ID NO: 349 - protein, 17 aa, Unknown
  Possibly HERV
YQNRLALDYLLAVERGV

SEQ ID NO: 350 - protein, 17 aa, Unknown
  Possibly HERV
YQNRLALDYLLVAEGGV

SEQ ID NO: 351 - protein, 17 aa, Unknown
  Possibly HERV
DQKRLALDYLLAAEGRV

SEQ ID NO: 352 - protein, 17 aa, Unknown
  Possibly HERV
YQNRLALDYLLAAEGGV

SEQ ID NO: 353 - protein, 17 aa, Unknown
  Possibly HERV
YQNRLALDYLLAAEGGV

SEQ ID NO: 354 - protein, 17 aa, Unknown
  Possibly HERV
YQNRLALDYLLAAEGGV

SEQ ID NO: 355 - protein, 17 aa, Unknown
  Possibly HERV
YQNRLALDYLLAAEGGV

SEQ ID NO: 356 - protein, 17 aa, Unknown
  Possibly HERV
YQNRLSLDYLLAAKGVI

SEQ ID NO: 357 - protein, 17 aa, Unknown
  Possibly HERV
YQNRLALDYLLAAEGRV

SEQ ID NO: 358 - protein, 17 aa, Unknown
  Possibly HERV
SQNRLALDYLLAAEGGV

SEQ ID NO: 359 - protein, 17 aa, Unknown
  Possibly HERV
YQNGFTLDYLLAAEGGV

SEQ ID NO: 360 - protein, 17 aa, Unknown
  Possibly HERV
YQSRLALDYLLAVEGGV

SEQ ID NO: 361 - protein, 17 aa, Unknown
  Possibly HERV
YQNRLALDYLLAAEGGV

SEQ ID NO: 362 - protein, 17 aa, Unknown
  Possibly HERV
YENRLALDYLLAAEGGA

SEQ ID NO: 363 - protein, 17 aa, Unknown
  Possibly HERV
YQNRLALDYLLAAEGGV

SEQ ID NO: 364 - protein, 17 aa, Unknown
  Possibly HERV
YQNRLALDYLLAAEGGV

SEQ ID NO: 365 - protein, 17 aa, Unknown
  Possibly HERV
SQNRLALDYLLAAEGEV

SEQ ID NO: 366 - protein, 17 aa, Unknown
  Possibly HERV
YQDRLALDYLLAAEGGV

SEQ ID NO: 367 - protein, 17 aa, Unknown
  Possibly HERV
YKYRLALDYLLTAKGGV

SEQ ID NO: 368 - protein, 17 aa, Unknown
  Possibly HERV
YQNRLALDYLLAAEGGV

SEQ ID NO: 369 - protein, 17 aa, Unknown
  Possibly HERV
YQNRLALGYLLAAEGGV

SEQ ID NO: 370 - protein, 17 aa, Unknown
  Possibly HERV
CQNRLALDYLLAAEGGV

SEQ ID NO: 371 - protein, 17 aa, Unknown
  Possibly HERV
YQNRLAFNYLLAAEGGV

SEQ ID NO: 372 - protein, 17 aa, Unknown
  Possibly HERV
YQNRLALDYLLAAEGGG

SEQ ID NO: 373 - protein, 17 aa, Unknown
  Possibly HERV
YQNRLALDYLLAAEEVV

SEQ ID NO: 374 - protein, 17 aa, Unknown
  Possibly HERV
YQNRLALDYLLAAKGGV

SEQ ID NO: 375 - protein, 17 aa, Unknown
  Possibly HERV
YQNRLALYYLLAAEGGV

SEQ ID NO: 376 - protein, 17 aa, Unknown
  Possibly HERV
YQNRLALDYLLAAEGGV

SEQ ID NO: 377 - protein, 17 aa, Unknown
  Possibly HERV
YQNRLALEYLLAAEGGV

SEQ ID NO: 378 - protein, 17 aa, Unknown
  Possibly HERV
YQNGLALNYLLAAEGGV

SEQ ID NO: 379 - protein, 17 aa, Unknown
  Possibly HERV
YQNRLALNYLLAAEGGV

SEQ ID NO: 380 - protein, 17 aa, Unknown
  Possibly HERV
YKNRLTLDYLLAAEGGV

SEQ ID NO: 381 - protein, 17 aa, Unknown
  Possibly HERV
YQNRLVLNYLLAAEGGV

SEQ ID NO: 382 - protein, 17 aa, Unknown
  Possibly HERV
YQNRWALNYLLAAEGGV

SEQ ID NO: 383 - protein, 17 aa, Unknown
  Possibly HERV
YQNRLALDYLLAAEEEI

SEQ ID NO: 384 - protein, 17 aa, Unknown
  Possibly HERV
YQNRLALDYLLAAEGGV

SEQ ID NO: 385 - protein, 17 aa, Unknown
  Possibly HERV
YQSRLALDYLLAVEGGV

SEQ ID NO: 386 - protein, 17 aa, Unknown
  Possibly HERV
YQNRLALDYLLAAEGGV

SEQ ID NO: 387 - protein, 17 aa, Unknown
  Possibly HERV
YQNRLALDYLLAAEGGV

SEQ ID NO: 388 - protein, 17 aa, Unknown
  Possibly HERV
YQNRLALDYLLAAEGRV

SEQ ID NO: 389 - protein, 17 aa, Unknown
  Possibly HERV
YQNRLAFDYLLAAEGGI

SEQ ID NO: 390 - protein, 17 aa, Unknown
  Possibly HERV
YQKRLALNYLLAAEGEV

SEQ ID NO: 391 - protein, 17 aa, Unknown
  Possibly HERV
YQNKLALDDLLAAEEGV

SEQ ID NO: 392 - protein, 17 aa, Unknown
  Possibly HERV
YQNRLALNYSLAAEGGV

SEQ ID NO: 393 - protein, 17 aa, Unknown
  Possibly HERV
YQNRLVLDYLLAAEGGV

SEQ ID NO: 394 - protein, 17 aa, Unknown
  Possibly HERV
YQNRLALNYLLAAEGGK

SEQ ID NO: 395 - protein, 17 aa, Unknown
  Possibly HERV
YQNRLALDYLLAAEGGV

SEQ ID NO: 396 - protein, 17 aa, Unknown
  Possibly HERV
YQNRLALDYLLAAEGAV

SEQ ID NO: 397 - protein, 17 aa, Unknown
  Possibly HERV
HQNRLALNYLVAAEGKI

SEQ ID NO: 398 - protein, 17 aa, Unknown
  Possibly HERV
YQNRLALDYLLAAEGEV

SEQ ID NO: 399 - protein, 17 aa, Unknown
  Possibly HERV
YQNRLALDYLLAAEGEV

SEQ ID NO: 400 - protein, 17 aa, Unknown
  Possibly HERV
NQNRLALDYLPAAEGGI

SEQ ID NO: 401 - protein, 17 aa, Unknown
  Possibly HERV
YQNRLVLDYLLAAEGGV

SEQ ID NO: 402 - protein, 17 aa, Unknown
  Possibly HERV
YQNRLALDYLLAAKGRV

SEQ ID NO: 403 - protein, 17 aa, Unknown
  Possibly HERV
YQNRLALDYLLAAEEGV

SEQ ID NO: 404 - protein, 17 aa, Unknown
  Possibly HERV
YQNRLALDYLLAAEGGV

SEQ ID NO: 405 - protein, 17 aa, Unknown
  Possibly HERV
YQNRLALDYLLAAEGGV

SEQ ID NO: 406 - protein, 17 aa, Unknown
  Possibly HERV
LQNQRRLGLLTAEKGGL

SEQ ID NO: 407 - protein, 17 aa, Unknown
  Possibly HERV
LQNRGGLDLIMAEKGDL

SEQ ID NO: 408 - protein, 17 aa, Unknown
  Possibly HERV
LQNRLGLDLTMAEKGGL

SEQ ID NO: 409 - protein, 17 aa, Unknown
  Possibly HERV
LQNRQGLNLITAENWSL

SEQ ID NO: 410 - protein, 17 aa, Unknown
  Possibly HERV
LQNRWGLDLIMAEKRDL

SEQ ID NO: 411 - protein, 17 aa, Unknown
  Possibly HERV
LQNRQELDVITTERGDL

SEQ ID NO: 412 - protein, 17 aa, Unknown
  Possibly HERV
LQNRWGLDLITAEKGAS

SEQ ID NO: 413 - protein, 17 aa, Unknown
  Possibly HERV
LQNRWGLDLIMAEKGTS

SEQ ID NO: 414 - protein, 17 aa, Unknown
  Possibly HERV
LQNRQRLDLIMVEREGI

SEQ ID NO: 415 - protein, 17 aa, Unknown
  Possibly HERV
LQNRQRLDLIMVEREGI

SEQ ID NO: 416 - protein, 17 aa, Unknown
  Possibly HERV
LQNRQRLDLIMVEKEGI

SEQ ID NO: 417 - protein, 17 aa, Unknown
  Possibly HERV
LQNRQRLDLIMVEKEGI

SEQ ID NO: 418 - protein, 17 aa, Unknown
  Possibly HERV
LQNRQRLDLIMVEKEGI

SEQ ID NO: 419 - protein, 17 aa, Unknown
  Possibly HERV
LQNRQRLDLIMVEREGI

SEQ ID NO: 420 - protein, 17 aa, Unknown
  Possibly HERV
LQNRQRLDLIMVEREGI

SEQ ID NO: 421 - protein, 17 aa, Unknown
  Possibly HERV
LQNKWGLNLITAEKGGL

SEQ ID NO: 422 - protein, 17 aa, Unknown
  Possibly HERV
LQNRRGLDLLTTEKGGS

SEQ ID NO: 423 - protein, 17 aa, Unknown
  Possibly HERV
LQNRWGLNLIMVEKGGL

SEQ ID NO: 424 - protein, 17 aa, Unknown
  Possibly HERV
LQNRRGLDLLLKKVAYV

SEQ ID NO: 425 - protein, 17 aa, Unknown
  Possibly HERV
LQNRKGLDLLTAEKGSL

SEQ ID NO: 426 - protein, 17 aa, Unknown
  Possibly HERV
LQNHRDLDLLTAEKGEL

SEQ ID NO: 427 - protein, 17 aa, Unknown
  Possibly HERV
LQNHRVLDFLTAEKGGL

SEQ ID NO: 428 - protein, 17 aa, Unknown
  Possibly HERV
LQNHRGVDRLTAKKGGL

SEQ ID NO: 429 - protein, 17 aa, Unknown
  Possibly HERV
LQNHRDLDLLTAEKGGL

SEQ ID NO: 430 - protein, 17 aa, Unknown
  Possibly HERV
LQNRRGLDLLTAEKGGL

SEQ ID NO: 431 - protein, 17 aa, Unknown
  Possibly HERV
LQSRRGLDLLTAEKGGI

SEQ ID NO: 432 - protein, 17 aa, Unknown
  Possibly HERV
LQNHRGLNLLTAKKGGF

SEQ ID NO: 433 - protein, 17 aa, Unknown
  Possibly HERV
LKNRRGLDLLTADKGGL

SEQ ID NO: 434 - protein, 17 aa, Unknown
  Possibly HERV
LQNQRGLNLLTTDKGGF

SEQ ID NO: 435 - protein, 17 aa, Unknown
  Possibly HERV
LQNHRGLNLLTAEKGRL

SEQ ID NO: 436 - protein, 17 aa, Unknown
  Possibly HERV
LQNHRGLHLLTADKGGL

SEQ ID NO: 437 - protein, 17 aa, Unknown
  Possibly HERV
LQNHRGLDLLTADKGGL

SEQ ID NO: 438 - protein, 17 aa, Unknown
  Possibly HERV
LQNHRGLDLLTAEKGGL

SEQ ID NO: 439 - protein, 17 aa, Unknown
  Possibly HERV
LQNRRGLHLLTAEKEGL

SEQ ID NO: 440 - protein, 17 aa, Unknown
  Possibly HERV
LQNRRGLGLLTAEKGGL

SEQ ID NO: 441 - protein, 17 aa, Unknown
  Possibly HERV
LQNCRGLDLFTAKKGEL

SEQ ID NO: 442 - protein, 17 aa, Unknown
  Possibly HERV
LQNRKGLNLLTAEKGGL

SEQ ID NO: 443 - protein, 17 aa, Unknown
  Possibly HERV
LQNRRGLDLLTAEKGGL

SEQ ID NO: 444 - protein, 17 aa, Unknown
  Possibly HERV
LQNRLGLNLLIAEKGGL

SEQ ID NO: 445 - protein, 17 aa, Unknown
  Possibly HERV
QGQLRGLDLLTAEKGGL

SEQ ID NO: 446 - protein, 17 aa, Unknown
  Possibly HERV
LQNRQGLDLLTAKKRGP

SEQ ID NO: 447 - protein, 17 aa, Unknown
  Possibly HERV
LQNCQGLDLLTAKKRGF

SEQ ID NO: 448 - protein, 17 aa, Unknown
  Possibly HERV
LQNRRGLDLLTAEKGGL

SEQ ID NO: 449 - protein, 17 aa, Unknown
  Possibly HERV
LQNRGGLDLLTAKKRGL

SEQ ID NO: 450 - protein, 17 aa, Unknown
  Possibly HERV
LQNHRGLDLLTAGKGRL

SEQ ID NO: 451 - protein, 17 aa, Unknown
  Possibly HERV
LQNRQGLDLLTAKKGGL

SEQ ID NO: 452 - protein, 17 aa, Unknown
  Possibly HERV
LQNHQGLDLLTAEKGEL

SEQ ID NO: 453 - protein, 17 aa, Unknown
  Possibly HERV
LQNRGGLDLLTAKKRGL

SEQ ID NO: 454 - protein, 17 aa, Unknown
  Possibly HERV
LQNRRGLDLLTAEKGGL

SEQ ID NO: 455 - protein, 17 aa, Unknown
  Possibly HERV
LQNHRGLDLLTAEKGGL

SEQ ID NO: 456 - protein, 17 aa, Unknown
  Possibly HERV
LQNHQGLDLLTAEKGGL

SEQ ID NO: 457 - protein, 17 aa, Unknown
  Possibly HERV
LQNCQGLVLLTAEKGGL

SEQ ID NO: 458 - protein, 17 aa, Unknown
  Possibly HERV
LQNRRGLDLLTAEKGGL

SEQ ID NO: 459 - protein, 17 aa, Unknown
  Possibly HERV
LQNCQGLDLLTAEKGGL

SEQ ID NO: 460 - protein, 17 aa, Unknown
  Possibly HERV
LQNCRGLDLLIAEKGGL

SEQ ID NO: 461 - protein, 17 aa, Unknown
  Possibly HERV
LQNRRGLDLLLKKEDSA

SEQ ID NO: 462 - protein, 17 aa, Unknown
  Possibly HERV
LQNRRGLDLLTAEKGGL

SEQ ID NO: 463 - protein, 17 aa, Unknown
  Possibly HERV
LQNCRGLDLLTAEKGGL

SEQ ID NO: 464 - protein, 17 aa, Unknown
  Possibly HERV
LQNRRGLDLLTAEKGGL

SEQ ID NO: 465 - protein, 17 aa, Unknown
  Possibly HERV
LQNCQGLDLLTAEKGRR

SEQ ID NO: 466 - protein, 17 aa, Unknown
  Possibly HERV
LQNCRGLDLLTAEKGGL

SEQ ID NO: 467 - protein, 17 aa, Unknown
  Possibly HERV
LQNRRGLDLLTAEKGGL

SEQ ID NO: 468 - protein, 17 aa, Unknown
  Possibly HERV
LQNRRGLDILTVEEGGL

SEQ ID NO: 469 - protein, 17 aa, Unknown
  Possibly HERV
LQNRRGLNLLIAEKGGL

SEQ ID NO: 470 - protein, 17 aa, Unknown
  Possibly HERV
LQNRQGLNLLIAEKGRL

SEQ ID NO: 471 - protein, 17 aa, Unknown
  Possibly HERV
LQNRQGLNLLIAEKGRL

SEQ ID NO: 472 - protein, 17 aa, Unknown
  Possibly HERV
LQNRQGLNLLIAEKGRL

SEQ ID NO: 473 - protein, 17 aa, Unknown
  Possibly HERV
LQNRQGLNLLIAEKGRL

SEQ ID NO: 474 - protein, 17 aa, Unknown
  Possibly HERV
LQNRQGLNLLIAEKGRL

SEQ ID NO: 475 - protein, 17 aa, Unknown
  Possibly HERV
LQNRQGLNLLIAEKGRL

SEQ ID NO: 476 - protein, 17 aa, Unknown
  Possibly HERV
LQNHRGPDLLTAEKGGL

SEQ ID NO: 477 - protein, 17 aa, Unknown
  Possibly HERV
LQNHQGLDLLTAEKGGL

SEQ ID NO: 478 - protein, 17 aa, Unknown
  Possibly HERV
LQNGRGLDLLTAEKGGL

SEQ ID NO: 479 - protein, 17 aa, Unknown
  Possibly HERV
LQNHQGLNLLIAEKGRL

SEQ ID NO: 480 - protein, 17 aa, Unknown
  Possibly HERV
LQNRQGLDLLTAEKGGL

SEQ ID NO: 481 - protein, 17 aa, Unknown
  Possibly HERV
LQNLQGLDLLTAEERGL

SEQ ID NO: 482 - protein, 17 aa, Unknown
  Possibly HERV
LQNGQGRDLLTAEKGGL

SEQ ID NO: 483 - protein, 17 aa, Unknown
  Possibly HERV
LQNCQGLNLLIAEKGGL

SEQ ID NO: 484 - protein, 17 aa, Unknown
  Possibly HERV
LQNCRGLDLLTVEKGGF

SEQ ID NO: 485 - protein, 17 aa, Unknown
  Possibly HERV
LQNRRGLDLLLEKENIC

SEQ ID NO: 486 - protein, 17 aa, Unknown
  Possibly HERV
LQNCQGLDLLTAEKGGL

SEQ ID NO: 487 - protein, 17 aa, Unknown
  Possibly HERV
LHNRRGLNLLTVGKGGL

SEQ ID NO: 488 - protein, 17 aa, Unknown
  Possibly HERV
LQNCRGLDLLTAEKGGP

SEQ ID NO: 489 - protein, 17 aa, Unknown
  Possibly HERV
LQNRRGFDLLLKKKGTL

SEQ ID NO: 490 - protein, 17 aa, Unknown
  Possibly HERV
LQNCQGLDLLTAKKKKD

SEQ ID NO: 491 - protein, 17 aa, Unknown
  Possibly HERV
LQNHGGLDLLIAEKGGL

SEQ ID NO: 492 - protein, 17 aa, Unknown
  Possibly HERV
FQNPRGLDLLTAEKGGL

SEQ ID NO: 493 - protein, 17 aa, Unknown
  Possibly HERV
LQNRRGLDLLTAEKGGL

SEQ ID NO: 494 - protein, 17 aa, Unknown
  Possibly HERV
LQNRRGLDLLTALKKGD

SEQ ID NO: 495 - protein, 17 aa, Unknown
  Possibly HERV
LQNRRDLDLLTAEKGGL

SEQ ID NO: 496 - protein, 17 aa, Unknown
  Possibly HERV
LQNCQGLDLLTAKEKGT

SEQ ID NO: 497 - protein, 17 aa, Unknown
  Possibly HERV
LQNRQGLDLLTAKKRGP

SEQ ID NO: 498 - protein, 17 aa, Unknown
  Possibly HERV
LQNRRGLDLLNAEKGGL

SEQ ID NO: 499 - protein, 17 aa, Unknown
  Possibly HERV
LQNCRGLDLLAAEKGGL

SEQ ID NO: 500 - protein, 17 aa, Unknown
  Possibly HERV
LQNRRGLDLLTAEKGGL

SEQ ID NO: 501 - protein, 17 aa, Unknown
  Possibly HERV
LQNHRGLDLLTAEKGGL

SEQ ID NO: 502 - protein, 17 aa, Unknown
  Possibly HERV
LQNCRGIDLLTAKKKRT

SEQ ID NO: 503 - protein, 17 aa, Unknown
  Possibly HERV
LQNRQGLDLLLKMEDSV

SEQ ID NO: 504 - protein, 17 aa, Unknown
  Possibly HERV
LQNCQGLDLVTAKKRRL

SEQ ID NO: 505 - protein, 17 aa, Unknown
  Possibly HERV
LRNCQGLDLLTAEKGGL

SEQ ID NO: 506 - protein, 17 aa, Unknown
  Possibly HERV
LQNHQGLDLLTAEKGGL

SEQ ID NO: 507 - protein, 17 aa, Unknown
  Possibly HERV
LQNRPGLDLLTAKKKGG

SEQ ID NO: 508 - protein, 17 aa, Unknown
  Possibly HERV
LQNRRGLDLLTAEKGGL

SEQ ID NO: 509 - protein, 17 aa, Unknown
  Possibly HERV
LQNRQGLDLLTAEKGGL

SEQ ID NO: 510 - protein, 17 aa, Unknown
  Possibly HERV
LQNHIGLDLLTARKRGL

SEQ ID NO: 511 - protein, 17 aa, Unknown
  Possibly HERV
LQNRRGLHLLTAEKGGL

SEQ ID NO: 512 - protein, 17 aa, Unknown
  Possibly HERV
LQNRQSLDLLTAEKGGL

SEQ ID NO: 513 - protein, 17 aa, Unknown
  Possibly HERV
LQNHQGLNLLTAEKGGL

SEQ ID NO: 514 - protein, 17 aa, Unknown
  Possibly HERV
LQNRQSLNLLTAKKRGL

SEQ ID NO: 515 - protein, 17 aa, Unknown
  Possibly HERV
LQNRRGLDLLLKKEDSV

SEQ ID NO: 516 - protein, 17 aa, Unknown
  Possibly HERV
LQNHQGLDLLTAEKGGL

SEQ ID NO: 517 - protein, 17 aa, Unknown
  Possibly HERV
LQNCRGLDLPTAEKGGL

SEQ ID NO: 518 - protein, 17 aa, Unknown
  Possibly HERV
LQNRRGLDLLTAEKGGL

SEQ ID NO: 519 - protein, 17 aa, Unknown
  Possibly HERV
LQNRQGLDLLTAEKGGL

SEQ ID NO: 520 - protein, 17 aa, Unknown
  Possibly HERV
LQNCRGLDLLTAEKEGR

SEQ ID NO: 521 - protein, 17 aa, Unknown
  Possibly HERV
LQNRRGLDLLTAEKGGL

SEQ ID NO: 522 - protein, 17 aa, Unknown
  Possibly HERV
SKNRRGLDLLTAEKGEL

SEQ ID NO: 523 - protein, 17 aa, Unknown
  Possibly HERV
LQNCQGLDLLTAKKGRL

SEQ ID NO: 524 - protein, 17 aa, Unknown
  Possibly HERV
LQNHRGPDLLTAKKRGL

SEQ ID NO: 525 - protein, 17 aa, Unknown
  Possibly HERV
LQNRRGLNLFTAEKGGL

SEQ ID NO: 526 - protein, 17 aa, Unknown
  Possibly HERV
LQNRRGLDLLLKKEDSV

SEQ ID NO: 527 - protein, 17 aa, Unknown
  Possibly HERV
LQNRRGLDLLTAEKGGL

SEQ ID NO: 528 - protein, 17 aa, Unknown
  Possibly HERV
LQNRRGLNLLTAEKGGL

SEQ ID NO: 529 - protein, 17 aa, Unknown
  Possibly HERV
LQNHQGLDLLTAKKEKK

SEQ ID NO: 530 - protein, 17 aa, Unknown
  Possibly HERV
LQNLQGLDLLTAKKKRT

SEQ ID NO: 531 - protein, 17 aa, Unknown
  Possibly HERV
LQNRRGLDLLTAEKGGL

SEQ ID NO: 532 - protein, 17 aa, Unknown
  Possibly HERV
LQNHRGLDLLTAEKGGL

SEQ ID NO: 533 - protein, 17 aa, Unknown
  Possibly HERV
LQNHRGLDLLTAEKGGL

SEQ ID NO: 534 - protein, 17 aa, Unknown
  Possibly HERV
LQDRQGLDLLTAKKGDS

SEQ ID NO: 535 - protein, 17 aa, Unknown
  Possibly HERV
LQNRRGLDLLLKKKDSI

SEQ ID NO: 536 - protein, 17 aa, Unknown
  Possibly HERV
LQNRRGLDLLTAEKGGL

SEQ ID NO: 537 - protein, 17 aa, Unknown
  Possibly HERV
LQNRQGLDLLTADKGGL

SEQ ID NO: 538 - protein, 17 aa, Unknown
  Possibly HERV
LQNRRGLDLLTAEKGGL

SEQ ID NO: 539 - protein, 17 aa, Unknown
  Possibly HERV
LQNHRGLDLLTAEKGGL

SEQ ID NO: 540 - protein, 17 aa, Unknown
  Possibly HERV
PQNLQGLYLLTAEKGGL

SEQ ID NO: 541 - protein, 17 aa, Unknown
  Possibly HERV
LQNHRGLDLLTAEKGGL

SEQ ID NO: 542 - protein, 17 aa, Unknown
  Possibly HERV
LQNHRGLDLLTAEKGGL

SEQ ID NO: 543 - protein, 17 aa, Unknown
  Possibly HERV
LQNHRGLDLLTAEKGGL

SEQ ID NO: 544 - protein, 17 aa, Unknown
  Possibly HERV
LQNHRGLDLLTAEKGGL

SEQ ID NO: 545 - protein, 17 aa, Unknown
  Possibly HERV
LQNCRGLNLLTAEKGGL

SEQ ID NO: 546 - protein, 17 aa, Unknown
  Possibly HERV
LQNRRGLDLLTAEKGGL

SEQ ID NO: 547 - protein, 17 aa, Unknown
  Possibly HERV
LQNHRGLDLLTVEKGGL

SEQ ID NO: 548 - protein, 17 aa, Unknown
  Possibly HERV
PQNHRGLDLLTAERGLC

SEQ ID NO: 549 - protein, 17 aa, Unknown
  Possibly HERV
LQNRQGLDLLLKKEDSV

SEQ ID NO: 550 - protein, 17 aa, Unknown
  Possibly HERV
LQNRRGLDLLTAKNKIK

SEQ ID NO: 551 - protein, 17 aa, Unknown
  Possibly HERV
LQNRRGLNLLTAKKKKK

SEQ ID NO: 552 - protein, 17 aa, Unknown
  Possibly HERV
FQNRRGLDLLTAKKNET

SEQ ID NO: 553 - protein, 17 aa, Unknown
  Possibly HERV
LQNHGGLDLLTAKKRGL

SEQ ID NO: 554 - protein, 17 aa, Unknown
  Possibly HERV
LQNRRGLDLLTAEKGGL

SEQ ID NO: 555 - protein, 17 aa, Unknown
  Possibly HERV
LQNHQGLDLLTAGKGGL

SEQ ID NO: 556 - protein, 17 aa, Unknown
  Possibly HERV
LQNRQGLDLVTAEKGRL

SEQ ID NO: 557 - protein, 17 aa, Unknown
  Possibly HERV
LQNHRGLDLLTSEKGGL

SEQ ID NO: 558 - protein, 17 aa, Unknown
  Possibly HERV
LQNRRGLDLLTAEKGGL

SEQ ID NO: 559 - protein, 17 aa, Unknown
  Possibly HERV
LQNHRGLDLLTAEKGGL

SEQ ID NO: 560 - protein, 17 aa, Unknown
  Possibly HERV
LQNSRGLDLLTAEKGGL

SEQ ID NO: 561 - protein, 17 aa, Unknown
  Possibly HERV
LQNHRGLDLLIAKKRGL

SEQ ID NO: 562 - protein, 17 aa, Unknown
  Possibly HERV
LQNRQGLNLLTAEKGGL

SEQ ID NO: 563 - protein, 17 aa, Unknown
  Possibly HERV
LQSRQGLDLLTAEKGGL

SEQ ID NO: 564 - protein, 17 aa, Unknown
  Possibly HERV
LQNRQGLDLLLKKEDSV

SEQ ID NO: 565 - protein, 17 aa, Unknown
  Possibly HERV
LQNRRGPDLLTAKKKKS

SEQ ID NO: 566 - protein, 17 aa, Unknown
  Possibly HERV
FQNSRGHELLTAEKGGL

SEQ ID NO: 567 - protein, 17 aa, Unknown
  Possibly HERV
LQNCQGLDLLTAEKGGL

SEQ ID NO: 568 - protein, 17 aa, Unknown
  Possibly HERV
YQNRLALDYLLAEEGGV

SEQ ID NO: 569 - protein, 17 aa, Unknown
  Possibly HERV
YQNMLALDYLLAEEGGV

SEQ ID NO: 570 - protein, 17 aa, Unknown
  Possibly HERV
YQNRLTLDYLLAEEGGV

SEQ ID NO: 571 - protein, 17 aa, Unknown
  Possibly HERV
YQNGPALDYLLAEESGV

SEQ ID NO: 572 - protein, 17 aa, Unknown
  Possibly HERV
LSNRLALNYLLAEKGGV

SEQ ID NO: 573 - protein, 17 aa, Unknown
  Possibly HERV
YQNRLALDYLLAEGGGV

SEQ ID NO: 574 - protein, 17 aa, Unknown
  Possibly HERV
YQNRPALGYLLAGKGGI

SEQ ID NO: 575 - protein, 17 aa, Unknown
  Possibly HERV
YQNRLALDYLLAEKGGV

SEQ ID NO: 576 - protein, 17 aa, Unknown
  Possibly HERV
YQNRLALNYLLEEEGGV

SEQ ID NO: 577 - protein, 17 aa, Unknown
  Possibly HERV
YQNRLALDYLLTEEGAV

SEQ ID NO: 578 - protein, 17 aa, Unknown
  Possibly HERV
YQNRLALDYLLAEEGAV

SEQ ID NO: 579 - protein, 17 aa, Unknown
  Possibly HERV
YQNRLALDYLLAEEGAV

SEQ ID NO: 580 - protein, 17 aa, Unknown
  Possibly HERV
YQNRLALDYLLAEEGAG

SEQ ID NO: 581 - protein, 17 aa, Unknown
  Possibly HERV
YQNRLALDYLLAEEGGV

SEQ ID NO: 582 - protein, 17 aa, Unknown
  Possibly HERV
YQNRLALDCLLAEEGGA

SEQ ID NO: 583 - protein, 17 aa, Unknown
  Possibly HERV
YPNRLALDYLLAEEGGV

SEQ ID NO: 584 - protein, 17 aa, Unknown
  Possibly HERV
YQNRLALDYLLAEEGGV

SEQ ID NO: 585 - protein, 17 aa, Unknown
  Possibly HERV
YQNRLALPCLLAEEGGV

SEQ ID NO: 586 - protein, 17 aa, Unknown
  Possibly HERV
YQNRPAPDYLSAEEGGV

SEQ ID NO: 587 - protein, 17 aa, Unknown
  Possibly HERV
YQNRLALDYLLAEEGGV

SEQ ID NO: 588 - protein, 17 aa, Unknown
  Possibly HERV
YQNRLALDYLLAEEGGV

SEQ ID NO: 589 - protein, 17 aa, Unknown
  Possibly HERV
YQNRLALDYLLAEEGGV

SEQ ID NO: 590 - protein, 17 aa, Unknown
  Possibly HERV
YQNRLALDYLLAEEGEV

SEQ ID NO: 591 - protein, 17 aa, Unknown
  Possibly HERV
YQNRLPLDYLLAEGGVC

SEQ ID NO: 592 - protein, 17 aa, Unknown
  Possibly HERV
FQNRLPLDYLLAEEGGV

SEQ ID NO: 593 - protein, 17 aa, Unknown
  Possibly HERV
YQNRLALDYLLAEEGGV

SEQ ID NO: 594 - protein, 17 aa, Unknown
  Possibly HERV
YQNRLALDYLLAEEGGV

SEQ ID NO: 595 - protein, 17 aa, Unknown
  Possibly HERV
WENRIALDIISAAEGGG

SEQ ID NO: 596 - protein, 17 aa, Unknown
  Possibly HERV
WENRITLDVILTEKGGV

SEQ ID NO: 597 - protein, 17 aa, Unknown
  Possibly HERV
WENRITLDMILADKGEV

SEQ ID NO: 598 - protein, 17 aa, Unknown
  Possibly HERV
WENRMALDMILAEKGGV

SEQ ID NO: 599 - protein, 17 aa, Unknown
  Possibly HERV
WENRIALDTMLAEKGGV

SEQ ID NO: 600 - protein, 17 aa, Unknown
  Possibly HERV
WENRIALDTMLAEKGGV

SEQ ID NO: 601 - protein, 17 aa, Unknown
  Possibly HERV
WENRIALDTMLAEKGGV

SEQ ID NO: 602 - protein, 17 aa, Unknown
  Possibly HERV
WENRIALDTMLAEKGGV

SEQ ID NO: 603 - protein, 17 aa, Unknown
  Possibly HERV
WENRIALDTMLAEKGGV

SEQ ID NO: 604 - protein, 17 aa, Unknown
  Possibly HERV
CENRMALDMILAEKDGV

SEQ ID NO: 605 - protein, 17 aa, Unknown
  Possibly HERV
WENRRALNIMLAKKGKV

SEQ ID NO: 606 - protein, 17 aa, Unknown
  Possibly HERV
SENRIALDMILAEKGGV

SEQ ID NO: 607 - protein, 17 aa, Unknown
  Possibly HERV
WENRITLDMVLAKKGRV

SEQ ID NO: 608 - protein, 17 aa, Unknown
  Possibly HERV
WENRIALDMILAEKRGG

SEQ ID NO: 609 - protein, 17 aa, Unknown
  Possibly HERV
WKNRIALDMILAEKGGV

SEQ ID NO: 610 - protein, 17 aa, Unknown
  Possibly HERV
WENRMALDMIVAEKGRV

SEQ ID NO: 611 - protein, 17 aa, Unknown
  Possibly HERV
WENRIALDMILAEKGGV

SEQ ID NO: 612 - protein, 17 aa, Unknown
  Possibly HERV
WGNRIALDMLLAEKGRV

SEQ ID NO: 613 - protein, 17 aa, Unknown
  Possibly HERV
WENRMALDMILAEKGGV

SEQ ID NO: 614 - protein, 17 aa, Unknown
  Possibly HERV
WKNRMALDMILAEKGGV

SEQ ID NO: 615 - protein, 17 aa, Unknown
  Possibly HERV
WENRIALDMMLAKKGGV

SEQ ID NO: 616 - protein, 17 aa, Unknown
  Possibly HERV
WENRMALDMVLAEKGGV

SEQ ID NO: 617 - protein, 17 aa, Unknown
  Possibly HERV
RENRIALDMILAKEGGI

SEQ ID NO: 618 - protein, 17 aa, Unknown
  Possibly HERV
QDNRITLDMMLAEKGGV

SEQ ID NO: 619 - protein, 17 aa, Unknown
  Possibly HERV
WENRIALNVILAKEGGV

SEQ ID NO: 620 - protein, 17 aa, Unknown
  Possibly HERV
WENRIALDMILAEKGRV

SEQ ID NO: 621 - protein, 17 aa, Unknown
  Possibly HERV
WENRIALDMMLAEKGRV

SEQ ID NO: 622 - protein, 17 aa, Unknown
  Possibly HERV
WENRIALDMMLAEKGRV

SEQ ID NO: 623 - protein, 17 aa, Unknown
  Possibly HERV
WGNRIALDMILAKEGGV

SEQ ID NO: 624 - protein, 17 aa, Unknown
  Possibly HERV
WENRMALDMILAKKGGV

SEQ ID NO: 625 - protein, 17 aa, Unknown
  Possibly HERV
WENRIALEMILAERGGV

SEQ ID NO: 626 - protein, 17 aa, Unknown
  Possibly HERV
WENRIALDMTLAKEGGV

SEQ ID NO: 627 - protein, 17 aa, Unknown
  Possibly HERV
WENRMALDMMLAKKRGV

SEQ ID NO: 628 - protein, 17 aa, Unknown
  Possibly HERV
WENRTALDMILAERGGV

SEQ ID NO: 629 - protein, 17 aa, Unknown
  Possibly HERV
WENRIALDIKLAEKGGV

SEQ ID NO: 630 - protein, 17 aa, Unknown
  Possibly HERV
WENRMALDMILAAKGGV

SEQ ID NO: 631 - protein, 17 aa, Unknown
  Possibly HERV
WESRMALDMILAEKGGV

SEQ ID NO: 632 - protein, 17 aa, Unknown
  Possibly HERV
WENRVTLDMMLAEKGGV

SEQ ID NO: 633 - protein, 17 aa, Unknown
  Possibly HERV
WENRIALDMIPAEKSEV

SEQ ID NO: 634 - protein, 17 aa, Unknown
  Possibly HERV
QENRIALDMTLAERGRV

SEQ ID NO: 635 - protein, 17 aa, Unknown
  Possibly HERV
WENRMALDTILAEKVVF

SEQ ID NO: 636 - protein, 17 aa, Unknown
  Possibly HERV
WGNRIAFDIILAERGGV

SEQ ID NO: 637 - protein, 17 aa, Unknown
  Possibly HERV
WENRMALDVILAEKGSV

SEQ ID NO: 638 - protein, 17 aa, Unknown
  Possibly HERV
WENRMALDMILAEKGGV

SEQ ID NO: 639 - protein, 17 aa, Unknown
  Possibly HERV
WENRMALDMILAEKGEV

SEQ ID NO: 640 - protein, 17 aa, Unknown
  Possibly HERV
WENRIALDTMLAEKGGV

SEQ ID NO: 641 - protein, 17 aa, Unknown
  Possibly HERV
WENRIDLDMILAERGGV

SEQ ID NO: 642 - protein, 17 aa, Unknown
  Possibly HERV
WENRLALDMILAGKGRV

SEQ ID NO: 643 - protein, 17 aa, Unknown
  Possibly HERV
WENGIALDIILAERGGV

SEQ ID NO: 644 - protein, 17 aa, Unknown
  Possibly HERV
WENRMALDMILAEKGGV

SEQ ID NO: 645 - protein, 17 aa, Unknown
  Possibly HERV
WENRIALDMVLAERGGV

SEQ ID NO: 646 - protein, 17 aa, Unknown
  Possibly HERV
WENRIALDVILAESGGV

SEQ ID NO: 647 - protein, 17 aa, Unknown
  Possibly HERV
WENRIALDIMLAEKGGV

SEQ ID NO: 648 - protein, 17 aa, Unknown
  Possibly HERV
WENRMALDMILVEKGGI

SEQ ID NO: 649 - protein, 17 aa, Unknown
  Possibly HERV
WENRIALDMMLAKKGGV

SEQ ID NO: 650 - protein, 17 aa, Unknown
  Possibly HERV
WENRIALDRILAEKAGA

SEQ ID NO: 651 - protein, 17 aa, Unknown
  Possibly HERV
RENRIALDMILTEKGGV

SEQ ID NO: 652 - protein, 17 aa, Unknown
  Possibly HERV
WENRTALDMILAERGGV

SEQ ID NO: 653 - protein, 17 aa, Unknown
  Possibly HERV
WENRIALDMILAKKGGS

SEQ ID NO: 654 - protein, 17 aa, Unknown
  Possibly HERV
WENRMALDMILAEKGGV

SEQ ID NO: 655 - protein, 17 aa, Unknown
  Possibly HERV
WGNRIALDIILAERGGV

SEQ ID NO: 656 - protein, 17 aa, Unknown
  Possibly HERV
GKNRIALDMILAEKGRI

SEQ ID NO: 657 - protein, 17 aa, Unknown
  Possibly HERV
WENRMALDTILAEKVVF

SEQ ID NO: 658 - protein, 17 aa, Unknown
  Possibly HERV
WENRKALDMMLAKKGGV

SEQ ID NO: 659 - protein, 17 aa, Unknown
  Possibly HERV
WENRIALDMVLADREGV

SEQ ID NO: 660 - protein, 17 aa, Unknown
  Possibly HERV
WENRRALDMILAESGGV

SEQ ID NO: 661 - protein, 17 aa, Unknown
  Possibly HERV
WENRIALDMILAEKCGG

SEQ ID NO: 662 - protein, 17 aa, Unknown
  Possibly HERV
WENRMALDMILAEKGGV

SEQ ID NO: 663 - protein, 17 aa, Unknown
  Possibly HERV
WENRMALDIILAAKGSV

SEQ ID NO: 664 - protein, 17 aa, Unknown
  Possibly HERV
WRNKMALDMILATKGGV

SEQ ID NO: 665 - protein, 17 aa, Unknown
  Possibly HERV
GKNRIALDVILAEKGGV

SEQ ID NO: 666 - protein, 17 aa, Unknown
  Possibly HERV
WENRTALDMILAERGGV

SEQ ID NO: 667 - protein, 17 aa, Unknown
  Possibly HERV
WENRIALDMILAEKGGV

SEQ ID NO: 668 - protein, 17 aa, Unknown
  Possibly HERV
WENRIALDMLLAEKGGV

SEQ ID NO: 669 - protein, 17 aa, Unknown
  Possibly HERV
WENRMALDMTLAEKGGV

SEQ ID NO: 670 - protein, 17 aa, Unknown
  Possibly HERV
WENRIALDMILAERGGV

SEQ ID NO: 671 - protein, 17 aa, Unknown
  Possibly HERV
WENRRALDMILAEKGTV

SEQ ID NO: 672 - protein, 17 aa, Unknown
  Possibly HERV
WETRIALDMMLAEKGRL

SEQ ID NO: 673 - protein, 17 aa, Unknown
  Possibly HERV
WENRIALNIILAKEAGV

SEQ ID NO: 674 - protein, 17 aa, Unknown
  Possibly HERV
WENRIVLDMSLAEKGGV

SEQ ID NO: 675 - protein, 17 aa, Unknown
  Possibly HERV
WENRIALDMILAERGGV

SEQ ID NO: 676 - protein, 17 aa, Unknown
  Possibly HERV
WENRIALNKILAEKGGV

SEQ ID NO: 677 - protein, 17 aa, Unknown
  Possibly HERV
WENRTALDMILAEKGKV

SEQ ID NO: 678 - protein, 17 aa, Unknown
  Possibly HERV
WENRTALDMILAEKGKV

SEQ ID NO: 679 - protein, 17 aa, Unknown
  Possibly HERV
WENRTALDMILAEKGKV

SEQ ID NO: 680 - protein, 17 aa, Unknown
  Possibly HERV
WENRTALDMILAEKGKV

SEQ ID NO: 681 - protein, 17 aa, Unknown
  Possibly HERV
WESRIALDMLLAKKGGF

SEQ ID NO: 682 - protein, 17 aa, Unknown
  Possibly HERV
WENRIALDMILAEKGRV

SEQ ID NO: 683 - protein, 17 aa, Unknown
  Possibly HERV
WENRMALAMILSEKGDV

SEQ ID NO: 684 - protein, 17 aa, Unknown
  Possibly HERV
GKNRIALDMIFAKKGGV

SEQ ID NO: 685 - protein, 17 aa, Unknown
  Possibly HERV
WKDRIALDMILAERGGV

SEQ ID NO: 686 - protein, 17 aa, Unknown
  Possibly HERV
WENRMALVMILAENGGV

SEQ ID NO: 687 - protein, 17 aa, Unknown
  Possibly HERV
WENRMALVMILAENGGV

SEQ ID NO: 688 - protein, 17 aa, Unknown
  Possibly HERV
WENRMALDMMLAEKGGV

SEQ ID NO: 689 - protein, 17 aa, Unknown
  Possibly HERV
WENRIAFDMILAERGGV

SEQ ID NO: 690 - protein, 17 aa, Unknown
  Possibly HERV
WENRIGLDMILAESGGV

SEQ ID NO: 691 - protein, 17 aa, Unknown
  Possibly HERV
WENRIQLDTLLAEKVGV

SEQ ID NO: 692 - protein, 17 aa, Unknown
  Possibly HERV
RENRIALDMILAKQGGV

SEQ ID NO: 693 - protein, 17 aa, Unknown
  Possibly HERV
WENRIALDMILVDRGGV

SEQ ID NO: 694 - protein, 17 aa, Unknown
  Possibly HERV
GKNRIALDTILAEKEGV

SEQ ID NO: 695 - protein, 17 aa, Unknown
  Possibly HERV
HENRIALDMLLAERGAV

SEQ ID NO: 696 - protein, 17 aa, Unknown
  Possibly HERV
WENKIALDMILAERVGV

SEQ ID NO: 697 - protein, 17 aa, Unknown
  Possibly HERV
WENRTALNMILAERGGV

SEQ ID NO: 698 - protein, 17 aa, Unknown
  Possibly HERV
WENRTALDMILATKGTV

SEQ ID NO: 699 - protein, 17 aa, Unknown
  Possibly HERV
WENRIALDMILAERGVC

SEQ ID NO: 700 - protein, 17 aa, Unknown
  Possibly HERV
WENRIALDMTLAENGRV

SEQ ID NO: 701 - protein, 17 aa, Unknown
  Possibly HERV
CENRIALDMISAERGGV

SEQ ID NO: 702 - protein, 17 aa, Unknown
  Possibly HERV
WENRIALDIILAKKGGV

SEQ ID NO: 703 - protein, 17 aa, Unknown
  Possibly HERV
WENRIALDMILAESGGV

SEQ ID NO: 704 - protein, 17 aa, Unknown
  Possibly HERV
WENRIALDMILTERGGV

SEQ ID NO: 705 - protein, 17 aa, Unknown
  Possibly HERV
WENRIALNMILAESGGV

SEQ ID NO: 706 - protein, 17 aa, Unknown
  Possibly HERV
WENRRALDTVLAERGVC

SEQ ID NO: 707 - protein, 17 aa, Unknown
  Possibly HERV
WENRIGLDMIIAERGGV

SEQ ID NO: 708 - protein, 17 aa, Unknown
  Possibly HERV
WENRMALDMILAEKGEV

SEQ ID NO: 709 - protein, 17 aa, Unknown
  Possibly HERV
WENRIALDMLLAKKGRV

SEQ ID NO: 710 - protein, 17 aa, Unknown
  Possibly HERV
WENRIALDMILAERGGV

SEQ ID NO: 711 - protein, 17 aa, Unknown
  Possibly HERV
GENRIALDMILTETGGV

SEQ ID NO: 712 - protein, 17 aa, Unknown
  Possibly HERV
WENRTALDMILAKRGGV

SEQ ID NO: 713 - protein, 17 aa, Unknown
  Possibly HERV
WENRIALDMIVAKRGRV

SEQ ID NO: 714 - protein, 17 aa, Unknown
  Possibly HERV
WENRIALDMILAERGGV

SEQ ID NO: 715 - protein, 17 aa, Unknown
  Possibly HERV
WENRIALDMVLAERGGV

SEQ ID NO: 716 - protein, 17 aa, Unknown
  Possibly HERV
WENRIALDMILAERGGV

SEQ ID NO: 717 - protein, 17 aa, Unknown
  Possibly HERV
WENRIPLDMIFAERGGV

SEQ ID NO: 718 - protein, 17 aa, Unknown
  Possibly HERV
WENRIALDMILTERGGV

SEQ ID NO: 719 - protein, 17 aa, Unknown
  Possibly HERV
WENRITLDMILTERGGV

SEQ ID NO: 720 - protein, 17 aa, Unknown
  Possibly HERV
WENRIALDMILAERRGV

SEQ ID NO: 721 - protein, 17 aa, Unknown
  Possibly HERV
GENRTALDMILAERGGV

SEQ ID NO: 722 - protein, 17 aa, Unknown
  Possibly HERV
GENRIALDMILAERGGV

SEQ ID NO: 723 - protein, 17 aa, Unknown
  Possibly HERV
RKNRITLDMILAEKGGV

SEQ ID NO: 724 - protein, 17 aa, Unknown
  Possibly HERV
WENRTALDMILAERGGV

SEQ ID NO: 725 - protein, 17 aa, Unknown
  Possibly HERV
WENRIASDMILAESGGV

SEQ ID NO: 726 - protein, 17 aa, Unknown
  Possibly HERV
WENRIALDMILAESGGV

SEQ ID NO: 727 - protein, 17 aa, Unknown
  Possibly HERV
WENRIALDMTLAERGGV

SEQ ID NO: 728 - protein, 17 aa, Unknown
  Possibly HERV
LQNQMALDILTAPQGGT

SEQ ID NO: 729 - protein, 17 aa, Unknown
  Possibly HERV
LQNWMAIDIVTAAQGGI

SEQ ID NO: 730 - protein, 17 aa, Unknown
  Possibly HERV
LQNRMALDIITAAQGGT

SEQ ID NO: 731 - protein, 17 aa, Unknown
  Possibly HERV
VQNQRALDIITAAQGAV

SEQ ID NO: 732 - protein, 17 aa, Unknown
  Possibly HERV
LQNQMALDIVAAAQGGT

SEQ ID NO: 733 - protein, 17 aa, Unknown
  Possibly HERV
LKNCMALDILPAAQGGT

SEQ ID NO: 734 - protein, 17 aa, Unknown
  Possibly HERV
LQNWMASDILTAAQGGT

SEQ ID NO: 735 - protein, 17 aa, Unknown
  Possibly HERV
LQNRMDLDIVTAAQGGT

SEQ ID NO: 736 - protein, 17 aa, Unknown
  Possibly HERV
WQNRMALGIITAAQGGT

SEQ ID NO: 737 - protein, 17 aa, Unknown
  Possibly HERV
LQNRMALNIVTVAQGGT

SEQ ID NO: 738 - protein, 17 aa, Unknown
  Possibly HERV
LQNQMALDIITTARGGT

SEQ ID NO: 739 - protein, 17 aa, Unknown
  Possibly HERV
WQNRMALDIVIASQGGT

SEQ ID NO: 740 - protein, 17 aa, Unknown
  Possibly HERV
MQNRMALDIVTAAQGGT

SEQ ID NO: 741 - protein, 17 aa, Unknown
  Possibly HERV
LQNQIALDIETAAQGGT

SEQ ID NO: 742 - protein, 17 aa, Unknown
  Possibly HERV
LQTRMALDILTAAQGGT

SEQ ID NO: 743 - protein, 17 aa, Unknown
  Possibly HERV
LQNRIALDIITAAQGGT

SEQ ID NO: 744 - protein, 17 aa, Unknown
  Possibly HERV
LQNQMALDILTAAQGGT

SEQ ID NO: 745 - protein, 17 aa, Unknown
  Possibly HERV
LQNEMALDIVTAAQGST

SEQ ID NO: 746 - protein, 17 aa, Unknown
  Possibly HERV
LQNRMALDIVTAAQGGI

SEQ ID NO: 747 - protein, 17 aa, Unknown
  Possibly HERV
LQNQMALDTVTATQGGT

SEQ ID NO: 748 - protein, 17 aa, Unknown
  Possibly HERV
LQNRMALDIATAAQGGT

SEQ ID NO: 749 - protein, 17 aa, Unknown
  Possibly HERV
LQNRMALDMVRAAQCGS

SEQ ID NO: 750 - protein, 17 aa, Unknown
  Possibly HERV
LQKWMVLDILTAAQGGT

SEQ ID NO: 751 - protein, 17 aa, Unknown
  Possibly HERV
LQNRMSLDIVTTAQGGT

SEQ ID NO: 752 - protein, 17 aa, Unknown
  Possibly HERV
LQNWMALDIVTADQGGT

SEQ ID NO: 753 - protein, 17 aa, Unknown
  Possibly HERV
LQNRMALDMRTAAQGGV

SEQ ID NO: 754 - protein, 17 aa, Unknown
  Possibly HERV
LQNCMAQDILTAYQGRT

SEQ ID NO: 755 - protein, 17 aa, Unknown
  Possibly HERV
VANGKALDMLTAAQGGV

SEQ ID NO: 756 - protein, 17 aa, Unknown
  Possibly HERV
LQNQMALKMLTAAQGGV

SEQ ID NO: 757 - protein, 17 aa, Unknown
  Possibly HERV
LQNQMALDMLTATQGGV

SEQ ID NO: 758 - protein, 17 aa, Unknown
  Possibly HERV
LQNQMALDMLTAAQGGI

SEQ ID NO: 759 - protein, 17 aa, Unknown
  Possibly HERV
LQNQMALDMLTAAQGGV

SEQ ID NO: 760 - protein, 17 aa, Unknown
  Possibly HERV
LQNQMALDMLTTAQGGV

SEQ ID NO: 761 - protein, 17 aa, Unknown
  Possibly HERV
LQNQMALDMLTTAQGGV

SEQ ID NO: 762 - protein, 17 aa, Unknown
  Possibly HERV
LQNRMTLDMLTAAQGEF

SEQ ID NO: 763 - protein, 17 aa, Unknown
  Possibly HERV
LQHGMALDMLTAVQGGV

SEQ ID NO: 764 - protein, 17 aa, Unknown
  Possibly HERV
LQNQMALDTLTSAQGGF

SEQ ID NO: 765 - protein, 17 aa, Unknown
  Possibly HERV
LQNQMALHILTAAQGRV

SEQ ID NO: 766 - protein, 17 aa, Unknown
  Possibly HERV
LENRMTLDMLTTAQGGV

SEQ ID NO: 767 - protein, 17 aa, Unknown
  Possibly HERV
LQNRMALDMLTAAQGGV

SEQ ID NO: 768 - protein, 17 aa, Unknown
  Possibly HERV
LQNRMALGMFTAAQGGV

SEQ ID NO: 769 - protein, 17 aa, Unknown
  Possibly HERV
LQNRMTLNTLTTAQGGV

SEQ ID NO: 770 - protein, 17 aa, Unknown
  Possibly HERV
LQNREALDMLTTAQGGV

SEQ ID NO: 771 - protein, 17 aa, Unknown
  Possibly HERV
LQNHVAPDMLTAAQGGV

SEQ ID NO: 772 - protein, 17 aa, Unknown
  Possibly HERV
WENRLALHKILAEKGGV

SEQ ID NO: 773 - protein, 17 aa, Unknown
  Possibly HERV
WEKRLELDMILADKGGI

SEQ ID NO: 774 - protein, 17 aa, Unknown
  Possibly HERV
WENRLALNMILLEKGGI

SEQ ID NO: 775 - protein, 17 aa, Unknown
  Possibly HERV
WENRLALDKILAEKGGI

SEQ ID NO: 776 - protein, 17 aa, Unknown
  Possibly HERV
LGNRLVLDIIIEEKGGI

SEQ ID NO: 777 - protein, 17 aa, Unknown
  Possibly HERV
WENRLALDMILEKKGAC

SEQ ID NO: 778 - protein, 17 aa, Unknown
  Possibly HERV
WENRCALDMILAEKGAY

SEQ ID NO: 779 - protein, 17 aa, Unknown
  Possibly HERV
WENRLVLDIILAEKRGI

SEQ ID NO: 780 - protein, 17 aa, Unknown
  Possibly HERV
WENRLSLDIIPAEKGGI

SEQ ID NO: 781 - protein, 17 aa, Unknown
  Possibly HERV
WENRLALNIILADKGDI

SEQ ID NO: 782 - protein, 17 aa, Unknown
  Possibly HERV
WENRLVLDMMLAEKGGI

SEQ ID NO: 783 - protein, 17 aa, Unknown
  Possibly HERV
WENRLQLDIILAEKGVV

SEQ ID NO: 784 - protein, 17 aa, Unknown
  Possibly HERV
WENRITLDIILAEKGDI

SEQ ID NO: 785 - protein, 17 aa, Unknown
  Possibly HERV
WENRIALDMILAEKGSV

SEQ ID NO: 786 - protein, 17 aa, Unknown
  Possibly HERV
WENRITLDIILAEKGDI

SEQ ID NO: 787 - protein, 17 aa, Unknown
  Possibly HERV
WENRITLDIILAEKGDI

SEQ ID NO: 788 - protein, 17 aa, Unknown
  Possibly HERV
WENRITLDIILAEKGDI

SEQ ID NO: 789 - protein, 17 aa, Unknown
  Possibly HERV
WENRITLDIILAEKGDI

SEQ ID NO: 790 - protein, 17 aa, Unknown
  Possibly HERV
WENRITLDIILAEKGDI

SEQ ID NO: 791 - protein, 17 aa, Unknown
  Possibly HERV
WENRITLDIILAEKGDI

SEQ ID NO: 792 - protein, 17 aa, Unknown
  Possibly HERV
WENRLALDMILAEKGGV

SEQ ID NO: 793 - protein, 17 aa, Unknown
  Possibly HERV
WENRLTLHIILAEKVGV

SEQ ID NO: 794 - protein, 17 aa, Unknown
  Possibly HERV
WENKLALNIILAEKCGI

SEQ ID NO: 795 - protein, 17 aa, Unknown
  Possibly HERV
WENRLALNIILAEKGSV

SEQ ID NO: 796 - protein, 17 aa, Unknown
  Possibly HERV
LGNRLVLDMILAEKGGV

SEQ ID NO: 797 - protein, 17 aa, Unknown
  Possibly HERV
LQNCRGLDLLFLSQRGL

SEQ ID NO: 798 - protein, 17 aa, Unknown
  Possibly HERV
LQNCRGLDLLFLSQGGL

SEQ ID NO: 799 - protein, 17 aa, Unknown
  Possibly HERV
LQTTKGLDLLFLSQGGL

SEQ ID NO: 800 - protein, 17 aa, Unknown
  Possibly HERV
LQNRRGLDLLFLSQGGL

SEQ ID NO: 801 - protein, 17 aa, Unknown
  Possibly HERV
LQNCRCLDLLFLSQGGL

SEQ ID NO: 802 - protein, 17 aa, Unknown
  Possibly HERV
LQNRRGLDLLFLSQGGL

SEQ ID NO: 803 - protein, 17 aa, Unknown
  Possibly HERV
LQNPRGLDLLFLSQGGL

SEQ ID NO: 804 - protein, 17 aa, Unknown
  Possibly HERV
LQNRRGLDLLFISQGGL

SEQ ID NO: 805 - protein, 17 aa, Unknown
  Possibly HERV
LQNRRGLDLLFLSQGGL

SEQ ID NO: 806 - protein, 17 aa, Unknown
  Possibly HERV
MQNRQALDILMAKVGGT

SEQ ID NO: 807 - protein, 17 aa, Unknown
  Possibly HERV
AQNRRGLDVLTAEVGGT

SEQ ID NO: 808 - protein, 17 aa, Unknown
  Possibly HERV
AQNRWALDVLTAEVGST

SEQ ID NO: 809 - protein, 17 aa, Unknown
  Possibly HERV
MQNRQTLDVLMAEVRGA

SEQ ID NO: 810 - protein, 17 aa, Unknown
  Possibly HERV
AQNRQALGVLIAEVGGT

SEQ ID NO: 811 - protein, 17 aa, Unknown
  Possibly HERV
AQNRQDLDVLTAEIGST

SEQ ID NO: 812 - protein, 17 aa, Unknown
  Possibly HERV
AQNRRALDVLTGEVGGT

SEQ ID NO: 813 - protein, 17 aa, Unknown
  Possibly HERV
AQNRWALDVLMAEIGGT

SEQ ID NO: 814 - protein, 17 aa, Unknown
  Possibly HERV
IQNRQALDVLTAKGGGP

SEQ ID NO: 815 - protein, 17 aa, Unknown
  Possibly HERV
LQNMRGLDLLTAEKCDL

SEQ ID NO: 816 - protein, 17 aa, Unknown
  Possibly HERV
VPNRQALDVLTAEVGGT

SEQ ID NO: 817 - protein, 17 aa, Unknown
  Possibly HERV
AQNRQALDVLTAEVGGT

SEQ ID NO: 818 - protein, 17 aa, Unknown
  Possibly HERV
AQNRQALDYLMAEVRDT

SEQ ID NO: 819 - protein, 17 aa, Unknown
  Possibly HERV
AQNRQALDVLTTEVGVL

SEQ ID NO: 820 - protein, 17 aa, Unknown
  Possibly HERV
MQNRRALDVLTAEVGAL

SEQ ID NO: 821 - protein, 17 aa, Unknown
  Possibly HERV
AQNRWALDVLTAEVGGT

SEQ ID NO: 822 - protein, 17 aa, Unknown
  Possibly HERV
AQNRWALDVLTAEVGGT

SEQ ID NO: 823 - protein, 17 aa, Unknown
  Possibly HERV
YQTRLALDYLLAAEGGV

SEQ ID NO: 824 - protein, 17 aa, Unknown
  Possibly HERV
YQNILALDYLLAAEGEV

SEQ ID NO: 825 - protein, 17 aa, Unknown
  Possibly HERV
YQNRLALNYLLAAEGEV

SEQ ID NO: 826 - protein, 17 aa, Unknown
  Possibly HERV
YQNRLALEYLLAAEEGV

SEQ ID NO: 827 - protein, 17 aa, Unknown
  Possibly HERV
YQNRLAPNYLLAAEGGV

SEQ ID NO: 828 - protein, 17 aa, Unknown
  Possibly HERV
YQNSLALDCLLAAEGEV

SEQ ID NO: 829 - protein, 17 aa, Unknown
  Possibly HERV
CQNRLALDYLLAAEGGV

SEQ ID NO: 830 - protein, 17 aa, Unknown
  Possibly HERV
YQNRLALNYLLAAEGKV

SEQ ID NO: 831 - protein, 17 aa, Unknown
  Possibly HERV
YQNRLALDYLLAAEEGV

SEQ ID NO: 832 - protein, 17 aa, Unknown
  Possibly HERV
YENRLALDYLLAAEGGI

SEQ ID NO: 833 - protein, 17 aa, Unknown
  Possibly HERV
HQNRLALDYLLAAEGGV

SEQ ID NO: 834 - protein, 17 aa, Unknown
  Possibly HERV
YQNRLALHYLLAAEEGV

SEQ ID NO: 835 - protein, 17 aa, Unknown
  Possibly HERV
YQNRLALDNLLAAEGGV

SEQ ID NO: 836 - protein, 17 aa, Unknown
  Possibly HERV
YQNRLALDYLLAAEGEV

SEQ ID NO: 837 - protein, 17 aa, Unknown
  Possibly HERV
YQNRLALDYLLAAEGGV

SEQ ID NO: 838 - protein, 17 aa, Unknown
  Possibly HERV
YQNRLALDYLLAAEGEV

SEQ ID NO: 839 - protein, 17 aa, Unknown
  Possibly HERV
YQNRLALDYLLAAEGEV

SEQ ID NO: 840 - protein, 17 aa, Unknown
  Possibly HERV
YQNRLALDYLLAAEGEV

SEQ ID NO: 841 - protein, 17 aa, Unknown
  Possibly HERV
YKSRLALDYLLAAEGGI

SEQ ID NO: 842 - protein, 17 aa, Unknown
  Possibly HERV
YQNRLALNYLLAAEGGG

SEQ ID NO: 843 - protein, 17 aa, Unknown
  Possibly HERV
YQNRLALDYLLAAEGKV

SEQ ID NO: 844 - protein, 17 aa, Unknown
  Possibly HERV
SQNRPALDYLLAAEGGV

SEQ ID NO: 845 - protein, 17 aa, Unknown
  Possibly HERV
YQNRLALDYLLAAEGGI

SEQ ID NO: 846 - protein, 17 aa, Unknown
  Possibly HERV
YQNRLALNYLLAAEGEV

SEQ ID NO: 847 - protein, 17 aa, Unknown
  Possibly HERV
WENRIALDMTLAERGGV

SEQ ID NO: 848 - protein, 17 aa, Unknown
  Possibly HERV
YQNRLALDYLLAAEREV

SEQ ID NO: 849 - protein, 17 aa, Unknown
  Possibly HERV
HKNRLALDYLLAAEGGV

SEQ ID NO: 850 - protein, 17 aa, Unknown
  Possibly HERV
YQNRLALDYLLAAERGV

SEQ ID NO: 851 - protein, 17 aa, Unknown
  Possibly HERV
YQNRPALDYLLAAEFNL

SEQ ID NO: 852 - protein, 17 aa, Unknown
  Possibly HERV
YQNRLALDYLLAAEGGV

SEQ ID NO: 853 - protein, 17 aa, Unknown
  Possibly HERV
YQNRLALDYLLAAEGEV

SEQ ID NO: 854 - protein, 17 aa, Unknown
  Possibly HERV
CQNRLALNYLLAAEGKV

SEQ ID NO: 855 - protein, 17 aa, Unknown
  Possibly HERV
YQNRLVLDHLLAAEGEV

SEQ ID NO: 856 - protein, 17 aa, Unknown
  Possibly HERV
YQNRLAFDYLLAAKGGV

SEQ ID NO: 857 - protein, 17 aa, Unknown
  Possibly HERV
YRNRLALDYLLAAEGEV

SEQ ID NO: 858 - protein, 17 aa, Unknown
  Possibly HERV
YQNRSALDYLLAAEGEV

SEQ ID NO: 859 - protein, 17 aa, Unknown
  Possibly HERV
YQNRLALDYLLAAEGGV

SEQ ID NO: 860 - protein, 17 aa, Unknown
  Possibly HERV
YQNRLTLDYLLATEGEV

SEQ ID NO: 861 - protein, 17 aa, Unknown
  Possibly HERV
YQNKLALNYLLAAEGRV

SEQ ID NO: 862 - protein, 17 aa, Unknown
  Possibly HERV
YQNRLALDYLLAAEGEV

SEQ ID NO: 863 - protein, 17 aa, Unknown
  Possibly HERV
YQNRLALNYLLAAEGGV

SEQ ID NO: 864 - protein, 17 aa, Unknown
  Possibly HERV
YQNRLALDYLLAAEGKV

SEQ ID NO: 865 - protein, 17 aa, Unknown
  Possibly HERV
YQNGLALDYLLAAEGEV

SEQ ID NO: 866 - protein, 17 aa, Unknown
  Possibly HERV
YQNRLALDYLLAAEREV

SEQ ID NO: 867 - protein, 17 aa, Unknown
  Possibly HERV
YQNRLALDYLLAAEGGV

SEQ ID NO: 868 - protein, 17 aa, Unknown
  Possibly HERV
YQNRLALDYLLAAEGGV

SEQ ID NO: 869 - protein, 17 aa, Unknown
  Possibly HERV
YQNRLALNYLLAAEGGV

SEQ ID NO: 870 - protein, 17 aa, Unknown
  Possibly HERV
YQNRLALDYLLAAEGGV

SEQ ID NO: 871 - protein, 17 aa, Unknown
  Possibly HERV
YQNRLALDYLLAAEGEV

SEQ ID NO: 872 - protein, 17 aa, Unknown
  Possibly HERV
YQNRLALDYLLAAEGGG

SEQ ID NO: 873 - protein, 17 aa, Unknown
  Possibly HERV
YQNRLALDYLLAAEREV

SEQ ID NO: 874 - protein, 17 aa, Unknown
  Possibly HERV
YQNRIVLDNLVAAEGGV

SEQ ID NO: 875 - protein, 17 aa, Unknown
  Possibly HERV
YQNRLALNYLLAAERRV

SEQ ID NO: 876 - protein, 17 aa, Unknown
  Possibly HERV
CQNRLALDYLLAAERGV

SEQ ID NO: 877 - protein, 17 aa, Unknown
  Possibly HERV
YQNRLALNYFLAAEGGV

SEQ ID NO: 878 - protein, 17 aa, Unknown
  Possibly HERV
YQNGLALDYLLATEGGV

SEQ ID NO: 879 - protein, 17 aa, Unknown
  Possibly HERV
YQNRLALDYLLAAEREV

SEQ ID NO: 880 - protein, 17 aa, Unknown
  Possibly HERV
YQNRLALDYLLAAERGV

SEQ ID NO: 881 - protein, 17 aa, Unknown
  Possibly HERV
YQNRLALDYLVAAERKV

SEQ ID NO: 882 - protein, 17 aa, Unknown
  Possibly HERV
YQNRLALDYLLLAEGGV

SEQ ID NO: 883 - protein, 17 aa, Unknown
  Possibly HERV
YQNRLALDYLLAAEGEV

SEQ ID NO: 884 - protein, 17 aa, Unknown
  Possibly HERV
WENRTALDMILAERGDV

SEQ ID NO: 885 - protein, 17 aa, Unknown
  Possibly HERV
YQNRLALDYLLAAGGGV

SEQ ID NO: 886 - protein, 17 aa, Unknown
  Possibly HERV
YQNRLALDYLLAAEGEV

SEQ ID NO: 887 - protein, 17 aa, Unknown
  Possibly HERV
YQNRLALDYLLAAEGEV

SEQ ID NO: 888 - protein, 17 aa, Unknown
  Possibly HERV
YQNRLALDYLLAAEREV

SEQ ID NO: 889 - protein, 17 aa, Unknown
  Possibly HERV
YQNRLALNYLLAAEGEV

SEQ ID NO: 890 - protein, 17 aa, Unknown
  Possibly HERV
YQNRLALDYLLAAEGEA

SEQ ID NO: 891 - protein, 17 aa, Unknown
  Possibly HERV
YQNRLVLDYLLAAEGGV

SEQ ID NO: 892 - protein, 17 aa, Unknown
  Possibly HERV
YQNRLALDCLPAAEEVC

SEQ ID NO: 893 - protein, 17 aa, Unknown
  Possibly HERV
YQNRLALDYLLATEGGV

SEQ ID NO: 894 - protein, 17 aa, Unknown
  Possibly HERV
YQNKLALDYLLEAKGGV

SEQ ID NO: 895 - protein, 17 aa, Unknown
  Possibly HERV
YQNRLALDYLPAAEGGG

SEQ ID NO: 896 - protein, 17 aa, Unknown
  Possibly HERV
YQNRLALNYLLAAEGAV

SEQ ID NO: 897 - protein, 17 aa, Unknown
  Possibly HERV
YQNRLALDYLLAAEGGV

SEQ ID NO: 898 - protein, 17 aa, Unknown
  Possibly HERV
YQKRLALDYLPAAEGGI

SEQ ID NO: 899 - protein, 17 aa, Unknown
  Possibly HERV
YQNRLALDYLLAAEGEV

SEQ ID NO: 900 - protein, 17 aa, Unknown
  Possibly HERV
VQNRTALDRILISQGGE

SEQ ID NO: 901 - protein, 17 aa, Unknown
  Possibly HERV
FQNCCTLDTLTASKGGV

SEQ ID NO: 902 - protein, 17 aa, Unknown
  Possibly HERV
FQNCCTLDTLTASKGGV

SEQ ID NO: 903 - protein, 17 aa, Unknown
  Possibly HERV
LQNCRELDMLMAEQGGI

SEQ ID NO: 904 - protein, 17 aa, Unknown
  Possibly HERV
LQNCQDLDMLLAAQGEI

SEQ ID NO: 905 - protein, 17 aa, Unknown
  Possibly HERV
LQNCRRLDTLTAAQGGI

SEQ ID NO: 906 - protein, 17 aa, Unknown
  Possibly HERV
MDNRLALDYLLAEQSRL

SEQ ID NO: 907 - protein, 17 aa, Unknown
  Possibly HERV
LQNRRGLDMLTAAQGGI

SEQ ID NO: 908 - protein, 17 aa, Unknown
  Possibly HERV
LQNCQGLDMLMAAQGGI

SEQ ID NO: 909 - protein, 17 aa, Unknown
  Possibly HERV
CQNCQGVDMLTAAQGEI

SEQ ID NO: 910 - protein, 17 aa, Unknown
  Possibly HERV
LDNRTALDCLLAEQAEI

SEQ ID NO: 911 - protein, 17 aa, Unknown
  Possibly HERV
LNNRIALDYLLAKQKYL

SEQ ID NO: 912 - protein, 17 aa, Unknown
  Possibly HERV
LNNRIALDYLVAEQKSI

SEQ ID NO: 913 - protein, 17 aa, Unknown
  Possibly HERV
LNNRIALDYLLAKQKYL

SEQ ID NO: 914 - protein, 17 aa, Unknown
  Possibly HERV
LNNRIALDYLLAKQRNT

SEQ ID NO: 915 - protein, 17 aa, Unknown
  Possibly HERV
LNNRIALDYLLAKQGSI

SEQ ID NO: 916 - protein, 17 aa, Unknown
  Possibly HERV
LNNRIALDYLLAKQRSI

SEQ ID NO: 917 - protein, 17 aa, Unknown
  Possibly HERV
LNNRIALDYLLAKQEYL

SEQ ID NO: 918 - protein, 17 aa, Unknown
  Possibly HERV
INNRIALDDLLAEKRSV

SEQ ID NO: 919 - protein, 17 aa, Unknown
  Possibly HERV
LNNRTALDYLLAKQQYL

SEQ ID NO: 920 - protein, 17 aa, Unknown
  Possibly HERV
LNNRIALDYLLAKQRSI

SEQ ID NO: 921 - protein, 17 aa, Unknown
  Possibly HERV
LNNRIALDYLLAKQGSI

SEQ ID NO: 922 - protein, 17 aa, Unknown
  Possibly HERV
LDNRIALDYLLAEQGRT

SEQ ID NO: 923 - protein, 17 aa, Unknown
  Possibly HERV
LNNRIALDYLLAKQRIT

SEQ ID NO: 924 - protein, 17 aa, Unknown
  Possibly HERV
MDNRITLDYILAEQGEV

SEQ ID NO: 925 - protein, 17 aa, Unknown
  Possibly HERV
LDNRITLDDLMAERGRI

SEQ ID NO: 926 - protein, 17 aa, Unknown
  Possibly HERV
LDNRIALDYLLAKQKYL

SEQ ID NO: 927 - protein, 17 aa, Unknown
  Possibly HERV
LNNRITLDYLLAVQGNV

SEQ ID NO: 928 - protein, 17 aa, Unknown
  Possibly HERV
LDKRIALNYLLAEQGRG

SEQ ID NO: 929 - protein, 17 aa, Unknown
  Possibly HERV
FDNRIALDCLLAEQGGI

SEQ ID NO: 930 - protein, 17 aa, Unknown
  Possibly HERV
LDNQIALDHLLAEKGEV

SEQ ID NO: 931 - protein, 17 aa, Unknown
  Possibly HERV
LDNRIVLDYLLAEQRGI

SEQ ID NO: 932 - protein, 17 aa, Unknown
  Possibly HERV
LDNRIVLDYLLAENARI

SEQ ID NO: 933 - protein, 17 aa, Unknown
  Possibly HERV
IDNRIALDYLLAEKRRI

SEQ ID NO: 934 - protein, 17 aa, Unknown
  Possibly HERV
LDNRIALDYLLAKQKGI

SEQ ID NO: 935 - protein, 17 aa, Unknown
  Possibly HERV
SYNRIALDYLLAEQGGI

SEQ ID NO: 936 - protein, 17 aa, Unknown
  Possibly HERV
LNNRIALNYLLAEQERI

SEQ ID NO: 937 - protein, 17 aa, Unknown
  Possibly HERV
LNNRIALDYLLAKQRCI

SEQ ID NO: 938 - protein, 17 aa, Unknown
  Possibly HERV
LDNRIALDYLLAKWGRI

SEQ ID NO: 939 - protein, 17 aa, Unknown
  Possibly HERV
LNNRIALDYLLAKQRSI

SEQ ID NO: 940 - protein, 17 aa, Unknown
  Possibly HERV
LDNRLALDFLLAEQKII

SEQ ID NO: 941 - protein, 17 aa, Unknown
  Possibly HERV
VNNRVALDYLLAEQGRI

SEQ ID NO: 942 - protein, 17 aa, Unknown
  Possibly HERV
LDNRIALDYLLAEEGDV

SEQ ID NO: 943 - protein, 17 aa, Unknown
  Possibly HERV
LDNRVALDYLLANQSNV

SEQ ID NO: 944 - protein, 17 aa, Unknown
  Possibly HERV
IDNRLALDHLLTKQGRI

SEQ ID NO: 945 - protein, 17 aa, Unknown
  Possibly HERV
LNNRLVLNYLLAKQDGV

SEQ ID NO: 946 - protein, 17 aa, Unknown
  Possibly HERV
LGNRLALDYLLAEQGGV

SEQ ID NO: 947 - protein, 17 aa, Unknown
  Possibly HERV
LDKRLALDYLLAEQGGG

SEQ ID NO: 948 - protein, 17 aa, Unknown
  Possibly HERV
LCNRLALDYLLAEQGEV

SEQ ID NO: 949 - protein, 17 aa, Unknown
  Possibly HERV
LNNRIASDYLLAEKGGV

SEQ ID NO: 950 - protein, 17 aa, Unknown
  Possibly HERV
LNNRIVLDYILTERDGV

SEQ ID NO: 951 - protein, 17 aa, Unknown
  Possibly HERV
LNNTQALDYLLAEQGEV

SEQ ID NO: 952 - protein, 17 aa, Unknown
  Possibly HERV
LDNQLALDYLLAEQGGV

SEQ ID NO: 953 - protein, 17 aa, Unknown
  Possibly HERV
LNNRIALDYLLAKQGGV

SEQ ID NO: 954 - protein, 17 aa, Unknown
  Possibly HERV
LDNCIALDCLLAEQGGG

SEQ ID NO: 955 - protein, 17 aa, Unknown
  Possibly HERV
LNSRLALDYLPAKQGGL

SEQ ID NO: 956 - protein, 17 aa, Unknown
  Possibly HERV
LDNRLALDYLLADQGSI

SEQ ID NO: 957 - protein, 17 aa, Unknown
  Possibly HERV
MNNRLALDYLLAEQGGV

SEQ ID NO: 958 - protein, 17 aa, Unknown
  Possibly HERV
MDNRLALDYLLAEQGGV

SEQ ID NO: 959 - protein, 17 aa, Unknown
  Possibly HERV
LNNTLALDYLLAEQGKV

SEQ ID NO: 960 - protein, 17 aa, Unknown
  Possibly HERV
FHNRLSLDCLLAEQGRV

SEQ ID NO: 961 - protein, 17 aa, Unknown
  Possibly HERV
LDNRIAFDYVLAEKGGV

SEQ ID NO: 962 - protein, 17 aa, Unknown
  Possibly HERV
LNNRLALDYLLAEQGRV

SEQ ID NO: 963 - protein, 17 aa, Unknown
  Possibly HERV
PDNRLVLDCLLTEKGRV

SEQ ID NO: 964 - protein, 17 aa, Unknown
  Possibly HERV
LNNRLALDYLLAEQGRV

SEQ ID NO: 965 - protein, 17 aa, Unknown
  Possibly HERV
LDNHLAFDYLLAEQGGI

SEQ ID NO: 966 - protein, 17 aa, Unknown
  Possibly HERV
MDNRLALHYLLVEQGGV

SEQ ID NO: 967 - protein, 17 aa, Unknown
  Possibly HERV
LDNRLALNYLLAESSGV

SEQ ID NO: 968 - protein, 17 aa, Unknown
  Possibly HERV
VDNRLALDYLLAEQGGV

SEQ ID NO: 969 - protein, 17 aa, Unknown
  Possibly HERV
LNHKLALDYLLVEQGGV

SEQ ID NO: 970 - protein, 17 aa, Unknown
  Possibly HERV
LNNRIALDYILAGRDGV

SEQ ID NO: 971 - protein, 17 aa, Unknown
  Possibly HERV
LNNRLALDYLLATQSGV

SEQ ID NO: 972 - protein, 17 aa, Unknown
  Possibly HERV
LDNRLALDYLLAEQDRV

SEQ ID NO: 973 - protein, 17 aa, Unknown
  Possibly HERV
LDNELALHYLLAEQGGI

SEQ ID NO: 974 - protein, 17 aa, Unknown
  Possibly HERV
LNNRLVLGHLLAEQGGV

SEQ ID NO: 975 - protein, 17 aa, Unknown
  Possibly HERV
LDNRLVLDYLLAKQGGD

SEQ ID NO: 976 - protein, 17 aa, Unknown
  Possibly HERV
LINRLALDYLLAEHGGV

SEQ ID NO: 977 - protein, 17 aa, Unknown
  Possibly HERV
LYKRLVLNYLLAEQGGV

SEQ ID NO: 978 - protein, 17 aa, Unknown
  Possibly HERV
LNNRLMLDYLLAEQGGV

SEQ ID NO: 979 - protein, 17 aa, Unknown
  Possibly HERV
IKNRLVLDYLLAEQVGV

SEQ ID NO: 980 - protein, 17 aa, Unknown
  Possibly HERV
LNNRLVLDYLPAEQSKV

SEQ ID NO: 981 - protein, 17 aa, Unknown
  Possibly HERV
LKNRLALDCLLAERGGV

SEQ ID NO: 982 - protein, 17 aa, Unknown
  Possibly HERV
PDNRLALDYLLVEQGGV

SEQ ID NO: 983 - protein, 17 aa, Unknown
  Possibly HERV
LDNRLALDYLLAIQGRV

SEQ ID NO: 984 - protein, 17 aa, Unknown
  Possibly HERV
LNNRLALDYLLVEQDGV

SEQ ID NO: 985 - protein, 17 aa, Unknown
  Possibly HERV
VDNRLALNYLLSEQGGI

SEQ ID NO: 986 - protein, 17 aa, Unknown
  Possibly HERV
LDNHLDLDYLLAEQGGI

SEQ ID NO: 987 - protein, 17 aa, Unknown
  Possibly HERV
FDNRLTLDYLPVEQEGV

SEQ ID NO: 988 - protein, 17 aa, Unknown
  Possibly HERV
LDNRIAFDYVLAEKGGV

SEQ ID NO: 989 - protein, 17 aa, Unknown
  Possibly HERV
LNNRITLDYLLAVQGNV

SEQ ID NO: 990 - protein, 17 aa, Unknown
  Possibly HERV
LDNRITLDRLLAQLGGV

SEQ ID NO: 991 - protein, 17 aa, Unknown
  Possibly HERV
LNNRIALNCLLAKQRRV

SEQ ID NO: 992 - protein, 17 aa, Unknown
  Possibly HERV
LQNQHALDVLTTKAGGT

SEQ ID NO: 993 - protein, 17 aa, Unknown
  Possibly HERV
LQNRTTLDVLTAEAGAH

SEQ ID NO: 994 - protein, 17 aa, Unknown
  Possibly HERV
FNNRQALDYPLAEQGGV

SEQ ID NO: 995 - protein, 17 aa, Unknown
  Possibly HERV
FGKRCALDYLLAEEGGV

SEQ ID NO: 996 - protein, 17 aa, Unknown
  Possibly HERV
LDNRRVSDYLLSAQGGV

SEQ ID NO: 997 - protein, 17 aa, Unknown
  Possibly HERV
LDNRQALDYLLAEQGGV

SEQ ID NO: 998 - protein, 17 aa, Unknown
  Possibly HERV
LNDRNALDFLLASQDGV

SEQ ID NO: 999 - protein, 17 aa, Unknown
  Possibly HERV
LDNHLALEYLLAAQGGV

SEQ ID NO: 1000 - protein, 17 aa, Unknown
  Possibly HERV
LKNCMALDILPAAQGGT

SEQ ID NO: 1001 - protein, 17 aa, Unknown
  Possibly HERV
LQNRMALDILTAAEGGT

SEQ ID NO: 1002 - protein, 17 aa, Unknown
  Possibly HERV
MQNRMSLDTLTAAQGGT

SEQ ID NO: 1003 - protein, 17 aa, Unknown
  Possibly HERV
LDNIIALDSILAEQGGI

SEQ ID NO: 1004 - protein, 17 aa, Unknown
  Possibly HERV
LQNQMALEMLTSVQGGT

SEQ ID NO: 1005 - protein, 17 aa, Unknown
  Possibly HERV
LQNRMGLDILTAAQGGS

SEQ ID NO: 1006 - protein, 17 aa, Unknown
  Possibly HERV
LQNRMALDIITTAQGGT

SEQ ID NO: 1007 - protein, 17 aa, Unknown
  Possibly HERV
LQNCMALDMLTAAQGGT

SEQ ID NO: 1008 - protein, 17 aa, Unknown
  Possibly HERV
LQNHMALDILTAAQSGT

SEQ ID NO: 1009 - protein, 17 aa, Unknown
  Possibly HERV
LQNRMAFDILTEAQGGT

SEQ ID NO: 1010 - protein, 17 aa, Unknown
  Possibly HERV
RPNCMALDILTAAQGGT

SEQ ID NO: 1011 - protein, 17 aa, Unknown
  Possibly HERV
LQKHMALDVLTVTQGGL

SEQ ID NO: 1012 - protein, 17 aa, Unknown
  Possibly HERV
LQNRMALHRLTAAQGRT

SEQ ID NO: 1013 - protein, 17 aa, Unknown
  Possibly HERV
LQNHMALDTLTAAQGGT

SEQ ID NO: 1014 - protein, 17 aa, Unknown
  Possibly HERV
IQNSVALDILTAAQEGT

SEQ ID NO: 1015 - protein, 17 aa, Unknown
  Possibly HERV
LQNCMVLDILTAAQGGI

SEQ ID NO: 1016 - protein, 17 aa, Unknown
  Possibly HERV
LQNFMALDILTGAQKRT

SEQ ID NO: 1017 - protein, 17 aa, Unknown
  Possibly HERV
LQNCMALDIFMAAQEGT

SEQ ID NO: 1018 - protein, 17 aa, Unknown
  Possibly HERV
LQNCMALEILMATQGGN

SEQ ID NO: 1019 - protein, 17 aa, Unknown
  Possibly HERV
LYNHMALDILIAAQGGT

SEQ ID NO: 1020 - protein, 17 aa, Unknown
  Possibly HERV
LQNCMALDILTAAQGGI

SEQ ID NO: 1021 - protein, 17 aa, Unknown
  Possibly HERV
LQNPMALDILTAAQEGT

SEQ ID NO: 1022 - protein, 17 aa, Unknown
  Possibly HERV
LQNQTALDILTAAQGGA

SEQ ID NO: 1023 - protein, 17 aa, Unknown
  Possibly HERV
LDNRFALEYLLAEQGRV

SEQ ID NO: 1024 - protein, 17 aa, Unknown
  Possibly HERV
LQNHMALDILTVAQGGT

SEQ ID NO: 1025 - protein, 17 aa, Unknown
  Possibly HERV
LQNCMCLDILTAAQGRT

SEQ ID NO: 1026 - protein, 17 aa, Unknown
  Possibly HERV
LQNRMALDILTAAPGGT

SEQ ID NO: 1027 - protein, 17 aa, Unknown
  Possibly HERV
SEKRLALDYLLAEREGC

SEQ ID NO: 1028 - protein, 17 aa, Unknown
  Possibly HERV
LQNHMALDILTAAQGGN

SEQ ID NO: 1029 - protein, 17 aa, Unknown
  Possibly HERV
LQNHVTLNILIAVQGGT

SEQ ID NO: 1030 - protein, 17 aa, Unknown
  Possibly HERV
LQNCMALVILTAIQGIT

SEQ ID NO: 1031 - protein, 17 aa, Unknown
  Possibly HERV
LQNHMALDILTAAQGGT

SEQ ID NO: 1032 - protein, 17 aa, Unknown
  Possibly HERV
LQNRMAMDTLIAAQGRT

SEQ ID NO: 1033 - protein, 17 aa, Unknown
  Possibly HERV
LQNCMALDILTVAQRGT

SEQ ID NO: 1034 - protein, 17 aa, Unknown
  Possibly HERV
LRNCMALDILTAAQGRS

SEQ ID NO: 1035 - protein, 17 aa, Unknown
  Possibly HERV
LRNHMALDILMAAQGNT

SEQ ID NO: 1036 - protein, 17 aa, Unknown
  Possibly HERV
LQNRVALDILTAAQREL

SEQ ID NO: 1037 - protein, 17 aa, Unknown
  Possibly HERV
LQNHMALDILTAAEKGT

SEQ ID NO: 1038 - protein, 17 aa, Unknown
  Possibly HERV
LQNCMALDTLAAAQGGA

SEQ ID NO: 1039 - protein, 17 aa, Unknown
  Possibly HERV
LQNCMALDILTAAQVRT

SEQ ID NO: 1040 - protein, 17 aa, Unknown
  Possibly HERV
LQNHIALDILTALKGGT

SEQ ID NO: 1041 - protein, 17 aa, Unknown
  Possibly HERV
LQNCMALDIVTAAQEGT

SEQ ID NO: 1042 - protein, 17 aa, Unknown
  Possibly HERV
LQNCGALDILAAAQGGT

SEQ ID NO: 1043 - protein, 17 aa, Unknown
  Possibly HERV
LDNRFALDYLLAEKGDT

SEQ ID NO: 1044 - protein, 555 aa, Unknown
  Possibly HERV
MILAGRAPSNTSTLMKFYSLLLYSLLFSFPFLYHPLPLPSYLHHTINLTHSLPAASNPSLANNCWLCISLSSSAYIAVPTLQTDRATSPVSLHLRTSFNSPHLYPPEELIYFLDRSSKTSPDISHQPAAALLHIYLKNLSPYINSTPPIFGPLTTQTTIPVAAPLCISRQRPTGIPLGNISPSRCSFTLHLQSPTTHVTETIGVFQLHIIDKPSINTDKLKNVSSNYCLGRHLPYISLHPWLPSPCSSDSPPRPSSCLLTPSPQNNSERLLVDTQRFLIHHENRTSSSMQLAHQSPLQPLTAAALAGSLGVWVQDTPFSTPSHPFSLHLQFCLTQGLFFLCGSSTYMCLPANWTGTCTLVFLTPKIQFANGTKELPVPLMTLTPQKRVIPLIPLMVGLGLSASTIALSTGIAGISTSVTTFRSPSNDFSASITDISQTLSVLQAQVDSLAAVVLQNRRGLGLSILLNEECCFYLNQSGLVYENIKKLKDRAQKLANQASNYAESPWALSNWMSWVLPILSPLIPIFLLLLFGPCIFHLVSQFIQNRIQAITNHSI

SEQ ID NO: 1045 - DNA, 1665 bp, Unknown
  Possibly HERV
atgatcttggctggcagggcaccctccaatacttccaccctgatgaagttctattctttacttttatactcactcttattctcattcccattcttataccaccctttacctctccccagctatctccaccacactatcaaccttacccattctctcccagctgcttctaatccctccttagcaaacaactgctggctttgcatttccctttcttccagtgcctacatagctgtccccaccttacagacagaccgggcaacatctcctgtctccttacacctccgaacttcctttaacagccctcacctttaccctcctgaagaactcatttactttctagacaggtccagcaaaacctccccagacatttcacatcagccagctgccgccctcctccacatttatttgaaaaacctttctccttatattaactctactccccccatatttggacctctcacaacacaaactactattcctgtggccgctcctttatgtatctctcggcaaagacccaccggaattcccctaggtaatatttcaccttctcgatgttcctttactctccatctccaaagcccaactacacacgtcactgaaacaattggagtcttccagctccatattatagacaagccctctatcaatactgacaaactcaaaaatgttagcagtaattattgcttaggaagacacttaccctatatttcactccatccttggctaccttccccttgctcgtcagactctcctcccaggccctcctcttgtttacttacacccagcccccaaaataacagtgaaaggttgctcgtagatactcaacgttttctcatacaccatgaaaatcgaacctcctcctctatgcagttagcccatcagtccccattacaacctttgacagctgccgccctagctggatccctaggagtctgggtacaagacacccctttcagcactccttctcacccttttagtttacatctccagttttgcctcacacaaggtctcttcttcctctgtggatcctctacctacatgtgtctacctgctaattggacaggcacatgcacactagtattccttacccccaaaattcaatttgcaaatgggaccaaagagctccctgttccccttatgacactgacaccacaaaaaagagttattccactaattcccttgatggtcggtttaggactttctgcctccactattgctctcagtactggaatagcaggcatttcaacctctgtcacgaccttccgtagcccgtctaatgacttctctgctagcatcacagacatatcacaaactttatcagtcctccaggcccaagttgactctttagctgcagttgtcctccaaaaccgccgaggccttggactcagtatattgttaaatgaagagtgttgtttttacctaaatcaatctggcttggtgtatgagaacataaaaaaactcaaggatagagcccaaaaacttgccaaccaagcaagtaattatgctgaatccccttgggcactctctaattggatgtcctgggtcctcccaattcttagtcctttaatacctatttttctccttcttttatttggaccttgtatcttccatttagtttctcaattcatccaaaaccgtatccaggctatcaccaatcattctata

SEQ ID NO: 1046 - DNA, 7721 bp, Unknown
  Possibly HERV
gcatgttagggaaaactgaataaggtgatttaagttatgcatgatatggaattgcgtggggctaaaaaagatgggaagatcttgaagagtattttatagtaaagaatactggaattgcattttagaagacttgaataaattacttagacaatgaaaaggaggaaaagtaaagaaagaagcctatacagaatttggaaacaatatttgtcaggcctgagcccaagccaagccattgcatcccctgtgacttgcacatatacgcccagatggcctgaagtaactgaagaaccacaaaagaagtgaatatgccctgccccgccttaaatgacattccaccacaaaaaaagtgaaaatggcctgttcccgccttaactgatgacattccaccacaaaaaaagtgaaaatggcctgttcctgccttaactgatgacattgtcttgtgaaattccttctcctggctcatcctgtctcaaaagctcccccactgagcaccttgtgacccccccactctgcccgccagagaacaactccccttcgactgtaattttcctttatttacccaaatcctataaaacggccccacccttatctcccttccctgactctcttttcggactcagcccacctgcacccaagtgattaaaagcttttattgctcacacaaagcctgtttggtggtctcctcacatggacgcgcatgaaatttggtgccatgactcggatcaggggacctcccttgggagatccaatcccctgtcctcctgttctttgctccatgaaaaagatccacctacgacctcaggtcctcagacccaccagcccaaggaacatttcaccaattttaaatcgggtaagcgacctctttttactctcttctccagccttcctcactatccctcaacctctttctcctttcaatcttggcgccacacttcaatctctcccttatcttaatttcaattcctttcattttctggtagagacaaaggagacacgttttatccatggacccaaaactccggcgccggtcacggactagggaaggcagccttcccttggtgtttaatcattgcagggatgccgctctgattattcccccatgtttcagaggtgtcagaccacacagggacgcctgccttggtccttcacccttagtggcaaatcccgcttttctggggaaggggcaagtaccccaaccccttctctccgtgtctctaccccttctctgcctttctgggggtcaagaaacccccaaccccttctccttcaccctgagtggcaagtcccacttttctgggggagggtcaagtaccccaacctcctatctctgtaccctgatcccttatttccacaccccaacctcttatatctctgcgccctgatcccttatttctgcaccccaacctcttatatctctgcaccccgatcccttatttccatgccccgacctcgtatctctgtgccccgacctcttctctgcttttctggagggcaagaaccccccaaccccttctctgtgtctctactcttttctctgggcttgcctccttcactatgggcaagcttccaccttccattcctccttcttctcccttagcctgtgttcttaagaacttaaaacctcttcaaccctcacctgacctaaaatctaagcatcttattttcttctgcaatgccgcttgaccccaatacaaacttgacagtagttccaaatagccggaaaacagcactttcaatttttccatcctacaagatctaaataattcttgtcgtaaaatgggcaaatgatctgacgtgcctgacgtccaggcattcttttacacattggtccctctctagtctctgttccgaatgcaactcatcccaaatcttccatttttccctcccacctgtcccctcagtcccaaccccaagcgtcgctgagtctttctaatcttccttttctacagacccatctgacctctcccctcctccccaggctgctcctcgccaggccgagctaggtcccaattcttcctcagcctccgctcctccaccctataatccttttatcacctgccctcctcacacccagtccggcttacagttttgttccatgactagccctcccccacctgcccagcaatttactcttaaaaaggtggttggagttaaggcatagtcaaggttaatgctcctttttctttatcccaaatcaggtagcgtttgggctctttttcatcaaatataaaaatccagcccagttcatgacttgtttggcagcaaccctgagacactttacagccctagaccctaaaatgtcaaaaggccgtcttattctcaaaatacattttattacccaatctgctcccgacattaaatgaaactccaaaaattaaattctggctctcaaaccccacaacaggatttaattaacctcgccttcaaggtgtacaataatagaaaaaagttgcaattccttgcctccactgtgagacaaaccccagccacatctccagcacacaagaacttccaaacgcctgaaccacagcagccaggcgttcctccagaacctcctcccccaggagcttgctacaagtgccagaaatctgaccaccaggccaaggaatgcttgcagcccaggattcctcttaagccgtgtcccatctgtgcgggaccccactggaaatcggactgttcaactcacctggcagccactcccagagcccctggatctctggcccaacgctgtgtgactgactccttttcggcttagcggctgaagactgactctgcctgatcacctcggaagccccgtagaccatcacagacgccaagctttaggtaactctcacagtggaaggtaagtccttccccttcttaatccatacggaggctacccactccacattaccttattttcaaggacctgtttcccttgcctccataactgttgtggatattgacagccaggcttctaaacctcttaaaactccccaactctggtgccaacttagacaatactcttttaagcactctttttagttatccccacctgcccagttcccttattaggccgagacactttaactaaattatctgcttccctgactagtcctggattacagctgcatctcattgctgcccttcttcccaatccaaagcctcctttgcgtcctcctcttgcattcccccaccttaacccacaagtgtaagatacctctactccctccttggcgactgatcatgcaccccttaccatctcattaaaacctaatcacccttaccccgctcaatgccaatatcccatcccacagcatgctttgaaaagattaaagcctgttatcacttgcctgctacagcatggccttttaaagcctataaactctccttacaattcccccattttacctgtcctaaaaccagacaagccttacaagttagttcaggatctatgccttatcaaccaaattgttttgcctatccaccccatggtgccaaacccatatactctcctatcctcaatacctccctccacaatccattattctgttgtggatctcaaacatgctttctttactattccttcacacccatcatcccagcctctcttcgctttcacttggactgaccctgacacccatcaggctcagcaaattacctgggctgactgccgcaaagcttcacagacagcccccattacttcaatcaagcccaaatttcttccttatctggtacctatctcagcataattctcataaaaacacacgtgctctccctgctgatcctgtccaattaatctcccaaacctcaatcccttacaaaacaacaactttccttcctaggcatggtagtgcagttggaattcttacacaagaaccgggaccacgccctgtagccttttatccatacaacttgaccttactggtttggcctagccctaaagttgtgtgcagcggccgccgccgccctaatacttttagaggcccttaaaatcacaaactatgctcaactcactctctacagttctcctaacttccaaaatctattttcttcctcacacctgacacatatattttctgctccccggctccttcagctgtactcactctttgttgagtctcccacagttgccattgttcctggcccggacttcaatccggccttccactttattcctgataccacacctgacccccatgactgtatctctctgatccacctgacattcaccccatttccccatatttccttctttcccattcctcaccctgatcacgcttgatttattgatggccgttccaccaggcctaatcgccacacaccagcaaaggcaggctatgctatagtacaagccactagcccgccttcttagaacctctcattttctttccatcgtggaaatctatcctcaaggaaataacttcgcagtgttccatctgctattctactactcctcagggattattcatgccccctcccttccctacacatcaagctcaaggatttgcacccacccaggactggcaaattagctttactcaacatgccccaagtcagataactaaaacaccttagtctaggtagacactttcactggataggtacaggcctttcctacagggtctgagaaggccaccacagtcatttcttcccttctgtcagacataattcctcagtttagccttcccacctctatacagtctgataacagaccagcctttattagtcaaatcagccaagcagtttttcaggctcttagtattcagtgaaacctttatatcccttacggtcctccgtcttcaggaaaagtagaacggactaaagatcttttaaaaacacaccttaccaagctcagccaccaactgaaaaaggactggacaatacttttaccactttcccttctcagaagtcagacctgtcctcagaatactacagggtacagcccatttgagctcccatatagatgctccttttattaggcccaagtctcattccagacaccagaccaacttagactgtgccccaaaataacttgtcatccctactattttctgtctagtcatactcctattctccgttctcaactacttataaatgccctacacttgtttacactgccggtttacactgccggttacactgtttcttcatgccatcacagctgatatctcttggtgctatccccaaaccaccactcttaattccctcttagagtgggtagatgatcttggctggcagggcaccctccaatacttccaccctgatgaagttctattctttacttttatactcactcttattctcattcccattcttataccaccctttacctctccccagctatctccaccacactatcaaccttacccattctctcccagctgcttctaatccctccttagcaaacaactgctggctttgcatttccctttcttccagtgcctacatagctgtccccaccttacagacagaccgggcaacatctcctgtctccttacacctccgaacttcctttaacagccctcacctttaccctcctgaagaactcatttactttctagacaggtccagcaaaacctccccagacatttcacatcagccagctgccgccctcctccacatttatttgaaaaacctttctccttatattaactctactccccccatatttggacctctcacaacacaaactactattcctgtggccgctcctttatgtatctctcggcaaagacccaccggaattcccctaggtaatatttcaccttctcgatgttcctttactctccatctccaaagcccaactacacacgtcactgaaacaattggagtcttccagctccatattatagacaagccctctatcaatactgacaaactcaaaaatgttagcagtaattattgcttaggaagacacttaccctatatttcactccatccttggctaccttccccttgctcgtcagactctcctcccaggccctcctcttgtttacttacacccagcccccaaaataacagtgaaaggttgctcgtagatactcaacgttttctcatacaccatgaaaatcgaacctcctcctctatgcagttagcccatcagtccccattacaacctttgacagctgccgccctagctggatccctaggagtctgggtacaagacacccctttcagcactccttctcacccttttagtttacatctccagttttgcctcacacaaggtctcttcttcctctgtggatcctctacctacatgtgtctacctgctaattggacaggcacatgcacactagtattccttacccccaaaattcaatttgcaaatgggaccaaagagctccctgttccccttatgacactgacaccacaaaaaagagttattccactaattcccttgatggtcggtttaggactttctgcctccactattgctctcagtactggaatagcaggcatttcaacctctgtcacgaccttccgtagcccgtctaatgacttctctgctagcatcacagacatatcacaaactttatcagtcctccaggcccaagttgactctttagctgcagttgtcctccaaaaccgccgaggccttggactcagtatattgttaaatgaagagtgttgtttttacctaaatcaatctggcttggtgtatgagaacataaaaaaactcaaggatagagcccaaaaacttgccaaccaagcaagtaattatgctgaatccccttgggcactctctaattggatgtcctgggtcctcccaattcttagtcctttaatacctatttttctccttcttttatttggaccttgtatcttccatttagtttctcaattcatccaaaaccgtatccaggctatcaccaatcattctatatgacaaatgtttcttctaacaaccccacaatatcaccccttaccacaagatctcccttcagctgaatctctccgactctaggttcccatgccacgcctaatcccgcttgaagcagccctgagaaacatcgcccattctctctctctataccccccaaaaattttcgccggcccaacacttcaacactatttggtttatttttcttattactataaggaggcaggaatgtcaggcctctgagcccaagccaagccatcgcatcccctgtgacttgcacatatacgcccagatggcctgaagtaacagaagaaccacaaaagaagtgaatatgccctgccccaccttaactgatgacattccaccacaaaaaaagtgaaaatggcctgttcttgccttaactgatgacattgtcttgtgaaattccttctcctggctcatcctgtctcaaaagctcccccactgagcaccttgtgaccccccactctgcccgccagagaacaactccccttcgactgtaattttcctttatttacccaaatcctataaaacggccccacccttatctcccttcccggactctcttttcagactcagcccgcctgcacccaggtgattaaaagcttttattgctcacacaaagcctgtttggtggtctcttcacagggacgctcatgaaaatattatgttttgcaaataaaagtttaggaagactagaacatggggtgtgagaagtgggtcaatatgggacatacatagagaaacaagtagggacattgacaatgtgtactataacctacaggaaatagttggatttttatgttcaaagcaactttttttagatgttaaaagggat

(19) United States
(12) Patent Application Publication
(10) Pub. No.: US 2019/0040106 A1
(43) Pub. Date: Feb. 7, 2019

(54) USE OF HUMAN DERIVED IMMUNOSUPPRESSIVE PROTEINS AND PEPTIDES AS MEDICAMENTS

(71) Applicant: AIMVION A/S, Aarhus N (DK)

(72) Inventors: Magdalena Janina Laska, Aarhus C (DK); Anne Margrethe Troldborg, Aarhus V (DK); Kristian Stengaard-Pedersen, Risskov (DK); Shervin Bahrami, Tilst (DK)

(73) Assignee: AIMVION A/S, Aarhus N (DK)

(21) Appl. No.: 15/764,714

(22) PCT Filed: Sep. 30, 2016
(86) PCT No.: PCT/DK2016/050316

§ 371 Date: Mar. 29, 2018

(30) Foreign Application Priority Data
Oct. 1, 2015 (DK) PA 2015 70620
Aug. 23, 2016 (DK) PA 2016 70634

(51) Int. Cl. C07K 14/005; A61P 37/06; A61P 19/02

(52) U.S. Cl. CPC C07K 14/005; A61P 37/06; A61K 38/00; C12N 2740/10022; C12N 2740/10033; A61P 19/02

(57) ABSTRACT

The present invention concerns uses of immune modulating and immune suppressing peptides from human endogenous retroviruses. In particular, the present invention concerns a use of an immune suppressive peptide for immune suppression and for reduction of inflammation.

The present invention relates to proteins related to human endogenous retrovirus and peptides derived from such proteins and use of the same for therapeutic applications. In particular, the present invention relates to immune modulating activity and immune suppressive domains (ISDs) related to human endogenous retrovirus and their use for immune modulation and for reduction of inflammation. Further, the invention relates to a class of multifunctional drugs for treatment of autoimmune diseases as well as inflammatory diseases. Additionally, the invention relates to pharmaceutical compositions comprising immune modulating proteins and peptides (proteins and peptides hereinafter generically referred to as polypeptides) that are derived from endogenous retroviruses.

Additionally the present invention relates to materials, surfaces and/or particles that are coupled to such a polypeptide. The present invention further relates to methods for producing the proteins, peptides and pharmaceutical compositions, as well as the usage of the same.

### TECHNICAL BACKGROUND

Retroviruses are a group of viruses that are characterized by containing an RNA genome, which upon infection is reverse transcribed into a DNA copy, which is subsequently integrated into the genome of the host cells. As a consequence hereof, all the progeny of such an infected cell will contain the viral genome (referred to as a pro-virus). All retroviruses include the following three genes/coding sequences: gag—which contains the structural proteins of the virus, pol—which contains the enzymes including the reverse transcriptase, and finally env—which encodes the viral surface glycoprotein, which is primarily responsible for viral entry into host cells as well as the immune suppressive activity demonstrated by many retroviruses. The present invention primarily relates the env gene and its protein product the ENV protein, derivatives thereof, peptides derived from this as well as the use of any of these compounds or entities.

Human Endogenous Retro Viruses (HERVs) are ancient retroviral integrations, which have been permanently fixed in the genome of humans. Although most of HERVs elements have accumulated numerous mutations and deletions, the existence of functional proteins for most viral components of HERVs have been demonstrated, including the viral protease and the envelope surface protein (Schmitt, Reichrath, Roesch, Meese, & Mayer, 2013; Tonjes et al., 1996; Willer et al., 1997). The selective pressure obviously exerted by evolution to maintain some functional HERV envelope open reading frames (ORFs) and restrict their expression to specific tissues suggests that HERV derived proteins may have developed to exhibit a significant physiological potential. For example, HERV derived envelope glycoproteins are abundantly expressed in placenta tissue (Boyd, Bax, Bax, Bloxam, & Weiss, 1993) and have been proposed to participate in syncytiotrophoblast differentiation by fusing the underlying cytotrophoblast cell layer (Venables, Brookes, Griffiths, Weiss, & Boyd, 1995).

Retroviral infections in general can cause significant immunosuppression. In particular some human endogenous retroviruses show immune suppressive activity and can for example antagonize the immune-dependent elimination of tumor cells transplanted into immunocompetent mice after transduction of these tumor cells by an envelope-expression vector (Mangeney & Heidmann, 1998).

The HERV-H family is one of the most abundant groups among human endogenous retroviruses, with approximately 1000 elements per haploid genome. Most of the HERV-H proviruses include deletions and/or mutations, rendering them without significant open reading frame activity. However, a small subset are structurally intact and have full-length gag, pol, and env domains. Among the approximately 100 HERV-H derived envelope genes, only three, including HERV-H Env59 (hereafter also referred to as “Env 59”), have the capacity to encode a large protein encompassing an immune suppressive domain (hereafter also referred to as an ISU domain or just ISD). Previous knowledge regarding ISD or ISU sequences derives primarily from exogenous murine gamma retroviruses. In this case the ISU sequence is located close to the C-terminal of the envelope protein.

The immune suppressive domain constitutes a small segment of the viral glycoprotein and is a major mediator of immune suppression by retroviruses. It is well known that retroviral envelope proteins have significant immunosuppressive activity. In gamma retroviruses, this activity is located to a well-defined structure (the so called ISD) in the retroviral transmembrane (TM) protein which is conserved among retroviruses of several species (including murine, feline, and human retroviruses including human T-cell leukemia virus).

### Autoimmunity and Autoimmune Diseases

Autoimmunity is the system of immune responses of an organism against its own healthy cells and tissues. While low levels of autoimmunity help the body maintenance, high levels of autoimmunity may cause disease. Any disease that results from such an aberrant immune response is termed an autoimmune disease. Autoimmune diseases have a wide variety of different effects. However the occurrence of one of three following characteristic pathological effects define a disease as autoimmune: damage to or destruction of tissue, altered organ growth or altered organ function.

There are more than 80 illnesses caused by autoimmunity and autoimmune diseases affects approximately 2-5% of the western world's population. Thus a substantial minority of the population suffers from these diseases, which are often chronic, debilitating, and life-threatening. Women are found to be more commonly affected than men and it has been estimated that autoimmune diseases are among the leading causes of death among women in the United States in all age groups up to 65 years. Environmental events can trigger some cases of autoimmune diseases such as exposure to radiation or certain drugs, which can damage tissues of the body. Infections can also be a trigger of some autoimmune diseases for example Lupus which is thought to be a milder version of an idiopathic disorder causing increased production of antihistone antibodies.

The treatment of autoimmune diseases is typically involves immunosuppressive medication that decreases the immune response. Novel treatments include Cytokine Blockade (therapeutic inhibition of cytokine signaling pathways), removal of effector T-cells and B-cells (e.g. anti-CD20 therapy can be effective at removing instigating B-cells) and intravenous immunoglobulin, which has been helpful in treating some antibody mediated autoimmune diseases as well.

A large number of autoimmune diseases have been recognized. Due to genome wide association scans, new insight into the underlying pathophysiology of autoimmune diseases has been obtainable. As an example, this technique have identified a striking degree of genetic sharing among the autoimmune diseases.

### Arthritis

Arthritis is a form of joint disorder that involves inflammation of one or more joints.

There are over 100 different forms of arthritis. The most common form, osteoarthritis (degenerative joint disease), is a result of trauma to the joint, infection of the joint, or age. Other arthritis forms are rheumatoid arthritis, psoriatic arthritis, and related autoimmune diseases. Septic arthritis is caused by joint infection.

A denominator of arthritis is joint pain. Pain is often a constant and may be localized to the joint affected. The pain from arthritis is due to inflammation that occurs around the joint, damage to the joint from disease, daily wear and tear of joint, muscle strains caused by forceful movements against stiff, painful joints and fatigue.

### Rheumatoid Arthritis

Rheumatoid arthritis (RA) is a long lasting autoimmune disorder that primarily affects joints. It typically results in warm, swollen, and painful joints. Pain and stiffness often worsen following rest. Most commonly the wrist and hands are involved with typically the same joints involved on both sides of the body. The disease may also affect other parts of the body. This may result in low red blood cells, inflammation around the lungs, and inflammation around the heart. Fever and low energy may also be present. Often symptoms come on gradually over weeks to months.

While the cause of rheumatoid arthritis is not clear, it is believed to involve a combination of genetic and environmental factors. The underlying mechanism involves the body's immune system attacking the joints. This results in inflammation and thickening of the joint capsule. It also affects the underlying bone and cartilage. The diagnosis is made mostly on the basis of a person's physical signs and symptoms, while X-rays and laboratory testing may support a diagnosis or exclude other diseases with similar symptoms. Other diseases that may present similarly include systemic lupus erythematosus, psoriatic arthritis, and fibromyalgia among others.

The goal of treatment is to decrease pain and inflammation, and improve a person's overall functioning. This may be helped by balancing rest and exercise, the use of splints and braces, or the use of assistive devices. Pain medications, steroids, and NSAIDs are frequently used to help with symptoms. A group of medications called disease-modifying antirheumatic drugs (DMARDs) may be used to try to slow the progression of disease. They include the medications hydroxychloroquine and methotrexate Biological DMARDs may be used when disease does not respond to other treatments. However, they may have a greater rate of adverse effects. Surgery to repair, replace, or fusion joints may help in certain situations. Most alternative medicine treatments are not supported by evidence.

RA affects between 0.5 and 1% of adults in the developed world with between 5 and 50 per 100,000 people newly developing the condition each year. Onset is most frequent during middle age and women are affected 2.5 times as frequently as men. In 2013 it resulted in 38,000 deaths up from 28,000 deaths in 1990. The term rheumatoid arthritis is based on the Greek for watery and inflamed joints.

Inflammatory synovitis in rheumatoid arthritis (and possibly in other inflammatory arthritidies) appears to be the result of an imbalance in the cytokine network with either an excess production of pro-inflammatory cytokines or from inadequacy of the natural anti-inflammatory mechanisms. In RA, several cytokines, e.g. interleukin (IL)-1, IL-6, IL-8, IL-12, IL-17, tumour necrosis factor-α (TNF-α), interferon-γ (IFN-γ) and granulocyte-macrophage colony-stimulating factor (GM-CSF), are involved in almost all aspects of articular inflammation and destruction.

Interleukin 6 (IL-6) plays a pivotal role in the pathophysiology of rheumatoid arthritis (RA). It is found in abundance in the synovial fluid and serum of patients with RA and the level correlates with the disease activity and joint destruction. IL-6 can promote synovitis and joint destruction by stimulating neutrophil migration, osteoclast maturation and vascular endothelial growth factor (VEGF)-stimulated pannus proliferation. IL-6 may also be mediating many of the systematic manifestations of RA including inducing the acute-phase reaction [including C-reactive protein (CRP)], anaemia through hecipidin production, fatigue via the hypothalamic-pituitary-adrenal (HPA) axis) and osteoporosis from its effect on osteoclasts. In addition, IL-6 may contribute to the induction and maintenance of the autoimmune process through B-cell maturation and TH-17 differentiation. All of the above makes IL-6 blockade a desirable therapeutic option in the treatment of RA. Following successful animal studies, a humanized anti-interleukin-6 receptor (anti-IL-6R) monoclonal antibody, tocilizumab (TCZ), entered into clinical trials and it has been shown to be an effective treatment in several large phase III clinical trials in RA with rapid and sustained improvement in disease activity, reducing radiographic joint damage and improving physical function (Srirangan & Choy, 2010).

### Systemic Lupus Erythematosus (SLE)

Systemic lupus erythematosus (SLE) is a chronic inflammatory disease of generalized autoimmunity characterized by B cell hyperactivity, abnormally activated T cells and defects in the clearance of apoptotic cells and immune complexes. The pathogenesis is still unclear, but a myriad of innate and adaptive immune system aberrations in SLE have been identified as major contributors of the disease.

An association between IL-6 and progression of lupus has been published for several murine models of SLE. Additionally data from several studies suggest that IL-6 plays a critical role in the B cell hyperactivity and immunopathology of human SLE, and may have direct role in mediating tissue damage. Lupus patients have elevated levels of serum IL-6 that correlated with disease activity or anti-DNA (anti-nuclear antibodies) levels in some, but not all studies (Peterson, Robertson, & Emlen, 1996). The most compelling evidence supporting a critical role for IL-6 in the pathogenesis of SLE was demonstrated by the beneficial effects of IL-6 receptor blockade and the exacerbating effect of IL-6 in NZB/WF₁ mice (Mihara, Takagi, Takeda, & Ohsugi, 1998).

### Inflammatory Bowel Disease

Inflammatory bowel disease (IBD) is a group of inflammatory conditions of the colon and small intestine. Crohn's disease and ulcerative colitis are the principal types of inflammatory bowel disease. It is important to note that not only does Crohn's disease affect the small intestine and large intestine, it can also affect the mouth, esophagus, stomach and the anus whereas ulcerative colitis primarily affects the colon and the rectum

Cytokines play a central role in the modulation of the intestinal immune system. They are produced by lymphocytes (especially T cells of the Th1 and Th2 phenotypes), monocytes, intestinal macrophages, granulocytes, epithelial cells, endothelial cells, and fibroblasts. They have proinflammatory functions [interleukin-1 (IL-1), tumor necrosis factor (TNF), IL-6, IL-8, IL-12] or anti-inflammatory functions [interleukin-1 receptor antagonist (IL-1ra), IL-4, IL-10, IL-11, transforming growth factor beta (TGF beta)]. Mucosal and systemic concentrations of many pro- and antiinflammatory cytokines are elevated in inflammatory bowel disease (IBD). An imbalance between proinflammatory and antiinflammatory cytokines was found for the IL-1/IL-1ra ratio in the inflamed mucosa of patients with Crohn's disease, ulcerative colitis, diverticulitis, and infectious colitis. Furthermore, the inhibition of proinflammatory cytokines and the supplementations with antiinflammatory cytokines reduced inflammation in animal models, such as the dextran sulfate colitis (DSS) model, the trinitrobenzene sulfonic acid (TNBS) model, or the genetically engineered model of IL-10 knockout mice. Based on these findings a rationale for cytokine treatment was defined. The first clinical trials using neutralizing monoclonal antibodies against TNF alpha (cA2) or the antiinflammatory cytokine IL-10 have shown promising results. However, many questions must be answered before cytokines can be considered standard therapy for IBD (Rogler & Andus, 1998).

Ulcerative colitis and Crohn's disease are chronic inflammatory disorders of the GI tract. Although the disorders can usually be distinguished on clinical and pathological criteria, there are similarities in natural history and response to therapy.

There is growing evidence that the pro-inflammatory cytokine interleukin IL-6 plays a crucial part in the uncontrolled intestinal inflammatory process, which is a main characteristic of IBD. There is elevated production of IL-6 and its soluble receptor (sIL-6R) by intestinal macrophages and CD4+ T-cells. The increased formation of IL-6-sIL-6R complexes that interact with gp130 on the membrane of CD4+ T-cells (trans-signaling) lead to an increased expression and nuclear translocation of STAT3, which causes the induction of anti-apoptotic genes, such as Bcl-xl. This leads to an augmented resistance of lamina propria T-cells to apoptosis. The ensuing T-cell expansion contributes to the perpetuation of chronic intestinal inflammation. This understanding concerning the predominant pathogenic role of an IL-6-dependent inflammatory cascade may lead to the development of new therapeutic strategies in the treatment of this disease.

### Sepsis

Sepsis is a potentially deadly medical condition characterized by a whole-body inflammatory state (called a systemic inflammatory response syndrome or SIRS) that is triggered by an infection. The body may develop this inflammatory response by the immune system to microbes in the blood, urine, lungs, skin, or other tissues. A lay term for sepsis is blood poisoning, also used to describe septicemia. Severe sepsis is the systemic inflammatory response, infection and the presence of organ dysfunction.

Severe sepsis is usually treated in the intensive care unit with intravenous fluids and antibiotics. If fluid replacement isn't sufficient to maintain blood pressure, specific vasopressor medications can be used. Mechanical ventilation and dialysis may be needed to support the function of the lungs and kidneys, respectively. To guide therapy, a central venous catheter and an arterial catheter may be placed; measurement of other hemodynamic variables (such as cardiac output, mixed venous oxygen saturation, or stroke volume variation) may also be used. Sepsis patients require preventive measures for deep vein thrombosis, stress ulcers and pressure ulcers, unless other conditions prevent this. Some patients might benefit from tight control of blood sugar levels with insulin (targeting stress hyperglycemia). The use of corticosteroids (low dose or otherwise) is controversial. Activated drotrecogin alfa (recombinant protein C) has not been found to be helpful, and has recently been withdrawn from sale.

In addition to symptoms related to the provoking infection, sepsis is characterized by presence of acute inflammation present throughout the entire body, and is, therefore, frequently associated with fever and elevated white blood cell count (leukocytosis) or low white blood cell count (leukopenia) and lower-than-average temperature, and vomiting. The modern concept of sepsis is that the host's immune response to the infection causes most of the symptoms of sepsis, resulting in hemodynamic consequences and damage to organs. This host response has been termed systemic inflammatory response syndrome (SIRS) and is characterized by an elevated heart rate (above 90 beats per minute), high respiratory rate (above 20 breaths per minute or a partial pressure of carbon dioxide in the blood of less than 32), abnormal white blood cell count (above 12,000, lower than 4,000, or greater than 10% band forms) and elevated or lowered body temperature, i.e. under 36° C. (96.8° F.) or over 38° C. (100.4° F.).

This immunological response causes widespread activation of acute-phase proteins, affecting the complement system and the coagulation pathways, which then cause damage to the vasculature as well as to the organs. Various neuroendocrine counter-regulatory systems are then activated as well, often compounding the problem. Even with immediate and aggressive treatment, this may progress to multiple organ dysfunction syndrome and eventually death.

Proinflammatory cytokines play a major role in the complications caused by sepsis.

In one study plasma levels of critically ill patients of resistin, active PAI-1, MCP-1, IL-1 alpha, IL-6, IL-8, IL-10, and TNF-alpha were significantly elevated compared to 60 healthy blood donors. Making these cytokines targets for downregulation by immunosuppressive peptides (Hillenbrand et al., 2010).

In a second study a prospective observational study was used to determine the predictive role of Tumor Necrosis Factor alpha (TNF-α), Interleukin (IL)-113 and IL-6 as three main pro-inflammatory cytokines in mortality of critically ill patients with severe sepsis.

It was found that among the three measured cytokines, sequential levels of TNF-α and IL-6 showed significant differences between survivors and nonsurvivors. IL-6 had a good correlation with outcome and scoring systems during the period of this study. Results of this study suggest that IL-6 is a useful cytokine in prediction of mortality and clinical evaluation of severe septic patients (Hamishehkar et al., 2010).

Autoimmune diseases also include Acute disseminated encephalomyelitis (ADEM), Addison's disease, Agammaglobulinemia, Alopecia areata, Amyotrophic Lateral Sclerosis, ANCA Vasculitis, Ankylosing Spondylitis, Antiphospholipid syndrome, Antisynthetase syndrome, Arteriosclerosis, Atopic allergy, Atopic dermatitis, Autoimmune aplastic anemia, Autoimmune cardiomyopathy, Autoimmune enteropathy, Autoimmune hemolytic anemia, Autoimmune hepatitis, Autoimmune inner ear disease, Autoimmune lymphoproliferative syndrome, Autoimmune peripheral neuropathy, Autoimmune pancreatitis, Autoimmune polyendocrine syndrome, Autoimmune progesterone dermatitis, Autoimmune thrombocytopenic purpura, Autoimmune urticaria, Autoimmune uveitis, Balo disease/Balo concentric sclerosis, Behget's disease, Berger's disease, Bickerstaff's encephalitis, Blau syndrome, Bullous pemphigoid, Cancer, Castleman's disease, Celiac disease, Chagas disease, Chronic inflammatory demyelinating polyneuropathy, Chronic recurrent multifocal osteomyelitis, Chronic obstructive pulmonary disease, Churg-Strauss syndrome, Cicatricial pemphigoid, Cogan syndrome, Cold agglutinin disease, Complement component 2 deficiency, Contact dermatitis, Cranial arteritis, CREST syndrome, Crohn's disease, Cushing's Syndrome, Cutaneous leukocytoclasticangiitis, Dego's disease, Dercum's disease, Dermatitis herpetiformis, Dermatomyositis, Diabetes mellitus type 1, Diffuse cutaneous systemic sclerosis, Dressler's syndrome, Drug-induced lupus, Discoid lupus erythematosus, Eczema, Endometriosis, Enthesitis-related arthritis, Eosinophilic fasciitis, Eosinophilic gastroenteritis, Epidermolysis bullosa acquisita, Erythema nodosum, Erythroblastosis fetalis, Essential mixed cryoglobulinemia, Evan's syndrome, Fibrodysplasia ossificans progressiva, Fibrosing alveolitis, Gastritis, Gastrointestinal pemphigoid, Glomerulonephritis, Goodpasture's syndrome, Graves' disease, Guillain-Barré syndrome (GBS), Hashimoto's encephalopathy, Hashimoto's thyroiditis, Henoch-Schonlein purpura, Herpes gestationis, hepatitis, Hidradenitis suppurativa, Hughes-Stovin syndrome, Hypogammaglobulinemia, Idiopathic inflammatory demyelinating diseases, Idiopathic pulmonary fibrosis, Idiopathic thrombocytopenic purpura, IgA nephropathy, Inclusion body myositis, Chronic inflammatory demyelinating polyneuropathy, Interstitial cystitis, Juvenile idiopathic arthritis, Kawasaki's disease, Lambert-Eaton myasthenic syndrome, Leukocytoclastic vasculitis, Lichen planus, Lichen sclerosus, Linear IgA disease (LAD), Lou Gehrig's disease, Lupoid hepatitis, Lupus erythematosus, Majeed syndrome, Ménière's disease, Microscopic polyangiitis, Miller-Fisher syndrome, Mixed connective tissue disease, Morphea, Mucha-Habermann disease, Multiple sclerosis, Myasthenia gravis, Myositis, Narcolepsy, Neuromyelitisoptica, Neuromyotonia, non-alcoholic steatohepatitis (NASH), Occularcicatricial pemphigoid, Opsoclonus myoclonus syndrome, Ord's thyroiditis, Palindromic rheumatism, PANDAS (pediatric autoimmune neuropsychiatric disorders associated with streptococcus), Paraneoplastic cerebellar degeneration, Paroxysmal nocturnal hemoglobinuria (PNH), Parry Romberg syndrome, Parsonage-Turner syndrome, Pars planitis, Pemphigus vulgaris, Pernicious anaemia, Perivenous encephalomyelitis, POEMS syndrome, Polyarteritisnodosa, Polymyalgia rheumatica, Polymyositis, Primary biliary cirrhosis, Primary sclerosing cholangitis, Progressive inflammatory neuropathy, Psoriasis, Psoriatic arthritis, Pyoderma gangrenosum, Pure red cell aplasia, Rasmussen's encephalitis, Raynaud phenomenon, Relapsing polychondritis, Reiter's syndrome, Restless leg syndrome, Retroperitoneal fibrosis, Rheumatoid arthritis, Rheumatic fever, Sarcoidosis, Schizophrenia, Schmidt syndrome, Schnitzler syndrome, Scleritis, Scleroderma, Serum Sickness, Sjögren's syndrome, Spondyloarthropathy, Still's disease, Stiff person syndrome, Subacute bacterial endocarditis (SBE), Susac's syndrome, Sweet's syndrome, Sydenham chorea, Sympathetic ophthalmia, Systemic lupus erythematosis, Takayasu's arteritis, Temporal arteritis, Thrombocytopenia, Tolosa-Hunt syndrome, Transverse myelitis, Ulcerative colitis, Undifferentiated connective tissue disease, Undifferentiated spondyloarthropathy, Urticarial vasculitis, Vasculitis, Vitiligo, and Wegener's granulomatosis.

### SUMMARY OF THE INVENTION

The inventors of the present invention have been able to show that proinflammatory cytokines, such as IL-6 and TNF-α, may be suppressed or activated by peptides and proteins of the present invention. Peptides and proteins of the present invention may provide active ingredients for the prophylaxis or treatment of conditions associated with autoimmune diseases or for immunotherapy e.g. when used as vaccine adjuvants.

According to an aspect, the present invention concerns a polypeptide consisting of or comprising a sequence having at least 62%, more preferred at least 75%, preferably at least 87%, more preferred 100% sequence identity to the sequence LSILLNEE (SEQ ID NO: 26).

According to an another aspect, the present invention concerns a polypeptide which includes a peptide sequence having at least 70% sequence identity or homology to the sequence LSILLNEE (SEQ ID NO: 26), and derivatives thereof, fragments thereof, complexes thereof, any tertiary structures thereof in the form of monomers, dimers, trimers, multimeres, helix structures and globular structures, and crosslinkings, and any chemical modifications thereof to increase physical chemical form and properties and bioavailability.

A polypeptide of the invention may e.g. be in the form of or part of a single peptide chain, an aggregate, complex and/or nanoparticle.

According to an aspect, the present invention concerns a protein comprising a polypeptide according to the invention.

According to an aspect, the present invention concerns an isolated nucleic acid coding for a polypeptide or protein according to the invention.

According to an aspect, the present invention concerns an expression vector, said vector comprising a nucleic acid of the invention as well as the elements necessary for the expression of said nucleic acid.

According to an aspect, the present invention concerns a recombinant cell, said cell comprising a nucleic acid according to the invention, and/or an expression vector according to the invention.

According to an aspect, the present invention concerns a pharmaceutical composition comprising at least one polypeptide, protein, nucleic acid, expression vector, or recombinant cell according to the invention, and further at least one diluent, carrier, binder, solvent or excipient.

According to an aspect, the present invention concerns a method for the preparation of a pharmaceutical composition comprising the steps of:
a. Providing one or more polypeptide, protein, nucleic acid, expression vector, or recombinant cell according to the invention, and optionally cross-linking said one or more polypeptides;
b. Optionally providing a diluent, carrier, binder, solvent or excipient;
c. Providing a substance;
d. Mixing the provided one or more peptides with any carrier of optional step b. and the substance of step d. to obtain the pharmaceutical composition.

According to an aspect, the present invention concerns a pharmaceutical composition obtainable according to the invention.

According to an aspect, the present invention concerns a biomaterial comprising a polypeptide, protein, nucleic acid, expression vector, recombinant cell, or pharmaceutical composition according to the invention.

According to an aspect, the present invention concerns a medical use of at least one polypeptide, protein, nucleic acid, expression vector, recombinant cell, pharmaceutical composition, or biomaterial according to the invention.

According to an aspect, the present invention concerns a use of a polypeptide, protein, nucleic acid, expression vector, recombinant cell, pharmaceutical composition, or implant according to the invention for the manufacture of an anti-inflammatory medicament or a medicament for immune suppression or immune modulation.

According to an aspect, the present invention concerns a method of prophylactically or therapeutically treating an autoimmune disease and/or an inflammatory condition by administering to a subject in need thereof a prophylactically or therapeutically effective amount of at least one polypeptide, protein, nucleic acid, expression vector, recombinant cell, or pharmaceutical composition according to the invention through one or more or several administrations.

According to an aspect, the present invention concerns a method of immune therapy for treating cancer or other diseases by administering to a subject in need thereof a prophylactically or therapeutically effective amount of at least one polypeptide, protein, nucleic acid, expression vector, recombinant cell, or pharmaceutical composition according to the invention through one or more or several administrations.

According to an aspect, the present invention concerns an adjuvant for use in combination with a vaccine or other immunogens in order to increase the immunogenicity of said vaccine or immunogen by administering to a subject in need thereof a prophylactically or therapeutically effective amount of at least one polypeptide, protein, nucleic acid, expression vector, recombinant cell, or pharmaceutical composition according to the invention through one or more or several administrations.

According to an aspect, the present invention concerns a pharmaceutical composition including an active component wherein the active component includes a peptide sequence having at least 70% sequence identity or homology to the sequence LSILLNEE (SEQ ID NO: 26) or derivatives thereof, fragments thereof, as well as the HERV-H Env59 proteins from which it was derived, derivatives thereof, fragments thereof, complexes thereof, any tertiary structures thereof in the form of monomers, dimers, trimers, multimeres, helix structures and globular structures, crosslinkings, and any chemical modifications thereof which increase physical and/or chemical form, properties and bioavailability of the compound.

According to an aspect, the present invention concerns a pharmaceutical composition, wherein the active component includes a peptide sequence and/or is a chemical derivative thereof and/or is part of a larger polypeptide or protein including as a monomer, dimer or as a whole or partly takes part of tertiary structures such as globular or helical structure(s) including monomers, dimers, trimers, multimers including helical structures, beta-sheets, triple helical structures all in whole or in part.

According to an aspect, the present invention concerns a pharmaceutical composition, wherein the active component or peptide is part of an aggregate, complex or nanoparticle.

According to an aspect, the present invention concerns a pharmaceutical composition for injectional, topical, transdermal or oral application.

According to an aspect, the present invention concerns a pharmaceutical composition for immune therapy treatment of cancer or other diseases.

According to an aspect, the present invention concerns a pharmaceutical composition for use in vaccination.

According to an aspect, the present invention concerns a pharmaceutical composition for the treatment or prophylaxis of an autoimmune disease.

According to an aspect, the present invention concerns a pharmaceutical composition for the treatment or prophylaxis of an inflammatory condition.

According to an aspect, the present invention concerns a pharmaceutical composition for the treatment or prophylaxis of an autoimmune disease, wherein the autoimmune disease is SLE or arthritis including rheumatoid arthritis

According to an aspect, the present invention concerns a pharmaceutical composition including a peptide sequence and/or derivatives thereof selected among the groups consisting of GLSILLNEEC (SEQ ID NO: 25), LQNRRGLGLSILLNEECEEGPGPGP (SEQ ID NO: 27), LQNRRGLDLSILLNEECGPGPGP (SEQ. ID NO: 28), GLSILLNEECGPGPGP (SEQ ID NO: 29) and LQNRRGLLQNRRGLGLSILLNEE (SEQ ID NO: 30).

According to an aspect, the present invention concerns a polypeptide as above including a peptide sequence selected among GLSILLNEEC (SEQ ID NO: 25), LQNRRGLGLSILLNEECEEGPGPGP (SEQ ID NO: 27), LQNRRGLDLSILLNEECGPGPGP (SEQ ID NO: 28), GLSILLNEECGPGPGP (SEQ ID NO: 29) and LQNRRGLLQNRRGLGLSILLNEE (SEQ ID NO: 30).

According to an aspect, the present invention concerns a polypeptide sequence, which contains the sequence LSILLNEE (SEQ ID NO: 26) attached to a sequence or a fragment thereof chosen among Seq ID 1 to Seq ID 1043. The attachment can be through N-terminal, C-terminal peptide bonds or any other chemical covalent and/or non-covalent bonds between any chemical moieties in either peptide fragment.

According to an aspect, the present invention concerns an expression vector including a nucleic acid sequence encoding a peptide having at least 70% sequence identity or homology to the sequence LSILLNEE (SEQ ID NO: 26).

According to an aspect, the present invention concerns an expression vector including a nucleic acid sequence encoding any of the peptides of the invention.

According to an aspect, the present invention concerns an expression vector as above, which utilizes an expression system based on a microorganism such as a retrovirus, an adeno virus, a pox virus, a measles virus, or a salmonella, E. coli or yeast based vector.

According to an aspect, the present invention concerns a pharmaceutical composition including any expression vector of the invention.

According to an aspect, the present invention concerns a method of prophylactically or therapeutically treating an autoimmune disease and/or an inflammatory condition by administering to a subject in need thereof a prophylactically or therapeutically effective amount of a pharmaceutical composition of the invention through one or more administration routes.

According to an aspect, the present invention concerns a biomaterial, such as a surface, particle, mesh, device, tube, etc., which contains a polypeptide of the invention. The polypeptide can be chemically bound to the biomaterial or be physically associated with it such as within its interior.

In yet another aspect, the present invention relates to diagnosis of SLE by means of measuring the expression level of HERV-H DNA. The expression level may be expressed by mean copy number or mean RNA.

In yet another aspect, the present invention relates to diagnosis of SLE by means of measuring the expression level of ENV-59 DNA and/or RNA.

In yet another aspect, the present invention relates to the use of a human endogenous retrovirus which is wholly or partly transcribed into RNA and which has either lower or higher transcription level in persons with a condition as compared to persons without said condition, for the treatment or diagnosis of said condition.

In yet another aspect, the present invention relates to the use of a human endogenous retrovirus which is wholly or partly transcribed into RNA and which has either lower or higher transcription level in persons with an autoimmune condition as compared to persons without said condition, for treatment or diagnosis of said autoimmune condition.

In yet another aspect, the present invention relates to the use of a human HERV-H which is wholly or partly transcribed into RNA and which has either lower or higher transcription level in persons with a condition as compared to persons without said condition, for treatment for diagnosis of said condition or disease.

In yet another aspect, the present invention relates to the use of HERV-H 59 derived DNA, RNA or proteins for diagnosis of the said condition or disease. An ENV 59 peptide sequence is provided as SEQ ID NO: 1044, an ENV 59 DNA sequence is provided as SEQ ID NO: 1045, and an HERV-H 59 complete provirus sequence is provided as SEQ ID NO: 1046.

According to an aspect, the invention concerns a polypeptide comprising a peptide sequence having at least 62.5%, more preferred 75%, more preferred 87.5%, more preferred at least 100% sequence identity to the sequence LSILLNEE (SEQ ID NO: 26).

According to an aspect, the invention concerns polypeptide as above comprising one or more peptide sequences having at least 70%, preferably at least 76%, more preferred at least 82%, preferably at least 88%, more preferred at least 94%, preferably 100% sequence identity to sequences selected among LQNRRGLGLSILLNEEC (SEQ ID NO: 1), GLSILLNEEC (SEQ ID NO: 25), LQNRRGLGLSILLNEECEEGPGPGP (SEQ ID NO: 27), LQNRRGLDLSILLNEECGPGPGP (SEQ ID NO: 28), GLSILLNEECGPGPGP (SEQ ID NO: 29) and LQNRRGLLQNRRGLGLSILLNEE (SEQ ID NO: 30).

According to an aspect, the invention concerns a polypeptide as any above, said polypeptide comprising a peptide sequence having at least 76%, more preferred at least 82%, preferably at least 88%, more preferred at least 94%, preferably 100% sequence identity to a sequence selected among the sequences SEQ ID NO: 1-41.

According to an aspect, the invention concerns a polypeptide as above selected among polypeptides having at least 76%, more preferred at least 82%, preferably at least 88%, more preferred at least 94%, preferably 100% sequence identity to sequences selected among LSILLNEE (SEQ ID NO: 26), LQNRRGLGLSILLNEEC (SEQ ID NO: 1), GLSILLNEEC (SEQ ID NO: 25), LQNRRGLGLSILLNEECEEGPGPGP (SEQ ID NO: 27), LQNRRGLDLSILLNEECGPGPGP (SEQ ID NO: 28), GLSILLNEECGPGPGP (SEQ ID NO: 29) and LQNRRGLLQNRRGLGLSILLNEE (SEQ ID NO: 30).

According to an aspect, the invention concerns a polypeptide of claim 1 selected among LSILLNEE (SEQ ID NO: 26), LQNRRGLGLSILLNEEC (SEQ ID NO: 1), GLSILLNEEC (SEQ ID NO: 25), LQNRRGLGLSILLNEECEEGPGPGP (SEQ ID NO: 27), LQNRRGLDLSILLNEECGPGPGP (SEQ ID NO: 28), GLSILLNEECGPGPGP (SEQ ID NO: 29) and LQNRRGLLQNRRGLGLSILLNEE (SEQ ID NO: 30).

According to an aspect, the invention concerns a polypeptide entity comprising a polypeptide as any above, said polypeptide entity comprising less than 250 aminoacids, preferably less than 200 amino acids, more preferred less than 175 amino acids, preferably less than 150 amino acids, more preferred less than 125 amino acids, preferably less than 100 amino acids, more preferred less than 75 amino acids, preferably less than 60 amino acids, more preferred less than 50 amino acids, preferably less than 40 amino acids, more preferred less than 35 amino acids, preferably less than 30 amino acids, more preferred less than 25 amino acids, preferably less than 20 amino acids, more preferred less than 19 amino acids, preferably less than 18 amino acids, more preferred less than 17 amino acids, preferably less than 16 amino acids, more preferred less than 15 amino acids, preferably less than 14 amino acids, more preferred less than 13 amino acids, preferably less than 12 amino acids, more preferred less than 11 amino acids, preferably less than 10 amino acids, more preferred less than 9 amino acids, preferably less than 8 amino acids, more preferred less than 7 amino acids, preferably less than 6 amino acids.

According to an aspect, the invention concerns a polypeptide entity comprising a polypeptide as any above, said polypeptide entity comprising at least 5, more preferred at least 6, preferably at least 7, more preferred at least 8, preferably at least 9, more preferred at least 10, preferably at least 11, more preferred at least 12, preferably at least 13, more preferred at least 14, preferably at least 15, more preferred at least 16, preferably at least 17, more preferred at least 18, preferably at least 19, more preferred at least 20, preferably at least 25, more preferred at least 30, preferably at least 35, more preferred at least 40, preferably at least 50, more preferred at least 60, preferably at least 75, more preferred at least 100, preferably at least 125, more preferred at least 150, preferably at least 175, more preferred at least 200, preferably at least 250 amino acids.

According to an aspect, the invention concerns a polypeptide with a length of 17 amino acids, wherein the sequence of the first 7 amino acids is identical to the sequence of the first 7 amino acids of a sequence selected among the sequences of SEQ ID NO: 26-1027, and wherein the last 10 amino acids are GLSILLNEEC (SEQ ID NO: 25).

According to an aspect, the invention concerns the polypeptide as above, comprising 1, 2, 3 or 4 point mutations.

According to an aspect, the invention concerns the polypeptide as any above, which is glycolysed.

According to an aspect, the invention concerns the polypeptide as any above, which is acylated.

According to an aspect, the invention concerns the polypeptide as any above, which is a monomer.

According to an aspect, the invention concerns the polypeptide as any above, which is dimerized or trimerized.

According to an aspect, the invention concerns a protein comprising a polypeptide as any above, wherein said protein comprises less than 250 aminoacids, preferably less than 200 amino acids, more preferred less than 175 amino acids, preferably less than 150 amino acids, more preferred less than 125 amino acids, preferably less than 100 amino acids, more preferred less than 75 amino acids, preferably less than 60 amino acids, more preferred less than 50 amino acids, preferably less than 40 amino acids, more preferred less than 35 amino acids, preferably less than 30 amino acids, more preferred less than 25 amino acids, preferably less than 20 amino acids, more preferred less than 19 amino acids, preferably less than 18 amino acids, more preferred less than 17 amino acids, preferably less than 16 amino acids, more preferred less than 15 amino acids, preferably less than 14 amino acids, more preferred less than 13 amino acids, preferably less than 12 amino acids, more preferred less than 11 amino acids, preferably less than 10 amino acids, more preferred less than 9 amino acids, preferably less than 8 amino acids, more preferred less than 7 amino acids, preferably less than 6 amino acids.

According to an aspect, the invention concerns a protein or polypeptide as any above or a protein comprising a polypeptide as any above, said protein or polypeptide comprising at least 5, more preferred at least 6, preferably at least 7, more preferred at least 8, preferably at least 9, more preferred at least 10, preferably at least 11, more preferred at least 12, preferably at least 13, more preferred at least 14, preferably at least 15, more preferred at least 16, preferably at least 17, more preferred at least 18, preferably at least 19, more preferred at least 20, preferably at least 25, more preferred at least 30, preferably at least 35, more preferred at least 40, preferably at least 50, more preferred at least 60, preferably at least 75, more preferred at least 100, preferably at least 125, more preferred at least 150, preferably at least 175, more preferred at least 200, preferably at least 250 amino acids.

According to an aspect, the invention concerns a protein comprising a polypeptide as any above, wherein said protein is not fusion active.

According to an aspect, the invention concerns the polypeptide or protein as any above, wherein said polypeptide or protein inhibits IL-6 expression in a mammalian cell system or an animal model.

According to an aspect, the invention concerns an isolated nucleic acid coding for a polypeptide or protein according to any of the preceding claims.

According to an aspect, the invention concerns an expression vector, said vector comprising a nucleic acid as above as well as the elements necessary for the expression of said nucleic acid.

According to an aspect, the invention concerns an expression vector as above, wherein said vector is an eukaryotic or prokaryotic or viral expression vector.

According to an aspect, the invention concerns an expression vectoras above, wherein said vector is selected among the group consisting of yeast, e-coli and baculo.

According to an aspect, the invention concerns a pharmaceutical composition comprising at least one polypeptide, protein, nucleic acid, or expression vector according to any of the preceding claims, and further at least one diluent, carrier, binder, solvent or excipient.

According to an aspect, the invention concerns the pharmaceutical composition according to any of the claims, wherein said at least one polypeptide, protein, nucleic acid, expression vector, or recombinant cell is the active ingredient or sole active ingredient of said pharmaceutical product.

According to an aspect, the invention concerns a method for the preparation of a pharmaceutical composition comprising the steps of:
a. Providing one or more polypeptide, protein, nucleic acid, expression vector, or recombinant cell according to any of the preceding claims, and optionally cross-linking said one or more polypeptides;
b. Optionally providing a diluent, carrier, binder, solvent or excipient;
c. Providing a substance;
d. Mixing the provided one or more peptides with any carrier of optional step b. and the substance of step d. to obtain the pharmaceutical composition.

According to an aspect, the invention concerns the method as above, wherein said substance of step c. is selected from the group consisting of creams, lotions, ointments, gels, balms, salves, oils, foams, and shampoos.

According to an aspect, the invention concerns a pharmaceutical composition obtainable as above.

According to an aspect, the invention concerns a pharmaceutical composition as any above, wherein said pharmaceutical composition is selected among the group consisting of creams, lotions, shake lotions, ointments, gels, balms, salves, oils, foams, shampoos, sprays, aerosols, transdermal patches and bandages.

According to an aspect, the invention concerns a medical use of at least one polypeptide, protein, nucleic acid, expression vector, recombinant cell, or pharmaceutical composition according to any of the preceding claims.

According to an aspect, the invention concerns a use of at least one polypeptide, protein, nucleic acid, expression vector, recombinant cell, or pharmaceutical composition according to any of the preceding claims for immune suppression or immune modulation.

According to an aspect, the invention concerns the polypeptide, protein, nucleic acid, expression vector, recombinant cell, or pharmaceutical composition according to any of the preceding claims for use in surgery, prophylaxis, therapy, a diagnostic method, treatment and/or amelioration of disease.

According to an aspect, the invention concerns the polypeptide, protein, nucleic acid, expression vector, recombinant cell, or pharmaceutical composition according to any of the preceding claims for the treatment, amelioration or prophylaxis of an autoimmune disease.

According to an aspect, the invention concerns the polypeptide, protein, nucleic acid, expression vector, recombinant cell, or pharmaceutical composition as above, wherein the autoimmune disease is SLE (systemic lupus erythematosus) or arthritis, such as rheumatoid arthritis, spondyloarthritis, or multiple sclerosis (MS).

According to an aspect, the invention concerns the polypeptide, protein, nucleic acid, expression vector, recombinant cell, or pharmaceutical composition according to any of the preceding claims for the treatment, amelioration or prophylaxis of an inflammatory condition or a disorder associated with inflammation, such as acute or chronic inflammation.

According to an aspect, the invention concerns the polypeptide, protein, nucleic acid, expression vector, recombinant cell, or pharmaceutical composition according to any of the preceding claims for use as a medicament.

According to an aspect, the invention concerns the polypeptide, protein, nucleic acid, expression vector, recombinant cell, or pharmaceutical composition for a use according to any of the preceding claims, comprising prophylaxis or treatment of sepsis.

According to an aspect, the invention concerns the polypeptide, protein, nucleic acid, expression vector, recombinant cell, or pharmaceutical composition for a use according to any of the preceding claims, comprising prophylaxis or treatment of spondyloarthritis.

According to an aspect, the invention concerns the polypeptide, protein, nucleic acid, expression vector, recombinant cell, or pharmaceutical composition for a use according to any of the preceding claims, comprising prophylaxis or treatment of asthma and/or allergy.

According to an aspect, the invention concerns the polypeptide, protein, nucleic acid, expression vector, recombinant cell, or pharmaceutical composition according to any of the preceding claims for a use in as an adjuvant, such as in a vaccine.

According to an aspect, the invention concerns a method of prophylactically or therapeutically treating an autoimmune disease and/or an inflammatory condition by administering to a subject in need thereof a prophylactically or therapeutically effective amount of at least one polypeptide, protein, nucleic acid, expression vector, recombinant cell, or pharmaceutical composition according to any of the preceding claims through one or more or several administrations.

According to an aspect, the invention concerns a use of a polypeptide, protein, nucleic acid, expression vector, recombinant cell, or pharmaceutical composition according to any of the preceding claims, for prophylaxis or treatment of a condition or disease by an administration route selected among injection, inhalation, topical, transdermal, oral, nasal, vaginal, or anal delivery.

According to an aspect, the invention concerns the use as above, wherein the mode of injection is selected among intravenous (IV), intraperironeal (IP), subcutaneous (SC) and (intramuscular) IM.

According to an aspect, the invention concerns the use as above, for treatment of a disease by direct injection at a site affected by a disorder, such as inflammation.

According to an aspect, the invention concerns the use of a polypeptide, protein, nucleic acid, expression vector, recombinant cell, or pharmaceutical composition according to any of the preceding claims, for treatment of Arthritis where the composition is injected directly at site of inflammation.

### DETAILED DISCLOSURE

The present invention is inter alia the result of studying the involvement of the HERV genes and their influence on the immunological response in autoimmune diseases. It was found that in patients with SLE, the expression of HERV-H Env59 mRNA is negatively correlated with the levels of IL-6 and TLR7 expression (p=0.0065, p=0.02, respectively). It was demonstrated that HERV-H Env59 encodes a functional membrane glycoprotein and make infectious pseudotyped virions with a lentiviral vector system. Moreover an ISD in ENV-59 with a unique sequence compared to known ISDs og ISD like sequences was identified.

This ENV-59 ISD seems to be unique to humans (although a similar ISD with one point mutation is also found in chimpanzees and might be present in other primates).

The peptide, GLSILLNEEC (SEQ ID NO: 25), derived from the Env59 ISU domain has significant immune regulatory activity both in vitro, ex vivo and in vivo. Surprisingly the virus-derived immunosuppressive peptide inhibits, among other effects, the production of IL-6, confirming the negative correlation seen between IL-6 and ENV59 expression levels in SLE patients. This further suggests that that the endogenous envelope protein has adapted to perform a pivotal role in the human immune system and has an advantageous function in controlling autoimmune diseases.

In vivo the ISD peptide is capable of strongly reducing the symptoms of arthritis induced in two validated and recognized animal models, namely the Sakaguchi mice model and the Collagen Induced Arthritis—CIA-mouse model. This highly suggests a potential for the ISD peptide to have anti-Rhuematoid-Arthrtitis activity in humans;

According to an embodiment the invention relates to the peptide sequence LSILLNEE (SEQ ID NO: 26), or derivatives thereof, fragments thereof, as well as the HERV-H Env59 proteins from which it was derived, derivatives thereof, fragments thereof, complexes thereof, any tertiary structures thereof in the form of monomers, dimers, trimers, multimeres, helix structures and globular structures, crosslinkings, and any chemical modifications thereof which increase physical and/or chemical form, properties and bioavailability of the compound of the present invention or each separately or in any combination the polypeptides or peptides of the invention. Additionally the invention also relates to any pharmaceutical formulations suitable for the application of the above described peptide and proteins to a patient in need thereof.

According to an embodiment, the invention concerns a polypeptide consisting of or comprising a sequence having at least 62%, more preferred at least 75%, preferably at least 87%, more preferred 100% sequence identity to the sequence LSILLNEE (SEQ ID NO: 26).

According to an embodiment, a polypeptide of the invention is glycolysed.

According to an embodiment, a polypeptide of the invention is acylated.

According to an embodiment, a polypeptide of the invention is dimerized or trimerized.

A polypeptide is obtainable from the sequence e.g. by 1, 2 or 3 point deletions, point insertions and/or point mutations. A point mutation is used here about a change of a single amino acid, a point insertion is the insertion of a single amino acid, and a point deletion is the removal of a single amino acid.

The invention also concerns a polypeptide which includes a peptide sequence having at least 70% sequence identity or homology to the sequence LSILLNEE (SEQ ID NO: 26), and derivatives thereof, fragments thereof, complexes thereof, any tertiary structures thereof in the form of monomers, dimers, trimers, multimeres, helix structures and globular structures, and crosslinkings, and any chemical modifications thereof to increase physical chemical form and properties and bioavailability.

A polypeptide of the invention may e.g. be in the form of or part of a single peptide chain, an aggregate, complex and/or nanoparticle.

According to an embodiment, the invention concerns the polypeptide, said polypeptide comprising the sequence LSILLNEE (SEQ ID NO: 26) attached to a sequence or a fragment thereof chosen among Seq ID 1 to Seq ID 1043.

The attachment can be through N-terminal, C-terminal peptide bonds or any other chemical covalent and/or non-covalent bonds between any chemical moieties in either peptide fragment.

According to an embodiment, the polypeptide comprises or consists of a peptide sequence selected among GLSILLNEEC (SEQ ID NO: 25), LQNRRGLGLSILLNEECEEGPGPGP (SEQ ID NO: 27), LQNRRGLDLSILLNEECGPGPGP (SEQ ID NO: 28), GLSILLNEECGPGPGP (SEQ ID NO: 29) and LQNRRGLLQNRRGLGLSILLNEE (SEQ ID NO: 30).

According to an embodiment, the polypeptide comprises or consists of a sequence having at least 70% sequence identity to the sequence: LQNRRGLGLSILLNEEC (SEQ ID NO: 1).

According to an embodiment, the polypeptide comprises or consists of a sequence having at least 76%, more preferred at least 82%, preferably at least 88%, more preferred at least 94%, preferably 100% sequence identity to SEQ ID NO: 1.

According to an embodiment, the polypeptide comprises or consists of a sequence having at least 76%, more preferred at least 82%, preferably at least 88%, more preferred at least 94%, preferably 100% sequence identity to a sequence selected among the sequences SEQ ID NO: 1-41.

According to an embodiment, the polypeptide comprises less than 250 aminoacids, preferably less than 200 amino acids, more preferred less than 175 amino acids, preferably less than 150 amino acids, more preferred less than 125 amino acids, preferably less than 100 amino acids, more preferred less than 75 amino acids, preferably less than 60 amino acids, more preferred less than 50 amino acids, preferably less than 40 amino acids, more preferred less than 35 amino acids, preferably less than 30 amino acids, more preferred less than 25 amino acids, preferably less than 20 amino acids, more preferred less than 19 amino acids, preferably less than 18 amino acids, more preferred less than 17 amino acids, preferably less than 16 amino acids, more preferred less than 15 amino acids, preferably less than 14 amino acids, more preferred less than 13 amino acids, preferably less than 12 amino acids, more preferred less than 11 amino acids, preferably less than 10 amino acids, more preferred less than 9 amino acids, preferably less than 8 amino acids, more preferred less than 7 amino acids, preferably less than 6 amino acids.

According to an embodiment, the polypeptide comprises at least 5, more preferred at least 6, preferably at least 7, more preferred at least 8, preferably at least 9, more preferred at least 10, preferably at least 11, more preferred at least 12, preferably at least 13, more preferred at least 14, preferably at least 15, more preferred at least 16, preferably at least 17, more preferred at least 18, preferably at least 19, more preferred at least 20, preferably at least 25, more preferred at least 30, preferably at least 35, more preferred at least 40, preferably at least 50, more preferred at least 60, preferably at least 75, more preferred at least 100, preferably at least 125, more preferred at least 150, preferably at least 175, more preferred at least 200, preferably at least 250 amino acids.

According to an embodiment, the polypeptide has a length of 17 amino acids, wherein the sequence of the first 7 amino acids is identical to the sequence of the first 7 amino acids of a sequence selected among the sequences of SEQ ID NO: 42-1043, and wherein the last 10 amino acids are GLSILLNEEC (SEQ ID NO: 25).

According to an embodiment, the polypeptide has 1, 2, 3 or 4 point mutations.

According to an embodiment, the polypeptide is or acts as an immune suppressive domain. Such a polypeptide may be referred to as an immunosuppressive peptide. According to an embodiment, the immune suppressive domain is obtainable from a polypeptide according to the invention, by at least one point mutation, deletion or insertion. According to an embodiment, the total number of point mutations, deletions or insertions is selected among 1, 2, 3 and 4. According to an embodiment, the total number of point mutations, deletions or insertions is more than 4. According to an embodiment, the polypeptide is a monomeric peptide. According to an embodiment, the polypeptide is cross-linked to at least one additional immunosuppressive peptide and/or connected to a protein, said protein being connected to at least one additional immune suppressive domain. According to an embodiment, the polypeptide is connected to at least one additional immunosuppressive peptide to form a dimer. According to an embodiment, the dimer is homologous and comprises at least two immunosuppressive peptides, which are cross-linked by a disulfide bond, N-terminal to N-terminal or C-terminal to C-terminal, and/or a tandem repeat. According to an embodiment, the polypeptide is connected to at least one additional immunosuppressive peptide to form a heterologous dimer or a homologous dimer. According to an embodiment, the polypeptide is connected to at least two additional immunosuppressive peptides to form a multimer or polymer. According to an embodiment, the polypeptide comprises one or more modifications. According to an embodiment, the modifications are selected from the group consisting of chemical derivatizations, L-amino acid substitutions, D-amino acid substitutions, synthetic amino acid substitutions, deaminations and decarboxylations. According to an embodiment, the polypeptide has increased resistance against proteolysis compared to peptides or proteins not comprising said at least one modification.

In particular embodiments the length of the active component of the immunosuppressive peptides is 35 amino acids, or 34, or 33, or 32, or 31, or 30, or 29, or 28, or 27, or 26, or 25, or 24, or 23, or 22, or 21, or 20, or 19, or 18, or 17, or 16, or 15, or 14, or 13, or 12, or 11, or 10, or 9, or 8, or 7, or 6, or 5, or 4, or 3 amino acids long. Thus, the immunosuppressive peptides of the present invention have lengths and amino acid sequences corresponding to any known ISD. A special feature of the immunosuppressive peptides of the present invention is that they may contain an extra cysteine (Cys or C) residue, either in the N-terminal or C-terminal of the polypeptide. In a particular embodiment the cysteine residue is located in the C-terminal of the peptides. The presence and function of this cysteine residue is primarily so as to crosslink two or more polypeptides together, preferable via disulfide bonds, as described herein below. However, the function of the extra cysteine may be other than that of cross-linking. Thus, the immunosuppressive peptides of the present invention may have amino acid sequences corresponding to any of SEQ ID: 1 to 1043, and wherein the immunosuppressive peptides further contain an extra cystein (Cys og C) residue at either the N-terminal or C-terminal of the peptide.

According to an embodiment additional aminoacids or molecules may be added or linked to an immunosuppressive peptide in order to improve the solubility characteristics of said immusuppressive peptide.

According to an embodiment, the invention concerns a protein comprising a polypeptide according to the invention.

According to an embodiment, the protein is an envelope protein.

According to an embodiment, the protein is not a functional membrane glycoprotein.

According to an embodiment, the protein is not fusion active. The expression “not fusion active” means the protein is not capable of mediating fusion of two biological membranes.

According to an embodiment, the protein is not bound or linked to a membrane.

According to an embodiment, the protein is not a membrane integral protein.

According to an embodiment, the polypeptide or protein according to the invention inhibits IL-6 expression in a mammalian cell system or an animal model.

According to an embodiment, the polypeptide or protein according to the invention induces IL-6 expression in a mammalian cell system or an animal model.

According to an embodiment, the invention concerns an isolated nucleic acid coding for a polypeptide or protein according to the invention.

According to an embodiment, the invention concerns an expression vector, said vector comprising a nucleic acid of the invention as well as the elements necessary for the expression of said nucleic acid.

According to an embodiment, the invention concerns an expression vector, wherein said vector is an eukaryotic or prokaryotic or viral expression vector.

According to an embodiment, the invention concerns an expression vector including a nucleic acid sequence encoding for a peptide having at least 62% sequence identity or homology to the sequence LSILLNEE (SEQ ID NO: 26).

According to an embodiment, the invention concerns an expression vector including a nucleic acid sequence encoding for a polypeptide or protein according to the invention.

The expression vector may be based upon a microorganism such as a retrovirus, an adeno virus, a pox virus, a measles virus, a salmonella based vector, an E. coli vector, yeast.

According to an embodiment, the invention concerns a recombinant cell, said cell comprising a nucleic acid according of the invention, and/or an expression vector according to the invention.

According to an embodiment, the invention concerns a pharmaceutical composition comprising at least one polypeptide, protein, nucleic acid, expression vector, or recombinant cell according to the invention, and further at least one diluent, carrier, binder, solvent or excipient.

### Administration Forms, Formulations and Dosage Regimes

Pharmaceutically useful compositions comprising the compounds of the invention may be formulated according to known methods such as by the admixture of a pharmaceutically acceptable carriers and/or additional active compounds. Examples of such carriers and methods of formulation may be found in Remington's Pharmaceutical Sciences. To form a pharmaceutically acceptable composition suitable for effective administration, such compositions will contain an effective amount of a compound of the invention. Such compositions may also contain more than one compound of the invention.

Pharmaceutical compositions or compounds of the invention are administered to an individual in therapeutic effective amounts. The effective amount may vary according to a variety of factors such as the individual's condition, weight, sex and age. Other factors include the mode of administration. Generally, the compositions will be administered in dosages ranging from about 1 μg to about 100 mg, and especially from about 10 μg to about 10 mg.

The pharmaceutical compositions may be provided to the individual by a variety of routes and especially such as, subcutaneous, topical, oral, mucosal, intravenous, parenterally, and intramuscular.

Such formulations are generally safe, do not have toxic side effects; can be administered by an effective route; are stable; and are compatible with the pharmaceutically carriers.

The pharmaceutical formulations and compounds of the invention may be used in dosage forms such as capsules, suspensions, elixirs, or liquid solutions.

The pharmaceutical formulations and compounds of the invention may be administered in single or multiple doses.

Whilst it is possible for the compositions or salts of the present invention to be administered as the raw chemical, it is preferred to present them in the form of a pharmaceutical formulation. Accordingly, the present invention further provides a pharmaceutical formulation, for medicinal application, which comprises an entity of the present invention or a pharmaceutically acceptable salt thereof, and a pharmaceutically acceptable carrier therefore.

Pharmaceutically acceptable salts of the instant compositions, where they can be prepared, are also intended to be covered by this invention.

According to an embodiment, the invention concerns the pharmaceutical composition, wherein said at least one polypeptide, protein, nucleic acid, expression vector, or recombinant cell is the active ingredient or sole active ingredient of said pharmaceutical product.

A pharmaceutical composition of the invention may include as the active component a peptide sequence and/or a chemical derivative thereof, and said sequence or derivative thereof may be part of a larger polypeptide or protein, e.g. as a monomer, dimer, or may as a whole or partly take part of tertiary structures such as globular or helical structure(s), including as monomers, dimers, trimers, multimers, and including helical structures, beta-sheets, triple helical structures, all in whole or in part.

A pharmaceutical composition of the invention may include as the active component a peptide, which is part of an aggregate, complex or nanoparticle.

According to an embodiment, the invention concerns a method for the preparation of a pharmaceutical composition comprising the steps of:
a. Providing one or more polypeptide, protein, nucleic acid, expression vector, or recombinant cell according to the invention, and optionally cross-linking said one or more polypeptides;
b. Optionally providing a diluent, carrier, binder, solvent or excipient;
c. Providing a substance;
d. Mixing the provided one or more peptides with any carrier of optional step b. and the substance of step d. to obtain the pharmaceutical composition.

According to an embodiment, the invention concerns the method, wherein said substance of step c. is selected from the group consisting of creams, lotions, ointments, gels, balms, salves, oils, foams, and shampoos.

According to an embodiment, the invention concerns the pharmaceutical composition obtainable according to a method of the invention.

According to an embodiment, the invention concerns the pharmaceutical composition, wherein said pharmaceutical composition is selected among the group consisting of creams, lotions, shake lotions, ointments, gels, balms, salves, oils, foams, shampoos, sprays, aerosols, transdermal patches and bandages.

According to an embodiment, the invention concerns a biomaterial comprising a polypeptide, protein, nucleic acid, expression vector, recombinant cell, or pharmaceutical composition according to the invention.

According to an embodiment, the invention concerns the biomaterial, wherein said biomaterial is selected among a surface, particle, mesh, device, tube, or an implant.

The polypeptide can be chemically bound to the biomaterial or be physically associated with it such as in its interior.

According to an embodiment, the invention concerns a medical use of at least one polypeptide, protein, nucleic acid, expression vector, recombinant cell, pharmaceutical composition, or biomaterial according to the invention.

According to an embodiment, the invention concerns a use of at least one polypeptide, protein, nucleic acid, expression vector, recombinant cell, pharmaceutical composition, or implant according to the invention for immune suppression or immune modulation.

According to an embodiment, the invention concerns the polypeptide, protein, nucleic acid, expression vector, recombinant cell, pharmaceutical composition, or implant according to the invention for use in surgery, prophylaxis, therapy, a diagnostic method, treatment and/or amelioration of disease.

According to an embodiment, the invention concerns the polypeptide, protein, nucleic acid, expression vector, recombinant cell, pharmaceutical composition, or implant according to the invention for the treatment, amelioration or prophylaxis of an autoimmune disease.

According to an embodiment, the invention concerns the polypeptide, protein, nucleic acid, expression vector, recombinant cell, pharmaceutical composition, or implant, wherein the autoimmune disease is SLE (systemic lupus erythematosus) or arthritis, such as rheumatoid arthritis.

According to an embodiment, the invention concerns the polypeptide, protein, nucleic acid, expression vector, recombinant cell, pharmaceutical composition, or implant according to the invention for the treatment, amelioration or prophylaxis of an inflammatory condition or a disorder associated with inflammation, such as acute or chronic inflammation.

According to an embodiment, the invention concerns the polypeptide, protein, nucleic acid, expression vector, recombinant cell, pharmaceutical composition, or implant according to the invention for use as a medicament.

According to an embodiment, the invention concerns the polypeptide, protein, nucleic acid, expression vector, recombinant cell, pharmaceutical composition, or implant for a use according to the invention, wherein the subject is a human or an animal.

According to an embodiment, the invention concerns the polypeptide, protein, nucleic acid, expression vector, recombinant cell, pharmaceutical composition, or implant according to the invention, for use on an organ.

According to an embodiment, the invention concerns the polypeptide, protein, nucleic acid, expression vector, recombinant cell, pharmaceutical composition, or implant according to the invention, in the preparation or treatment of transplantation patients.

According to an embodiment, the invention concerns the polypeptide, protein, nucleic acid, expression vector, recombinant cell, pharmaceutical composition, or implant for a use according to the invention, comprising prophylaxis or treatment of a condition selected among Acute disseminated encephalomyelitis (ADEM), Addison's disease, Agammaglobulinemia, Alopecia areata, Amyotrophic Lateral Sclerosis, Ankylosing Spondylitis, Antiphospholipid syndrome, Antisynthetase syndrome, Atopic allergy, Atopic dermatitis, Autoimmune aplastic anemia, Autoimmune cardiomyopathy, Autoimmune enteropathy, Autoimmune hemolytic anemia, Autoimmune hepatitis, Autoimmune inner ear disease, Autoimmune lymphoproliferative syndrome, Autoimmune peripheral neuropathy, Autoimmune pancreatitis, Autoimmune polyendocrine syndrome, Autoimmune progesterone dermatitis, Autoimmune thrombocytopenic purpura, Autoimmune urticaria, Autoimmune uveitis, Balo disease/Balo concentric sclerosis, Behget's disease, Berger's disease, Bickerstaff's encephalitis, Blau syndrome, Bullous pemphigoid, Cancer, Castleman's disease, Celiac disease, Chagas disease, Chronic inflammatory demyelinating polyneuropathy, Chronic recurrent multifocal osteomyelitis, Chronic obstructive pulmonary disease, Churg-Strauss syndrome, Cicatricial pemphigoid, Cogan syndrome, Cold agglutinin disease, Complement component 2 deficiency, Contact dermatitis, Cranial arteritis, CREST syndrome, Crohn's disease, Cushing's Syndrome, Cutaneous leukocytoclastic angiitis, Dego's disease, Dercum's disease, Dermatitis herpetiformis, Dermatomyositis, Diabetes mellitus type 1, Diffuse cutaneous systemic sclerosis, Dressler's syndrome, Drug-induced lupus, Discoid lupus erythematosus, Eczema, Endometriosis, Enthesitis-related arthritis, Eosinophilic fasciitis, Eosinophilic gastroenteritis, Epidermolysis bullosa acquisita, Erythema nodosum, Erythroblastosis fetalis, Essential mixed cryoglobulinemia, Evan's syndrome, Fibrodysplasia ossificans progressiva, Fibrosing alveolitis, Gastritis, Gastrointestinal pemphigoid, Glomerulonephritis, Goodpasture's syndrome, Graves' disease, Guillain-Barré syndrome (GBS), Hashimoto's encephalopathy, Hashimoto's thyroiditis, Henoch-Schonlein purpura, Herpes gestationis, hepatitis, Hidradenitis suppurativa, Hughes-Stovin syndrome, Hypogammaglobulinemia, Idiopathic inflammatory demyelinating diseases, Idiopathic pulmonary fibrosis, Idiopathic thrombocytopenic purpura, IgA nephropathy, Inclusion body myositis, Chronic inflammatory demyelinating polyneuropathy, Interstitial cystitis, Juvenile idiopathic arthritis, Kawasaki's disease, Lambert-Eaton myasthenic syndrome, Leukocytoclastic vasculitis, Lichen planus, Lichen sclerosus, Linear IgA disease (LAD), Lou Gehrig's disease, Lupoid hepatitis, Lupus erythematosus, Majeed syndrome, Ménière's disease, Microscopic polyangiitis, Miller-Fisher syndrome, Mixed connective tissue disease, Morphea, Mucha-Habermann disease, Multiple sclerosis, Myasthenia gravis, Myositis, Narcolepsy, Neuromyelitis optica, Neuromyotonia, non-alcoholic steatohepatitis (NASH), Occular cicatricial pemphigoid, Opsoclonus myoclonus syndrome, Ord's thyroiditis, Palindromic rheumatism, PANDAS (pediatric autoimmune neuropsychiatric disorders associated with streptococcus), Paraneoplastic cerebellar degeneration, Paroxysmal nocturnal hemoglobinuria (PNH), Parry Romberg syndrome, Parsonage-Turner syndrome, Pars planitis, Pemphigus vulgaris, Pernicious anaemia, Perivenous encephalomyelitis, POEMS syndrome, Polyarteritis nodosa, Polymyalgia rheumatica, Polymyositis, Primary biliary cirrhosis, Primary sclerosing cholangitis, Progressive inflammatory neuropathy, Psoriasis, Psoriatic arthritis, Pyoderma gangrenosum, Pure red cell aplasia, Rasmussen's encephalitis, Raynaud phenomenon, Relapsing polychondritis, Reiter's syndrome, Restless leg syndrome, Retroperitoneal fibrosis, Rheumatoid arthritis, Rheumatic fever, Sarcoidosis, Schizophrenia, Schmidt syndrome, Schnitzler syndrome, Scleritis, Scleroderma, Serum Sickness, Sjögren's syndrome, Spondyloarthropathy, Still's disease, Stiff person syndrome, Subacute bacterial endocarditis (SBE), Susac's syndrome, Sweet's syndrome, Sydenham chorea, Sympathetic ophthalmia, Systemic lupus erythematosis, Takayasu's arteritis, Temporal arteritis, Thrombocytopenia, Tolosa-Hunt syndrome, Transverse myelitis, Ulcerative colitis, Undifferentiated connective tissue disease, Undifferentiated spondyloarthropathy, Urticarial vasculitis, Vasculitis, Vitiligo, and Wegener's granulomatosis.

According to an embodiment, the invention concerns the polypeptide, protein, nucleic acid, expression vector, recombinant cell, pharmaceutical composition, or implant for a use according to the invention, for the treatment or prevention of a disorder selected among Acne vulgaris, Allergy, Allergic rhinitis, Asthma, Atherosclerosis, Autoimmune disease, Celiac disease, Chronic prostatitis, Glomerulonephritis, Hypersensitivities, Inflammatory bowel diseases, Pelvic inflammatory disease, Reperfusion injury, Rheumatoid arthritis, Sarcoidosis, Transplant rejection, Vasculitis, interstitial cystitis, Cancer, Depression, Myopathies, and Leukocyte defects.

According to an embodiment, the invention concerns the polypeptide, protein, nucleic acid, expression vector, recombinant cell, pharmaceutical composition, or implant for a use according to the invention, comprising prophylaxis or treatment of sepsis.

According to an embodiment, the invention concerns the polypeptide, protein, nucleic acid, expression vector, recombinant cell, pharmaceutical composition, or implant for a use according to the invention, comprising prophylaxis or treatment of spondyloarthritis.

According to an embodiment, the invention concerns the polypeptide, protein, nucleic acid, expression vector, recombinant cell, pharmaceutical composition, or implant for a use according to the invention, comprising prophylaxis or treatment of asthma and/or allergy.

According to an embodiment, the invention concerns the polypeptide, protein, nucleic acid, expression vector, recombinant cell, pharmaceutical composition, or implant for a use according to the invention, comprising prophylaxis or treatment of cancer.

According to an embodiment, the invention concerns the polypeptide, protein, nucleic acid, expression vector, recombinant cell, pharmaceutical composition, or implant for a use according to the invention, comprising enhancing the immunogenicity of vaccines or any antigen including those used in vaccines.

According to an embodiment, the invention concerns the polypeptide, wherein said polypeptide is or acts as an immune suppressive domain, for use in a method of prophylaxis or treatment or amelioration of a condition associated with an autoimmune disease, wherein a gene sequence expressing said immune suppressive domain exhibits increased or decreased expression in a group of patients suffering from said autoimmune disease as compared to a healthy control group. According to an embodiment, the invention concerns the polypeptide, wherein said immune suppressive domain is from an endogenous retrovirus, preferably a human endogenous retrovirus. According to an embodiment, the invention concerns the polypeptide, wherein said immune suppressive domain is selected among the sequences of SEQ ID NO: 1-1043.

According to an embodiment, the invention concerns a use of a polypeptide selected among the sequences of SEQ ID NO: 1-1043 for the prophylaxis or treatment or amelioration of an autoimmune disease or at least one symptom associated with said autoimmune disease.

According to an embodiment, the invention concerns a use of a polypeptide, protein, nucleic acid, expression vector, recombinant cell, pharmaceutical composition, or implant according to the invention for the manufacture of an anti-inflammatory medicament or a medicament for immune suppression or immune modulation.

According to an embodiment, the invention concerns the use of a polypeptide, protein, nucleic acid, expression vector, recombinant cell, pharmaceutical composition, or implant according to the invention for the manufacture of a medicament for the preparation or treatment of transplantation patients.

According to an embodiment, the invention concerns the use of a polypeptide, protein, nucleic acid, expression vector, recombinant cell, pharmaceutical composition, or implant according to the invention for the manufacture of a medicament for prophylaxis or treatment of an autoimmune or inflammatory disease.

According to an embodiment, the invention concerns the use of a polypeptide, protein, nucleic acid, expression vector, recombinant cell, pharmaceutical composition, or implant according to the invention for the manufacture of a medicament for prophylaxis or treatment of a condition selected among Acute disseminated encephalomyelitis (ADEM), Addison's disease, Agammaglobulinemia, Alopecia areata, Amyotrophic Lateral Sclerosis, ANCA Vasculitis, Ankylosing Spondylitis, Antiphospholipid syndrome, Antisynthetase syndrome, Arteriosclerosis, Atopic allergy, Atopic dermatitis, Autoimmune aplastic anemia, Autoimmune cardiomyopathy, Autoimmune enteropathy, Autoimmune hemolytic anemia, Autoimmune hepatitis, Autoimmune inner ear disease, Autoimmune lymphoproliferative syndrome, Autoimmune peripheral neuropathy, Autoimmune pancreatitis, Autoimmune polyendocrine syndrome, Autoimmune progesterone dermatitis, Autoimmune thrombocytopenic purpura, Autoimmune urticaria, Autoimmune uveitis, Balo disease/Balo concentric sclerosis, Behget's disease, Berger's disease, Bickerstaff's encephalitis, Blau syndrome, Bullous pemphigoid, Cancer, Castleman's disease, Celiac disease, Chagas disease, Chronic inflammatory demyelinating polyneuropathy, Chronic recurrent multifocal osteomyelitis, Chronic obstructive pulmonary disease, Churg-Strauss syndrome, Cicatricial pemphigoid, Cogan syndrome, Cold agglutinin disease, Complement component 2 deficiency, Contact dermatitis, Cranial arteritis, CREST syndrome, Crohn's disease, Cushing's Syndrome, Cutaneous leukocytoclastic angiitis, Dego's disease, Dercum's disease, Dermatitis herpetiformis, Dermatomyositis, Diabetes mellitus type 1, Diffuse cutaneous systemic sclerosis, Dressler's syndrome, Drug-induced lupus, Discoid lupus erythematosus, Eczema, Endometriosis, Enthesitis-related arthritis, Eosinophilic fasciitis, Eosinophilic gastroenteritis, Epidermolysis bullosa acquisita, Erythema nodosum, Erythroblastosis fetalis, Essential mixed cryoglobulinemia, Evan's syndrome, Fibrodysplasia ossificans progressiva, Fibrosing alveolitis, Gastritis, Gastrointestinal pemphigoid, Glomerulonephritis, Goodpasture's syndrome, Graves' disease, Guillain-Barré syndrome (GBS), Hashimoto's encephalopathy, Hashimoto's thyroiditis, Henoch-Schonlein purpura, Herpes gestationis, hepatitis, Hidradenitis suppurativa, Hughes-Stovin syndrome, Hypogammaglobulinemia, Idiopathic inflammatory demyelinating diseases, Idiopathic pulmonary fibrosis, Idiopathic thrombocytopenic purpura, IgA nephropathy, Inclusion body myositis, Chronic inflammatory demyelinating polyneuropathy, Interstitial cystitis, Juvenile idiopathic arthritis, Kawasaki's disease, Lambert-Eaton myasthenic syndrome, Leukocytoclastic vasculitis, Lichen planus, Lichen sclerosus, Linear IgA disease (LAD), Lou Gehrig's disease, Lupoid hepatitis, Lupus erythematosus, Majeed syndrome, Ménière's disease, Microscopic polyangiitis, Miller-Fisher syndrome, Mixed connective tissue disease, Morphea, Mucha-Habermann disease, Multiple sclerosis, Myasthenia gravis, Myositis, Narcolepsy, Neuromyelitis optica, Neuromyotonia, non-alcoholic steatohepatitis (NASH), Occular cicatricial pemphigoid, Opsoclonus myoclonus syndrome, Ord's thyroiditis, Palindromic rheumatism, PANDAS (pediatric autoimmune neuropsychiatric disorders associated with streptococcus), Paraneoplastic cerebellar degeneration, Paroxysmal nocturnal hemoglobinuria (PNH), Parry Romberg syndrome, Parsonage-Turner syndrome, Pars planitis, Pemphigus vulgaris, Pernicious anaemia, Perivenous encephalomyelitis, POEMS syndrome, Polyarteritis nodosa, Polymyalgia rheumatica, Polymyositis, Primary biliary cirrhosis, Primary sclerosing cholangitis, Progressive inflammatory neuropathy, Psoriasis, Psoriatic arthritis, Pyoderma gangrenosum, Pure red cell aplasia, Rasmussen's encephalitis, Raynaud phenomenon, Relapsing polychondritis, Reiter's syndrome, Restless leg syndrome, Retroperitoneal fibrosis, Rheumatoid arthritis, Rheumatic fever, Sarcoidosis, Schizophrenia, Schmidt syndrome, Schnitzler syndrome, Scleritis, Scleroderma, Serum Sickness, Sjögren's syndrome, Spondyloarthropathy, Still's disease, Stiff person syndrome, Subacute bacterial endocarditis (SBE), Susac's syndrome, Sweet's syndrome, Sydenham chorea, Sympathetic ophthalmia, Systemic lupus erythematosis, Takayasu's arteritis, Temporal arteritis, Thrombocytopenia, Tolosa-Hunt syndrome, Transverse myelitis, Ulcerative colitis, Undifferentiated connective tissue disease, Undifferentiated spondyloarthropathy, Urticarial vasculitis, Vasculitis, Vitiligo, and Wegener's granulomatosis.

According to an embodiment, the invention concerns the use of a polypeptide, protein, nucleic acid, expression vector, recombinant cell, pharmaceutical composition, or implant according to the invention for the manufacture of a medicament for prophylaxis or treatment of inflammation or a condition associated with inflammation, such as acute or chronic inflammation.

According to an embodiment, the invention concerns the use of a polypeptide, protein, nucleic acid, expression vector, recombinant cell, pharmaceutical composition, or implant according to the invention, for the manufacture of a medicament for prophylaxis or treatment of a condition selected among Acne vulgaris, Allergy, Allergic rhinitis, Asthma, Atherosclerosis, Autoimmune disease, Celiac disease, Chronic prostatitis, Glomerulonephritis, Hypersensitivities, Inflammatory bowel diseases, Pelvic inflammatory disease, Reperfusion injury, Rheumatoid arthritis, Sarcoidosis, Transplant rejection, Vasculitis, interstitial cystitis, Cancer, Depression, Myopathies, and Leukocyte defects.

According to an embodiment, the invention concerns the use of a polypeptide, protein, nucleic acid, expression vector, recombinant cell, pharmaceutical composition, or implant according to the invention for the manufacture of a medicament for prophylaxis or treatment of at least condition selection among sepsis, rheumatoid arthritis, systemic lupus erythematosus (SLE), and spondyloarthritis.

According to an embodiment, the invention concerns the use of a polypeptide, protein, nucleic acid, expression vector, recombinant cell, pharmaceutical composition, or implant according to the invention for the manufacture of a medicament for prophylaxis or treatment of asthma and/or allergy.

According to an embodiment, the invention concerns the use of a polypeptide, protein, nucleic acid, expression vector, recombinant cell, pharmaceutical composition, or implant according to the invention for the manufacture of an adjuvant.

According to an embodiment, the invention concerns the use of a polypeptide, protein, nucleic acid, expression vector, recombinant cell, or pharmaceutical composition according to the invention, for coating of nanoparticles and/or biomaterials.

A biomaterial is any matter, surface, particle or construct that interacts with biological systems. Biomaterials can be derived either from nature or synthesized in the laboratory using a variety of chemical approaches utilizing metallic components, ceramic, polymers or composite materials. Some biomaterials consist of inorganic crystallization within a largely organic matrix of naturally occurring compounds.

Biomaterials are often used and/or adapted for a medical application, and thus comprise whole or part of a living structure or biomedical device which performs, augments, or replaces a natural function. Such functions may be benign, like being used for a heart valve, or may be bioactive with a more interactive functionality such as hydroxy-apatite coated hip implants. Biomaterials are also used every day in dental applications, surgery, and drug delivery such as in the form of nanoparticles. A construct with impregnated pharmaceutical products can be placed into the body, which permits the prolonged release of a drug over an extended period of time. A biomaterial may also be an autograft, allograft or xenograft used as a transplant material.

Biomaterials are used for example in: Joint replacements, bone plates, bone cement, artificial ligaments and tendons, dental implants for tooth fixation, blood vessel prostheses, heart valves, skin repair devices (artificial tissue), cochlear replacements, contact lenses, breast implants.

According to an embodiment, the invention concerns the use of a polypeptide, protein, nucleic acid, expression vector, recombinant cell, or pharmaceutical composition according to the invention, for at least partial suppression of an immune response to at least one nanoparticle or biomaterial.

According to an embodiment, the invention concerns the use of a polypeptide, protein, nucleic acid, expression vector, recombinant cell, or pharmaceutical composition according to the invention, to increase the in vivo half-life of nanoparticles and/or biomaterials and/or medical devices and/or implants in the patient.

According to an embodiment, the invention concerns the use of an endogenous retrovirus for diagnosis of a disease.

According to an embodiment, the invention concerns the use of an endogenous retrovirus whose expression level or copy number is different in a subject with a condition as compared to a subject without said condition for diagnosis of a disease.

According to an embodiment, the invention concerns the use of an endogenous retrovirus whose expression level or copy number is different in a subject with an autoimmune condition as compared to a subject without the said condition for diagnosis of a disease.

According to an embodiment, the invention concerns the use of single nucleotide polymorphisms associated with HERV-H 59 for diagnosis of a disease.

According to an embodiment, the invention concerns the use of HERV-H 59 for diagnosis of SLE.

According to an embodiment, the invention concerns a method of prophylactically or therapeutically treating an autoimmune disease and/or an inflammatory condition by administering to a subject in need thereof a prophylactically or therapeutically effective amount of at least one polypeptide, protein, nucleic acid, expression vector, recombinant cell, or pharmaceutical composition according to the invention through one or more or several administrations.

According to an embodiment, the invention concerns a method of prophylaxis or treatment or amelioration of a condition associated with an autoimmune disease, comprising:
a. Measuring the expression or copy number of at least one endogenous retrovirus in a group of patients suffering from said autoimmune disease;
b. Comparing said expression with the expression of said at least one endogenous retrovirus in a healthy control group;
c. Identifying at least one endogenous retrovirus having different expression in said group of patients;
d. Optionally identifying at least one immune suppressive domain in said at least one endogenous retrovirus;
e. Treating at least one patient suffering from said condition by administration of at least one immune suppressive domain preferably contained in a protein containing said at least one immune suppressive domain and/or a protein expressed by said endogenous retrovirus.

According to an embodiment, the invention concerns a method of prophylaxis or treatment or amelioration of a condition associated with an autoimmune disease, comprising:
f. Measuring the concentration of at least one protein or polypeptide comprising at least one immune suppressive domain in a group of patients suffering from said autoimmune disease;
g. Comparing said concentration with the concentration in a healthy control group;
h. Identifying at least one immune suppressive domain having different expression in said group of patients;
i. Treating at least one patient suffering from said condition by administration of said at least one immune suppressive domain and/or a protein comprising said at least one immune suppressive domain.

According to an embodiment, the invention concerns the method, wherein said different expression is selected among increased and decreased expression.

According to an embodiment, the invention concerns the method, wherein said endogenous retrovirus is a human endogenous retrovirus.

According to an embodiment, the invention concerns the method, wherein said human endogenous retrovirus belongs to the HERV-H subfamily or the HERV-K subfamily.

According to an embodiment, the invention concerns the method, wherein said endogenous retrovirus contains at least one open reading frame capable of encoding a protein.

According to an embodiment, the invention concerns the method, wherein said at least one open reading frame has a length of at least 50, preferably at least 100, more preferred at least 150, preferably at least 200, more preferred at least 250, preferably at least 300, more preferred at least 350, preferably at least 400 nucleotides.

According to an embodiment, the invention concerns a use of a polypeptide, protein, nucleic acid, expression vector, recombinant cell, or pharmaceutical composition according to the invention, for prophylaxis or treatment of a condition or disease by an administration route selected among injection, inhalation, topical, transdermal, oral, nasal, vaginal, or anal delivery.

According to an embodiment, the invention concerns the use, wherein the mode of injection is selected among intravenous (IV), intraperironeal (IP), subcutaneous (SC) and (intramuscular) IM.

According to an embodiment, the invention concerns the use, for treatment of a disease by direct injection at a site affected by a disorder, such as inflammation.

According to an embodiment, the invention concerns the use of a polypeptide, protein, nucleic acid, expression vector, recombinant cell, or pharmaceutical composition according to the invention, for treatment of a condition selected among a skin disease, Psoriasis, Arthritis, Asthma, Sepsis, inflammatory bowel disease, rheumatoid arthritis, SLE, and spondyloarthritis.

According to an embodiment, the invention concerns the use of a polypeptide, protein, nucleic acid, expression vector, recombinant cell, or pharmaceutical composition according to the invention, for treatment of Arthritis where the composition is injected directly at site of inflammation.

According to an embodiment, the invention concerns the use of a polypeptide, protein, nucleic acid, expression vector, recombinant cell, or pharmaceutical composition according to the invention, for treatment of a condition selected among Gastrointestinal hyperresponsiveness, Food Allergy, Food intolerance and inflammatory bowel disease, preferably wherein the composition is delivered orally.

According to an embodiment, the invention concerns the use of a polypeptide, protein, nucleic acid, expression vector, recombinant cell, or pharmaceutical composition according to the invention, for treatment Asthma where the composition is delivered by inhalation.

According to an embodiment of the present invention, the entitie(s) (polypeptide(s), protein(s), nucleic acid(s), expression vector(s), recombinant cell(s), pharmaceutical composition(s) and/or implant(s)) of the present invention can be used to reduce or amerliorate the effects of inflammation and/or inflammatory and autoimmune diseases.

The entities of the present invention may for example exercise their immune modulatory activity through binding to a protein on or inside lymphocytes, monocytes or other cells of the immune system. Such proteins or receptors can belong to any protein family including but not limited to Toll like receptors (TLRs), G-protein coupled receptors (GPCRs), antibodies, adhesion molecules, transporters (including but not limited to amino acid, inorganic ion, organic ion or sugar transporters, transmembrane pumps, transporter proteins, escort proteins, add transport proteins, cation transport proteins, or anion transport proteins), channel proteins such as ion-channels including but not limited to sodium channels, potassium channels, calcium channels, phosphate channels and any other cation or anion transporters. Especially calcium and calcium activated potassium channels, which are involved in activation of lymphocytes and monocytes may be targeted by ISD peptides.

The entities of the present invention may also exercise their immune modulatory activity through introducing changes to the cellular membranes such as changing the membrane curvature or permeabilize or destabilize the membrane allowing metabolites or other molecules and ions to pass through, thereby disrupting the biologically relevant concentrations of such molecules inside the cells or interrupting gradients of such molecules across membranes, which might be important for the normal function of the cells. Other mechanisms may exist.

The Sakaguchi Mice Model and the Collagen Induced Arthritis—CIA—Mouse Model Models (Predictive for Anti-Rheumatoid Arthritis Activity of the ISD Peptide in Humans) and the In Vivo Validation of the Peptides, Proteins and Pharmaceutical Formulations of the Invention:

SKG mice spontaneously develop T cell-mediated chronic autoimmune arthritis. This is due to a mutation of the gene encoding a Src homology 2 (SH2) domain of ζ-associated protein of 70 kDa (ZAP-70), which is a key signal transduction molecule in T cells (Sakaguchi et al., Nature 2003). This mutation impairs positive and negative selection of T cells in the thymus, leading to thymic production of arthritogenic T cells. Clinically, joint swelling begins in small joints of the digits, progressing in a symmetrical fashion to larger joints including wrists and ankles. Histologically, the swollen joints show severe synovitis with formation of pannus invading and eroding adjacent cartilage and subchondral bone. SKG mice develop extra-articular lesions, such as interstitial pneumonitis, vasculitides, and subcutaneous necrobiotic nodules not unlike rheumatoid nodules in RA. Serologically, they develop high titers of RF and autoantibodies specific for type II collagen. Furthermore, CD4⁺ T cells can adoptively transfer arthritis in SKG mice, which have a BALB/c genetic background, to T cell-deficient BALB/c nude or T cell/B cell-deficient SCID mice, which indicates that the disease is a T cell-mediated autoimmune disease. In addition to the causative gene, the polymorphism of the MHC gene also contributes to the occurrence of SKG arthritis depending on environmental conditions. Thus, this spontaneous autoimmune arthritis in mice resembles human RA in clinical and histological characteristics of articular and extra-articular lesions, in serological characteristics, and in the key role of CD4⁺ T cells in initiating arthritis (Sakaguchi et al Nature 2003).

Cytokines play key roles in spontaneous CD4⁺ T cell-mediated chronic autoimmune arthritis in SKG mice. A study conducted by Hata et al. (J Clin Invest 2004) show that genetic deficiency in IL-6 completely suppressed the development of arthritis in SKG mice, irrespective of the persistence of circulating rheumatoid factor. Either IL-1 or TNF-α deficiency retarded the onset of arthritis and substantially reduced its incidence and severity. IL-10 deficiency, on the other hand, exacerbated disease, whereas IL-4 or IFN-γ deficiency did not alter the disease course. Synovial fluid of arthritic SKG mice contained high amounts of IL-6, TNF-α, and IL-1, in accordance with active transcription of these cytokine genes in the afflicted joints. Notably, immunohistochemistry revealed that distinct subsets of synovial cells produced different cytokines in the inflamed synovium: the superficial synovial lining cells mainly produced IL-1 and TNF-α, whereas scattered subsynovial cells produced IL-6. Thus, IL-6, IL-1, TNF-α, and IL-10 play distinct roles in the development of SKG arthritis. The results also indicate that targeting not only each cytokine but also each cell population secreting distinct cytokines could be an effective treatment of rheumatoid arthritis (Hata et al. j. Clin Invest 2004).

According to an embodiment of the present invention the entities of the present invention are capable of suppressing the development of inflammation, specially joint inflammation, in the Sakaguchi (SKG) mouse model for arthritis. According to the present invention, the entities of the present invention are capable of reducing the arthritis score in such animals, the score being reduced with at least 5%, such as least 10%, at least, 15%, at least 20%, such as at least 25%, at least 30%, at least 35%, such as at least 40%, at least 45%, such as at least 50%, at least 55%, such as at least 60%, at least 65%, such as at least 70%, at least 75%, such as at least 80%, at least 85% reduction of the score upon induction of inflammation by mannan injection.

The entities of the present invention are capable suppressing the immune response in an animal suffering from a general inflammation according to the SKG mouse model. According to the present invention, the entities of the present invention are capable of reducing IL-6 levels in Sakaguchi mice challenged with mannan, the IL-6 levels being reduced with at least 5%, such as at least 10%, at least, 15%, at least 20%, such as at least 25%, at least 30%, at least 35%, such as at least 40%, at least 45%, such as at least 50%, at least 55%, such as at least 60%, at least 65%, such as at least 70%, at least 75%, such as at least 80%, at least 85% reduction of the IL-6 levels in mannan challenged SKG-mice.

The collagen-induced arthritis (CIA) mouse model is the most commonly studied autoimmune model of rheumatoid arthritis. Autoimmune arthritis is induced in this model by immunization with an emulsion of complete Freund's adjuvant and type II collagen (CII).

The model shares several pathological features with RA, and type II collagen (CII) is a major protein in cartilage, the target tissue of RA. Additionally, of the antigen-defined models that are based on cartilage proteins, the CIA mouse model has the shortest duration between immunization and disease manifestation. The CIA model has been used extensively to identify potential pathogenic mechanisms of autoimmunity, including the role of individual cell types in disease onset and progression, as well as to design and test new therapeutics. In recent years, the CIA model has been instrumental in the testing and development of the new biologically based therapeutics, such as those that target tumor necrosis factor-α, a cytokine produced by macrophages and T cells that is a dominant inflammatory mediator in the pathogenesis of RA.

CIA is elicited in genetically susceptible strains of mice by immunization with CII emulsified in complete Freund's adjuvant (CFA). The ensuing pathogenesis shares several pathological features with RA, including synovial hyperplasia, mononuclear cell infiltration, cartilage degradation, and, like RA, susceptibility is linked to the expression of specific MHC class II genes. The most notable differences between this model and RA are that rheumatoid factor is not present in CIA, there is little or no sex bias in CIA and that the experimental disease is generally monophasic, although some relapsing mouse models of CIA have been described. While the presence of T-cell and B-cell immunity to CII has been reported in RA, it is not clear if this is a causative factor or a result of the pathogenesis associated with this disease. The original “gold standard” of the CIA model was the DBA/1 (H-2q) mouse strain; however, in recent years, several HLA-DR mouse models have been established in which transgenic expression of the HLA-DR1 or DR4 class II genes associated with susceptibility to RA confers susceptibility to CIA in the recipient mouse strain. These data indicate that the DR molecules associated with susceptibility to RA are at least involved in the immune response to CII.

The immunopathogenesis of CIA involves both a T-cell and B-cell specific response to CII. The immunodominant T-cell determinants of CII that mediate CIA have been identified for most of the class II molecules that are associated with susceptibility to this experimental disease, and a few have been studied in detail for their interaction with the class II molecule and T-cell receptor. Similarly, B-cell determinants targeted by the antibody response to CII have also been identified, and there is some evidence that antibodies from RA patients target the same areas of the CII molecule as those from CIA. Identification of pathogenic B-cell determinants has proven to be more difficult owing to the requirement that the pathogenic antibodies must be able to bind to the triple helical native CII. Unlike other autoimmune models such as experimental autoimmune encephalomyelitis (EAE), where T cells are the primary pathogenic mechanism, the pathogenesis of CIA is mediated, in a large part, by CII-specific antibodies that binds to the cartilage and is capable of fixing complement. Collectively, these data have enabled researchers to study a wide range of pathogenic mechanisms in this model, as well as to design and test novel therapeutics (Brand, Latham, & Rosloniec, 2007).

TNF-α plays an important role in CIA. Studies have shown that suppression of collagenarthritis was achieved both with neutralizing antibodies against TNFα and with soluble TNF receptors. Intriguingly, it was found that TNFα was crucial at the onset of the arthritis but appeared less dominant in the later stages. In fact, studies in TNF receptor knockout mice demonstrated that the incidence and severity of arthritis were less in such mice; once the joints became affected, however, full progression to erosive damage was noted in an apparently TNF-independent fashion.

Likewise, in CIA, it was shown that treatment with a set of neutralizing antibodies against both IL-1a and IL-1β was still highly effective in established arthritis, reducing both inflammation and the progression of cartilage destruction. Studies with antibodies to separate IL-1 isoforms revealed that IL-1β is more crucial. This is in line with the clear efficacy in this model of ICE (IL-1β-converting enzyme) inhibitors and the observation of reduced CIA in ICE-deficient mice. Similarly, the local overexpression of IL-1ra by retroviral gene transfer in inflamed knee joints was effective at the site. In line with the identification of TNFα and IL-1β as separate targets in animal models of arthritis, it has been convincingly demonstrated that combination therapy with both TNF and IL-1 blockers provides optimal protection.

IL-6 also plays an important role in the development of CIA, IL-6−/− mice are completely protected from CIA, accompanied by a reduced antibody response to type II collagen and the absence of inflammatory cells and tissue damage in knee joints. Both suppression of specific immune responses to CII and a tendency to a shift toward a Th2 cytokine profile might contribute in part to the attenuation of CIA in IL-6−/− mice (Sasai et al., 1999).

According to an additional embodiment of the present invention the entities of the present invention are capable of suppressing the development of inflammation, specially joint inflammation, in the Collagen Induced Arthritis (CIA) mouse model for arthritis.

According to an embodiment of the present invention, the immunosuppressive polypeptides of the invention are capable of reducing the arthritis score in such animals, the score being reduced with at least 5%, such as least 10%, at least, 15%, at least 20%, such as at least 25%, at least 30%, at least 35%, such as at least 40%, at least 45%, such as at least 50%, at least 55%, such as at least 60%, at least 65%, such as at least 70%, at least 75%, such as at least 80%, at least 85% reduction of the score from induction of inflammation by collagen injection.

### Protein and Peptides

In one embodiment of the present invention, the polypeptides are monomeric. In another embodiment of the present invention the polypeptides are dimeric. In another embodiment of the present invention the polypeptides are trimeric. In yet another embodiment of the present invention the polypeptides are multimeric. Thus, according to the present invention the polypeptides may be monomeric, homologous dimeric, heterologous dimeric, homologous trimeric, heterologous trimeric, homologous multimeric and/or heterologous multimeric. In a particular preferred embodiment the polypeptides of the present invention are homologous dimeric.

Additionally, the present invention may comprise combinations of di-, tri- and/or multimeric polypeptides. In one embodiment the present invention comprises homologues dimeric peptides in combination with other homologous dimeric peptides. In another embodiment the invention comprises homologous dimeric peptides in combination with heterologous dimeric peptides. The following combinations of peptides are also within the scope of this invention: homologous dimeric peptides with homologous trimeric, homologous dimeric with heterologous trimeric, heterologous dimeric with homologous trimeric, heterologous dimeric with heterologous trimeric, homologous dimeric with homologous multimeric, heterologous dimeric with homologous multimeric, homologous dimeric with heterologous multimeric, heterologous dimeric with heterologous multimeric, homologous trimeric with homologous multimeric, homologous trimeric with heterologous multimeric, heterologous trimeric with homologous multimeric and heterologous trimeric with heterologous multimeric immusuppressive peptides.

In certain embodiments of the present invention the polypeptides are homologous dimers, such as homologous dimers formed by two of the peptides selected among SEQ ID NO: 1-1043.

In one embodiment the monomeric peptides are cross-linked into a dimer by cross-linking the peptides N-terminal to N-terminal or C-terminal to C-terminal. In a preferred embodiment the peptides are cross-linked via a disulfide bond wherein the peptides are cross-linked C-terminal to C-terminal.

In an embodiment, a polypeptide of the invention is linked to at least one protein, which may act as a carrier protein. A multimer may be formed by linking to a carrier protein or other molecule and/or by linking several peptides to said carrier protein.

In one embodiment the monomeric peptides are chemically linked to a protein (such as a carrier protein) or any other molecule that can be coupled to more than one of peptide. The coupling can be through a covalent bond or through weaker bonds such as hydrogen bonds or van der Waals bonds. The peptides can be coupled through in its N-terminal, C-terminal or anywhere inside the peptide sequence. Any method described here in for cross-linking of peptides can be used to couple the peptide to the protein or the carrier molecule resulting in a molecule that contains several copies of the said peptide.

The polypeptides of the present invention may be of different length. However, it is appreciated that the active component of the immunosuppressive peptides have a maximum length of about 100 amino acids, such as about 90 amino acids, for example about 80 amino acids, such as about 70 amino acids, such as about 60 amino acids, for example about 50 amino acids, such as 40 amino acids, for example about 35 amino acids.

According to an embodiment, the polypeptide or the sequence of said polypeptide may form part of a larger peptide or molecule and still retain its biological properties. According to an embodiment additional aminoacids or molecules may be added to an immunosuppressive peptide in order to improve the solubility and/or bioavailability characteristics of said immusuppressive peptide.

Moreover, the present invention also encompasses polypeptides, wherein one or more amino acid residues are modified, wherein said one or more modification(s) are preferably selected from the group consisting of in vivo or in vitro chemical derivatization, such as but not limited to acetylation or carboxylation, glycosylation, such as glycosylation resulting from exposing the polypeptide to enzymes which affect glycosylation, for example mammalian glycosylating or deglycosylating enzymes, phosphorylation, such as modification of amino acid residues which results in phosphorylated amino acid residues, for example phosphotyrosine, phosphoserine and phosphothreonine. The polypeptides according to the invention can comprise one or more amino acids independently selected from the group consisting of naturally occurring L-amino acids, D-amino acids as well as non-naturally occurring, synthetic amino acids. One or more amino acid residues of the polypeptide of the present invention are modified so as to preferably improve the resistance to proteolytic degradation and stability or to optimize solubility properties or to render the polypeptide more suitable as a therapeutic agent. The invention also relates to polypeptides of the invention where blocking groups are introduced in order to protect and/or stabilize the N- and/or C-termini of the polypeptide from undesirable degradation. Such blocking groups may be selected from the group comprising but not limited to branched or non-branched alkyl groups and acyl groups, such as formyl and acetyl groups, as well substituted forms thereof, such as acetamidomethyl. The invention also relates to the following: The polypeptides according to present invention, wherein the one or more blocking groups are selected from N-terminal blocking groups comprising desamino analogs of amino acids, which are either coupled to the N-terminus of the peptide or used in place of the N-terminal amino acid residue. The polypeptide according to present invention, but not limited to wherein the one or more blocking groups are selected from C-terminal blocking groups wherein the carboxyl group of the C-terminus is either incorporated or not, such as esters, ketones, and amides, as well as descarboxylated amino acid analogues. The polypeptide according to present invention, wherein the one or more blocking groups are selected from C-terminal blocking groups comprising ester or ketoneforming alkyl groups, such as lower (C1 to C6) alkyl groups, for example methyl, ethyl and propyl, and amide-forming amino groups, such as primary amines (—NH2), and mono- and di-alkylamino groups, such as methylamino, ethylamino, dimethylamino, diethylamino, methylethylamino, and the like. The polypeptide according to present invention, wherein free amino group(s) at the N-terminal end and free carboxyl group(s) at the termini can be removed altogether from the polypeptide to yield desamino and descarboxylated forms thereof without significantly affecting the biological activity of the polypeptide. The desirable properties may be achieved for example by chemical protection, i.e. by reacting the proteins and peptides of the present invention with protecting chemical groups, or by the incorporation of non-naturally occurring amino acids, e.g. D-amino acids, with the result of prolonging the half-life of the proteins and peptides of the present invention.

According to an embodiment, the present invention concerns endogenous retrovirus derived genes or gene segments. Endogenous retroviruses are remnants of ancient retroviral integrations and readily identifiable because of their sequence homology to other retroviruses to a person who is skilled-in-the-art.

According to an embodiment, the present invention concerns endogenous retrovirus derived genes or gene segments which are transcribed to RNA.

According to an embodiment, the present invention concerns endogenous retrovirus derived genes or gene segments which are transcribed into RNA and whose transcription level is correlated to the transcription level of other genes involved in a disease or condition.

According to an embodiment, the present invention concerns endogenous retrovirus derived genes or gene segments which are transcribed into RNA and whose transcription level is correlated to the transcription level of other genes involved in autoimmunity.

According to an embodiment, the present invention concerns endogenous retrovirus derived genes or gene segments which are transcribed into RNA and whose transcription level is different in subjects with a condition as compared to subjects without such a condition. Such conditions can be diseases such as autoimmune diseases or congenital diseases.

According to an embodiment, the present invention concerns endogenous retrovirus derived genes or gene segments which are transcribed into RNA and whose transcription level is different in subjects with an autoimmune condition as compared to subjects without such a condition. Such conditions include SLE, Rheumatoid Arthritis, IBD and others.

According to an embodiment, the present invention concerns endogenous retrovirus derived genes or gene segments which have different copy numbers in different individuals.

According to an embodiment, the present invention concerns endogenous retrovirus derived genes or gene segments which have different copy numbers in individuals with a disease or condition as compared to individuals without said disease or condition

According to an embodiment, the present invention concerns endogenous retrovirus derived genes or gene segments which have different copy numbers in individuals with autoimmunity as compared to individuals without autoimmune conditions

According to an embodiment, the present invention concerns endogenous retrovirus derived genes or gene segments which have different copy numbers in individuals with autoimmunity or congenital diseases as compared to individuals without autoimmune conditions or congenital diseases.

According to an embodiment, the present invention concerns endogenous retrovirus derived genes or gene segments which have different copy numbers in individuals with conditions such as SLE, Rheumatoid Arthritis, IBD and others as compared to individuals without such conditions

According to an embodiment, the present invention concerns endogenous retrovirus derived genes or gene segments which contain single nucleotide polymorphisms (SNPs) in different individuals.

According to an embodiment, the present invention concerns endogenous retrovirus derived genes or gene segments which have single nucleotide polymorphisms (SNPs) correlated with occurrence of a disease or condition as compared to individuals without said disease or condition

According to an embodiment, the present invention concerns endogenous retrovirus derived genes or gene segments which have SNPs correlated with occurrence of autoimmunity as compared to individuals without autoimmune conditions

According to an embodiment, the present invention concerns endogenous retrovirus derived genes or gene segments which have SNPs which occur more or less frequently in individuals with autoimmunity or congenital diseases as compared to individuals without autoimmune conditions or congenital diseases.

According to an embodiment, the present invention concerns endogenous retrovirus derived genes or gene segments which have SNPs correlated with occurrence of conditions such as SLE, Rheumatoid Arthritis, IBD and others as compared to individuals without such conditions

According to an embodiment, the present invention concerns compositions of one or more immunosuppressive peptides. Immunosuppressive polypeptides are polypeptides that are capable of suppressing an immune response in animals, including human beings and other animal such as domestic or agricultural (cats, dogs, cows, sheep, horses, pigs, etc.) or test species such as mouse, rats, rabbits and the like.

In one embodiment of the present invention the immunosuppressive polypeptides are capable of at least 5% inhibition of T-lymphocyte proliferation, at least 10%, at least 20%, such as at least 30%, at least 40%, at least 50%, such as at least 60%, such as at least 70% inhibition of T-lymphocyte proliferation. In particular embodiments the immunosuppressive peptides of the present invention are capable of at least 75% inhibition of T-lymphocyte proliferation, at least 80%, such as at least 85%, at least 90%, such as at least 95%, at least 97%, such as at least 99%, at least 100% inhibition of T-lymphocyte proliferation.

According to another embodiment of the present invention the immunosuppressive polypeptides are capable of suppressing the immune response in an animal suffering from a general skin inflammation according to the TPA model, an irritant contact dermatitis model, as described herein below. According to the present invention, the immunosuppressive polypeptides of the present invention are capable of reducing the ear thickening in mice challenged with phorbol 12-myristate 13-acetate (TPA), the ear thickening being reduced with at least 5%, such as least 10%, at least, 15%, at least 20%, such as at least 25%, at least 30%, at least 35%, such as at least 40%, at least 45%, such as at least 50%, at least 55%, such as at least 60%, at least 65%, such as at least 70%, at least 75%, such as at least 80%, at least 85% reduction of ear thickening following TPA challenge.

Examples of pharmaceutically acceptable acid addition salts for use in the present inventive pharmaceutical composition include those derived from mineral acids, such as hydrochloric, hydrobromic, phosphoric, metaphosphoric, nitric and sulfuric acids, and organic acids, such as tartaric, acetic, citric, malic, lactic, fumaric, benzoic, glycolic, gluconic, succinic, p-toluenesulphonic acids, and arylsulphonic, for example.

### Some Definitions

### Protein and Peptides

The terms “peptide” and “polypeptide” refers to any molecule containing at least three amino acid residues coupled through peptide bonds. The term “polypeptide” is used here for peptides and/or proteins without necessarily being constricted to a specific length of said polypeptide.

The term “protein” is used interchangeably with polypeptide and is not limited to any specific length or size. Polypeptides and proteins can be in the form of fragments or complexes or can have any primary, secondary, tertiary or quaternary structure such as but not limited to monomer, dimer, trimer, tetramer or multimer, alpha helix, beta sheet or any other helix structures and/or globular structures. Polypeptides and proteins can contain crosslinkings or any chemical modifications.

Polypeptides and proteins can be modified. As non-restricting examples, a polypeptide of the invention may be glycolysed, acylased and/or dimerized or trimerized, but does not need to be glycolysed, acylased and/or dimerized or trimerized.

Polypeptides and proteins can comprise one or more amino acids independently selected from the group consisting of naturally occurring L-amino acids, D-amino acids as well as non-naturally occurring, synthetic amino acids.

The expression “cross-linker” or “cross-linking moiety” refers to a linking moiety conferred by an external cross-linking agent used to crosslink one polypeptide with one or more polypeptides as described further in detail herein below.

The term “carrier” refers to a compound that is conjugated to the polypeptide(s) either to increase the number of polypeptides, for increasing activity or immunosuppressive effect of the polypeptide(s), to confer stability to the molecules, to increase the biological activity of the peptides, or to increase its serum half-life, or to reduce it's immunogenicity. The “carrier” may be a protein carrier or a non-protein carrier. Non-limiting examples of non-protein carriers include liposomes, micelles, polymeric nanoparticles and diaminoethane. The liposome may comprise glycosaminoglycan hyaluronan (HA) and/or PEG. In one embodiment, the carrier is an immunoliposome. Other carriers include protamines, or polysaccharides e.g. aminodextran or chitosan. Non-limiting examples of protein carriers include, keyhole limpet hemocyanin, serum proteins such as transferrin, bovine serum albumin, human serum albumin, whale myoglobin, ovalbumin, immunoglobulins, lysozyme, carbonic anhydrase, or hormones, such as insulin. In other embodiments of the present invention, the carrier may be a pharmaceutical acceptable carrier as described herein below. The immune modulating peptides of the present invention may be coupled to the carrier by means of cross-linking as further described herein below.

The terms “protein modification”, “protein stability” and “peptide stability” is used to describe the state of the proteins and peptides, in particular the state wherein said proteins and/or peptides are more resistant to degradation, fibrillation, and aggregation and/or have increased properties towards hydrolysis and/or proteolysis or have improved shelf-life. In particular, proteolytic stability refers to the resistance toward the action of proteolytic enzymes, also known as proteases, i.e. enzymes that catalyzes the hydrolysis of the amide/peptide-bond of the protein or peptide. Moreover, the present invention also encompasses polypeptides, wherein one or more amino acid residues are modified, wherein said one or more modification(s) are preferably selected from the group consisting of in vivo or in vitro chemical derivatization, such as but not limited to acetylation or carboxylation, glycosylation, such as glycosylation resulting from exposing the polypeptide to enzymes which affect glycosylation, for example mammalian glycosylating or deglycosylating enzymes, phosphorylation, such as modification of amino acid residues which results in phosphorylated amino acid residues, for example phosphotyrosine, phosphoserine and phosphothreonine. The polypeptide according to the invention can comprise one or more amino acids independently selected from the group consisting of naturally occurring L-amino acids, naturally occurring D-amino acids as well as non-naturally occurring, synthetic amino acids. One or more amino acid residues of the polypeptide of the present invention are modified so as to preferably improve the resistance to proteolytic degradation and stability or to optimize solubility properties or to render the polypeptide more suitable as a therapeutic agent. The invention also relates to polypeptides of the invention where blocking groups are introduced in order to protect and/or stabilize the N- and/or C-termini of the polypeptide from undesirable degradation. Such blocking groups may be selected from the group comprising but not limited to branched or non-branched alkyl groups and acyl groups, such as formyl and acetyl groups, as well substituted forms thereof, such as acetamidomethyl. The invention also relates to the following: The polypeptides according to present invention, wherein the one or more blocking groups are selected from N-terminal blocking groups comprising desamino analogs of amino acids, which are either coupled to the N-terminus of the peptide or used in place of the N-terminal amino acid residue. The polypeptide according to present invention, but not limited to wherein the one or more blocking groups are selected from C-terminal blocking groups wherein the carboxyl group of the C-terminus is either incorporated or not, such as esters, ketones, and amides, as well as descarboxylated amino acid analogues. The polypeptide according to present invention, wherein the one or more blocking groups are selected from C-terminal blocking groups comprising ester or ketoneforming alkyl groups, such as lower (C1 to C6) alkyl groups, for example methyl, ethyl and propyl, and amide-forming amino groups, such as primary amines (—NH2), and mono- and di-alkylamino groups, such as methylamino, ethylamino, dimethylamino, diethylamino, methylethylamino, and the like. The polypeptide according to present invention, wherein free amino group(s) at the N-terminal end and free carboxyl group(s) at the termini can be removed altogether from the polypeptide to yield desamino and descarboxylated forms thereof without significantly affecting the biological activity of the polypeptide. The increased properties may be achieved for example by chemical protection, i.e. by reacting the proteins and peptides of the present invention with protecting chemical groups, or by the incorporation of non-naturally occurring amino acids, e.g. D-amino acids, with the result of prolonging the half-life of the proteins and peptides of the present invention.

### Single Nucleotide Polymorphism (SNP):

A single nucleotide polymorphism, also known as simple nucleotide polymorphism, (SNP, pronounced snip; plural snips) is a DNA sequence variation occurring commonly within a population (e.g. 1%) in which a single nucleotide—A, T, C or G—in the genome (or other shared sequence) differs between members of a biological species or paired chromosomes. For example, two sequenced DNA fragments from different individuals, AAGCCTA to AAGCTTA, contain a difference in a single nucleotide. In this case we say that there are two alleles. Almost all common SNPs have only two alleles. The genomic distribution of SNPs is not homogenous; SNPs occur in non-coding regions more frequently than in coding regions or, in general, where natural selection is acting and ‘fixing’ the allele (eliminating other variants) of the SNP that constitutes the most favorable genetic adaptation. Other factors, like genetic recombination and mutation rate, can also determine SNP density.

SNP density can be predicted by the presence of microsatellites: AT microsatellites in particular are potent predictors of SNP density, with long (AT)(n) repeat tracts tending to be found in regions of significantly reduced SNP density and low GC content.

Within a population, SNPs can be assigned a minor allele frequency—the lowest allele frequency at a locus that is observed in a particular population. This is simply the lesser of the two allele frequencies for single-nucleotide polymorphisms. There are variations between human populations, so a SNP allele that is common in one geographical or ethnic group may be much rarer in another.

These genetic variations between individuals (particularly in non-coding parts of the genome) are sometimes exploited in DNA fingerprinting, which is used in forensic science. Also, these genetic variations underlie differences in our susceptibility to disease. The severity of illness and the way our body responds to treatments are also manifestations of genetic variations. For example, a single base mutation in the APOE (apolipoprotein E) gene is associated with a higher risk for Alzheimer disease.

### Homology and Identity

The term “homology” refers to sequence similarity or, interchangeably, sequence identity, between two or more polynucleotide sequences or two or more polypeptide sequences.

The phrases “percent identity” and “% identity,” as applied to polypeptide sequences, refer to the percentage of residue matches between at least two polypeptide sequences aligned using a standardized algorithm. Methods of polypeptide sequence alignment are well-known. Some alignment methods take into account conservative amino acid substitutions. Such conservative substitutions, explained in more detail above, generally preserve the charge and hydrophobicity at the site of substitution, thus preserving the structure (and therefore function) of the polypeptide.

“Percent identity” may be measured over the length of an entire defined polypeptide sequence, for example, as defined by a particular SEQ ID number, or may be measured over a shorter length, for example, over the length of a fragment taken from a larger, defined polypeptide sequence, for instance, a fragment of at least 6, at least 8, at least 10, at least 15, at least 20, at least 30, at least 40, at least 50, at least 70 or at least 150 contiguous residues. Such lengths are exemplary only, and it is understood that any fragment length supported by the sequences shown herein, in the tables, figures or Sequence Listing, may be used to describe a length over which percentage identity may be measured.

### Other Terms

An adjuvant is a component that potentiates the immune responses to an antigen and/or modulates it towards the desired immune responses. An adjuvant is defined as any substance that acts to accelerate, prolong, or enhance antigen-specific immune responses when used in combination with specific vaccine antigens.

The term Immunotherapy refers to the treatment of disease by inducing, enhancing, or suppressing an immune response. Immunotherapies designed to elicit or amplify an immune response are classified as activation immunotherapies, while immunotherapies that reduce or suppress are classified as suppression immunotherapies.

The term “immunosuppressive polypeptides” is used about polypeptides, which may exhibit immune suppressive activity. The term “immunosuppressive polypeptides of the invention” is used about polypeptides of the invention, which may exhibit immune suppressive activity.

The term “immune modulation” is used here about alteration of the immune system or of an immune response by agent(s) that activate or suppress its function. The term “immuno-modulation” might refer to the process of an immune response being either suppressed, partly or completely, or triggered or induced or enhanced. This may include immunization or administration of immunomodulatory drugs.

The term “immune modulating peptides” is used about polypeptides, which may exhibit immune modulating activity. The term “immune modulating polypeptides of the invention” is used about polypeptides of the invention, which may exhibit immune modulating activity.

Likewise, the term “growth-modulation” as used herein refers to the process of were the cell proliferation is either suppressed, partly or completely, or where cell proliferation is induced or enhanced or promoted.

The term “substance” as used anywhere herein comprises any form of substance suitable for comprising the polypeptides of the present invention.

Non-limiting examples of such substances are creams, lotions, shake lotions, ointments, gels, balms, salves, oils, foams, shampoos, sprays, aerosols as well as transdermal patches and bandages.

The term “treatment”, as used anywhere herein comprises any type of therapy, which aims at terminating, preventing, ameliorating and/or reducing the susceptibility to a clinical condition as described herein. In a preferred embodiment, the term treatment relates to prophylactic treatment, i.e. a therapy to reduce the susceptibility of a clinical condition, a disorder or condition as defined herein.

Thus, “treatment,” “treating,” and the like, as used herein, refer to obtaining a desired pharmacologic and/or physiologic effect, covering any treatment of a pathological condition or disorder in a mammal, including a human. The effect may be prophylactic in terms of completely or partially preventing a disorder or symptom thereof and/or may be therapeutic in terms of a partial or complete cure for a disorder and/or adverse affect attributable to the disorder. That is, “treatment” includes (1) preventing the disorder from occurring or recurring in a subject, (2) inhibiting the disorder, such as arresting its development, (3) stopping or terminating the disorder or at least symptoms associated therewith, so that the host no longer suffers from the disorder or its symptoms, such as causing regression of the disorder or its symptoms, for example, by restoring or repairing a lost, missing or defective function, or stimulating an inefficient process, or (4) relieving, alleviating, or ameliorating the disorder, or symptoms associated therewith, where ameliorating is used in a broad sense to refer to at least a reduction in the magnitude of a parameter, such as inflammation, pain, and/or immune deficiency.

The term “animal” as used herein may be defined to include humans, domestic or agricultural (cats, dogs, cows, sheep, horses, pigs, etc.) or test species such as mouse, rats, rabbits and the like. Thus the animals may also be of bovine, equine, porcine, human, ovine, caprine or cervidae origin.

The expression “derived from an endogenous retrovirus” means that the domain is substantially identical to the immune suppressive domain of the endogenous retrovirus, optionally with mutations, insertions or deletions.

All cited references are incorporated by reference.

The accompanying Figures and Examples are provided to explain rather than limit the present invention. It will be clear to the person skilled in the art that aspects, embodiments, items and claims of the present invention may be combined; also with features of the technical background and the cited references.

### Example 1: The HERV-Env 59 is Overexpressed in SLE Patients as Compared to Healthy Individuals

Here we present data on investigating HERV-Env59 gene expression in PBMCs (peripheral blood mononuclear cells) from 45 healthy individuals and from 45 patients with Systemic Lupus Erythematosus here SLE, by real-time RT-PCR. The data was normalized to RPL13a or RPL37A housekeeping genes, Venous blood samples were collected in CPT™ tubes (BD Vacutainers®, BD Diagnostics, NJ USA), and processed within 1 h. Tube/blood samples were centrifuged at room temperature in a horizontal rotor for a minimum of 30 minutes at 1800 g (relative centrifuge force). After centrifugation, mononuclear cell layers were collected and transferred to 15 ml size conical tubes. Following two washing steps, cell pellets were resuspended in the desired medium for subsequent RNA extraction. RNAs from peripheral blood samples were isolated using RNeasy® Plus Mini Kit (Qiagen, DK) according to the manufacturer's protocol. Quality and integrity of isolated RNA samples was controlled by determining A₂₅₀/A₂₈₀, A₂₆₀/A₂₃₀ absorbance ratios and 285/18S rRNA ratios. 200 ng total RNA purified from PBMCs was used for cDNA synthesis using iScript™ cDNA synthesis kit (Bio-Rad, CA USA) according to the instructions of the manufacturers. Real-time Q-PCR analysis was performed using a Light Cycler 480 cycler (Roche Diagnostics, DK). 2 μl of cDNA (from a total 20 μl reaction volume) was used in 20 μl reaction. The real-time Q-PCR reactions contained 10 μl SybrGreen 2× Master Mix (Roche Diagnostics, DK), 2 μl forward primer (5 pmol/μl), 2 μl reverse primer (5 pmol/μl) and 4 μl water. After initial denaturation at 95° C. for 10 minutes, PCR amplifications were performed for 45 cycles. The crossing point (CP) for each transcript was measured and defined at constant fluorescence level in Light Cycler 480 software. The mRNA levels for the test gene were normalized to the RPL13a value and relative quantification was determined using the ΔCt model presented by PE Applied Biosystems (Perkins Elmer, Foster City, Calif. USA).

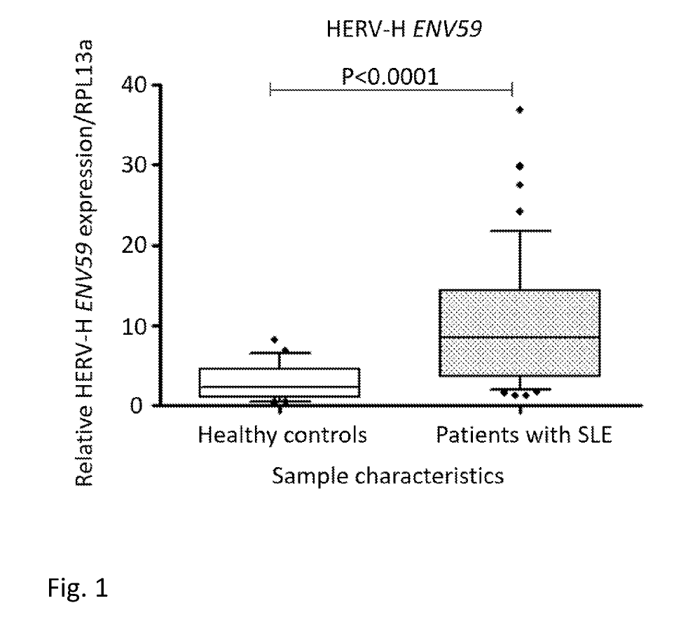

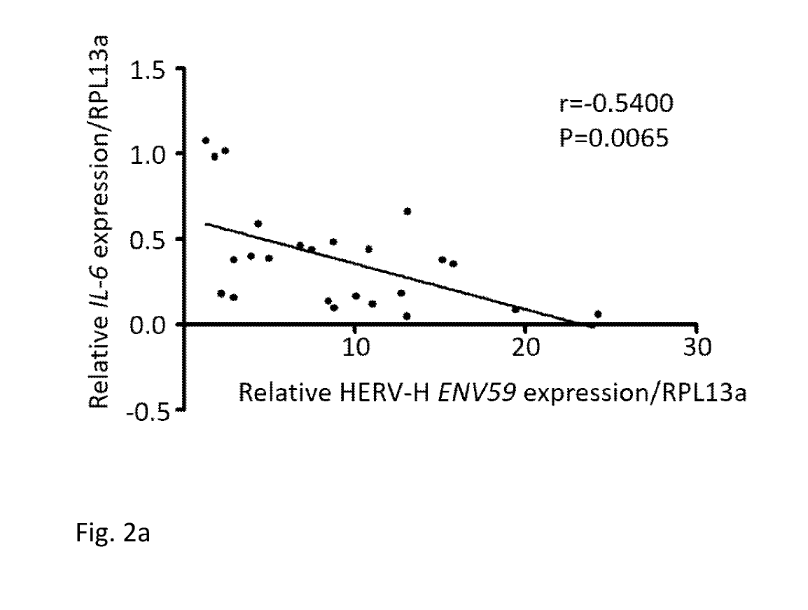

A specific amplification product (primer sets: Env 3 forward set 1 and Env 3 reverse set 1, ISD 59 forward and ISD59 reverse, EnvH3 forward and EnvH3 reverse) was observed in all SLE samples as well as in healthy controls. The relative HERV-Env 59 mRNA expression levels were significantly higher in patients with SLE than in healthy controls (P<0.001) FIG. 1. The bars show the median values and SD. The P value shows statistical differences (<0.05) between samples. The P value was calculated using nonparametric Mann-Whitney Utest. Furthermore significant variation in the HERV-Env59 mRNA expression level was observed in PBMCs samples obtained from patients with SLE. Differences were not due to the quality of retrotranscription, since the analysis of RNA RPL13a or RPL37A expression levels confirmed that total cDNA quantity was identical over all the samples tested.

The results are presented in FIG. 1.

### Example 2: Correlation Between IL-6 and/or TLR-7 mRNA and HERV-Env 59 Expression Levels in Patients with SLE

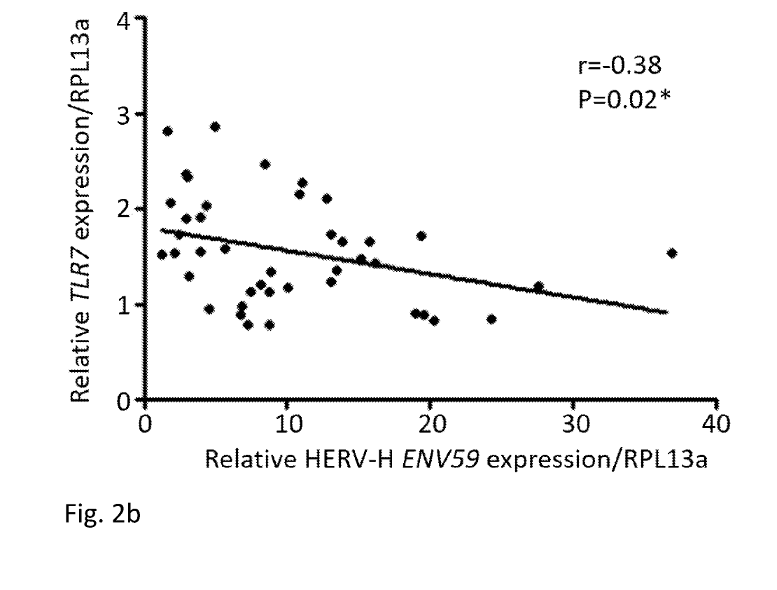

Here we present data on investigating a correlation between IL-6 and/or TLR7 mRNA and HERV-Env59 mRNA expression levels in patients with SLE. Among the sample population used in the study of Env59 expression levels, we examined IL-6 mRNA expression levels in PBMCs from patients with SLE by real-time RT-PCR (for details on the assay see Example 1). Additionally, we assessed TLR-7 mRNA expression levels in PBMCs from patients with SLE. The data were normalized to housekeeping gene RPL13a mRNA expression levels. We next performed a Spearman correlation analysis between the expression of HERV-Env 59 mRNA and IL-6 mRNA or TLR-7 mRNA, both of which showed a distinctive modulation. FIGS. 2a and 2b shows the HERV-Env59 gene expression levels, evaluated by real-time RT-PCR, in PBMCs obtained from patients with SLE, plotted against IL-6 or TLR-7 gene expressions. Statistical analysis demonstrated a significant negative correlation between the mRNA expressions of HERV-Env59 and IL-6 (P=0.0065, r=−0.5400) or TLR-7 (P=0.02, r=−0.38) in the SLE group. Thus, the correlation analysis suggests that higher levels of HERV-Env 59 are associated to lower levels of IL-6 or TLR-7 in SLE patients.

The results are presented in FIGS. 2a and 2b.

### Example 3: Characterization of a Functional Envelope Protein from the HERV-H3/Env-59 Locus

Here we present data on characterization of a functional envelope protein from HERV-Env 59 locus. The structural organization of the HERV-Env 59 was recognized previously, disclosing hydrophobicity profile as well as other characteristic feature of retroviral envelopes, i.e., a putative signal peptide located downstream of the M2 methionine, a CWLC motif, a furin cleavage site at the junction of the SU and TM subunits followed by hydrophobic fusion peptide and a hydrophobic membrane-spanning domain. In order to study the expression and activity of this protein further, the coding HERV-Env 59 cDNA was cloned into an expression vector, driven by a human cytomegalovirus (CMV) promoter. An HA-tag was added to the N-terminal of the protein after the putitative signal peptide identified through in silico methods. The PUC57Env59 plasmid was constructed by Genscript (NJ, USA), with a synthesized Env59 insert cloned into EcoRV site of PUC57 plasmid. Due to lack of commercial antibodies, the gene was fused to C-terminal HA-tag. For expression studies, Env59 was inserted into the 867 p-IRES-puro vector using EcoRI and NotI restriction sites to obtain final pEnv59IRESpuro construct. The correctness of the sequences was verified by sequencing.

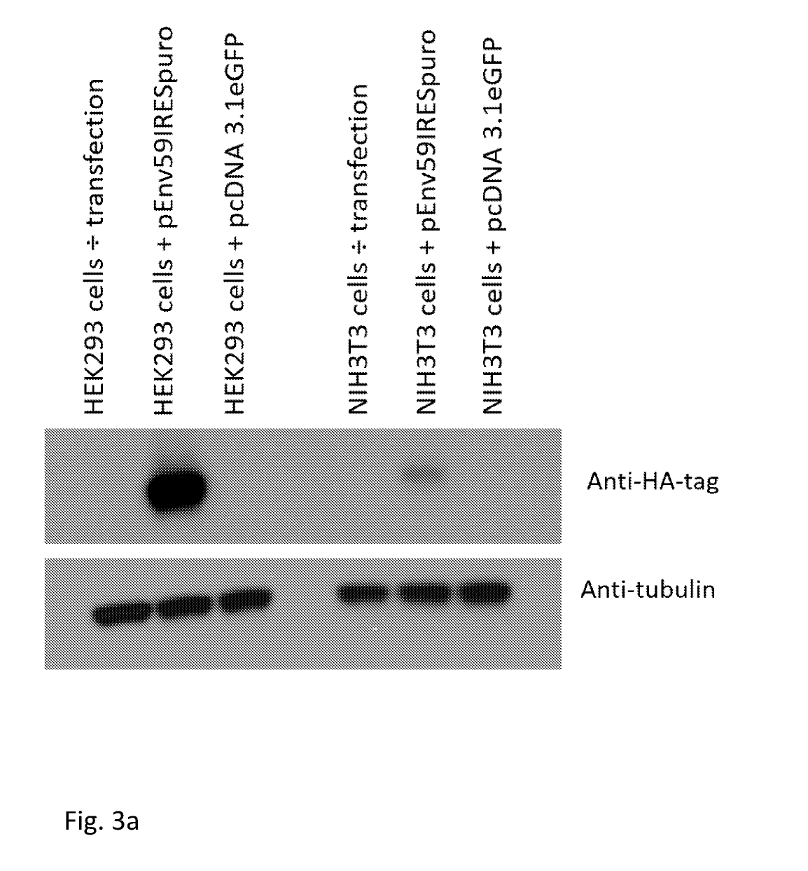

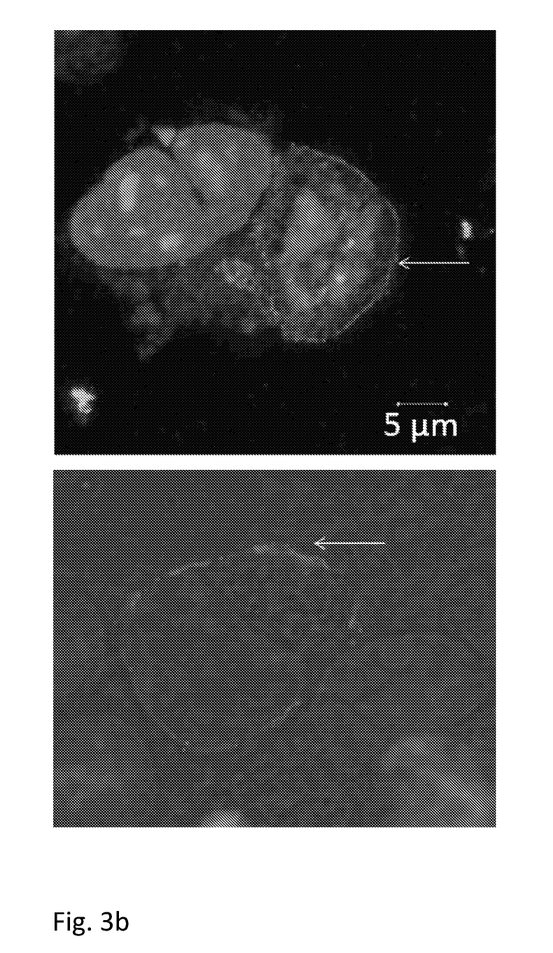

Expression of HERV-Env 59 gene was confirmed using a monoclonal antibody against the HA-tag in western blot in transiently transfected HEK293 or NIH3T3 cells using Lipofoctamine LTX® or Lipofectamine 2000® reagent (ThermoFisher Scientific) (FIG. 3a). 48 h after transfection cells were lysed and processed for Western blotting. Whole-cell extracts were prepared after lysis in NP-40 lysis buffer (10 mM Tris-HCL pH7.4, 137 mM NaCl, 10% v/v glycerol, 1% v/v Nonidet P-40) containing a protease inhibitor cocktail (Roche Diagnostics, DK). Cell debris were removed by centrifugation at 10.00×g for 25 min at 4° C. and protein concentration determined by BCA assay (Pierce, VWR/Bie&Berntsen, DK). Equal amounts of protein (20 μg/sample) were separated by SDS-polyacrylamide gel electrophoresis, transferred to nitrocellulose membranes, and incubated with specific antibodies, followed by incubation with HRP-conjugated secondary antibody. Immunoblots were developed by enhanced chemiluminescence using proprietary reagents (Millipore, DK).

As clearly evident, a band, corresponding to the molecular masses of ca. 62 kDa, was only detected in HEK293 and NIH3T3 cells transfected with the HERV-Env 59 expression vector, demonstrating that the HERV-Env 59 gene has the coding capacity for a full length protein and could be expressed ex vivo.

In order to determine correct surface expression of the envelope protein, we performed immunofluorescence confocal microscopy in transiently transfected HEK293 cells using a fluorescently labeled anti HA antibody. To visualize HERV-H Env59 surface expression in HEK293 cells transfected with the pEnv59IRESpuro and pcDNA-eGFP constructs by immunofluorescence, we stained formaldehyde-fixed, not permeabilized cells, with mouse anti-HA-tag antibody followed by fluorescence-labelled anti-mouse antibody, Alexa Fluor 568 nm. In all experiments, HERV-H Env59 detection was performed on cells grown on glass coverslips approximately 36-48 h post-transfection. Cells nuclei were stained using DAPI (4′,6-diamidino-2-phenylindole). No signal was detected with cells transfected with an irrelevant expression vector. Observations were made under a Zeiss LSM 510 laser scanning confocal microscope.

Staining of on nonpermeabilized cells showed that the HERV-Env 59 protein could be detected on the cell surface, confirming correct intracellular transport of the protein consistent with a retroviral envelope protein (FIG. 3b).

FIG. 3a depicts detection of HA-tag envelope glycoprotein in HERV-H Env59 HA-tag transfected cells.

Human HEK293 or mouse NIH3T3 cells were transfected with plasmids expressing either HERV-H Env59 HA-tag cDNA or control plasmid pcDNA 3.1 eGFP, or left untransfected in culture medium. 48 h later, the Env59-transfected cells, pcDNA 3.1 eGFP-transfected cells and untransfected cells were lysed and then proceed for Western blotting with antibodies (Ab) against HA-tag or tubulin.

FIG. 3b depicts expression of the HERV-Env59 protein. HERV-H Env59 proteins could be detected at the cell surface, a result consistent with the expected localization of a functional Env.

Confocal microscopy images from immunofluorescence analysis of human HEK293 cells transiently transfected with expression vector of the fully coding HERV-H Env59 gene (red) and control pcDNA 3.1 eGFP vector (green). Detection was performed on fixed and non-permeabalized HEK293 cells grown on glass coverslips approximately 36 h post transfection. HERV-H Env59 was detected (red) using a mouse anti HA-tag antibody. Cells nuclei are stained with DAPI.

### Example 4: HERV-Env 59 Encodes a Functional Envelope Protein

Here we present data on investigating functional properties of HERV-Env59 encoded protein. We investigated whether this envelope protein is still fusion active through pseudotyping of this protein with lentiviral core particles. Pseudotyped lentiviral/HERV-Env 59 particles were formed using a three plasmid vector system that includes a viral transfer vector encoding the eGFP protein. When co-transfected these three plasmids produce lentiviral particles that can be decorated with a surface protein if a fourth plasmid encoding such a protein is included in the transfection mixture. The plasmids for lentiviral vector packaging were kindly provided by Professor Jacob Giehm Mikkelsen. Lentiviral vectors pseudotyped with the vesicular stomatitis virus G-protein (VSV-G) encoded by pMD.2G, or pEnv59IRESpuro, or control pcDNA 3.1 were generated using the four plasmid expression lentiviral system containing the pCCL/PGK-eGFP.pMDLg/p-RRE.pRSV-REV. In our system, to further decrease the risk of recombination and production of replication-competent viruses, the Rev gene was inserted on the pRSV-REV plasmid. Virus was produced by transient transfection into 293T cells using standard calcium phosphate-mediated method. The total amount of DNA used per 6-well plate was 4 μg of lentiviral vector plasmids 1.59 μg of pCLL/PGK-eGFP, 1.59 μg of pMDIg/p-RRE, 0.37 μg of pRSV-Rev and 0.46 μg of pMDM.2G/pEnv59IRESpuro/pcDNA3.1. Forty eight hours after transfection, the vector-containing medium was collected and spun at 500×g for 5 min, filtered through a 0.45-μ pore size filter (Corning, N.Y. USA) and used fresh for transduction of target cells. Lentiviral titers were determined by seeding HEK293 cells in six-well plates at 5×10⁵ cells per well the day before infection with serial dilutions of the concentrated viral stock in the presence of polybrene (8 μg/ml). After 12 h incubation, the culture medium was changed and the cells were incubated for additional time.

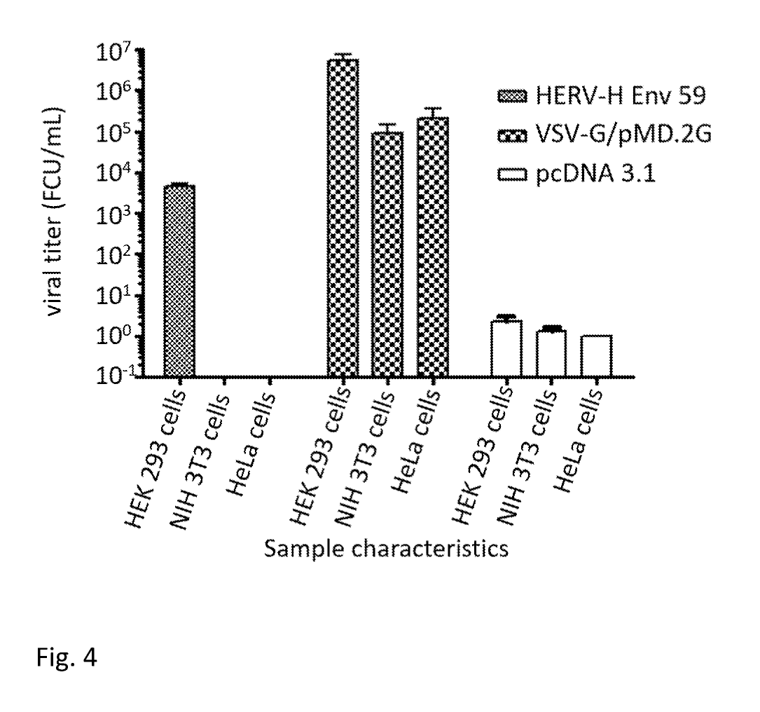

Cells expressing EGFP were identified using fluorescence microscopy (FIG. 4). As evident in FIG. 4, VSV-g pseudotyped particles infect all cell types showing that the assay is capable of detecting infection in all cell types, whereas the undecorated naked particles are not infectious. Most interestingly, ENV59 pseudotyped particles are only able to infect the HEK293 cells. The titers on human HEK293 cells were in the range of 3500- to 5000-CFU/ml range, which is quite significant. This suggests that ENV59 is incorporated into budding virions and constitutes an active fusion capable envelope protein. Furthermore, it seems that this envelope protein uses a receptor that is only found on HEK293 cells and not on murine NIH3T3 or HeLa cells.

Infection assay of the HERV-H Env59 envelope. Formation of the infectious HERV-H Env59 hybrid viral particles. Lentiviral vectors pseudotyped with the vesicular stomatitis virus G-protein (VSV-G) encoded by pMD.2G, or pEnv59IRESpuro, or control pcDNA 3.1 were generated using the four plasmid expression lentiviral system. Pseudotyped virions were assayed for infectivity and the target cells were human HEK293 cells. Viral titers are the means from two independent experiments.

### Example 5: Immunomodulatory Function Induced by HERV-Env 59 Retroviral Peptide with Impact on the Pathogenesis of SLE and Other Autoimmune Diseases

Inflammatory shock as a consequence of LPS release remains a serious clinical concern. In humans, inflammatory responses to LPS result in the release of cytokines and other cell mediators from monocytes and macrophages, which can cause fever, shock, organ failure and death. Pro- and anti-LPS is widely used as a potent and prototypical inducer of cytokine production in innate immunity which begins with the orchestration of monocytes. Pathogen associated molecular patterns (PAMPs), like lipopolysaccharide (LPS), play a pivotal role in initiation of variety of host responses caused by infection with Gram-negative bacteria. Such action leads to systemic inflammatory response, for instance up-regulation of pro- and anti-inflammatory cytokines, resulting in secretion of cytokine proteins into the blood stream.

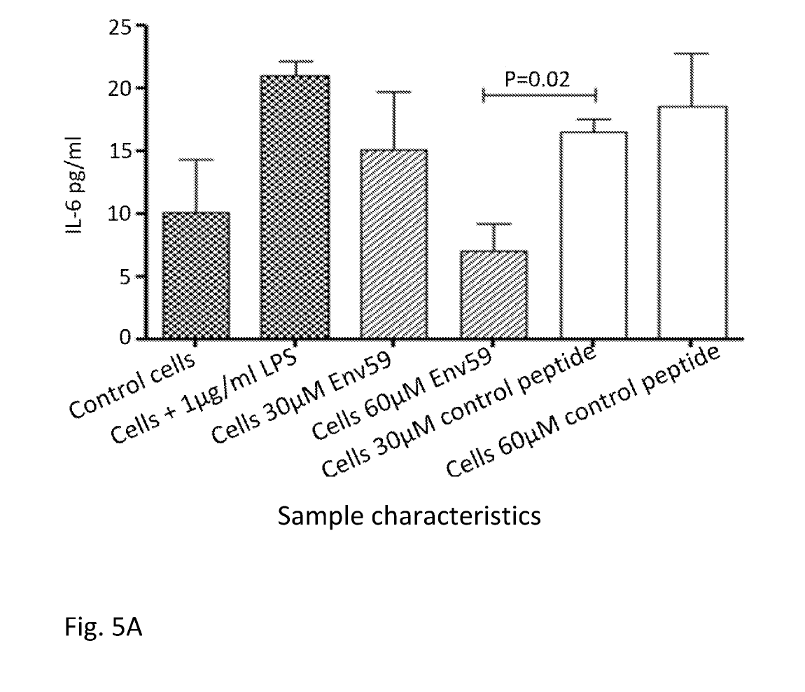

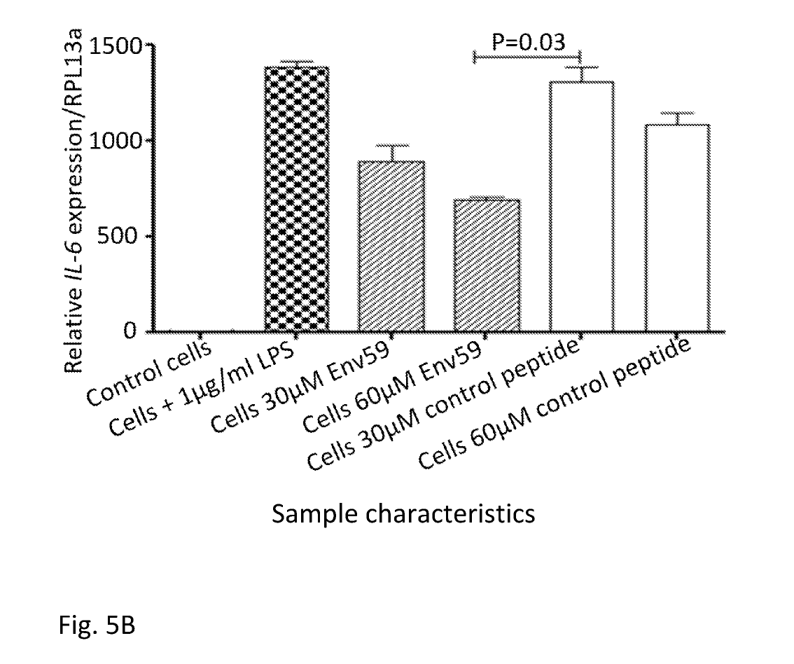

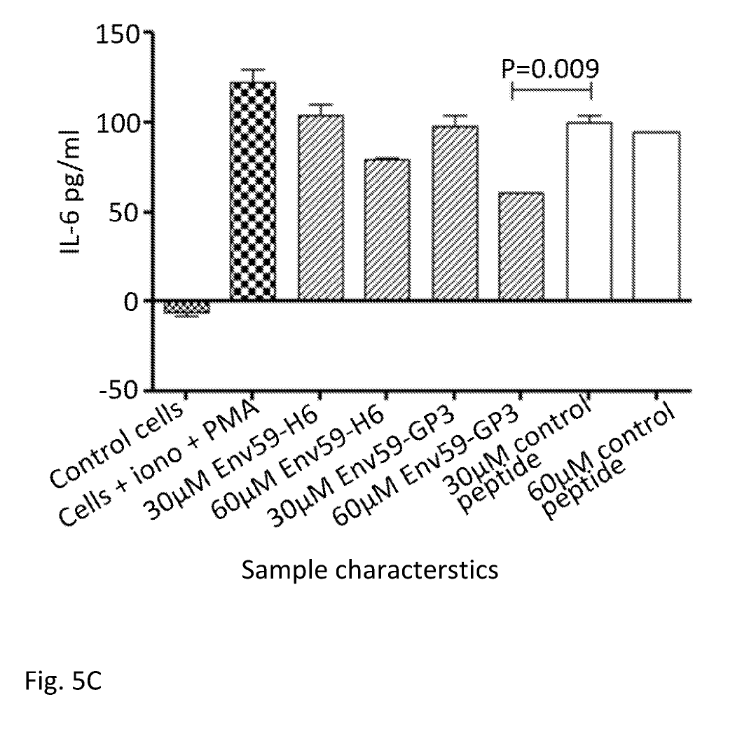

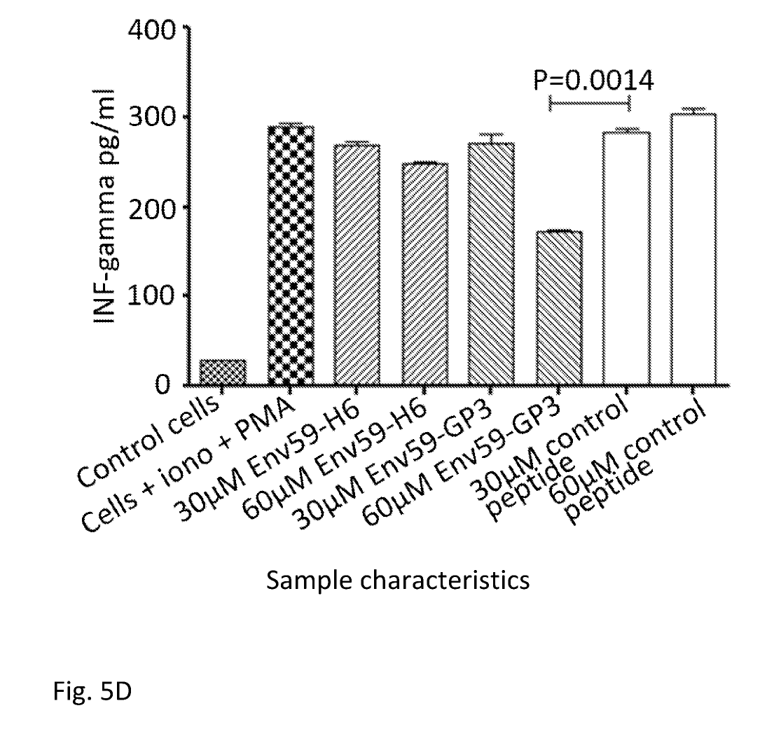

Here we present data showing that pretreatment of cells with ENV59 peptide results in a decrease in the release of cytokines including pro-inflammatory cytokines such as IL-6. Therefore, treatment of patients, in the risk of developing sepsis or other inflammatory condition, with ENV59 peptide could act beneficially to decrease production of proinflammatory cytokines and hereby lessen the risk of developing shock, organ failure and death. Here, we examined the modulatory function of Env 59 ISD on the expression levels of IL-6 in human acute monocytic leukemia cell line THP-1 and PBMCs obtained from healthy donors or patients with SLE. THP-1 cells and PBMCs were maintained in RPMI 1640 supplemented with 10% FBS, 100 U/ml penicillin, 100 μg/ml streptomycin and 2 mM L-glutamine at 37° C. in a 5% CO₂ incubator. THP-1 cells are known to induce IL-6 mRNA and protein in response to lipopolysaccharide (LPS) treatment. THP-1 cells were left untreated or incubated with 0 uM, 30 μM or 60 μM of Env-59 ISD and stimulated with 1 μg/μl LPS for 4 h, based on the previous analyses to find the optimal dose and incubation times. FIGS. 5a and 5b are representative for the results of real-time RT-PCR (for assay details see example 1) and ELISA analyses on stimulant-induced IL-6 mRNA protein expression.

For IL-6 ELISA analyses the supernatant from THP-1 cells or PBMCs (FIGS. 5 c and d) treated with peptides was assayed on human IL-6 ELISA Max™ Deluxe Set (Biolegend, #430505). ELISA assay was performed according to the manufacturer's protocol, as follows. Each incubation step was followed by sealing and shaking on the rotating table at 150-200 rpm, except the overnight incubation with the Capture Antibody, where plates were not shaken. One day prior running ELISA the 96-well assay plates were covered with the Capture Antibody, diluted 1:200 in 1× Coating Buffer (5× Coating Buffer diluted in ddH₂O). 100 μL of this Capture Antibody solution was added into all wells, sealed and incubated overnight (16-18 hrs) at 4° C. The next day all reagents from the set were brought to the room temperature (RT) before use. The plate was washed 4 times with minimum 300 μL Wash Buffer (1×PBS, 0.05% Tween 20) per well. The residual buffer in the following washing was removed by blotting the plates against the absorbent paper. Next 200 μL of the 1× Assay Diluent A (5× Assay Diluent A diluted in PBS pH=7.4) was added for 1 h to block non-specific binding. While the plate was being blocked, all samples and standards (mandatory for each plate) were prepared. Standards and samples were run in triplicates. 1 mL of the top standard 250 pg/mL was prepared in 1× Assay Diluent A (1× AD) from the IL-6 stock solution. The six two-fold serial dilutions of the 250 pg/mL top standard were performed, with the human IL-6 standard concentration: 250 pg/mL, 125 pg/mL, 62.5 pg/mL, 31.2 pg/mL, 15.6 pg/mL, 7.8 pg/mL and 3.9 pg/mL, respectively. 1× AD serves as the zero standard (0 pg/mL). After blocking the plate, washing was performed and 100 μL standards and samples were assayed in triplicates and incubated for 2 h in RT. Samples were not diluted, the whole supernatant from the THP-1 or PBMCs cells was assayed. After washing, 100 μL of the Detection Antibody was applied to each well, diluted 1:200 in 1× AD, and incubated for 1 hour. Plate was washed and followed by 30 minutes incubation with 100 μL of Avidin-HRP solution per well, diluted 1:1000 in 1× AD. The final washing was performed 5 times with at least 30 seconds interval between the washings, to decrease the background. Next 100 μL of the freshly mixed TMB Substrate Solution (10 mL per plate, 5 mL of each from 2 substrates provided in the set) was applied and left in the dark for 15 min. It needs to be observed to prevent signal saturation, positive wells turned blue. After incubation in the dark the reaction was stopped with 100 μL of 2N H₂SO₄ per well. Positive wells turned yellow. Absorbance was read at 450 nm and 570 nm (background) within 30 minutes. The data were analyzed in the Microsoft Excel 2010 program.

The results are presented in FIG. 5A-D.

FIGS. 5A and 5B. Inhibitory effect of Env59 (ISU) peptide on expression of IL-6 mRNA and IL-6 protein in LPS-stimulated THP-1 cells. THP-1 cells were incubated with either complete growth medium, or 30 μM Env59 peptide, 60 μM Env59 peptide, 30 μM control peptide, 60 μM control peptide, and simultaneously stimulated for 4 h with 1 μg/ml LPS. After incubation samples were proceed for RNA extraction and supernatant was used for ELISA experiments. The experiment was repeated five times and one of the five typical results is shown in the FIGS. 5A and 5B. Incubation time and peptides concentrations were optimized in time course and dose-response curve experiment (Wasaporn 2010).

FIGS. 5C and 5D. Inhibitory effect of Env59 (ISU) peptides on IL-6 protein and INF-gamma protein in PMA/ionomycin stimulated human PBMCs. Human PBMCs obtained from healthy donors or patients with SLE, were incubated with either complete growth medium, or 30 μM Env59-H6 peptide, 60 μM Env59-H6 peptide, 30 μM Env59-GP3 peptide, 60 μM Env59-GP3 peptide, 30 μM control peptide, 60 μM control peptide, and simultaneously stimulated for 4 h with 50 ng/ml PMA and 1 μg/ml ionomycin. After incubation supernatants were collected and used for ELISA experiments. The experiment was repeated five times and one of the five typical results is shown in the FIGS. 5C and 5D. Incubation time and peptides concentrations were optimized in time course and dose-response curve experiment.

Env 59 ISD suppressed strongly the expression of the mRNA and protein for IL-6 in LPS-stimulated THP-1 cells. The control peptide showed no suppressive effect on either the IL-6 mRNA or protein expression levels. The expression of IL-6 protein was minimal in THP-1 cells incubated with medium alone (FIG. 5B). The level of housekeeping gene RPL13a was used for mRNA normalization was not influenced by peptides and/or LPS treatment. The ability of Env 59 ISD to inhibit IL-6 in a cell line is very significant since IL-6 is believed to be involved in SLE and its reduction is expected to constitute a novel treatment strategy for autoimmune diseases. PBMCs were stimulated with 50 ng/ml PMA plus 1 μg/ml ionomycin. This stimulation was selected as giving the most consistent results for IL-6 protein induction in human PBMCs (data not shown). Real-time RT-PCR quantification was not performed due to low concentration of purified RNA which is obtainable from PBMCs. The results are shown in FIG. 5C. The IL-6 levels were significantly lower in PBMCs incubated with of any of the Env-59 ISD peptides. The control peptide had no effect on the synthesis of IL-6 protein. The level of IL-6 protein was below lowest detection limit in PBMCs incubated with medium alone. In the last series of experiments we were interested to see if Env 59 ISD had an effect on the production of other inflammatory cytokines. e.g. interferon gamma (IFN-gamma). Excessive production of IFN-gamma has been implicated in the pathogenesis of systemic lupus erythematosus, and a deficiency in INF-gamma receptor totally abates the disease process. The synthetic Env 59 ISD peptides inhibit the production of INF-gamma by PMA/ionomycin-stimulated human PBMCs (FIG. 5D) although on different levels. In those studies inhibition of effector molecules was not merely secondary to a nonspecific toxicity of the peptides to PBMCs as assessed by trypan blue dye exclusion.

### Example 6: Immunomodulatory Function Induced by SG#1-SG#17 (ID1031 to ID1047)

Pretreatment of cells with peptides SG#1 to SG#17 (ID1031 to ID1047) affects the release of cytokines including pro-inflammatory cytokines such as IL-6, TNF-alpha, IL-10 and IL-8.

Here, we examined the modulatory function of peptides, peptides SG#1 to SG#17 (here ID1031 to ID1047) on the expression levels of IL-6, TNF-alpha, IL-10 and IL-8 in human acute monocytic leukemia cell line THP-1. THP-1 cells were maintained in RPMI 1640 supplemented with 10% FBS, 100 U/ml penicillin, 100 μg/ml streptomycin and 2 mM L-glutamine at 37° C. in a 5% CO₂ incubator. THP-1 cells are known to induce IL-6, TNF-alpha, IL-10 and IL-8 mRNA and protein in response to lipopolysaccharide (LPS) treatment. THP-1 cells were left untreated or incubated with 0 uM, 7.5 μM, 15 μM, 30 μM, 60 μM or 100 μm of each of the peptides, peptides SG#1 to SG#17 (here ID1031 to ID1047), and stimulated with 1 μg/μl LPS for 6 h, based on the previous analyses to find the optimal dose and incubation times.

TABLE 1
Table 1: Modulatory function of peptides, peptides SG#1 to SG#17 (here
ID1031 to ID1047) on the expression levels of IL-6 in human acute monocytic
leukemia cell line THP-1. (−) inhibition indicates percentage (%) of inhibition
as compared to only LPS treated samples (arbitrary set at 100%). As such (−) 98.93875
for SG#17 at 100 μM indicates that compared to only LPS treated cells,
98.93875 percenatage (%) of IL-6 secretion was inhibited (or less than 0.1%
IL-6 was secreted) by the treatment with a peptide SG#17 (ID1047). Accordingly
(+) 36.9828 percentage (%) for SG#13 at 100 μM indicates that the
level of secreted cytokine was 36.9828 percentage (%) above the only LPS treated
samples (100%) or 136.,98285%.

| Name | 7.5 μM | 15 μM | 30 μM | 60 μM | 100 μM |
|---|---|---|---|---|---|
| SG#1 | (+)0.6842 | (+)7.19017 | (−) 3.53917 | (−)25.11339 | (−)26.67839 |
| SG#2 | (−)15.6948 | (−)25.9059 | (−)30.5144 | (−)51.3958 | (−)73.1873 |
| SG#3 | (−)2.24997 | (−)10.222 | (−)12.2657 | (−)37.5909 | (−)39.5264 |
| SG#4 | (+)12.98853 | (+)20.76308 | (+)38.19179 | (+)36.39436 | (+)13.26897 |
| SG#5 | (−)7.63816 | (−)4.51669 | (−)9.3891 | (−)50.4993 | (−)61.1801 |
| SG#6 | (+)3.08227 | (+)15.47627 | (−)2.16739 | (−)13.4223 | (−)28.9119 |
| SG#7 | N.D | N.D | N.D | N.D | N.D |
| SG#8 | (+)1.471583 | (+)14.54056 | (+)6.835367 | (+)7.654897 | (+)20.85891 |
| SG#9 | (+)3.354645 | (+)2.904014 | (+)2.63574 | (−)9.23121 | N.D. |
| SG#10 | (−)6.0571 | (+)8.264384 | (+)18.28915 | (+)5.122111 | (−)0.45913 |
| SG#11 | (+)12.10097 | (−)15.0445 | (−)3.07211 | (−)15.9818 | (−)0.73926 |
| SG#12 | N.D. | N.D. | N.D. | N.D | N.D. |
| SG#13 | (−)5.76479 | (+)6.907031 | (−)5.28609 | (+)8.616429 | (+)36.9828 |
| SG#14 | (−)2.98108 | (−)0.44762 | (+)0.740257 | (+)28.51696 | (+)15.20844 |
| SG#15 | (−)14.0971 | (−)3.04025 | (−)28.6554 | (−)78.1353 | (−)99.0097 |
| SG#16 | (+)1.082038 | (−)2.72293 | (+)3.642883 | (+)4.832977 | (−)19.1099 |
| SG#17 | (−)42.71029 | (−)41.98221 | (−) 52.01779 | (−) 69.48402 | (−) 98.93875 |

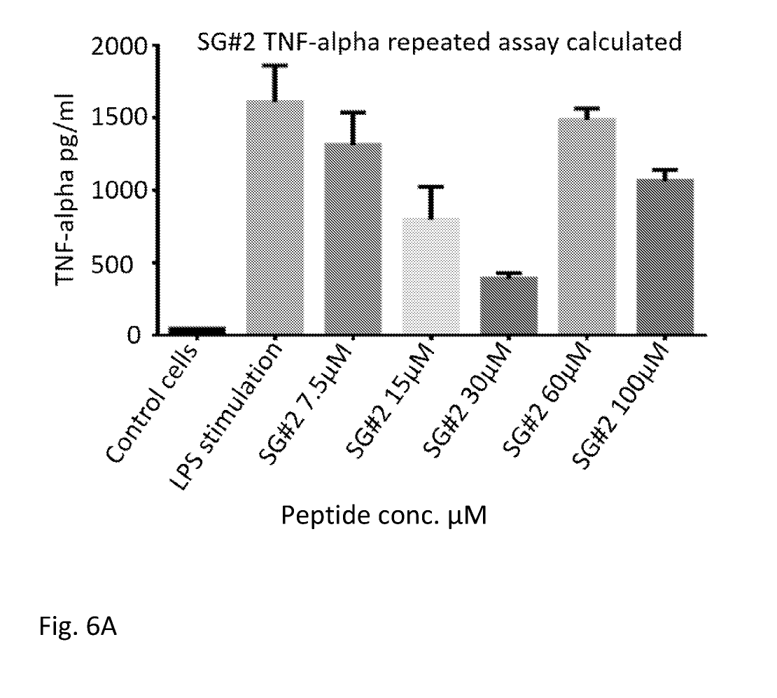

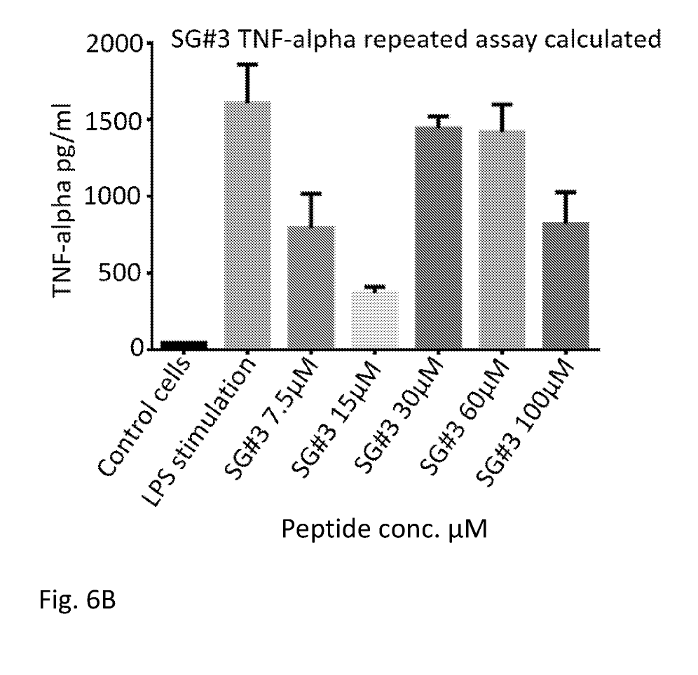

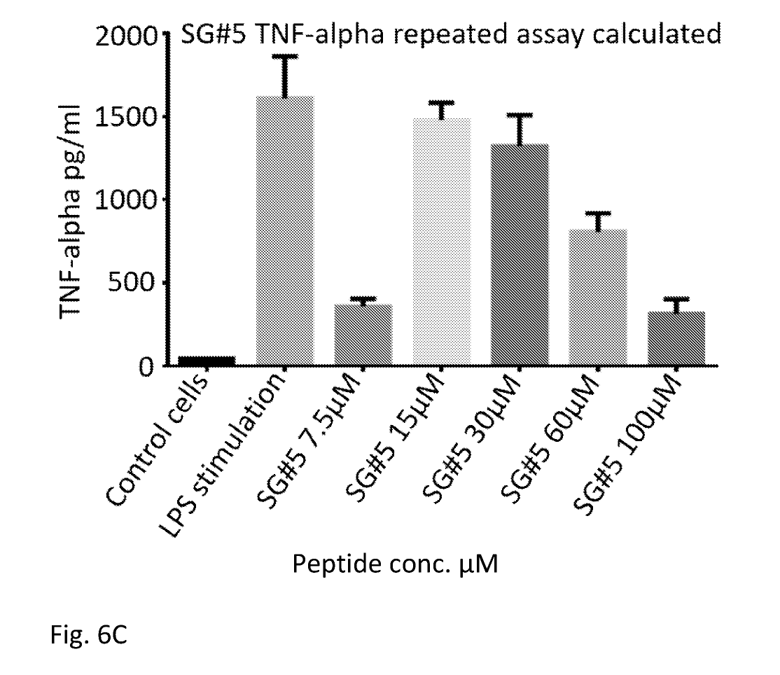

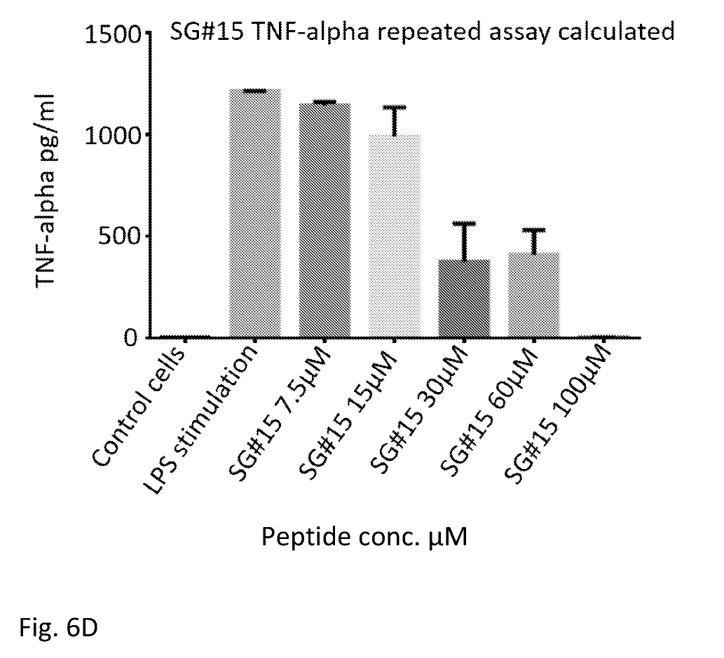

FIG. 6 (A, B, C, D): Immunomodulatory function induced by SG#2, SG#3, SG#15, SG#15 (ID1032, ID1033, ID1035 and ID1045) on the expression levels of TNF-alpha protein secretion.

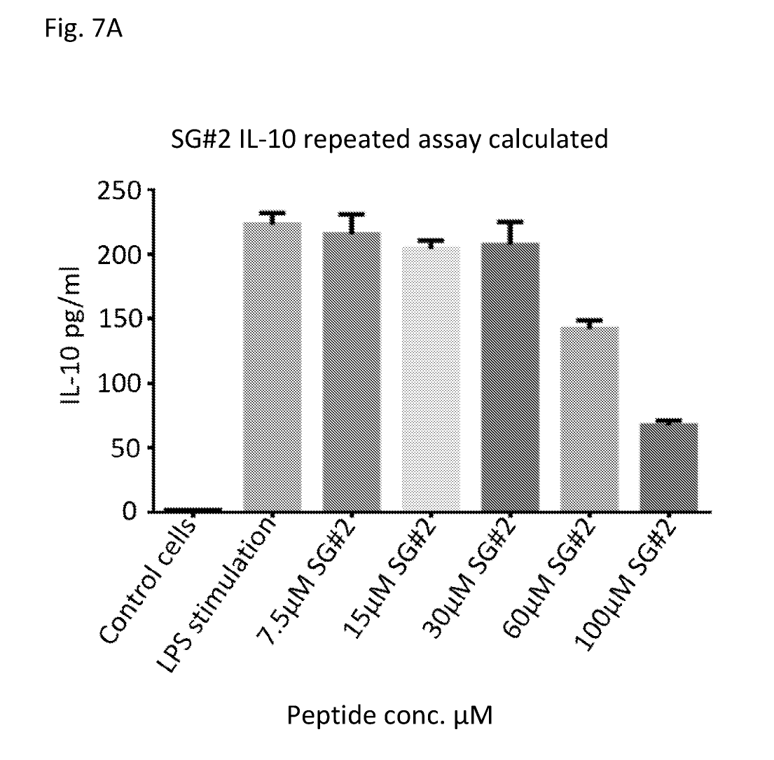

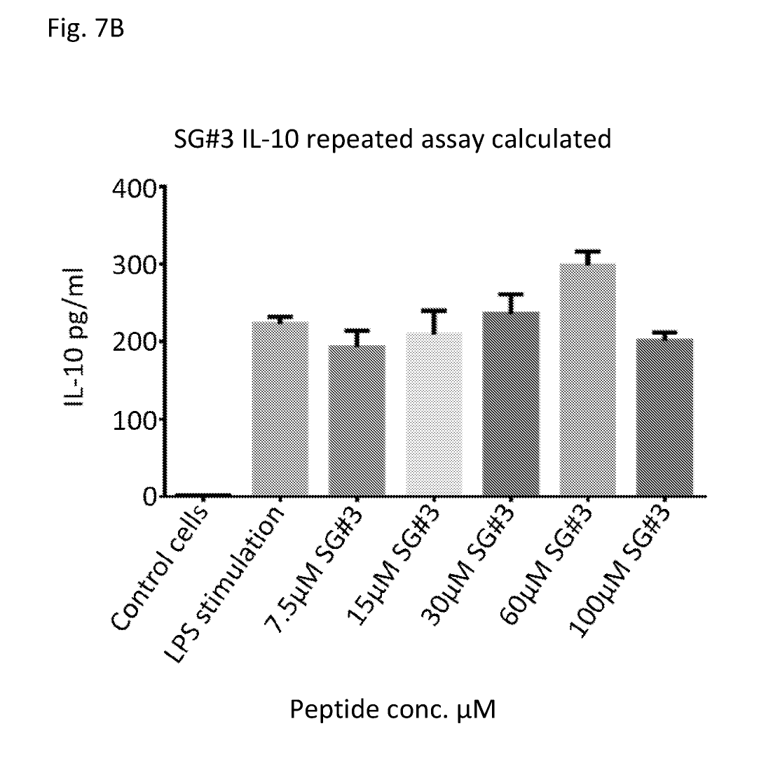

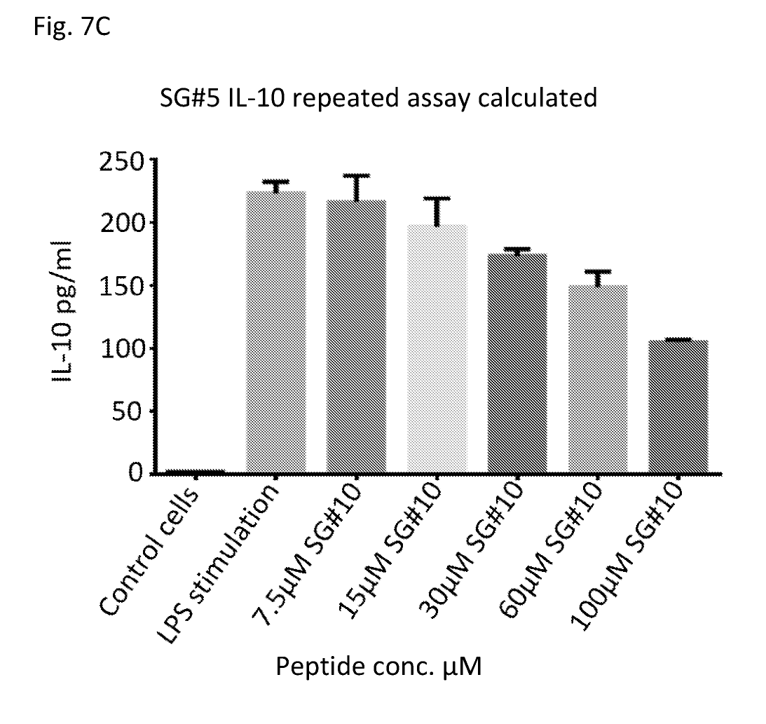

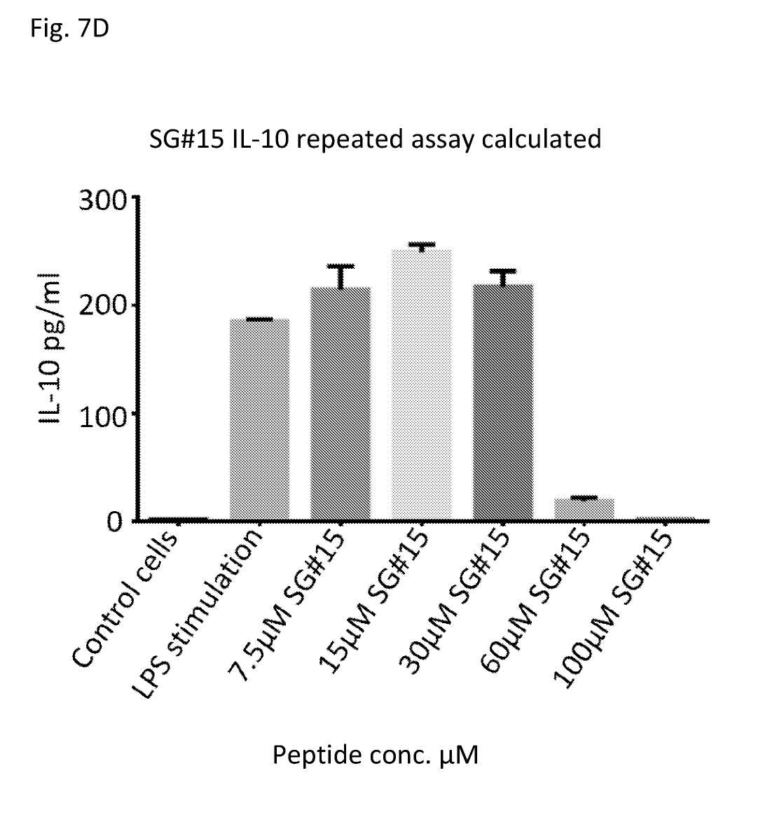

FIG. 7 (A, B, C, D): Immunomodulatory function induced by SG#2, SG#3, SG#15, SG#15 (ID1032, ID1033, ID1035 and ID1045) on the expression levels of IL-10.

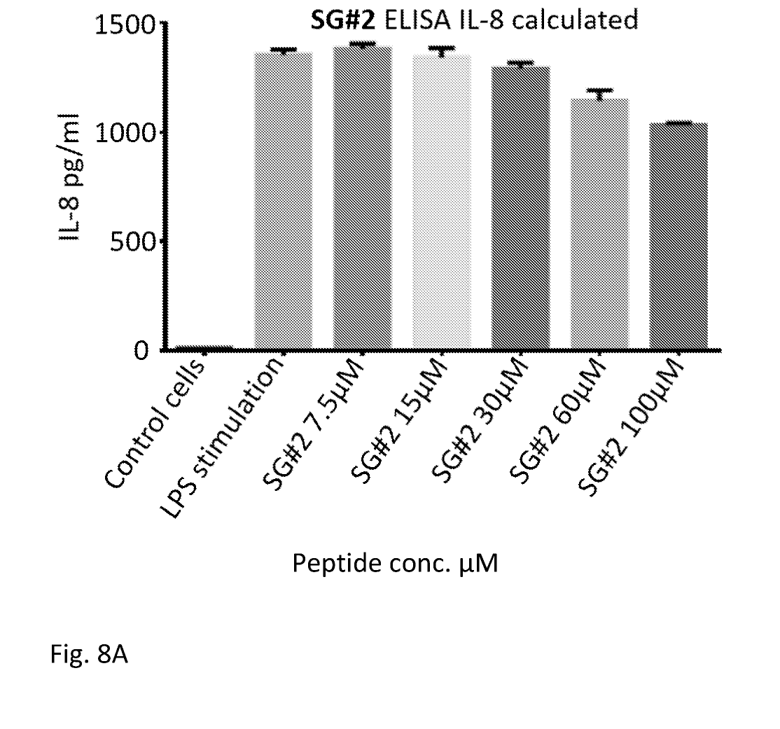

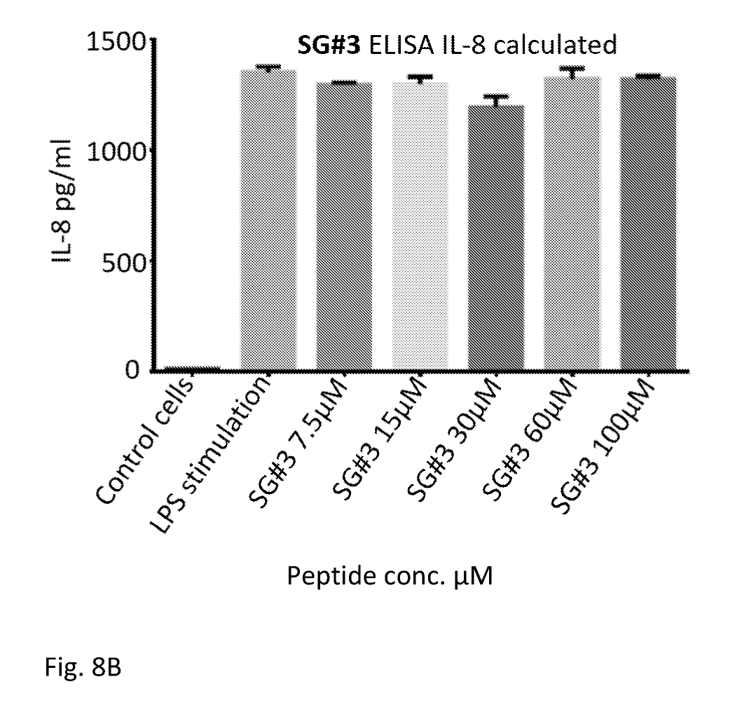

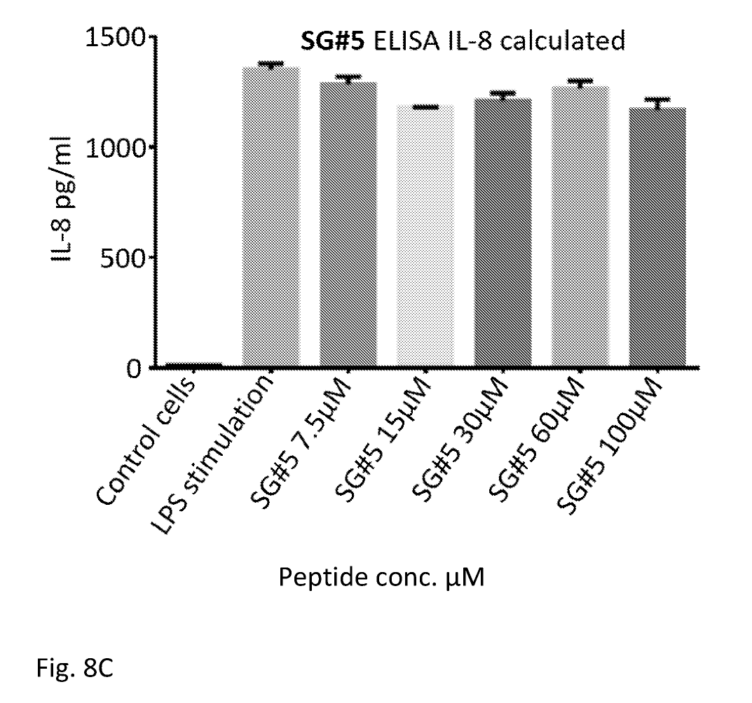

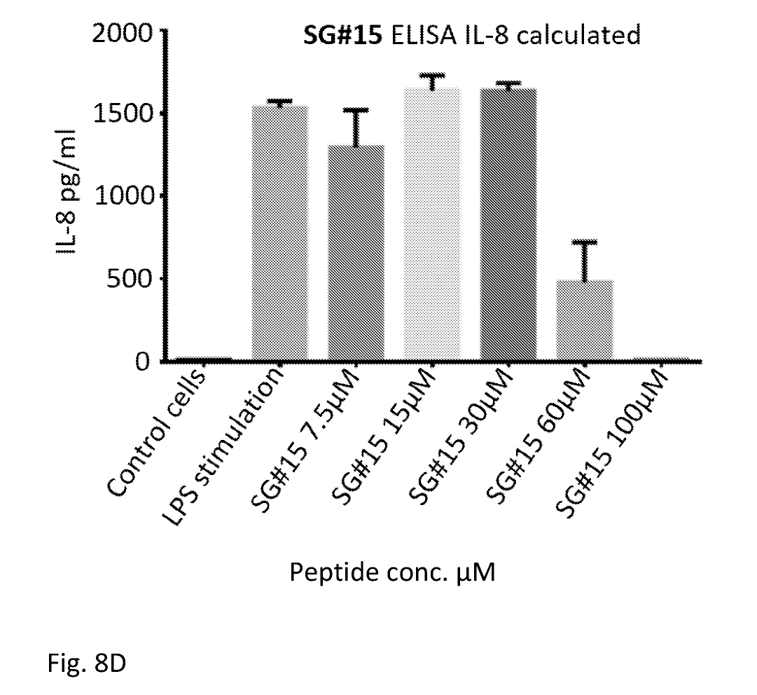

FIG. 8 (A, B, C, D): Immunomodulatory function induced by SG#2, SG#3, SG#15, SG#15 (ID1032, ID1033, ID1035 and ID1045) on the expression levels of IL-8.

### Procedure for IL-6, TNF-Alpha, IL-10 and IL8 ELISA Quantification

ELISA assay was performed according to the manufacturer's protocol, as follows. Each incubation step was followed by sealing and shaking on the rotating table at 150-200 rpm, except the overnight incubation with the Capture Antibody, where plates were not shaken. One day prior running ELISA the 96-well assay plates were covered with the Capture Antibody, diluted 1:200 in 1× Coating Buffer (5× Coating Buffer diluted in ddH₂O). 100 μL of this Capture Antibody solution was added into all wells, sealed and incubated overnight (16-18 hrs) at 4° C. The next day all reagents from the set were brought to room temperature (RT) before use. The plate was washed 4 times with minimum 300 μL Wash Buffer (1×PBS, 0.05% Tween 20) per well. The residual buffer in the following washing was removed by blotting the plates against the absorbent paper. Next 200 μL of the 1× Assay Diluent A (5× Assay Diluent A diluted in PBS pH=7.4) was added for 1 h to block non-specific binding. While the plate was being blocked, all samples and standards (mandatory for each plate) were prepared. Standards and samples were run in triplicates. 1 mL of the top standard concentration (250 pg/mL or 300 pg/mL for IL-10 quantification) was prepared in 1× Assay Diluent A (1× AD) from the relevant IL-6, TNF-alpha, IL-10 or IL-8 stock solution. The six two-fold serial dilutions of the 250 pg/mL (or 300 pg/mL for IL-10 quantification) top standard were performed, with the human IL-6, TNF-alpha or IL-8 standard concentration: 250 pg/mL, 125 pg/mL, 62.5 pg/mL, 31.2 pg/mL, 15.6 pg/mL, 7.8 pg/mL and 3.9 pg/mL, respectively as well as with the IL-10 standard concentration: 300 pg/mL, 150 pg/mL, 75 pg/mL, 37.5 pg/mL, 18.75 pg/mL, 9.375 pg/mL and 4.6875 pg/mL, respectively. 1× AD serves as the zero standard (0 pg/mL). After blocking the plate, washing was performed and 100 μL standards and samples were assayed in triplicates and incubated for 2 h in RT. Samples were not diluted, the whole supernatant from the THP-1 or PBMCs cells was assayed. After washing, 100 μL of the Detection Antibody was applied to each well, diluted 1:200 in 1× AD, and incubated for 1 hour. Plate was washed and followed by 30 minutes incubation with 100 μL of Avidin-HRP solution per well, diluted 1:1000 in 1× AD. The final washing was performed 5 times with at least 30 seconds interval between the washings, to decrease the background. Next 100 μL of the freshly mixed TMB Substrate Solution (10 mL per plate, 5 mL of each from 2 substrates provided in the set) was applied and left in the dark for 15 min. It needs to be observed to prevent signal saturation, positive wells turned blue. After incubation in the dark the reaction was stopped with 100 μL of 2N H₂SO₄ per well. Positive wells turned yellow. Absorbance was measured at 450 nm and 570 nm (background) within 30 minutes. The data were analyzed in the Microsoft Excel 2010 program. Statistical analyses were performed using Microsoft Excell 2010 program.

### Example 7: The Effect of the Peptides on Arthritis Score and SAA-3 Expression in Sakaguchi Mice Model (Spontaneous CD4+ T Cell-Mediated Chronic Autoimmune Arthritis)

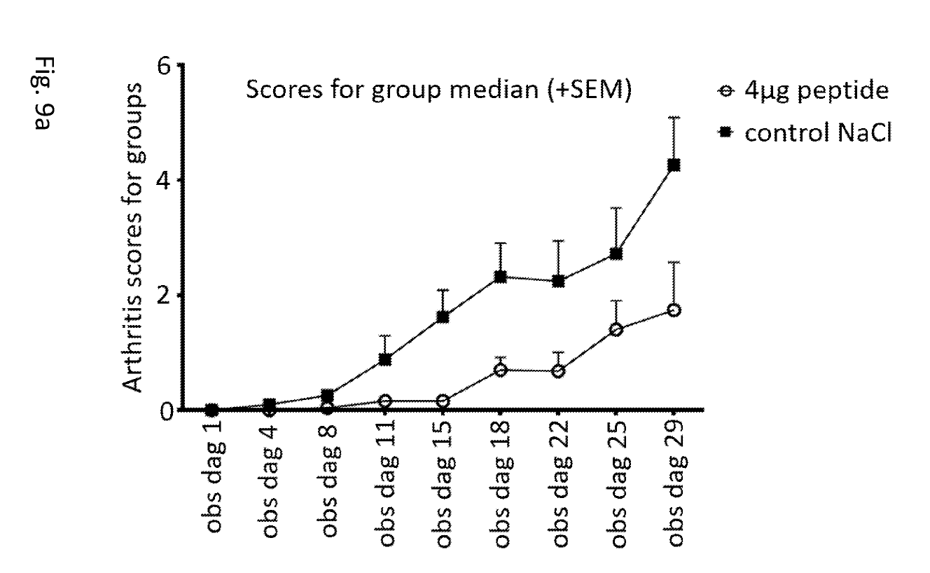

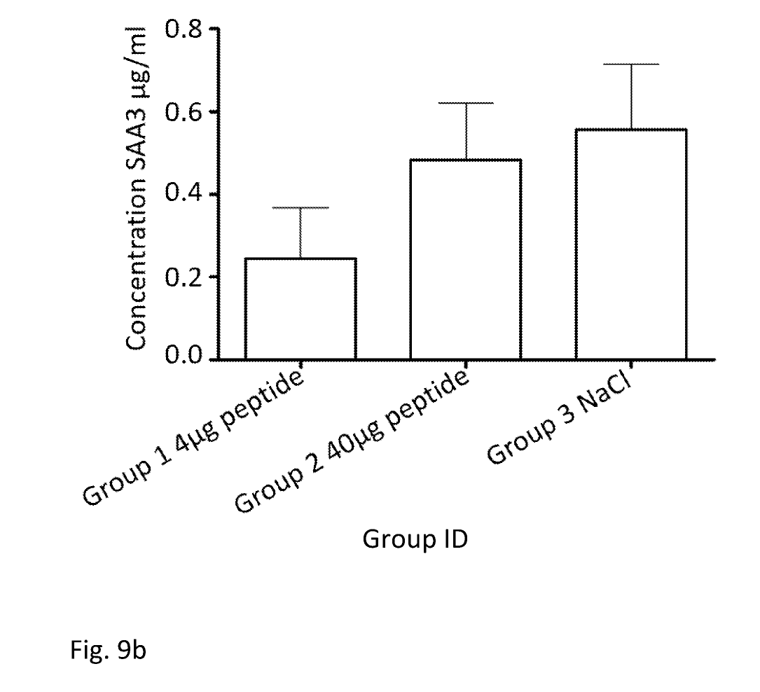

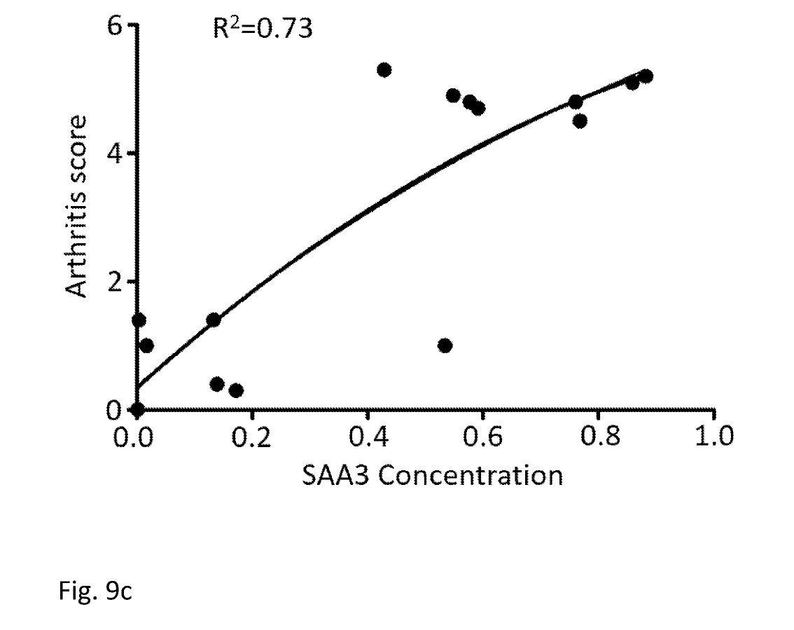

Here we present data on investigating in vivo effect of the HERV-Env59 peptide. The effect of the Env59 ISU peptides on disease development is compared with a scrambled peptide or saline treated control group. Arthritis was induced by intrapenile injection of 220 μl mannan at concentration 90 μg/ml. Animals were treated daily by subcutaneous injections with indicated concentration of Env-59 peptide, scrambled peptide control or NaCL saline control. Joint swelling was monitored by inspection and scored as follows: 0, no joint swelling; 0.1, swelling of one finger joint; 0.5, mild swelling of wrist or ankle; 1.0, severe swelling of wrist or ankle. Scores for all digits, wrists, and ankles were totaled for each mouse in FIG. 9A.

### FIG. 9A

Development of arthritis in SKG mice. Vertical bars represent the means+SD of the whole group of mice (5 animals each group). Arthritis scores are significantly different between Env3 peptide and NaCl control treated mice. Peptides or control saline NaCl were administrated subcutaneously 5 times a week starting with Day (−1, one day pre-treatment).

### Mouse Serum Amyloid A-3 ELISA

Acute-phase serum amyloid A proteins (A-SAAs) are secreted during the acute phase of inflammation. Similar to CRP, levels of acute-phase SAA increase within hours after inflammatory stimulus, and the magnitude of increase may be greater than that of CRP. SAA3 gene is regulated by proinflammatory cytokine IL-6. Quality of ELISA assay has been verified by including two reference QC samples (included in the kit with known expected range). Plasma samples were analyzed in duplicate for the presence of SAA3 using an ELISA according to the manufacturer's protocol (Millipore). Concentrations of SAA3 were determined in plasma collected the same time point when animals were bleeded and sacrificed (day 28 of trial) (FIG. 9B). Differences in medians were detected among treatment groups. SAA3 levels were elevated in SKG mice treated with saline NaCL as compared to animals treated with HERV-Env59 derived peptide. Next, we examined the correlation of circulating SAA3 levels with arthritis scores regardless of treatment group. There was a positive correlation between SAA3 plasma concentration and arthritis score using linear regression (FIG. 9). However, the relationship appears curvilinear and best fits a semi-logarithmic (log-X, linear-Y) model (R²=0.73). This suggests that log increases in circulating SAA3 corresponds to unit changes in arthritis score.

Results are presented in FIGS. 9b and 9c.

### Example 8: List of Primers Used for Real Time RT-PCR Analysis (Table 2)

TABLE 2

| Target gene/primer name | Primer sequence 5′-3′ |
|---|---|
| Env 3 forward set 1 | Agggtaaaggtgagggctgt |
| Env 3 reverse set 1 | agcaaacaactgctggcttt |
| IL-6 forward | AGCCACTCACCTCTTCAGAAC |
| IL-6 reverse | GCCTCTTTGCTGCTTTCACAC |
| ISD 59 forward | AGAGCTCCCTGTTCCCCTTA |
| ISD 59 reverse | CATTAGACGGGCTACGGAAG |
| RPL13a forward | CATCGTGGCTAAACAGGTACTG |
| RPL13a reverse | GCACGACCTTGAGGGCAGCA |
| RPL37A forward | ATTGAAATCAGCCAGCACGC |
| RPL37A reverse | AGGAACCACAGTGCCAGATCC |
| EnvH3 forward | gttgggctttggagatgg |
| EnvH3 reverse | ccctcctccacatttatttg |

### Example 9: The Effect of the Peptides on Arthritis Scores in Collagen-Induced Arthritis Model (CIA Model)

The CIA model is the “standard” animal model for evaluation of anti-arthritic activity based on immunization with bovine collagen to develop antibodies against bone and cartilage.

Here we present data on investigating in vivo effect of the HERV-Env59 peptide. The aim was to determine anti-arthritic dosing paradigm in a murine model of collagen-induced arthritis. This study was carried out in female DBA/1J mice. The effect of the Env59 ISU peptides on disease development is compared with a methotrexate MTX (positive control) or saline treated control group.

### Summary of Procedure:

Day 0: The mice were weighted and injected subcutaneously at the nape of the neck as in the table below (table 3):

TABLE 3

| Group | No. mice | Treatment | Dose | |
|---|---|---|---|---|
| 1 | 10 | saline | 10 | ml/kg |
| 2 | 10 | HERV-Env59 peptide | 4 | μg/mouse |
| 3 | 5 | Methotrexate | 3 | mg/kg |

An hour later, the mice were injected subcutaneously at the base of the tail with 50 μl of the collagen/CFA emulsion.

Mice were scored for signs of arthritis every Monday, Wednesday, and Friday during the next 41 days as follows:
Each paw receives a score
0=no visible effects of arthritis
1=edema and/or erythema of 1 digit
2=edema and/or erythema of 2 digits
3=edema and/or erythema of more than 2 digits
4=severe arthritis of entire paw and digits

The Arthritic Index (AI) was calculated by the addition of individual paw scores.

Day 42 The mice were weighted and scored for signs of arthritis.

Table 4 and FIG. 10 show the effect of treatment on average disease development based on AI.

Table 5 shows the effect of treatment on average terminal individual paw scores

TABLE 4

| | | STUDY DAY | | | | | | | | | | |
|---|---|---|---|---|---|---|---|---|---|---|---|---|
| Group | Mouse | 21 | 22 | 25 | 27 | 29 | 32 | 34 | 36 | 38 | 41 | 42 |
| 1 | Mean | 0 | 0.7 | 1.3 | 2.3 | 2.9 | 4.9 | 6.6 | 7.9 | 9.1 | 9.9 | 10.5 |
| | SEM | 0 | 0.5 | 1.0 | 1.1 | 1.0 | 0.9 | 0.7 | 0.5 | 0.4 | 0.3 | 0.4 |
| 2 | Mean | 0 | 0 | 0.8 | 1.1 | 1.2 | 2.0 | 2.1 | 2.7 | 3.1 | 3.2 | 3.3 |
| | SEM | 0 | 0 | 0.7 | 1.0 | 1.0 | 1.2 | 1.1 | 1.2 | 1.2 | 1.2 | 1.2 |
| | p-value | 1.00 | 0.17 | 0.68 | 0.42 | 0.25 | 0.06 | 0.003 | 0.001 | 0.0002 | 4 × 10⁻⁵ | 2 × 10⁻⁵ |
| 3 | Mean | 0 | 0 | 0 | 0 | 0 | 0 | 0 | 0 | 0 | 0 | 0 |
| | SEM | 0 | 0 | 0 | 0 | 0 | 0 | 0 | 0 | 0 | 0 | 0 |
| | p-value | 1.00 | 0.34 | 0.38 | 0.16 | 0.07 | 0.002 | 9 × 10⁻⁶ | 4 × 10⁻⁸ | 9 × 10⁻¹⁰ | 4 × 10⁻¹¹ | 5 × 10⁻¹¹ |

TABLE 5

| Group | Mouse | Front right | Front left | Rear right | Rear left |
|---|---|---|---|---|---|
| 1 | Mean | 1.7 | 2.6 | 2.9 | 3.1 |
| | SEM | 0.2 | 0.3 | 0.3 | 0.2 |
| 2 | Mean | 0.8 | 0.3 | 1.5 | 0.7 |
| | SEM | 0.4 | 0.3 | 0.6 | 0.5 |
| | p-value | 0.05 | 2 × 10⁻⁵ | 0.04 | 7 × 10⁻⁵ |
| 3 | Mean | 0 | 0 | 0 | 0 |
| | SEM | 0 | 0 | 0 | 0 |
| | p-value | 3 × 10⁻⁵ | 3 × 10⁻⁶ | 2 × 10⁻⁶ | 3 × 10⁻¹⁰ |

Summary of results: Prophylactic daily subcutaneous injection with 4 μg/mouse HERV-Env59 peptide resulted in 70% incidence of disease and a 70% reduction in disease severity.

Conclusion: Once daily subcutaneous injection with 4 μg/mouse of the test compound, starting one hour prior to disease induction resulted only 70% incidence on disease which was associated with a significant 70% reduction in disease severity at the termination of the study.

### Example 10: Hemolsysis Assay on Red Blood Cells

Drug-induced hemolysis is a relatively rare but serious toxicity liability. It occurs by two mechanisms:

Toxic hemolysis—direct toxicity of the drug, its metabolite, or an excipient in the formulation.

Allergic hemolysis—toxicity caused by an immunological reaction in patients previously sensitized to a drug.

Although the majority of normal individuals may suffer toxic hemolysis at sufficiently high concentrations of hemolytic drugs, for most drugs toxic hemolysis involves lower doses given to individuals who are genetically predisposed to hemolysis. The US FDA recommends that for excipients intended for injectable use, an in vitro hemolysis study should be performed at the indicated concentration for IV administration to test for hemolytic potential. In the hemolysis assay, human red blood cells and test materials are co-incubated in buffers at defined pHs that mimic extracellular, early endosomal, and late endo-lysosomal environments. Following a centrifugation step to pellet intact red blood cells, the amount of hemoglobin released into the medium is spectrophotometrically measured (405 nm for best dynamic range). The percent red blood cell disruption is then quantified relative to positive control samples lysed with a detergent. In this model system the erythrocyte membrane serves as a surrogate for the lipid bilayer membrane that encloses endo-lysosomal vesicles. The desired result is negligible hemolysis at physiologic pH (7.4) and robust hemolysis in the endo-lysosomal pH range from approximately pH 5-6.8.

Here we present data on investigating hemolysis as a function of Env 59 peptide concentration, using red blood cells from chicken. The hemolytic activity of Env3 peptide after 1 h incubation time at 37° C. is shown in FIG. 6. The concentration-response curves of peptides for percentage lysis of chicken red blood cells (RBC) are shown. Included as positive control, peptide from the glycoprotein of the Ebola virus, here assigned as Ebo Z. The control for 100% hemolysis was a sample of erythrocytes treated with NP-40 detergent. The peptide concentration is reported as μM.

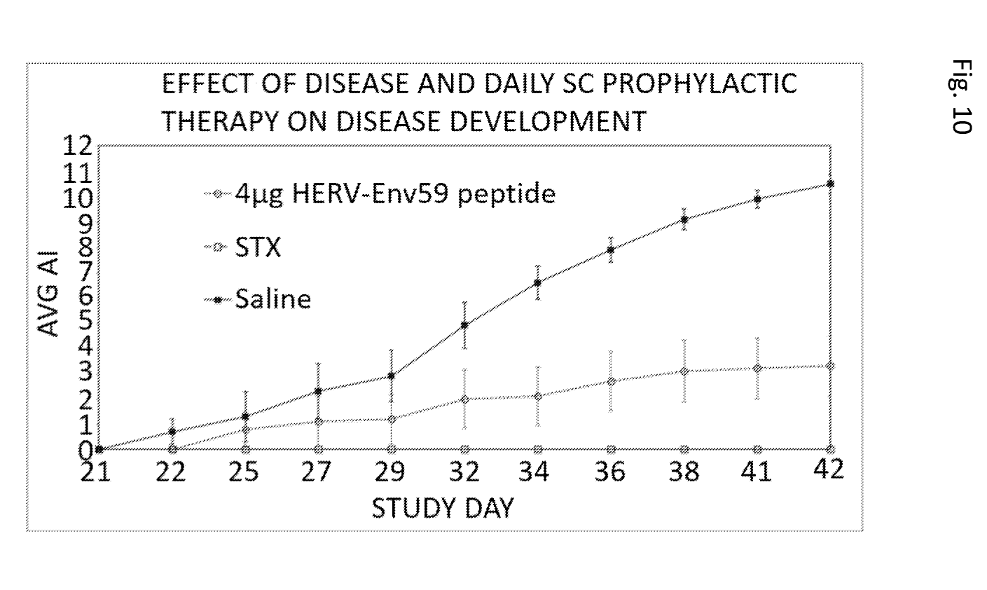

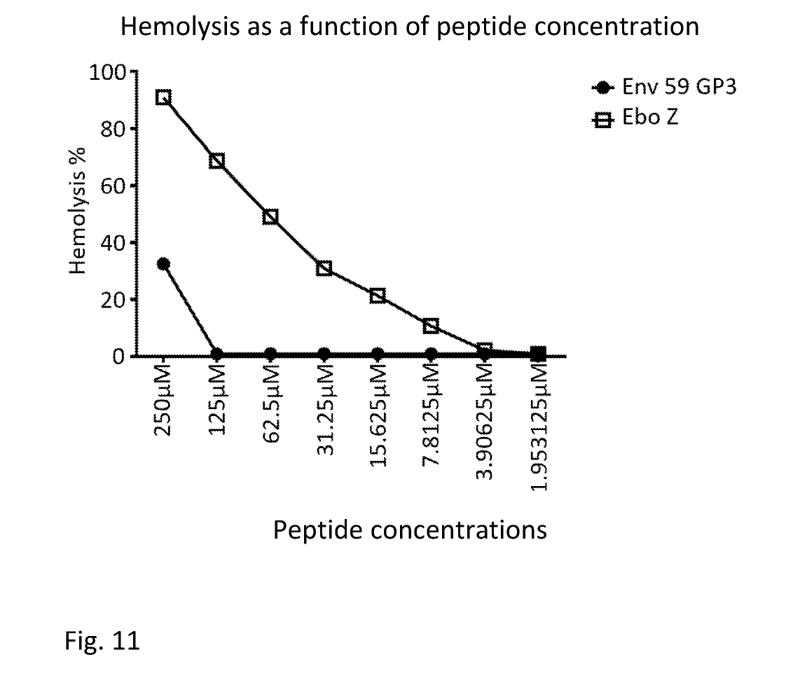

The results are presented in FIG. 11.

### Example 11: Toxicity Profile for Peptides SG#1 to SG#17 ((ID1031 to ID1047)

Here, we examined the cytotoxicity effect of peptides SG#1 to SG#17 (ID1031 to ID1047) on two human cell lines THP-1 or HT-1080 cells.

The CellTiter-Blue® Cell Viability Assay provides a homogenous, fluorometric method for estimating the number of viable cells present in multiwall plates. It uses the indicator dye resazurin to measure the metabolic capacity of cells—an indicator of cell viability. Viable cells retain the ability to reduce resazurin into resorufin, which is highly fluorescent.

The CellTiter-Blue® Reagent is a buffered solution containing highly purified resazurin. Resazurin is dark blue in color and has little intrinsic fluorescence. However, when it is reduced to resorufin, it becomes pink and highly fluorescent (579_{EX}/584_{EM}).

### Example Procedure:

1. Set up 96-well assay plates containing cells in culture medium. For HT-1080, plate 1.6×10⁴ cells per well on the Day 1 of the experiment. Cells are plated in 100 μl complete culture medium (DMEM) per well.
2. On day 2, gently remove medium and add 50 μl of complete DMEM medium, add peptides and vehicle controls to appropriate wells so the final volume is 100 μl in each well. Supplement with complete DMEM where necessary.
3. Culture cells for the desired test exposure period, about 20 h.
4. On day 3, remove assay plates from 37° C. incubator and add 20 μl/well of CellTiter-Blue® Reagent.
5. Shake for 10 seconds.
6. Incubate using standard cell culture conditions for 4 hours.
7. Shake plate for 10 seconds and record fluorescence at 560/590 nm

### Results:

Toxicity profile for peptides SG#1 to SG#17 (ID1031 to ID1047). All peptides were tested at the dosage increment (10 nm, 30 nM, 100 nM, 1 μM, 10 μM, 30 μM, 100 μM and 300 μM). A cytotoxic cell penetrating peptide with IC50 of 1 μM was included as a positive control at the same dosage increment.

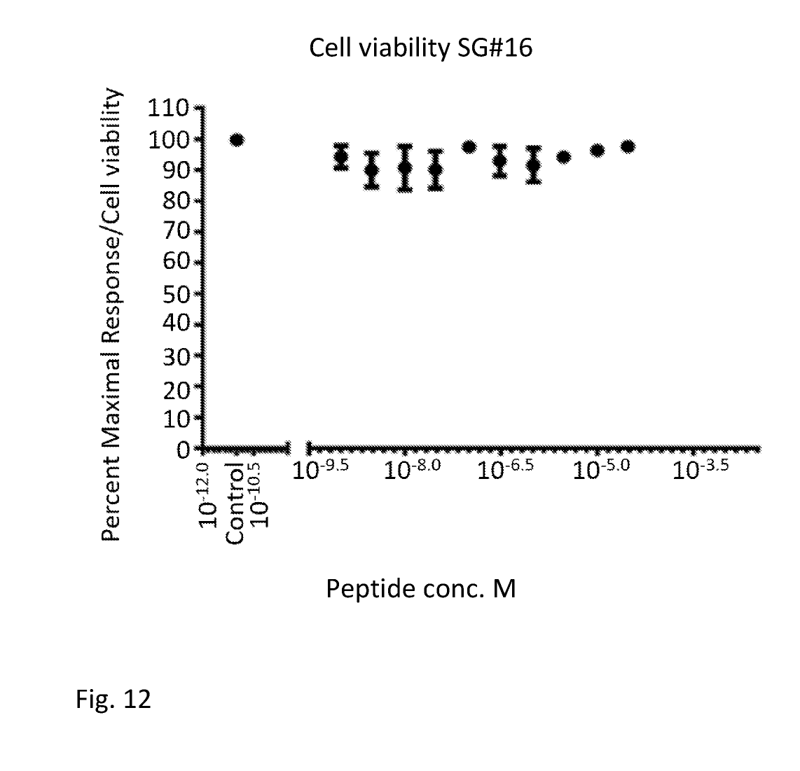

None of the tested peptides SG#1 to SG#17 (ID1031 to ID1047) show signs of toxic effect on THP-1 or HT-1080 cells at the dosage increment (10 nm, 30 nM, 100 nM, 1 μM, 10 μM, 30 μM, 100 μM and 300 μM). FIG. 12 is a representative graph for SG#16 (ID1046).

| | Toxicity yes/no at |
|---|---|
| | the 300 μM conc. |
| SG#1 | No |
| SG#2 | No |
| SG#3 | No |
| SG#4 | No |
| SG#5 | No |
| SG#6 | No |
| SG#8 | No |
| SG#9 | No |
| SG#10 | No |
| SG#11 | No |
| SG#12 | No |
| SG#13 | No |
| SG#14 | No |
| SG#15 | No |
| SG#16 | No |
| SG#17 | No |

EC50 is not calculated since none of the peptides SG#1 to SG#17 (ID1031 to ID1047) show signs of toxic effect on THP-1 or HT-1080 cells at the dosage increment (10 nm, 30 nM, 100 nM, 1 μM, 10 μM, 30 μM, 100 μM and 300 μM).

### Example 12: The Effect of the Peptides on Arthritis Scores in Collagen-Induced Arthritis Model (CIA Model), Study Number 2

The CIA model as described in an Example 9 is the “standard” animal model for evaluation of anti-arthritic activity based on immunization with bovine collagen to develop antibodies against bone and cartilage.

Here we present data on investigating in vivo effect of the HERV-Env59, SG#2, and SG#5 and SG#15 peptides. The aim was to determine anti-arthritic dosing paradigm in a murine model of collagen-induced arthritis. This study was carried out in female DBA/1J mice. The effect of the Env59 ISU peptide, SG#2, SG#5 or SG#15 peptide on disease development is compared with a methotrexate MTX (positive control) or saline treated control group.

### Summary of Procedure:

Day 0: The mice were weighted and injected subcutaneously at the nape of the neck as in the table below (table 6):

TABLE 6

| Group | No. mice | Treatment | Dose | |
|---|---|---|---|---|
| 1 | 8 | saline | 10 | ml/kg |
| 2 | 5 | Methotrexate | 1 | mg/kg |
| 3 | 8 | SG#2 | 4 | μg/mouse |
| 4 | 8 | SG#5 | 4 | μg/mouse |
| 5 | 8 | SG#15 | 4 | μg/mouse |
| 6 | 8 | HERV-Env59 peptide | 4 | μg/mouse |

An hour later, the mice were injected subcutaneously at the base of the tail with 50 μl of the collagen/CFA emulsion.

Mice were scored for signs of arthritis every Monday, Wednesday, and Friday during the next 41 days as follows:
Each paw receives a score
0=no visible effects of arthritis
1=edema and/or erythema of 1 digit
2=edema and/or erythema of 2 digits
3=edema and/or erythema of more than 2 digits
4=severe arthritis of entire paw and digits

The Arthritic Index (AI) was calculated by the addition of individual paw scores.

Day 42 The mice were weighted and scored for signs of arthritis.

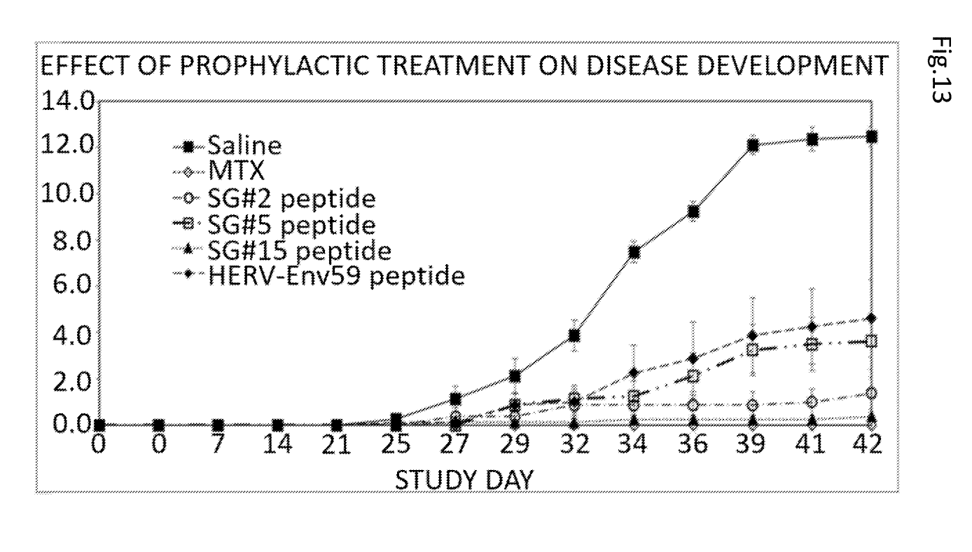

Table 7 and FIG. 13 show the effect of treatment on average disease development based on AI.

Table 8 shows the effect of treatment on average terminal individual paw scores

TABLE 7
Effect of Treatment on Avarage Disease Development (AI):
Effect of Treatment on Average Disease Development (AI):

| | | STUDY DAY | | | | | | | | | | |
|---|---|---|---|---|---|---|---|---|---|---|---|---|
| Group | Mouse | 14 | 21 | 25 | 27 | 29 | 32 | 34 | 36 | 39 | 41 | 42 |
| 1 | Mean | 0 | 0 | 0.3 | 1.1 | 2.1 | 3.9 | 7.5 | 9.3 | 12.1 | 12.4 | 12.5 |
| | SEM | 0 | 0 | 0.3 | 0.5 | 0.7 | 0.7 | 0.5 | 0.4 | 0.4 | 0.5 | 0.4 |
| 2 | Mean | 0 | 0 | 0 | 0 | 0 | 0 | 0 | 0 | 0 | 0 | 0 |
| | SEM | 0 | 0 | 0 | 0 | 0 | 0 | 0 | 0 | 0 | 0 | 0 |
| | p-value | 1.00 | 1.00 | 0.45 | 0.14 | 0.05 | 0.0009 | 7 × 10⁻⁸ | 2 × 10⁻⁹ | 9 × 10⁻¹¹ | 8 × 10⁻¹⁰ | 1 × 10⁻¹⁰ |
| 3 | Mean | 0 | 0 | 0 | 0.4 | 0.4 | 0.9 | 0.9 | 0.9 | 0.9 | 1.0 | 1.4 |
| | SEM | 0 | 0 | 0 | 0.4 | 0.4 | 0.6 | 0.6 | 0.6 | 0.6 | 0.6 | 0.7 |
| | p-value | 1.00 | 1.00 | 0.33 | 0.28 | 0.05 | 0.004 | 4 × 10⁻⁷ | 1 × 10⁻⁸ | 2 × 10⁻¹⁰ | 5 × 10⁻¹⁰ | 1 × 10⁻⁹ |
| 4 | Mean | 0 | 0 | 0 | 0.0 | 0.9 | 1.1 | 1.3 | 2.1 | 3.3 | 3.5 | 3.6 |
| | SEM | 0 | 0 | 0 | 0.0 | 0.5 | 0.6 | 0.6 | 1.0 | 1.1 | 1.1 | 1.2 |
| | p-value | 1.00 | 1.00 | 0.33 | 0.06 | 0.18 | 0.008 | 2 × 10⁻⁶ | 9 × 10⁻⁶ | 2 × 10⁻⁶ | 5 × 10⁻⁶ | 7 × 10⁻⁶ |
| 5 | Mean | 0 | 0 | 0 | 0.1 | 0.1 | 0.1 | 0.3 | 0.3 | 0.3 | 0.3 | 0.4 |
| | SEM | 0 | 0 | 0 | 0.1 | 0.1 | 0.1 | 0.2 | 0.2 | 0.2 | 0.2 | 0.2 |
| | p-value | 1.00 | 1.00 | 0.33 | 0.10 | 0.02 | 7 × 10⁻⁵ | 6 × 10⁻¹⁰ | 9 × 10⁻¹² | 1 × 10⁻¹³ | 1 × 10⁻¹² | 3 × 10⁻¹³ |
| 6 | Mean | 0 | 0 | 0.1 | 0.1 | 0.9 | 1.0 | 2.3 | 2.9 | 3.9 | 4.3 | 4.6 |
| | SEM | 0 | 0 | 0.1 | 0.1 | 0.5 | 0.6 | 1.2 | 1.6 | 1.6 | 1.6 | 1.7 |
| | p-value | 1.00 | 1.00 | 0.66 | 0.10 | 0.18 | 0.005 | 0.001 | 0.001 | 0.0002 | 0.0003 | 0.0005 |

TABLE 8
Effect of Treatment on Avarage Terminal Individual Paw Scores:

| Group | Mouse | Front right | Front left | Rear right | Rear left |
|---|---|---|---|---|---|
| 1 | Mean | 3.0 | 3.5 | 3.0 | 3.0 |
| | SEM | 0.3 | 0.3 | 0.3 | 0.0 |
| 2 | Mean | 0 | 0 | 0 | 0 |
| | SEM | 0 | 0 | 0 | 0 |
| | p-value | 2 × 10⁻⁵ | 6 × 10⁻⁷ | 3 × 10⁻⁶ | 9 × 10⁻¹⁰ |
| 3 | Mean | 0.4 | 0.4 | 0.5 | 0.1 |
| | SEM | 0.4 | 0.4 | 0.5 | 0.1 |
| | p-value | 0.0001 | 9 × 10⁻⁶ | 0.0006 | 2 × 10⁻¹² |
| 4 | Mean | 1.1 | 1.3 | 1.0 | 0.3 |
| | SEM | 0.4 | 0.5 | 0.4 | 0.2 |
| | p-value | 0.004 | 0.002 | 0.001 | 1 × 10⁻¹⁰ |
| 5 | Mean | 0 | 0.1 | 0 | 0.3 |
| | SEM | 0 | 0.1 | 0 | 0.2 |
| | p-value | 3 × 10⁻⁷ | 2 × 10⁻⁸ | 2 × 10⁻⁸ | 1 × 10⁻¹⁰ |
| 6 | Mean | 1.1 | 1.1 | 1.1 | 1.3 |
| | SEM | 0.5 | 0.5 | 0.5 | 0.5 |
| | p-value | 0.01 | 0.002 | 0.008 | 0.002 |

### Summary of Results and Conclusion:

Effect of Prophylactic Treatment with SG#2 Peptide (Group 3):

Once daily subcutaneous injection with 4 μg/mouse of SG#2 peptide, starting one hour prior to disease induction resulted in only 30% incidence of disease which was associated with a significant 90% reduction in disease severity at the termination of the study. This treatment regimen had no effect on diseased mouse weight.

Effect of Prophylactic Treatment with SG#5 Peptide (Group 4):

Once daily subcutaneous injection with 4 μg/mouse of SG#5 peptide, starting one hour prior to disease induction resulted in 80% incidence of disease which was associated with a significant 70% reduction in disease severity at the termination of the study. This treatment regimen had no effect on diseased mouse weight.

Effect of Prophylactic Treatment with SG#15 Peptide (Group 5):

Once daily subcutaneous injection with 4 μg/mouse of SG#15 peptide, starting one hour prior to disease induction resulted in only 30% incidence of disease which was associated with a significant 97% reduction in disease severity at the termination of the study. This treatment regimen had no effect on diseased mouse weight.

Effect of Prophylactic Treatment with HERV-Env59 Peptide (Group 6):

Once daily subcutaneous injection with 4 μg/mouse of test compound 4, starting one hour prior to disease induction resulted in 80% incidence of disease which was associated with a significant 67% reduction in disease severity at the termination of the study. This treatment regimen had no effect on diseased mouse weight.

Sequences

| | | | |
|---|---|---|---|
| SEQ ID NO: 1 | LQNRRGLGLSILLNEEC | | |
| SEQ ID NO: 2 | XQNRRGLGLSILLNEEC | | |
| SEQ ID NO: 3 | LXNRRGLGLSILLNEEC | | |
| SEQ ID NO: 4 | LQXRRGLGLSILLNEEC | | |
| SEQ ID NO: 5 | LQNXRGLGLSILLNEEC | | |
| SEQ ID NO: 6 | LQNRXGLGLSILLNEEC | | |
| SEQ ID NO: 7 | LQNRRXLGLSILLNEEC | | |
| SEQ ID NO: 8 | LQNRRGXGLSILLNEEC | | |
| SEQ ID NO: 9 | LQNRRGLXLSILLNEEC | | |
| SEQ ID NO: 10 | LQNRRGLGXSILLNEEC | | |
| SEQ ID NO: 11 | LQNRRGLGLXILLNEEC | | |
| SEQ ID NO: 12 | LQNRRGLGLSXLLNEEC | | |
| SEQ ID NO: 13 | LQNRRGLGLSIXLNEEC | | |
| SEQ ID NO: 14 | LQNRRGLGLSILXNEEC | | |
| SEQ ID NO: 15 | LQNRRGLGLSILLXEEC | | |
| SEQ ID NO: 16 | LQNRRGLGLSILLNXEC | | |
| SEQ ID NO: 17 | LQNRRGLGLSILLNEXC | | |
| SEQ ID NO: 18 | LQNRRGLGLSILLNEEX | | |
| SEQ ID NO: 19 | LQNRRGLGLSILLNEECGPGPGP | SG#17 | C-terminal amid Dimerized |
| SEQ ID NO: 20 | LQNRRGLGLSILLNEECGGGPGPGP | | |
| SEQ ID NO: 21 | LQNRRGLGLSILLNEECHHHHHH | | |
| SEQ ID NO: 22 | LQNRRGLGLSILLNEECGGHHHHHH | | |
| SEQ ID NO: 23 | LQNRRGLGLSILLNEECGGEKEKEK | | |
| SEQ ID NO: 24 | LQNRRGLGLS1FLNEEC | | |
| SEQ ID NO: 25 | GLSILLNEEC | | |
| SEQ ID NO: 26 | LSILLNEE | | |
| SEQ ID NO: 27 | LQNRRGLGLSILLNEECEEGPGPGP | SG#2 | Dimerized |
| SEQ ID NO: 28 | LQNRRGLDLSILLNEECGPGPGP | SG#3 | Dimerized |
| SEQ ID NO: 29 | GLSILLNEECGPGPGP | SG#5 | Dimerized |
| SEQ ID NO: 30 | LQNRRGLLQNRRGLGLSILLNEE | SG#15 | Dimerized |
| SEQ ID NO: 31 | LQNRRGLGLSILLNEECKKGPGPGP | SG#1 | Dimerized |
| SEQ ID NO: 32 | LQNRRGLGLSILLNC | SG#4 | Dimerized |
| SEQ ID NO: 33 | LNRKAIGLSILLNEECGPGPGP | SG#6 | Dimerized |
| SEQ ID NO: 34 | LQARILAGLSILLNEECGPGPGP | SG#7 | Dimerized |
| SEQ ID NO: 35 | LQNKRGLGLSILLNEECGPGPGP | SG#8 | Dimerized |
| SEQ ID NO: 36 | LQNKKGLGLSILLNEECGPGPGP | SG#9 | Dimerized |
| SEQ ID NO: 37 | LQNRRGLGLSILLNEECGPKK | SG#11 | Dimerized |
| SEQ ID NO: 38 | LQNRRGLGLSILLNEELQNRRGLC | SG#12 | Dimerized |
| SEQ ID NO: 39 | LQNRRGLGLSILLNEE | SG#13 | Monomeric |
| SEQ ID NO: 40 | LQNRRGLGLSILLNEELQNRRGL | SG#14 | Monomeric |
| SEQ ID NO: 41 | LQNRRGLLQNRRGLGLSILLNEEC | | Dimeric |

SG#16 has the structure of a branched peptide with two LQNRRGL peptides coupled C-terminally to α- and ε-amino groups of the Lysine residue in the peptide KGLSILLNEE. One way of depicting such a structure is as follows: (LQNRRGL)₂(>K)GLSILLNEE

SG#10 has the sequence LQNRRGLGLSILLNEECGPGPGP which is identical to SG#17 but has an extra NH2 group coupled to its C-terminal.

The One-letter and Three-letter symbols for amino acids are provided in Table 1 below.

TABLE 1

| One- | Three- | |
|---|---|---|
| letter | letter | Amino acid |
| symbol | symbol | (trivial name) |
| A | Ala | Alanine |
| B | Asx | aspartic acid or asparagine |
| C | Cys | Cysteine |
| D | Asp | aspartic acid |
| E | Glu | glutamic acid |
| F | Phe | Phenylalanine |
| G | Gly | Glycine |
| H | His | Histidine |
| I | Ile | isoleucine |
| K | Lys | lysine |
| L | Leu | leucine |
| M | Met | methionine |
| N | Asn | asparagine |
| P | Pro | proline |
| Q | Gln | glutamine |
| R | Arg | arginine |
| S | Ser | serine |
| T | Thr | threonine |
| U* | Sec | selenocysteine |
| V | Val | valine |
| W | Trp | tryptophan |
| X | Xaa | unknown or other amino acid, i.e. X can be |
| | | any of the conventional amino acids. |
| Y | Tyr | Tyrosine |
| Z | Glx | glutamic acid or glutamine (or substances such as |
| | | 4-carboxyglutamic acid and 5-oxoproline that |
| | | yield glutamic acid on acid hydrolysis of peptides) |

### Items

1. A polypeptide consisting of or comprising a sequence having at least 62%, more preferred at least 75%, preferably at least 87%, more preferred 100% sequence identity to the sequence LSILLNEE (SEQ ID NO: 26).
2. The polypeptide according to item 1, said polypeptide comprising the sequence LSILLNEE (SEQ ID NO: 26) attached to a sequence or a fragment thereof chosen among Seq ID 1 to Seq ID 1043.
3. The polypeptide of any of the preceding items, wherein said polypeptide comprises or consists of a peptide sequence selected among GLSILLNEEC (SEQ ID NO: 25), LQNRRGLGLSILLNEECEEGPGPGP (SEQ ID NO: 27), LQNRRGLDLSILLNEECGPGPGP (SEQ ID NO: 28), GLSILLNEECGPGPGP (SEQ ID NO: 29) and LQNRRGLLQNRRGLGLSILLNEE (SEQ ID NO: 30).
4. The polypeptide according to any of the preceding items, said polypeptide consisting of or comprising a sequence having at least 70% sequence identity to the sequence: LQNRRGLGLSILLNEEC (SEQ ID NO: 1).
5. The polypeptide according to any of the preceding items, said polypeptide consisting of or comprising a sequence having at least 76%, more preferred at least 82%, preferably at least 88%, more preferred at least 94%, preferably 100% sequence identity to SEQ ID NO: 1.
6. The polypeptide according to any of the preceding items, said polypeptide consisting of or comprising a sequence having at least 76%, more preferred at least 82%, preferably at least 88%, more preferred at least 94%, preferably 100% sequence identity to a sequence selected among the sequences SEQ ID NO: 1-25.
7. The polypeptide of any of the preceding items, said polypeptide comprising less than 250 aminoacids, preferably less than 200 amino acids, more preferred less than 175 amino acids, preferably less than 150 amino acids, more preferred less than 125 amino acids, preferably less than 100 amino acids, more preferred less than 75 amino acids, preferably less than 60 amino acids, more preferred less than 50 amino acids, preferably less than 40 amino acids, more preferred less than 35 amino acids, preferably less than 30 amino acids, more preferred less than 25 amino acids, preferably less than 20 amino acids, more preferred less than 19 amino acids, preferably less than 18 amino acids, more preferred less than 17 amino acids, preferably less than 16 amino acids, more preferred less than 15 amino acids, preferably less than 14 amino acids, more preferred less than 13 amino acids, preferably less than 12 amino acids, more preferred less than 11 amino acids, preferably less than 10 amino acids, more preferred less than 9 amino acids, preferably less than 8 amino acids, more preferred less than 7 amino acids, preferably less than 6 amino acids.
8. The polypeptide of any of the preceding items, said polypeptide comprising at least 5, more preferred at least 6, preferably at least 7, more preferred at least 8, preferably at least 9, more preferred at least 10, preferably at least 11, more preferred at least 12, preferably at least 13, more preferred at least 14, preferably at least 15, more preferred at least 16, preferably at least 17, more preferred at least 18, preferably at least 19, more preferred at least 20, preferably at least 25, more preferred at least 30, preferably at least 35, more preferred at least 40, preferably at least 50, more preferred at least 60, preferably at least 75, more preferred at least 100, preferably at least 125, more preferred at least 150, preferably at least 175, more preferred at least 200, preferably at least 250 amino acids.
9. A polypeptide with a length of 17 amino acids, wherein the sequence of the first 7 amino acids is identical to the sequence of the first 7 amino acids of a sequence selected among the sequences of SEQ ID NO: 26-1043, and wherein the last 10 amino acids are GLSILLNEEC (SEQ ID NO: 25).
10. The polypeptide according to item 8, comprising 1, 2, 3 or 4 point mutations.
11. The polypeptide according to any of the preceding items, wherein said polypeptide is or acts as an immune suppressive domain.
12. The polypeptide according to item 11, wherein the domain is obtainable from a polypeptide according to any of the items 1-9, by at least one point mutation, deletion or insertion.
13. The polypeptide according to item 11, wherein the total number of point mutations, deletions or insertions is selected among 1, 2, 3 and 4.
14. The polypeptide according to item 11, wherein the total number of point mutations, deletions or insertions is more than 4.
15. The polypeptide according to any of the items 11-14, which is a monomeric peptide.
16. The polypeptide according to any of the items 11-15, cross-linked to at least one additional immunosuppressive peptide and/or connected to a protein, said protein being connected to at least one additional immune suppressive domain according to any of the preceding items.
17. The polypeptide according to any of the items 11-16, connected to at least one additional immunosuppressive peptide to form a dimer.
18. The polypeptide according to item 17, wherein said dimer is homologous and comprises at least two immunosuppressive peptides according to any of the items 11-16, which are cross-linked by a disulfide bond, N-terminal to N-terminal or C-terminal to C-terminal, and/or a tandem repeat.
19. The polypeptide according to item 17 or 18, connected to at least one additional immunosuppressive peptide to form a heterologous dimer or a homologous dimer.
20. The polypeptide according to any of the items 11-19, connected to at least two additional immunosuppressive peptides to form a multimer or polymer.
21. The polypeptide according to any of the items 11-20, wherein said polypeptide comprises one or more modifications.
22. The polypeptide according to item 21, wherein said modifications are selected from the group consisting of chemical derivatizations, L-amino acid substitutions, D-amino acid substitutions, synthetic amino acid substitutions, deaminations and decarboxylations.
23. The polypeptide according to item 21 or 22, wherein the peptides or proteins have increased resistance against proteolysis compared to peptides or proteins not comprising said at least one modification.
24. A protein comprising a polypeptide according to any of the preceding items.
25. The protein according to item 24, which is an envelope protein.
26. A protein comprising a polypeptide according to any of the items 1-14, wherein said protein is not a functional membrane glycoprotein.
27. A protein comprising a polypeptide according to any of the items 1-14, wherein said protein is not fusion active.
28. A protein comprising a polypeptide according to any of the items 1-14, wherein said protein is not bound or linked to a membrane.
29. The polypeptide or protein according to any of the preceding items, wherein said polypeptide or protein inhibits IL-6 expression in a mammalian cell system or an animal model.
30. An isolated nucleic acid coding for a polypeptide or protein according to any of the preceding items.
31. An expression vector, said vector comprising a nucleic acid according to item 30 as well as the elements necessary for the expression of said nucleic acid.
32. An expression vector according to item 31, wherein said vector is an eukaryotic or prokaryotic or viral expression vector.
33. An expression vector including a nucleic acid sequence encoding for a peptide having at least 62% sequence identity or homology to the sequence LSILLNEE (SEQ ID NO: 26).
34. An expression vector including a nucleic acid sequence encoding for a polypeptide or protein according to any of the items 1-29.
35. A recombinant cell, said cell comprising a nucleic acid according to item 18, and/or an expression vector according to any of the items 31-34.
36. A pharmaceutical composition comprising at least one polypeptide, protein, nucleic acid, expression vector, or recombinant cell according to any of the preceding items, and further at least one diluent, carrier, binder, solvent or excipient.
37. The pharmaceutical composition according to item 36, wherein said at least one polypeptide, protein, nucleic acid, expression vector, or recombinant cell is the active ingredient or sole active ingredient of said pharmaceutical product.
38. A method for the preparation of a pharmaceutical composition comprising the steps of:
   a. Providing one or more polypeptide, protein, nucleic acid, expression vector, or recombinant cell according to any of the preceding items, and optionally cross-linking said one or more polypeptides;
   b. Optionally providing a diluent, carrier, binder, solvent or excipient;
   c. Providing a substance;
   d. Mixing the provided one or more peptides with any carrier of optional step b. and the substance of step d. to obtain the pharmaceutical composition.
39. The method of item 38, wherein said substance of step c. is selected from the group consisting of creams, lotions, ointments, gels, balms, salves, oils, foams, and shampoos.
40. A pharmaceutical composition obtainable according to item 38 or 39.
41. A pharmaceutical composition according to any of the items 36, 37 or 40, wherein said pharmaceutical composition is selected among the group consisting of creams, lotions, shake lotions, ointments, gels, balms, salves, oils, foams, shampoos, sprays, aerosols, transdermal patches and bandages.
42. A biomaterial comprising a polypeptide, protein, nucleic acid, expression vector, recombinant cell, or pharmaceutical composition according to any of the preceding items.
43. The biomaterial according to item 42, wherein said biomaterial is selected among a surface, particle, mesh, device, tube, or an implant.
44. A medical use of at least one polypeptide, protein, nucleic acid, expression vector, recombinant cell, pharmaceutical composition, or biomaterial according to any of the preceding items.
45. A use of at least one polypeptide, protein, nucleic acid, expression vector, recombinant cell, pharmaceutical composition, or implant according to any of the preceding items for immune suppression or immune modulation.
46. The polypeptide, protein, nucleic acid, expression vector, recombinant cell, pharmaceutical composition, or implant according to any of the preceding items for use in surgery, prophylaxis, therapy, a diagnostic method, treatment and/or amelioration of disease.
47. The polypeptide, protein, nucleic acid, expression vector, recombinant cell, pharmaceutical composition, or implant according to any of the preceding items for the treatment, amelioration or prophylaxis of an autoimmune disease.
48. The polypeptide, protein, nucleic acid, expression vector, recombinant cell, pharmaceutical composition, or implant according to item 47, wherein the autoimmune disease is SLE (systemic lupus erythematosus) or arthritis, such as rheumatoid arthritis.
49. The polypeptide, protein, nucleic acid, expression vector, recombinant cell, pharmaceutical composition, or implant according to any of the preceding items for the treatment, amelioration or prophylaxis of an inflammatory condition or a disorder associated with inflammation, such as acute or chronic inflammation.
50. The polypeptide, protein, nucleic acid, expression vector, recombinant cell, pharmaceutical composition, or implant according to any of the preceding items for use as a medicament
51. The polypeptide, protein, nucleic acid, expression vector, recombinant cell, pharmaceutical composition, or implant for a use according to any of the preceding items, wherein the subject is a human or an animal.
52. The polypeptide, protein, nucleic acid, expression vector, recombinant cell, pharmaceutical composition, or implant according to any of the preceding items, for use on an organ.
53. The polypeptide, protein, nucleic acid, expression vector, recombinant cell, pharmaceutical composition, or implant according to any of the preceding items, in the preparation or treatment of transplantation patients.
54. The polypeptide, protein, nucleic acid, expression vector, recombinant cell, pharmaceutical composition, or implant for a use according to any of the preceding items, comprising prophylaxis or treatment of a condition selected among Acute disseminated encephalomyelitis (ADEM), Addison's disease, Agammaglobulinemia, Alopecia areata, Amyotrophic Lateral Sclerosis, Ankylosing Spondylitis, Antiphospholipid syndrome, Antisynthetase syndrome, Atopic allergy, Atopic dermatitis, Autoimmune aplastic anemia, Autoimmune cardiomyopathy, Autoimmune enteropathy, Autoimmune hemolytic anemia, Autoimmune hepatitis, Autoimmune inner ear disease, Autoimmune lymphoproliferative syndrome, Autoimmune peripheral neuropathy, Autoimmune pancreatitis, Autoimmune polyendocrine syndrome, Autoimmune progesterone dermatitis, Autoimmune thrombocytopenic purpura, Autoimmune urticaria, Autoimmune uveitis, Balo disease/Balo concentric sclerosis, Behget's disease, Berger's disease, Bickerstaff's encephalitis, Blau syndrome, Bullous pemphigoid, Cancer, Castleman's disease, Celiac disease, Chagas disease, Chronic inflammatory demyelinating polyneuropathy, Chronic recurrent multifocal osteomyelitis, Chronic obstructive pulmonary disease, Churg-Strauss syndrome, Cicatricial pemphigoid, Cogan syndrome, Cold agglutinin disease, Complement component 2 deficiency, Contact dermatitis, Cranial arteritis, CREST syndrome, Crohn's disease, Cushing's Syndrome, Cutaneous leukocytoclastic angiitis, Dego's disease, Dercum's disease, Dermatitis herpetiformis, Dermatomyositis, Diabetes mellitus type 1, Diffuse cutaneous systemic sclerosis, Dressler's syndrome, Drug-induced lupus, Discoid lupus erythematosus, Eczema, Endometriosis, Enthesitis-related arthritis, Eosinophilic fasciitis, Eosinophilic gastroenteritis, Epidermolysis bullosa acquisita, Erythema nodosum, Erythroblastosis fetalis, Essential mixed cryoglobulinemia, Evan's syndrome, Fibrodysplasia ossificans progressiva, Fibrosing alveolitis, Gastritis, Gastrointestinal pemphigoid, Glomerulonephritis, Goodpasture's syndrome, Graves' disease, Guillain-Barré syndrome (GBS), Hashimoto's encephalopathy, Hashimoto's thyroiditis, Henoch-Schonlein purpura, Herpes gestationis, hepatitis, Hidradenitis suppurativa, Hughes-Stovin syndrome, Hypogammaglobulinemia, Idiopathic inflammatory demyelinating diseases, Idiopathic pulmonary fibrosis, Idiopathic thrombocytopenic purpura, IgA nephropathy, Inclusion body myositis, Chronic inflammatory demyelinating polyneuropathy, Interstitial cystitis, Juvenile idiopathic arthritis, Kawasaki's disease, Lambert-Eaton myasthenic syndrome, Leukocytoclastic vasculitis, Lichen planus, Lichen sclerosus, Linear IgA disease (LAD), Lou Gehrig's disease, Lupoid hepatitis, Lupus erythematosus, Majeed syndrome, Ménière's disease, Microscopic polyangiitis, Miller-Fisher syndrome, Mixed connective tissue disease, Morphea, Mucha-Habermann disease, Multiple sclerosis, Myasthenia gravis, Myositis, Narcolepsy, Neuromyelitis optica, Neuromyotonia, non-alcoholic steatohepatitis (NASH), Occular cicatricial pemphigoid, Opsoclonus myoclonus syndrome, Ord's thyroiditis, Palindromic rheumatism, PANDAS (pediatric autoimmune neuropsychiatric disorders associated with streptococcus), Paraneoplastic cerebellar degeneration, Paroxysmal nocturnal hemoglobinuria (PNH), Parry Romberg syndrome, Parsonage-Turner syndrome, Pars planitis, Pemphigus vulgaris, Pernicious anaemia, Perivenous encephalomyelitis, POEMS syndrome, Polyarteritis nodosa, Polymyalgia rheumatica, Polymyositis, Primary biliary cirrhosis, Primary sclerosing cholangitis, Progressive inflammatory neuropathy, Psoriasis, Psoriatic arthritis, Pyoderma gangrenosum, Pure red cell aplasia, Rasmussen's encephalitis, Raynaud phenomenon, Relapsing polychondritis, Reiter's syndrome, Restless leg syndrome, Retroperitoneal fibrosis, Rheumatoid arthritis, Rheumatic fever, Sarcoidosis, Schizophrenia, Schmidt syndrome, Schnitzler syndrome, Scleritis, Scleroderma, Serum Sickness, Sjögren's syndrome, Spondyloarthropathy, Still's disease, Stiff person syndrome, Subacute bacterial endocarditis (SBE), Susac's syndrome, Sweet's syndrome, Sydenham chorea, Sympathetic ophthalmia, Systemic lupus erythematosis, Takayasu's arteritis, Temporal arteritis, Thrombocytopenia, Tolosa-Hunt syndrome, Transverse myelitis, Ulcerative colitis, Undifferentiated connective tissue disease, Undifferentiated spondyloarthropathy, Urticarial vasculitis, Vasculitis, Vitiligo, and Wegener's granulomatosis.
55. The polypeptide, protein, nucleic acid, expression vector, recombinant cell, pharmaceutical composition, or implant for a use according to any of the preceding items, for the treatment or prevention of a disorder selected among Acne vulgaris, Allergy, Allergic rhinitis, Asthma, Atherosclerosis, Autoimmune disease, Celiac disease, Chronic prostatitis, Glomerulonephritis, Hypersensitivities, Inflammatory bowel diseases, Pelvic inflammatory disease, Reperfusion injury, Rheumatoid arthritis, Sarcoidosis, Transplant rejection, Vasculitis, interstitial cystitis, Cancer, Depression, Myopathies, and Leukocyte defects.
56. The polypeptide, protein, nucleic acid, expression vector, recombinant cell, pharmaceutical composition, or implant for a use according to any of the preceding items, comprising prophylaxis or treatment of sepsis.
57. The polypeptide, protein, nucleic acid, expression vector, recombinant cell, pharmaceutical composition, or implant for a use according to any of the preceding items, comprising prophylaxis or treatment of spondyloarthritis.
58. The polypeptide, protein, nucleic acid, expression vector, recombinant cell, pharmaceutical composition, or implant for a use according to any of the preceding items, comprising prophylaxis or treatment of asthma and/or allergy.
59. The polypeptide according to any of the items 11-23 for use in a method of prophylaxis or treatment or amelioration of a condition associated with an autoimmune disease, wherein a gene sequence expressing said immune suppressive domain exhibits increased or decreased expression in a group of patients suffering from said autoimmune disease as compared to a healthy control group.
60. The polypeptide according to item 59, wherein said immune suppressive domain is from an endogenous retrovirus, preferably a human endogenous retrovirus.
61. The polypeptide according to item 59 or 60, wherein said immune suppressive domain is selected among the sequences of SEQ ID NO: 1-1043.
62. Use of a polypeptide selected among the sequences of SEQ ID NO: 1-1043 for the prophylaxis or treatment or amelioration of an autoimmune disease or at least one symptom associated with said autoimmune disease.
63. A use of a polypeptide, protein, nucleic acid, expression vector, recombinant cell, pharmaceutical composition, or implant according to any of the preceding items for the manufacture of an anti-inflammatory medicament or a medicament for immune suppression or immune modulation.
64. The use of a polypeptide, protein, nucleic acid, expression vector, recombinant cell, pharmaceutical composition, or implant according to any of the preceding items for the manufacture of a medicament for the preparation or treatment of transplantation patients.
65. The use of a polypeptide, protein, nucleic acid, expression vector, recombinant cell, pharmaceutical composition, or implant according to any of the preceding items for the manufacture of a medicament for prophylaxis or treatment of an autoimmune or inflammatory disease.
66. The use of a polypeptide, protein, nucleic acid, expression vector, recombinant cell, pharmaceutical composition, or implant according to any of the preceding items for the manufacture of a medicament for prophylaxis or treatment of a condition selected among Acute disseminated encephalomyelitis (ADEM), Addison's disease, Agammaglobulinemia, Alopecia areata, Amyotrophic Lateral Sclerosis, ANCA Vasculitis, Ankylosing Spondylitis, Antiphospholipid syndrome, Antisynthetase syndrome, Arteriosclerosis, Atopic allergy, Atopic dermatitis, Autoimmune aplastic anemia, Autoimmune cardiomyopathy, Autoimmune enteropathy, Autoimmune hemolytic anemia, Autoimmune hepatitis, Autoimmune inner ear disease, Autoimmune lymphoproliferative syndrome, Autoimmune peripheral neuropathy, Autoimmune pancreatitis, Autoimmune polyendocrine syndrome, Autoimmune progesterone dermatitis, Autoimmune thrombocytopenic purpura, Autoimmune urticaria, Autoimmune uveitis, Balo disease/Balo concentric sclerosis, Behget's disease, Berger's disease, Bickerstaff's encephalitis, Blau syndrome, Bullous pemphigoid, Cancer, Castleman's disease, Celiac disease, Chagas disease, Chronic inflammatory demyelinating polyneuropathy, Chronic recurrent multifocal osteomyelitis, Chronic obstructive pulmonary disease, Churg-Strauss syndrome, Cicatricial pemphigoid, Cogan syndrome, Cold agglutinin disease, Complement component 2 deficiency, Contact dermatitis, Cranial arteritis, CREST syndrome, Crohn's disease, Cushing's Syndrome, Cutaneous leukocytoclastic angiitis, Dego's disease, Dercum's disease, Dermatitis herpetiformis, Dermatomyositis, Diabetes mellitus type 1, Diffuse cutaneous systemic sclerosis, Dressler's syndrome, Drug-induced lupus, Discoid lupus erythematosus, Eczema, Endometriosis, Enthesitis-related arthritis, Eosinophilic fasciitis, Eosinophilic gastroenteritis, Epidermolysis bullosa acquisita, Erythema nodosum, Erythroblastosis fetalis, Essential mixed cryoglobulinemia, Evan's syndrome, Fibrodysplasia ossificans progressiva, Fibrosing alveolitis, Gastritis, Gastrointestinal pemphigoid, Glomerulonephritis, Goodpasture's syndrome, Graves' disease, Guillain-Barré syndrome (GBS), Hashimoto's encephalopathy, Hashimoto's thyroiditis, Henoch-Schonlein purpura, Herpes gestationis, hepatitis, Hidradenitis suppurativa, Hughes-Stovin syndrome, Hypogammaglobulinemia, Idiopathic inflammatory demyelinating diseases, Idiopathic pulmonary fibrosis, Idiopathic thrombocytopenic purpura, IgA nephropathy, Inclusion body myositis, Chronic inflammatory demyelinating polyneuropathy, Interstitial cystitis, Juvenile idiopathic arthritis, Kawasaki's disease, Lambert-Eaton myasthenic syndrome, Leukocytoclastic vasculitis, Lichen planus, Lichen sclerosus, Linear IgA disease (LAD), Lou Gehrig's disease, Lupoid hepatitis, Lupus erythematosus, Majeed syndrome, Ménière's disease, Microscopic polyangiitis, Miller-Fisher syndrome, Mixed connective tissue disease, Morphea, Mucha-Habermann disease, Multiple sclerosis, Myasthenia gravis, Myositis, Narcolepsy, Neuromyelitis optica, Neuromyotonia, non-alcoholic steatohepatitis (NASH), Occular cicatricial pemphigoid, Opsoclonus myoclonus syndrome, Ord's thyroiditis, Palindromic rheumatism, PANDAS (pediatric autoimmune neuropsychiatric disorders associated with streptococcus), Paraneoplastic cerebellar degeneration, Paroxysmal nocturnal hemoglobinuria (PNH), Parry Romberg syndrome, Parsonage-Turner syndrome, Pars planitis, Pemphigus vulgaris, Pernicious anaemia, Perivenous encephalomyelitis, POEMS syndrome, Polyarteritis nodosa, Polymyalgia rheumatica, Polymyositis, Primary biliary cirrhosis, Primary sclerosing cholangitis, Progressive inflammatory neuropathy, Psoriasis, Psoriatic arthritis, Pyoderma gangrenosum, Pure red cell aplasia, Rasmussen's encephalitis, Raynaud phenomenon, Relapsing polychondritis, Reiter's syndrome, Restless leg syndrome, Retroperitoneal fibrosis, Rheumatoid arthritis, Rheumatic fever, Sarcoidosis, Schizophrenia, Schmidt syndrome, Schnitzler syndrome, Scleritis, Scleroderma, Serum Sickness, Sjögren's syndrome, Spondyloarthropathy, Still's disease, Stiff person syndrome, Subacute bacterial endocarditis (SBE), Susac's syndrome, Sweet's syndrome, Sydenham chorea, Sympathetic ophthalmia, Systemic lupus erythematosis, Takayasu's arteritis, Temporal arteritis, Thrombocytopenia, Tolosa-Hunt syndrome, Transverse myelitis, Ulcerative colitis, Undifferentiated connective tissue disease, Undifferentiated spondyloarthropathy, Urticarial vasculitis, Vasculitis, Vitiligo, and Wegener's granulomatosis.
67. The use of a polypeptide, protein, nucleic acid, expression vector, recombinant cell, pharmaceutical composition, or implant according to any of the preceding items for the manufacture of a medicament for prophylaxis or treatment of inflammation or a condition associated with inflammation, such as acute or chronic inflammation.
68. The use of a polypeptide, protein, nucleic acid, expression vector, recombinant cell, pharmaceutical composition, or implant according to item 67, for the manufacture of a medicament for prophylaxis or treatment of a condition selected among Acne vulgaris, Allergy, Allergic rhinitis, Asthma, Atherosclerosis, Autoimmune disease, Celiac disease, Chronic prostatitis, Glomerulonephritis, Hypersensitivities, Inflammatory bowel diseases, Pelvic inflammatory disease, Reperfusion injury, Rheumatoid arthritis, Sarcoidosis, Transplant rejection, Vasculitis, interstitial cystitis, Cancer, Depression, Myopathies, and Leukocyte defects.
69. The use of a polypeptide, protein, nucleic acid, expression vector, recombinant cell, pharmaceutical composition, or implant according to any of the preceding items for the manufacture of a medicament for prophylaxis or treatment of at least condition selection among sepsis, rheumatoid arthritis, systemic lupus erythematosus (SLE), and spondyloarthritis.
70. The use of a polypeptide, protein, nucleic acid, expression vector, recombinant cell, pharmaceutical composition, or implant according to any of the preceding items for the manufacture of a medicament for prophylaxis or treatment of asthma and/or allergy.
71. The use of a polypeptide, protein, nucleic acid, expression vector, recombinant cell, or pharmaceutical composition according to any of the preceding items, for coating of nanoparticles and/or biomaterials.
72. The use of a polypeptide, protein, nucleic acid, expression vector, recombinant cell, or pharmaceutical composition according to any of the preceding items, for at least partial suppression of an immune response to at least one nanoparticle or biomaterial.
73. The use of a polypeptide, protein, nucleic acid, expression vector, recombinant cell, or pharmaceutical composition according to any of the preceding items, to increase the in vivo half-life of nanoparticles and/or biomaterials and/or medical devices and/or implants in the patient.
74. The use of an endogenous retrovirus for diagnosis of a disease.
75. The use of an endogenous retrovirus whose expression level or copy number is different in a subject with a condition as compared to a subject without said condition for diagnosis of a disease.
76. The use of an endogenous retrovirus whose expression level or copy number is different in a subject with an autoimmune condition as compared to a subject without the said condition for diagnosis of a disease.
77. The use of single nucleotide polymorphisms associated with HERV-H 59 for diagnosis of a disease.
78. The use of HERV-H 59 for diagnosis of SLE.
79. A method of prophylactically or therapeutically treating an autoimmune disease and/or an inflammatory condition by administering to a subject in need thereof a prophylactically or therapeutically effective amount of at least one polypeptide, protein, nucleic acid, expression vector, recombinant cell, or pharmaceutical composition according to any of the preceding items through one or more or several administrations.
80. Method of prophylaxis or treatment or amelioration of a condition associated with an autoimmune disease, comprising:
   a. Measuring the expression or copy number of at least one endogenous retrovirus in a group of patients suffering from said autoimmune disease;
   b. Comparing said expression with the expression of said at least one endogenous retrovirus in a healthy control group;
   c. Identifying at least one endogenous retrovirus having different expression in said group of patients;
   d. Optionally identifying at least one immune suppressive domain in said at least one endogenous retrovirus;
   e. Treating at least one patient suffering from said condition by administration of at least one immune suppressive domain preferably contained in a protein containing said at least one immune suppressive domain and/or a protein expressed by said endogenous retrovirus.
81. Method of prophylaxis or treatment or amelioration of a condition associated with an autoimmune disease, comprising:
   f. Measuring the concentration of at least one protein or polypeptide comprising at least one immune suppressive domain in a group of patients suffering from said autoimmune disease;
   g. Comparing said concentration with the concentration in a healthy control group;
   h. Identifying at least one immune suppressive domain having different expression in said group of patients;
   i. Treating at least one patient suffering from said condition by administration of said at least one immune suppressive domain and/or a protein comprising said at least one immune suppressive domain.
82. Method according to item 80 or 81, wherein said different expression is selected among increased and decreased expression.
83. Method according to any of the items 80-82, wherein said endogenous retrovirus is a human endogenous retrovirus.
84. Method according to item 83, wherein said human endogenous retrovirus belongs to the HERV-H subfamily or the HERV-K subfamily.
85. Method according to any of the items 80-84, wherein said endogenous retrovirus contains at least one open reading frame capable of encoding a protein.
86. Method according to item 85, wherein said at least one open reading frame has a length of at least 50, preferably at least 100, more preferred at least 150, preferably at least 200, more preferred at least 250, preferably at least 300, more preferred at least 350, preferably at least 400 nucleotides.
87. A use of a polypeptide, protein, nucleic acid, expression vector, recombinant cell, or pharmaceutical composition according to any of the preceding items, for prophylaxis or treatment of a condition or disease by an administration route selected among injection, inhalation, topical, transdermal, oral, nasal, vaginal, or anal delivery.
88. The use according to item 87, wherein the mode of injection is selected among intravenous (IV), intraperironeal (IP), subcutaneous (SC) and (intramuscular) IM.
89. The use according to item 88, for treatment of a disease by direct injection at a site affected by a disorder, such as inflammation.
90. The use of a polypeptide, protein, nucleic acid, expression vector, recombinant cell, or pharmaceutical composition according to any of the preceding items, for treatment of a condition selected among a skin disease, Psoriasis, Arthritis, Asthma, Sepsis, inflammatory bowel disease, rheumatoid arthritis, SLE, and spondyloarthritis.
91. The use of a polypeptide, protein, nucleic acid, expression vector, recombinant cell, or pharmaceutical composition according to any of the preceding items, for treatment of Arthritis where the composition is injected directly at site of inflammation.
92. The use of a polypeptide, protein, nucleic acid, expression vector, recombinant cell, or pharmaceutical composition according to any of the preceding items, for treatment of a condition selected among Gastrointestinal hyperresponsiveness, Food Allergy, Food intolerance and inflammatory bowel disease, preferably wherein the composition is delivered orally.
93. The use of a polypeptide, protein, nucleic acid, expression vector, recombinant cell, or pharmaceutical composition according to any of the preceding items, for treatment Asthma where the composition is delivered by inhalation.

### REFERENCES

Boyd, M. T., Bax, C. M., Bax, B. E., Bloxam, D. L., & Weiss, R. A. (1993). The human endogenous retrovirus ERV-3 is upregulated in differentiating placental trophoblast cells. Virology, 196(2), 905-909. doi: 10.1006/viro.1993.1556
Brand, D. D., Latham, K. A., & Rosloniec, E. F. (2007). Collagen-induced arthritis. Nat Protoc, 2(5), 1269-1275. doi: 10.1038/nprot.2007.173
Hamishehkar, H., Beigmohammadi, M. T., Abdollahi, M., Ahmadi, A., Mahmoodpour, A., Mirjalili, M. R., . . . Mojtahedzadeh, M. (2010). Identification of enhanced cytokine generation following sepsis. Dream of magic bullet for mortality prediction and therapeutic evaluation. Daru, 18(3), 155-162.
Hillenbrand, A., Knippschild, U., Weiss, M., Schrezenmeier, H., Henne-Bruns, D., Huber-Lang, M., & Wolf, A. M. (2010). Sepsis induced changes of adipokines and cytokines—septic patients compared to morbidly obese patients. BMC Surg, 10, 26. doi: 10.1186/1471-2482-10-26 1471-2482-10-26 [pii]
Mangeney, M., & Heidmann, T. (1998). Tumor cells expressing a retroviral envelope escape immune rejection in vivo. Proc Natl Acad Sci USA, 95(25), 14920-14925.
Mihara, M., Takagi, N., Takeda, Y., & Ohsugi, Y. (1998). IL-6 receptor blockage inhibits the onset of autoimmune kidney disease in NZB/W F1 mice. Clin Exp Immunol, 112(3), 397-402.
Peterson, E., Robertson, A. D., & Emlen, W. (1996). Serum and urinary interleukin-6 in systemic lupus erythematosus. Lupus, 5(6), 571-575.
Rogler, G., & Andus, T. (1998). Cytokines in inflammatory bowel disease. World J Surg, 22(4), 382-389. Sasai, M., Saeki, Y., Ohshima, S., Nishioka, K., Mima, T., Tanaka, T., . . . Kishimoto, T. (1999). Delayed onset and reduced severity of collagen-induced arthritis in interleukin-6-deficient mice. Arthritis Rheum, 42(8), 1635-1643. doi: 10.1002/1529-0131(199908)42:8<1635::AID-ANR11>3.0.CO;2-Q
Schmitt, K., Reichrath, J., Roesch, A., Meese, E., & Mayer, J. (2013). Transcriptional profiling of human endogenous retrovirus group HERV-K (HML-2) loci in melanoma. Genome Biol Evol, 5(2), 307-328. doi: 10.1093/gbe/evt010
Srirangan, S., & Choy, E. H. (2010). The role of interleukin 6 in the pathophysiology of rheumatoid arthritis. Ther Adv Musculoskelet Dis, 2(5), 247-256. doi: 10.1177/1759720X10378372 10.1177_1759720 X10378372 [pii]
Tonjes, R. R., Lower, R., Boller, K., Denner, J., Hasenmaier, B., Kirsch, H., . . . Kurth, R. (1996). HERV-K: the biologically most active human endogenous retrovirus family. J Acquir Immune Defic Syndr Hum Retrovirol, 13 Suppl 1, S261-267.
Venables, P. J., Brookes, S. M., Griffiths, D., Weiss, R. A., & Boyd, M. T. (1995). Abundance of an endogenous retroviral envelope protein in placental trophoblasts suggests a biological function. Virology, 211(2), 589-592. doi: 10.1006/viro.1995.1442
Willer, A., Saussele, S., Gimbel, W., Seifarth, W., Kister, P., Leib-Mosch, C., & Hehlmann, R. (1997). Two groups of endogenous MMTV related retroviral env transcripts expressed in human tissues. Virus Genes, 15(2), 123-133.

1. An isolated polypeptide comprising a peptide sequence having at least 62.5%, more preferred 75%, more preferred 87.5%, more preferred at least 100% sequence identity to the sequence LSILLNEE (SEQ ID NO: 26).

2. A polypeptide having a maximum length of 130 amino acids comprising a peptide sequence having at least 62.5%, more preferred 75%, more preferred 87.5%, more preferred at least 100% sequence identity to the sequence LSILLNEE (SEQ ID NO: 26).

3. A polypeptide according to claim 1 or 2 comprising one or more peptide sequences having at least 70%, preferably at least 76%, more preferred at least 82%, preferably at least 88%, more preferred at least 94%, preferably 100% sequence identity to sequences selected among LQNRRGLGLSILLNEEC (SEQ ID NO: 1), LQNRRGLGLSILLNEECGPGPGP (SEQ ID NO: 19), GLSILLNEEC (SEQ ID NO: 25), LQNRRGLGLSILLNEECEEGPGPGP (SEQ ID NO: 27), LQNRRGLDLSILLNEECGPGPGP (SEQ ID NO: 28), GLSILLNEECGPGPGP (SEQ ID NO: 29) and LQNRRGLLQNRRGLGLSILLNEE (SEQ ID NO: 30).

4. A polypeptide according to any of the preceding claims, said polypeptide comprising a peptide sequence having at least 76%, more preferred at least 82%, preferably at least 88%, more preferred at least 94%, preferably 100% sequence identity to a sequence selected among the sequences SEQ ID NO: 1-25 and 27-41.

5. A polypeptide according to claim 1 or 2 selected among polypeptides having at least 76%, more preferred at least 82%, preferably at least 88%, more preferred at least 94%, preferably 100% sequence identity to sequences selected among LSILLNEE (SEQ ID NO: 26), LQNRRGLGLSILLNEEC (SEQ ID NO: 1), LQNRRGLGLSILLNEECGPGPGP (SEQ ID NO: 19), GLSILLNEEC (SEQ ID NO: 25), LQNRRGLGLSILLNEECEEGPGPGP (SEQ ID NO: 27), LQNRRGLDLSILLNEECGPGPGP (SEQ ID NO: 28), GLSILLNEECGPGPGP (SEQ ID NO: 29) and LQNRRGLLQNRRGLGLSILLNEE (SEQ ID NO: 30).

6. A polypeptide of claim 1 or 2 selected among LSILLNEE (SEQ ID NO: 26), LQNRRGLGLSILLNEEC (SEQ ID NO: 1), LQNRRGLGLSILLNEECGPGPGP (SEQ ID NO: 19), GLSILLNEEC (SEQ ID NO: 25), LQNRRGLGLSILLNEECEEGPGPGP (SEQ ID NO: 27), LQNRRGLDLSILLNEECGPGPGP (SEQ ID NO: 28), GLSILLNEECGPGPGP (SEQ ID NO: 29) and LQNRRGLLQNRRGLGLSILLNEE (SEQ ID NO: 30).

7. A polypeptide entity comprising a polypeptide of any of the preceding claims, said polypeptide entity comprising less than 250 aminoacids, preferably less than 200 amino acids, more preferred less than 175 amino acids, preferably less than 150 amino acids, more preferred less than 125 amino acids, preferably less than 100 amino acids, more preferred less than 75 amino acids, preferably less than 60 amino acids, more preferred less than 50 amino acids, preferably less than 40 amino acids, more preferred less than 35 amino acids, preferably less than 30 amino acids, more preferred less than 25 amino acids, preferably less than 20 amino acids, more preferred less than 19 amino acids, preferably less than 18 amino acids, more preferred less than 17 amino acids, preferably less than 16 amino acids, more preferred less than 15 amino acids, preferably less than 14 amino acids, more preferred less than 13 amino acids, preferably less than 12 amino acids, more preferred less than 11 amino acids, preferably less than 10 amino acids, more preferred less than 9 amino acids, preferably less than 8 amino acids, more preferred less than 7 amino acids, preferably less than 6 amino acids.

8. A polypeptide entity comprising a polypeptide of any of the preceding claims, said polypeptide entity comprising at least 5, more preferred at least 6, preferably at least 7, more preferred at least 8, preferably at least 9, more preferred at least 10, preferably at least 11, more preferred at least 12, preferably at least 13, more preferred at least 14, preferably at least 15, more preferred at least 16, preferably at least 17, more preferred at least 18, preferably at least 19, more preferred at least 20, preferably at least 25, more preferred at least 30, preferably at least 35, more preferred at least 40, preferably at least 50, more preferred at least 60, preferably at least 75, more preferred at least 100, preferably at least 125, more preferred at least 150, preferably at least 175, more preferred at least 200, preferably at least 250 amino acids.

9. A polypeptide with a length of 17 amino acids, wherein the sequence of the first 7 amino acids is identical to the sequence of the first 7 amino acids of a sequence selected among the sequences of SEQ ID NO: 42-1043, and wherein the last 10 amino acids are GLSILLNEEC (SEQ ID NO: 25).

10. The polypeptide according to claim 1 or 2, said polypeptide comprising a sequence having at least 62.5%, more preferred 75%, more preferred 87.5%, more preferred at least 100% sequence identity to the sequence LSILLNEE (SEQ ID NO: 26) attached to a sequence or a fragment thereof chosen among Seq ID 1 to Seq ID 1043.

11. The polypeptide according to claim 9 or 10, comprising 1, 2, 3 or 4 point mutations.

12. The polypeptide according to any of the preceding claims, which is glycosylated.

13. The polypeptide according to any of the preceding claims, which is acylated.

14. The polypeptide according to any of the preceding claims, which is a monomer.

15. The polypeptide according to any of the preceding claims, which is dimerized, trimerized or multimerized.

16. A protein comprising a polypeptide according to any of the preceding claims, wherein said protein comprises less than 250 aminoacids, preferably less than 200 amino acids, more preferred less than 175 amino acids, preferably less than 150 amino acids, more preferred less than 125 amino acids, preferably less than 100 amino acids, more preferred less than 75 amino acids, preferably less than 60 amino acids, more preferred less than 50 amino acids, preferably less than 40 amino acids, more preferred less than 35 amino acids, preferably less than 30 amino acids, more preferred less than 25 amino acids, preferably less than 20 amino acids, more preferred less than 19 amino acids, preferably less than 18 amino acids, more preferred less than 17 amino acids, preferably less than 16 amino acids, more preferred less than 15 amino acids, preferably less than 14 amino acids, more preferred less than 13 amino acids, preferably less than 12 amino acids, more preferred less than 11 amino acids, preferably less than 10 amino acids, more preferred less than 9 amino acids, preferably less than 8 amino acids, more preferred less than 7 amino acids, preferably less than 6 amino acids.

17. A protein or polypeptide of any of the preceding claims or a protein comprising a polypeptide according to any of the preceding claims, said protein or polypeptide comprising at least 5, more preferred at least 6, preferably at least 7, more preferred at least 8, preferably at least 9, more preferred at least 10, preferably at least 11, more preferred at least 12, preferably at least 13, more preferred at least 14, preferably at least 15, more preferred at least 16, preferably at least 17, more preferred at least 18, preferably at least 19, more preferred at least 20, preferably at least 25, more preferred at least 30, preferably at least 35, more preferred at least 40, preferably at least 50, more preferred at least 60, preferably at least 75, more preferred at least 100, preferably at least 125, more preferred at least 150, preferably at least 175, more preferred at least 200, preferably at least 250 amino acids.

18. A protein comprising a polypeptide according to any of the claims 1-15, wherein said protein is not fusion active.

19. The polypeptide or protein according to any of the preceding claims, wherein said polypeptide or protein inhibits IL-6 expression in a mammalian cell system or an animal model.

20. An isolated nucleic acid coding for a polypeptide or protein according to any of the preceding claims.

21. An expression vector, said vector comprising a nucleic acid according to claim 20 as well as the elements necessary for the expression of said nucleic acid.

22. An expression vector according to claim 21, wherein said vector is an eukaryotic or prokaryotic or viral expression vector.

23. An expression vector according to claim 21 or 22, wherein said vector is selected among the group consisting of yeast, e-coli and baculoviruses.

24. A pharmaceutical composition comprising a polypeptide comprising a peptide sequence having at least 62.5%, more preferred 75%, more preferred 87.5%, more preferred at least 100% sequence identity to the sequence LSILLNEE (SEQ ID NO: 26), and further at least one diluent, carrier, binder, solvent or excipient.

25. A pharmaceutical composition comprising a polypeptide according to claim 24 comprising one or more peptide sequences having at least 70%, preferably at least 76%, more preferred at least 82%, preferably at least 88%, more preferred at least 94%, preferably 100% sequence identity to sequences selected among LQNRRGLGLSILLNEEC (SEQ ID NO: 1), LQNRRGLGLSILLNEECGPGPGP (SEQ ID NO: 19), GLSILLNEEC (SEQ ID NO: 25), LQNRRGLGLSILLNEECEEGPGPGP (SEQ ID NO: 27), LQNRRGLDLSILLNEECGPGPGP (SEQ ID NO: 28), GLSILLNEECGPGPGP (SEQ ID NO: 29) and LQNRRGLLQNRRGLGLSILLNEE (SEQ ID NO: 30), and further at least one diluent, carrier, binder, solvent or excipient.

26. A pharmaceutical composition comprising a polypeptide according to claim 24 or 25, said polypeptide comprising a peptide sequence having at least 76%, more preferred at least 82%, preferably at least 88%, more preferred at least 94%, preferably 100% sequence identity to a sequence selected among the sequences SEQ ID NO: 1-41), and further at least one diluent, carrier, binder, solvent or excipient.

27. A pharmaceutical composition according to claim 24 comprising a polypeptide selected among polypeptides having at least 76%, more preferred at least 82%, preferably at least 88%, more preferred at least 94%, preferably 100% sequence identity to sequences selected among LSILLNEE (SEQ ID NO: 26), LQNRRGLGLSILLNEEC (SEQ ID NO: 1), LQNRRGLGLSILLNEECGPGPGP (SEQ ID NO: 19), GLSILLNEEC (SEQ ID NO: 25), LQNRRGLGLSILLNEECEEGPGPGP (SEQ ID NO: 27), LQNRRGLDLSILLNEECGPGPGP (SEQ ID NO: 28), GLSILLNEECGPGPGP (SEQ ID NO: 29) and LQNRRGLLQNRRGLGLSILLNEE (SEQ ID NO: 30)), and further at least one diluent, carrier, binder, solvent or excipient.

28. A pharmaceutical composition comprising a polypeptide of claim 24 selected among LSILLNEE (SEQ ID NO: 26), LQNRRGLGLSILLNEEC (SEQ ID NO: 1), LQNRRGLGLSILLNEECGPGPGP (SEQ ID NO: 19), GLSILLNEEC (SEQ ID NO: 25), LQNRRGLGLSILLNEECEEGPGPGP (SEQ ID NO: 27), LQNRRGLDLSILLNEECGPGPGP (SEQ ID NO: 28), GLSILLNEECGPGPGP (SEQ ID NO: 29) and LQNRRGLLQNRRGLGLSILLNEE (SEQ ID NO: 30)), and further at least one diluent, carrier, binder, solvent or excipient.

29. A medical use of at least one polypeptide, protein, nucleic acid, expression vector, recombinant cell, or pharmaceutical composition according to any of the preceding claims.

30. A use of at least one polypeptide, protein, nucleic acid, expression vector, recombinant cell, or pharmaceutical composition according to any of the preceding claims for immune suppression or immune modulation.

31. The polypeptide, protein, nucleic acid, expression vector, recombinant cell, or pharmaceutical composition according to any of the preceding claims for use in surgery, prophylaxis, therapy, a diagnostic method, treatment and/or amelioration of disease.

32. The polypeptide, protein, nucleic acid, expression vector, recombinant cell, or pharmaceutical composition according to any of the preceding claims for the treatment, amelioration or prophylaxis of an autoimmune disease.

33. The polypeptide, protein, nucleic acid, expression vector, recombinant cell, or pharmaceutical composition according to claim 32, wherein the autoimmune disease is SLE (systemic lupus erythematosus) or arthritis, such as rheumatoid arthritis, spondyloarthritis, or multiple sclerosis (MS).

34. The polypeptide, protein, nucleic acid, expression vector, recombinant cell, or pharmaceutical composition according to any of the preceding claims for the treatment, amelioration or prophylaxis of an inflammatory condition or a disorder associated with inflammation, such as acute or chronic inflammation.

35. The polypeptide, protein, nucleic acid, expression vector, recombinant cell, or pharmaceutical composition according to any of the preceding claims for use as a medicament.

36. The polypeptide, protein, nucleic acid, expression vector, recombinant cell, or pharmaceutical composition for a use according to any of the preceding claims, comprising prophylaxis or treatment of sepsis.

37. The polypeptide, protein, nucleic acid, expression vector, recombinant cell, or pharmaceutical composition for a use according to any of the preceding claims, comprising prophylaxis or treatment of spondyloarthritis.

38. The polypeptide, protein, nucleic acid, expression vector, recombinant cell, or pharmaceutical composition for a use according to any of the preceding claims, comprising prophylaxis or treatment of asthma and/or allergy.

39. The polypeptide, protein, nucleic acid, expression vector, recombinant cell, or pharmaceutical composition according to any of the preceding claims for a use in as an adjuvant, such as in a vaccine.

40. A method of prophylactically or therapeutically treating an autoimmune disease and/or an inflammatory condition by administering to a subject in need thereof a prophylactically or therapeutically effective amount of at least one polypeptide, protein, nucleic acid, expression vector, recombinant cell, or pharmaceutical composition according to any of the preceding claims through one or more or several administrations.

41. A use of a polypeptide, protein, nucleic acid, expression vector, recombinant cell, or pharmaceutical composition according to any of the preceding claims, for prophylaxis or treatment of a condition or disease by an administration route selected among injection, inhalation, topical, transdermal, oral, nasal, vaginal, or anal delivery.

42. The use according to claim 41, wherein the mode of injection is selected among intravenous (IV), intraperironeal (IP), subcutaneous (SC) and (intramuscular) IM.

43. The use according to claim 42, for treatment of a disease by direct injection at a site affected by a disorder, such as inflammation.

44. The use of a polypeptide, protein, nucleic acid, expression vector, recombinant cell, or pharmaceutical composition according to any of the preceding claims, for treatment of Arthritis where the composition is injected directly at site of inflammation.
